(19) United States
(12) Patent Application Publication
(10) Pub. No.: US 2022/0228157 A1
(43) Pub. Date: Jul. 21, 2022

(54) GENE EDITING IN DIVERSE BACTERIA

(71) Applicant: President and Fellows of Harvard College, Cambridge, MA (US)

(72) Inventors: George M. Church, Cambridge, MA (US); Timothy M. Wannier, Cambridge, MA (US); Gabriel T. Filsinger, Cambridge, MA (US)

(73) Assignee: President and Fellows of Harvard College, Cambridge, MA (US)

(21) Appl. No.: 17/613,216

(22) PCT Filed: May 21, 2020
(86) PCT No.: PCT/US2020/034025

§ 371 Date: Nov. 22, 2021

Related U.S. Application Data
(60) Provisional application No. 62/951,471, filed on Dec. 20, 2019.
(60) Provisional application No. 62/852,244, filed on May 23, 2019.

(51) Int. Cl. C12N 15/70; C12N 1/20; C12N 15/74; C12N 15/90; C12N 9/22; C07K 14/005

(52) U.S. Cl. CPC C12N 15/70; C12N 1/20; C12N 15/746; C12N 2510/00; C12N 9/22; C07K 14/005; C12N 2795/00043; C12N 15/90

(57) ABSTRACT

Provided herein, in some aspects are high efficiency gene editing methods in bacterial cells using single-stranded annealing proteins and/or single-stranded binding proteins.

### RELATED APPLICATIONS

This application is a U.S. national stage application claiming the benefit of international application number PCT/US2020/034025 filed on May 21, 2020, which claims the benefit under 35 U.S.C. § 119(e) of U.S. provisional application No. 62/852,244 filed on May 23, 2019 and U.S. provisional application Ser. No. 62/951,471 filed on Dec. 20, 2019, each of which is incorporated by reference herein in its entirety.

### GOVERNMENT LICENSE RIGHTS

This invention was made with government support under DE-FG02-02ER63445 awarded by the U.S. Department of Energy. The government has certain rights in the invention.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jan. 21, 2022, is named H049870689US02-SUBSEQ-FL.TXT and is 1,029,796 bytes in size.

### BACKGROUND

Recombineering was introduced as a term in 2001 to refer to a method for integrating linear double-stranded DNA¹ (dsDNA) or synthetic single-stranded DNA oligonucleotides (ssDNA or oligonucleotides (oligos))² into the Escherichia coli (E. coli) genome by expression of the Red operon from Enterobacteria phage λ. The Red operon comprises three genes: 1) λ Exo, a 5′ to 3′ dsDNA exonuclease that loads Redβ onto resected ssDNA^{3,4}; 2) Redβ, a single-stranded annealing protein (SSAP) that anneals ssDNA to genomic DNA at the replication fork⁵; and 3) λ Gam, a bacterial nuclease inhibitor that protects linear dsDNA from degradation⁶. Redβ, the SSAP, is required for recombineering of both ssDNA and dsDNA, whereas λ Exo and λ Gam are thought to be involved in recombineering of dsDNA. Improvements to the efficiency of ssDNA recombineering in E. coli have been made through the knockout of mismatch repair machinery⁷ and the protection of oligos from nucleolytic degradation⁸. These improvements spurred the development of multiplexed automatable genome engineering (MAGE), a technique that for the first time envisioned the bacterial genome as a massively editable template. MAGE was applied notably to the full genomic recoding of E. coli MG1655⁹ (removal of all amber stop codons—TAG), which has subsequently become a model chassis organism for biocontainment¹⁰ and non-standard amino acid (NSAA) studies^{11,12}.

### SUMMARY

The present disclosure is based, at least in part, on unexpected data showing that pairs of single-stranded annealing proteins (SSAPs) and single-stranded binding proteins (SSBs) can be used to efficiently edit the genomes of a variety of bacterial species (not only E. coli) with cross-species specificity. In some embodiments, the SSAPs and SSBs are from entirely different species of bacteriophage, relative to each other, yet can still be used together for efficient recombineering. The data herein also unexpectedly demonstrate that a pair of SSB and SSAP can be used to integrate into the genome of a host cell an exogenous double-stranded nucleic acid, even in the absence of an exogenous exonuclease (e.g., a cognate exogenous exonuclease). As used herein, an exonuclease is capable of removing successive nucleotides from the end of a nucleic acid. An exonuclease may be a double-stranded exonuclease that is useful in generating a nucleic acid comprising single-stranded nucleotide overhangs. An exogenous exonuclease is an exonuclease that is introduced into a cell. A cognate exogenous exonuclease is an exonuclease that is from the same species as a SSAP, SSB, or combination thereof that is introduced into a cell.

Accordingly, provided herein, in some embodiments, are SSAPs that may be used together with species-matched or species-unmatched SSBs for use in editing the genome of cells (e.g., recombineering).

Provided herein, in some embodiments, are recombineering tools for efficient gene editing (e.g., multiplex genomic editing) in microbial cells, such as bacterial cells. The principal limitation of recombineering technology is that Redβ, does not function well in non-E. coli bacterial species. Species-specific SSAPs have been reported for other hosts, but in comparison to E. coli, where ssDNA recombineering efficiency has been reported at over 20%¹³, reported editing efficiency in non-E. coli hosts is as low as 0.01% and no more than 1%^{14,15}. Applications such as genomic recoding, strain engineering, or other engineering goals that require the ability to massively edit a bacterial genome are not currently possible outside of E. coli (i.e., without bacterial species). Furthermore, even the efficiency that has been previously reported in E. coli (˜20-30%) remains a limiting factor to more advanced applications that utilize a more efficient gene-editing tool. For instance, 321 edits were made to the E. coli MG1655 genome to recode all TAGs to TAA, but this process took about 4 years and necessitated conjugation steps to assemble the genome from partially-recoded parts. To remove or alter another native codon, thousands of mutations would need to be made. Provided herein is a more efficient editing tool to make feasible the kinds of applications that require hundreds to thousands of mutations within a shorter period of time.

### BRIEF DESCRIPTION OF THE DRAWINGS

FIGS. 1A-1B show matrices testing all combinations of the top seven enriched SSBs against the top four enriched SSAPs in E. coli (FIG. 1A) and L. lactis (FIG. 1B).

FIGS. 2A-2C show results of editing efficiency testing for SSAPs and SSAP/single-stranded binding (SSB) pairs from experiments using E. coli (FIG. 2A), L. lactis (FIG. 2B), and M. smegmatis (FIG. 2C).

FIG. 3 show the results of multiplex incorporation of edits in E. coli populations expressing either an efficient SSAP (SEQ ID NO: 157), an efficient SSAP/SSB pair (SEQ ID NO: 157-SEQ ID NO: 384), or the widely-used Redβ (EC-Bet).

FIGS. 4A-4C show the results of various experiments testing the SSAP comprising the sequence of SEQ ID NO: 24, a high-efficiency SSAP from Pseudomonas aeruginosa (P. Aeruginosa) that was identified by an early experiment with E. coli. FIG. 4A shows that the SSAP SEQ ID NO: 24 displays improved annealing kinetics in vitro. FIG. 4B shows that the SSAP SEQ ID NO: 24 is improved over Redβ in many clinically relevant species of Gammaproteobacteria. FIG. 4C shows that in P. aeruginosa, the SSAP SEQ ID NO: 24 enables rapid multi-drug resistance profiling.

FIG. 5 shows top individual SSAPs SEQ ID NO: 157 and SEQ ID NO: 24 expressed in E. coli from a high-activity promoter. The mutational profile of edits are shown, including the efficiency of making 18-nucleotide (NT) and 30-NT mismatches.

FIGS. 6A-6B show that co-expression of an SSAP/SSB pair that facilitates the integration of double-stranded cassettes. FIG. 6A shows erythromycin colony forming units (CFUs) after expression of SSAP SEQ ID NO: 24 alone, or co-expressed with its corresponding SSB (PaSSB, SEQ ID NO: 472) or exonuclease. The SSAP/SSB pair alone is enough for cassette insertion. FIG. 6B shows that EcSSAP (Redβ) performs slightly better with its associated exonuclease, but the SSAP/SSB pair alone performs nearly as well.

FIG. 7 shows editing efficiency in Agrobacterium tumefaciens expressing SSAP SEQ ID NO: 143 in combination with either SSB SEQ ID NO: 310 or SSB SEQ ID NO: 368. Editing efficiency of close to 1% was measured in SSAP SEQ ID NO: 143/SSB SEQ ID NO: 310.

FIGS. 8A-8B include graphs showing frequency and enrichment of members of Broad RecT Library over ten rounds of SEER enrichment. FIG. 8A shows the frequency of the library members. FIG. 8B shows the enrichment of library members.

FIGS. 9A-9E show recombineering results with a broad RecT Library and CspRecT. FIG. 9A is a graph in which frequency is plotted against enrichment for each Broad RecT Library member after the tenth round of selection. One candidate protein, CspRecT (box), was the standout winner. In all subsequent panels, Redβ, PapRecT, and CspRecT are compared when expressed from a pORTMAGE-based construct (FIG. 10) in wild-type MG1655 E. coli. Significance values are indicated for a grouped parametric t-test, where ns and ***** indicate p >0.05 and p <0.0001 respectively. FIG. 9B is a graph in which editing efficiency was measured by blue/white screening at the LacZ locus for eight different single-base mismatches (n=3). FIG. 9C is a graph in which editing efficiency was measured by blue/white screening at the LacZ locus for 18-base and 30-base mismatches (n=3). FIG. 9D shows a sample MAGE experiment that tested editing at 1, 5, 10, 15, or 20 sites at once in triplicate, was read out by NGS. The solid lines represent the average editing efficiency across all sites, while the dashed lines represent the aggregate editing efficiency. FIG. 9E shows a 130-oligo DIvERGE experiment using oligos that were designed to tile four different genomic loci that encode the drug targets of fluoroquinolone antibiotics and are known hotspots for CIP resistance. The oligos contained 1.5% degeneracy at each nucleotide position along their entire length. All 130 oligos were mixed and transformed together into cells (n=3). Colony forming units were measured at three different CIP concentrations after plating 1/100^{th} of the final recovery volume.

FIGS. 10A-10B are schematics showing vector maps. FIG. 10A shows pARC8-DEST, which was created to have a pBAD regulatory region, beta lactamase, a p15a origin, and a lethal ccdB gene flanked by attR sites for Gateway cloning. Introduction by the LR Gateway reaction of for instance SR001, would create the vector on the right, with an arabinose-inducible SR001 followed by a barcode. FIG. 10B shows two pORTMAGE vectors are provided for broad-spectrum recombineering. pORTMAGE-Ec1 was demonstrated effective in E. coli, C. freundii, and K. pneumoniae, while pORTMAGE-Pa1 was demonstrated effective in P. aeruginosa.

FIGS. 11A-11C depict recombineering in Gammaproteobacteria. FIG. 11A shows results of recombineering experiments that were run with Redβ, PapRecT, and CspRecT expressed off of the pORTMAGE311B backbone, or with a pBBR1 origin in the case of P. aeruginosa. Editing efficiency was measured by colony counts on selective vs. non-selective plates (n=3; see methods). Vector optimization resulted in improved efficiency of PapRecT in P. aeruginosa (see FIG. 13) FIG. 11B is a diagram of a simple multi-drug resistance experiment in P. aeruginosa harboring an optimized PapRecT plasmid expression system, pORTMAGE-Pa1. In a single round of MAGE, a pool of five oligos was used to incorporate genetic modifications that would provide resistance to STR, RIF, and CIP (n=3). These populations were then selected by plating on all combinations of 1-, 2-, or 3-antibiotic agarose plates and compared with a non-selective control. FIG. 11C shows observed efficiencies that were calculated by comparing colony counts on selective vs. non-selective plates. Expected efficiencies for multi-locus events were calculated as the product of all relevant single-locus efficiencies.

FIG. 12 is a graph showing recombineering efficiency in P. aeruginosa was measured for PapRecT with E. coli codons, PapRecT with its wild-type codons, and two SSAPs that have been reported to work in Pseudomonas putida. This was measured both with the original pORTMAGE311B RBS and an RBS optimized for P. aeruginosa. Significance values are indicated for a parametric t-test between two groups, where ns, *, **, ***, and ***** indicate p >0.05, p <0.05, p <0.01, p <0.001, and p <0.0001 respectively.

FIG. 13 shows editing efficiency in making a single-base mutation at the rpsL locus in P. aeruginosa with various plasmid variants expressing PapRecT. An unoptimized plasmid (far left) was constructed by replacing, in pORTMAGE312B (Addgene), the RSF1010 origin of replication and the kanamycin resistance gene with a pBBR1 origin of replication and a gentamicin resistance gene. The best-performing plasmid variant (third from right) was renamed pORTMAGE-Pa1 (Addgene). Constructs examining the role of MutL in single-base recombineering efficiency were made by first restoring wild-type PaMutL and then by removing it entirely (second from right, and far right respectively).

FIG. 14 shows results with one round of MAGE with a pool of three oligos that confer Ciprofloxacin resistance was conducted in P. aeruginosa with pORTMAGE-Pa1. Editing efficiency is shown after plating on three different concentrations of antibiotic.

FIG. 15 shows the effect of codon-usage on Redβ editing efficiency in E. coli. The efficiency of Redβ from the Broad SSAP Library was compared with Redβ expressed off of its wild-type codons. Efficiency of making a single base pair mutation in a non-coding gene was measured by next generation sequencing (NGS).

FIGS. 16A-16B include data showing the editing efficiency and growth rates of bacteria expressing a candidate from the Broad SSAP Library or Redβ. FIG. 16A shows the efficiency of a candidate SSAP at incorporating a single-base-pair silent mutation at a non-essential gene, ynfF. Efficiency was read out by NGS. Significance values are indicated for a parametric t-test between two groups, where ns, *, **, ***, and ***** indicate p >0.05, p <0.05, p <0.01, p <0.001, and p <0.0001 respectively. FIG. 16B shows growth rates, which were measured by plate-reader growth assay and plotted against the maximum attained OD₆₀₀ of the culture.

FIGS. 17A-17H include data showing the editing efficiency in recombinant cells comprising RecTs, SSBs, or “cognate pairs.” FIG. 17A shows an in-vitro model of ssDNA annealing inhibition by EcSSB or L1SSB, and ability of λ-Red β to overcome annealing inhibition by EcSSB. FIG. 17B shows ssDNA annealing without SSB, precoated with EcSSB, or pre-coated with L1SSB. Shaded area represents the SEM of at least 2 replicates. FIG. 17C shows ssDNA annealing in the presence of λ-Red β when pre-coated with EcSSB or L1SSB. Shaded area represents the SEM of at least 2 replicates. FIG. 17D shows a model for RecT-mediated editing in the presence of SSB. An interaction between RecT and the host SSB enables oligo annealing to the lagging strand of the replication fork. **Co-expressing an exogenous SSB that is compatible with a particular RecT variant can in some species enable efficient homologous genome editing even if host compatibility does not exist. FIGS. 17E-17F show calculation of editing efficiency in L. lactis and E. coli is performed by introducing antibiotic resistance mutations into the genome using synthetic oligos, and then measuring the ratio of resistant cells to total cells. FIGS. 17G-17H show a comparison of the efficiency of editing in L. lactis and E. coli after the expression of either RecTs, SSBs, or “cognate pairs” (see, e.g., Example 10).

FIGS. 18A-18F include data showing genome editing efficiency using SSAP and chimeric SSB pairs. FIG. 18A shows a crystal structure of homotetrameric E. coli SSB bound to ssDNA (PDB-ID 1EYG)37. The amino acid sequence of the flexible C-terminal tail is diagramed in the right panel, along with the design of a 9AA C-terminal truncation to SSB. FIG. 18B shows a diagram of the L. lactis SSB C-terminal tail is diagramed, along with an example of an SSB C-terminal tail replacement. In this case, the 9 C-terminal amino acids of the L. lactis SSB are replaced with the corresponding residues from E. coli SSB. The notation “L1SSB C9:EcSSB” is used as shorthand. FIG. 18C shows editing efficiency in L. lactis of λ-Red β with a 9AA C-terminally truncated EcSSB mutant. The sequence shown for EcSSB (C10) corresponds to SEQ ID NO: 516. FIG. 18D shows editing efficiency in L. lactis of λ-Red β expressed with L1SSB, or mutants of L1SSB with C3, C7, C8, or C9 terminal residues replaced with the corresponding residues from EcSSB. The following sequences are shown from top to bottom: SEQ ID NOS: 532, 538-541 and 516. FIGS. 18E-18F show editing efficiency in L. lactis of PapRecT (FIG. 18E) or MspRecT (FIG. 18F) expressed with L1SSB, or mutants of L1SSB with the C7 or C8 terminal residues replaced with the corresponding residues from the cognate SSB. The following sequences are shown in FIG. 18E from top to bottom: SEQ ID NOS: 532, 542-543, and 520. The following sequences are shown in FIG. 18F from top to bottom: SEQ ID NOs: 532, 544-545, and 524.

FIGS. 19A-19F include data evaluating RecT compatibility with distinct bacterial SSBs and chimeric SSBs. FIGS. 19A-19B show heat maps showing the fold improvement in editing efficiency due to SSB coexpression in (FIG. 19A) L. lactis or (FIG. 19B) E. coli of RecT-SSB pairs as compared to the RecT alone. FIG. 19C shows C-terminal sequences of SSBs as well as RecT compatibility given FIGS. 19A and 19B.” The following sequences are shown from top to bottom: SEQ ID NOs: 516, 516, 516, 520, 524, 528, 532, and 535. FIG. 19D shows editing efficiency in L. lactis of PapRecT coexpressed with LrSSB, MsSSB, or mutants of LrSSB which had the C7 or C8 terminal residues replaced with the corresponding residues from the MsSSB. The following sequences are shown from top to bottom: SEQ ID NOS: 528, 546, 547, and 524. FIG. 19E shows editing efficiency in M. smegmatis of λ-Red β, PapRecT, MspRecT, and LrpRecT. FIG. 19F shows editing efficiency in L. rhamnosus of λ-Red β, PapRecT, MspRecT, and LrpRecT.

FIGS. 20A-20B show editing efficiency in C. crescentus using pairs of RecT and SSB. FIG. 20A shows editing efficiency in C. crescentus of two RecT-SSB protein pairs, λ-Red β+PaSSB and PapRecT+PaSSB which had high genome editing efficiency in both E. coli and L. lactis. FIG. 20B shows editing efficiency in C. crescentus of λ-Red β+PaSSB with ribosomal binding sites optimized for translation rate and using an oligo designed to evade mismatch repair.

FIG. 21 shows that in L. lactis, the internal RBS sequence affected recombination efficiency using the bicistronic Redβ and EcSSB construct. RBS 2, which enabled the highest efficiency genome editing in this experiment was selected used in all other bicistronic constructs unless otherwise indicated. The sequences for RBS1-RBS4 correspond to SEQ ID NOs: 509, 507, 510 and 511, respectively.

FIG. 22 shows design of RBSs for use in C. crescentus. Using the Salis et al. RBS calculator, RBSs were designed to confer a greater translation rate in order to increase RecT and SSB expression for the Caulobacter constructs. See, e.g., Salis et al. Nat. Biotechnol. 27, 946-50 (2009) and Borujeni et al. Nucleic Acids Res. 42, 2646-2659 (2014). The sequences shown correspond to SEQ ID NOS: 505, 506, 507, and 508 from top to bottom.

FIGS. 23A-23E includes data showing genome editing efficiency of L. lactis comprising PapRecT, and PaSSB. FIG. 23A shows that in L. lactis, optimization of nisin concentration contributed to a significant improvement in editing efficiency for the PapRecT protein and the PaSSB protein construct. 10 ng/mL nisin was much more effective than 1 ng/mL nisin and resulted in an increase in editing efficiency improvement from 0.5% to 8%. The optimal oligo amount plateaued at 50 μg of DNA, which corresponds 21.4 μM in 80 μL. FIG. 23B shows expression of the L. lactis MutL variant E33K allowed the efficient introduction of 1 bp mismatches at similar efficiency to 4 bp mismatches which evade MMR. FIG. 23C shows that after optimization from FIGS. 23A-23B, PapRecT+PaSSB+LlMutLE33K enabled ˜20% editing efficiency at the Rif locus, and multiplexed editing (FIG. 23D). FIG. 23E shows that co-expression of PapRecT+PaSSB enabled the efficient introduction of a 1 kb selectable marker as dsDNA even without the addition of the cognate phage exonuclease. This also was observed for Redb with EcSSB in L. lactis (Data not shown).

FIG. 24 shows the editing efficiency of SSAP candidates in Agrobacterium tumefaciens. Enrichment on the Y-axis is a measure of editing efficiency.

FIG. 25 shows the editing efficiency of SSAP candidates in Staphylococcus aureus. Enrichment on the Y-axis is a measure of editing efficiency.

### DETAILED DESCRIPTION

A library of 234 SSAPs was tested both individually and co-expressed with a library of 237 SSBs. These libraries were tested in E. coli and two model gram positive microbes: Lactococcus lactis (Firmicutes) and Mycobacterium smegmatis (Actinobacteria). L. lactis and M. smegmatis are important model systems, are distant relations of E. coli and of each other, and have had reports of low efficiency recombineering (L. lactis: ˜0.1%¹⁵; M. smegmatis: ˜0.01%¹⁴). L. lactis is an industrially-relevant microbe used in dairy production of kefir, buttermilk, and cheese, and is a human commensal. M. smegmatis is also a human commensal, and a fast-growing model system for M. tuberculosis. In fact, Firmicutes and Actinobacteria are two of the most highly-populated phyla of human commensals¹⁶.

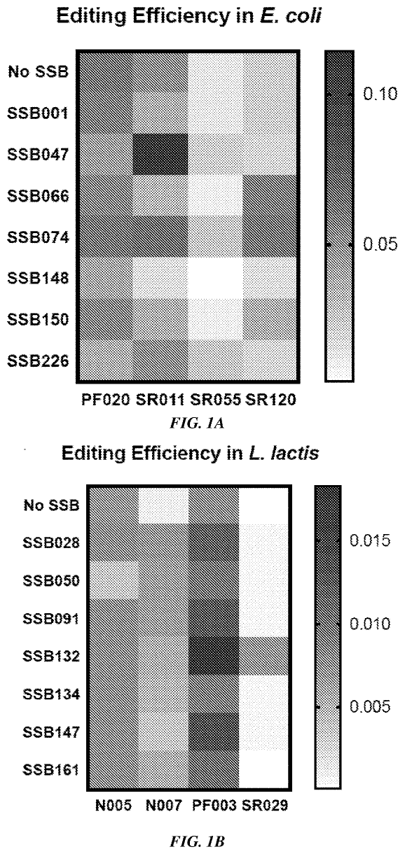

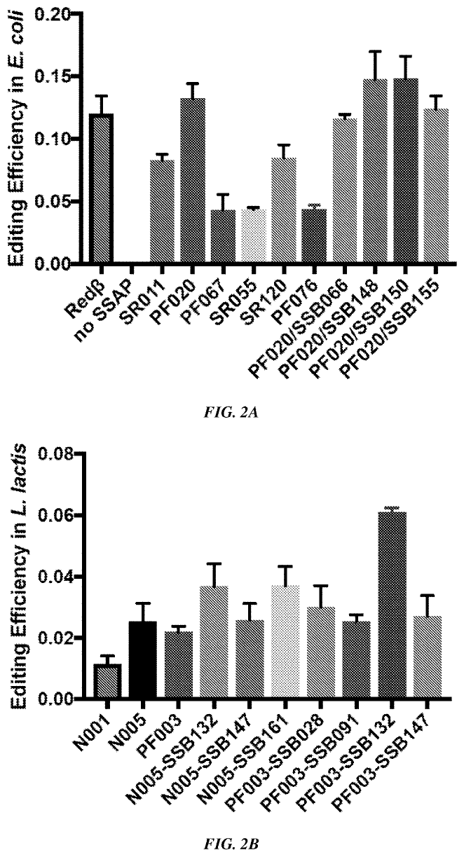

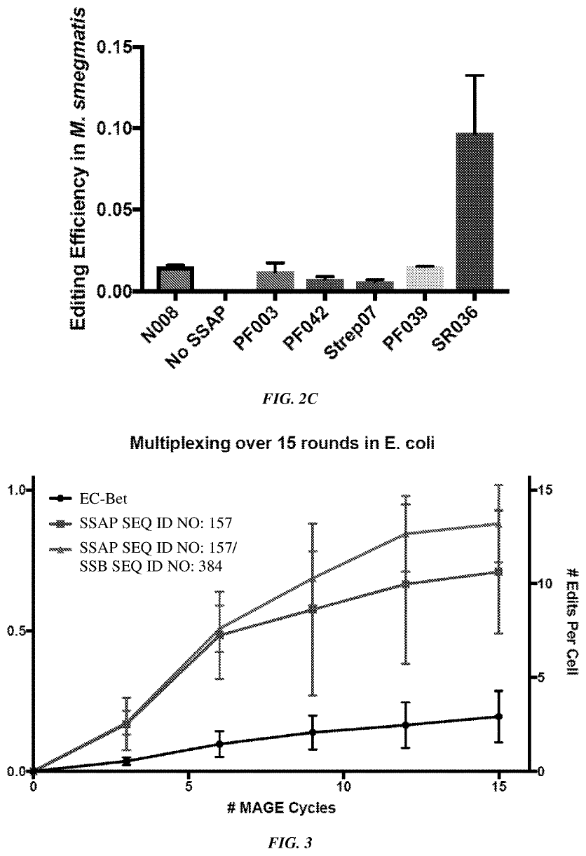

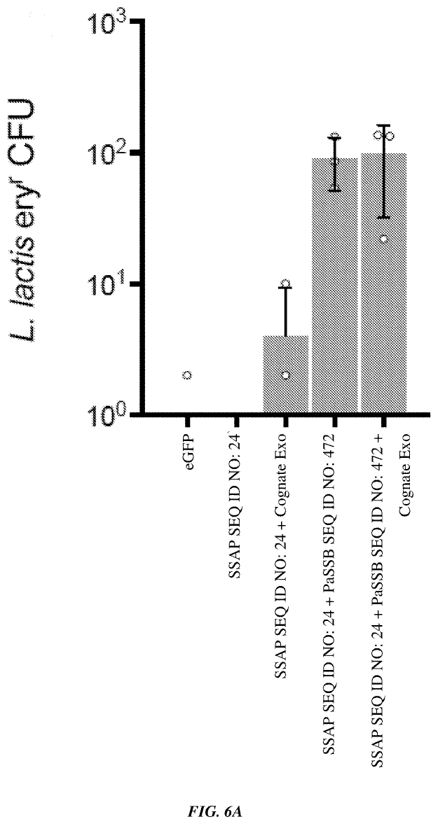

Oligo recombineering efficiency was improved, as shown herein, in all three bacterial species: E. coli (40%), L. lactis (20%), and M. smegmatis (5%) enough to support high-throughput experimentation by recombineering without the need for selection. Top SSAPs were tested in the three chassis organisms, and in all cases supported significantly improved rates of oligo-mediated recombineering (FIGS. 1A-1C, FIG. 5). In the SSAP/SSB library, SSAPs and SSBs were both individually enriched, and so matrices were constructed of every combination of high-performing SSAPs and high-performing SSBs (FIGS. 2A-2B). Through testing these in a high-throughput assay and reading out efficiency by next-generation sequencing (NGS), the highest efficiency pairs were identified. These pairs performed better than any individual SSAP (FIGS. 1A-1C) and allowed for double-stranded DNA cassette integration, even in the absence of an exogenous exonuclease (FIGS. 6A-6B).

Next, the multiplex incorporation of edits in E. coli was tested, which was demonstrative of some of the more important applications enabled by the technology provided herein. The most efficient SSAP/SSB pair in E. coli incorporated at close to 100% efficiency (15 edits simultaneously) after a week of MAGE cycling, as compared to Redβ, which only did so at 20% efficiency (FIG. 3).

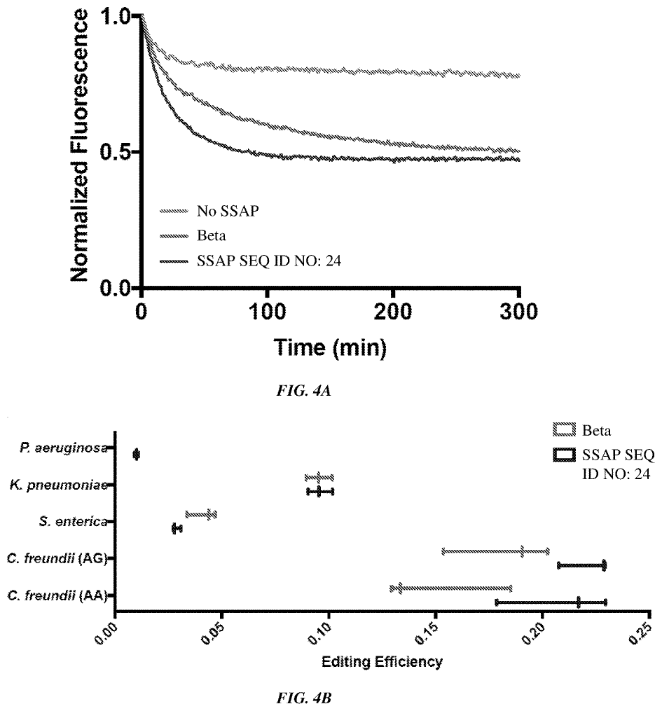

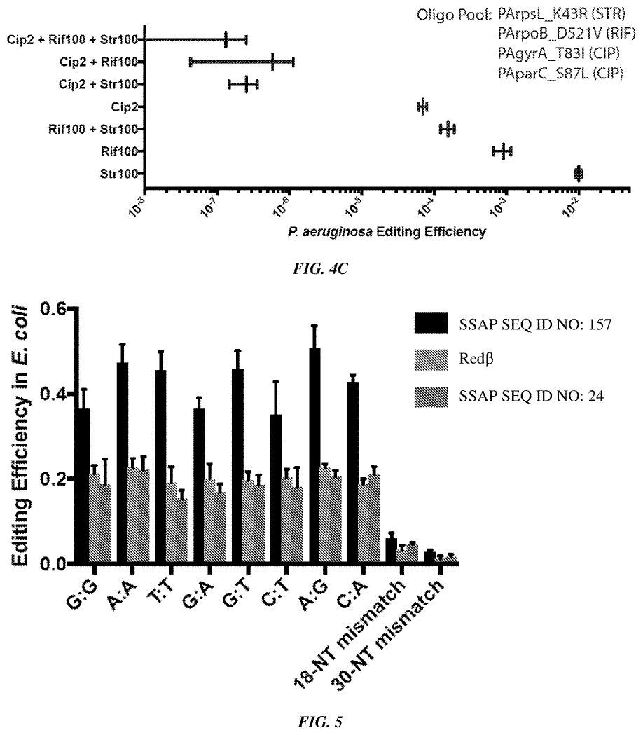

Finally, the efficiency of genome-editing was tested in species that had not been tested in the above-mentioned libraries. First, a highly-enriched SSAP from the E. coli experiments was tested in clinically relevant Gammaproteobacteria (FIGS. 4A-4C). It was found that the SSAP SEQ ID NO: 24 functions at high efficiency in Pseudomonas aeruginosa, where Redβ does not work. This allows for the reconstruction of antibiotic-resistant phenotypes at high efficiency in this host, which has developed significant resistance. Next, a library of co-expressed SSAP/SSB pairs (25 most-enriched SSBs and three most-enriched SSAPs across E. coli, L. lactis, and M. smegmatis) was tested in Agrobacterium tumefaciens. This 75 member library was enriched for the most active variants over two rounds of selective MAGE, and two of the most frequent pairs were isolated. The most active of these pairs showed close to 1% editing efficiency.

### Single-Stranded Annealing Protein (SSAP)

Aspects of the present disclosure provide single-stranded annealing proteins. Single-stranded annealing proteins (SSAPs) are recombinases that are capable of annealing an exogenous nucleic acid (any nucleic acid that is introduced into a cell) to a target locus in the genome of a cell. A SSAP may be from (e.g., derived from, obtained from, and/or isolated from) any SSAP superfamily, including RecT, ERF, RAD52, SAK, SAK4, and GP2.5. See, e.g., Iyer et al., BMC Genomics. 2002 Mar. 21; 3:8; Neamah et al., Nucleic Acids Res. 2017 Jun. 20; 45(11):6507-6519. In some instances, GP2.5 is from T7 phage. As a non-limiting example, SSAPs may be identified using the Pfam database. For example, RecT SSAPs may be identified under Pfam Accession No. PF03837, ERF SSAPs may be identified under Pfam Accession No. PF04404, and RAD 52 SSAPs may be identified under Pfam Accession No. PF04098.

As used herein, a SSAP may be from any source. For example, SSAPs may be from a virus or a bacteria. The source may be a eukaryote or a prokaryote. See, e.g., Table 1.

A SSAP may comprise a sequence that is least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to a sequence selected from SEQ ID NOS: 1-234. In some instances, a SSAP comprises a sequence selected from SEQ ID NOS: 1-234. In some instances, a SSAP consists of a sequence selected from SEQ ID NOS: 1-234.

### Single-Stranded Binding Protein (SSB)

The SSAPs of the present disclosure may be used with a single-stranded binding protein (SSB). SSBs bind to single-stranded nucleic acids (e.g., single-stranded nucleic acids comprising deoxyribonucleotides, ribonucleotides, or a combination thereof). The binding of a SSB to a single-stranded nucleic acid can serve numerous functions. For example, SSB binding may protect a nucleic acid from degradation. In some instances, SSB binding to a single-stranded nucleic acid reduces the secondary structure of the nucleic acid, which may increase the accessibility of the nucleic acid to other enzymes (e.g., recombinases). SSB binding can also prevent re-annealing of complementary strands during replication. As a non-limiting example, SSBs may be identified using the Pfam database under Accession Number PF00436.

The SSBs of the present disclosure may be from any source. For example, SSBs may be from a virus or a bacteria. The source may be a eukaryote or a prokaryote. See, e.g., Table 1.

A SSB may comprise a sequence that is least 50% (e.g., at least 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99%, including all values in between) identical to a sequence selected from SEQ ID NOS: 235-472. In some instances, a SSB comprises a sequence selected from SEQ ID NOS: 235-472. In some instances, a SSB consists of a sequence selected from SEQ ID NOS: 235-472.

In some embodiments, a SSB is a chimeric SSB and comprises SSB sequences from two different sources. To produce a chimeric SSB, one or more amino acids in the C-terminus of the SSB may be substituted. For example, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 amino acids from the C-terminus of an SSB may be substituted. The C-terminus of a SSB may be substituted with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 amino acids from the C-terminus of another SSB.

In some embodiments, a chimeric SSB is used together with an SSAP that is from a bacteriophage that is capable of infecting a type of bacteria. In such instances, the chimeric SSB may comprise a C-terminal sequence from an SSB from the same source as the source of the SSAP. In some embodiments, a chimeric SSB may comprise a C-terminal SSB sequence from a bacterium that the bacteriophage the SSAP is sourced from is capable of infecting. For example, a chimeric SSB may be used in a first type of bacterial cell with an SSAP from a bacteriophage that can infect, or from a prophage that is stably integrated into the genome of, a second type of bacterial cell. The chimeric SSB may comprise a sequence encoding an SSB from the first type of bacterial cell, in which the C-terminus of this first SSB is substituted with one or more amino acids from the C-terminus of a second SSB that is from the second type of bacterial cell that the bacteriophage can infect. As a non-limiting example, the SSAP PapRecT (SEQ ID NO: 24) may be used with a chimeric SSB comprising 7, 8, 9, or 10 amino acids of the C-terminus of PaSSB (SEQ ID NO: 472). In some instances, the chimeric SSB may comprise a C-terminal sequence that includes 1, 2, 3, 4, or 5 mutations relative to a C-terminal sequence from a SSB from a bacteriophage that is capable of infecting the same type of bacteria that the SSAP is capable of infecting.

In some embodiments, a chimeric SSB comprises a C-terminal sequence that is at least 70%, 80%, or at least 90% identical to a sequence selected from SEQ ID NOs: 516-547. In some embodiments, a chimeric SSB comprises a sequence selected from SEQ ID NOs: 516-547.

### Source of Proteins

The proteins of the present disclosure (e.g., SSAPs, SSBs, dominant negative mismatch repair enzymes, or exonucleases) may be from any source. As used herein, a source refers to any species existing in nature that naturally harbors the protein (e.g., SSAP, SSB, exonuclease, dominant negative mismatch repair enzyme, or a combination thereof). The term “naturally” refers to an event that occurs without human intervention. For example, certain bacteriophage naturally infect bacteria, delivering a SSAP and/or SSB; thus, some bacteria naturally harbor that SSAP and/or SSB. Non-limiting examples of suitable sources of SSAPs and SSBs are provided in Table 1.

Many viruses, including bacteriophages, naturally encode SSAP, SSB, exonuclease, dominant negative mismatch repair enzyme, or a combination thereof. Therefore, a source of a SSAP, SSB, exonuclease, dominant negative mismatch repair enzyme, or a combination thereof may be a virus. In some instances, the source of a SSAP, SSB, exonuclease, dominant negative mismatch repair enzyme, or a combination thereof is a bacteriophage. Bacteriophages or phages are viruses that infect bacteria and are often classified by the type of nucleic acid genome and morphology. For example, the genome of bacteriophages may be linear or circular, double-stranded or single-stranded, and may comprise deoxyribonucleotides (DNA) or ribonucleotides (RNA). After a phage inserts its genome into a bacterial host cell, the phage genome can be reproduced through the lysogenic cycle, lytic cycle, or the lysogenic cycle followed by the lytic cycle. During the lysogenic cycle, the phage genome is integrated into the host bacterium's genome. The infected bacterial cell remains intact during the lysogenic cycle and replicates the phage genome. In contrast, during the lytic cycle, the phage genome does not integrate into the host genome and the phage hijacks the host cell's machinery to replicate the phage genome, produce viral components, and assemble new viral phages. Once the new viral phages are formed, the phages lyse the host cell and are released. Viruses that infect non-bacterial host cells use similar mechanisms of replication. In some instances, the source of a SSAP, SSB, dominant negative mismatch repair enzyme, exonuclease, or a combination thereof is a virus that can infect a particular species. In some instances, the source of a SSAP, SSB, exonuclease, dominant negative mismatch repair enzyme, or a combination thereof is a bacteriophage that can infect, or from a prophage that is stably integrated into the genome of, a particular species of bacteria.

A source of a SSAP or SSB may also be a cell (e.g., a prokaryotic cell or a eukaryotic cell). As used herein, a cell that is a source of a SSAP or SSB is a cell existing in nature that harbors a gene encoding the SSAP or SSB. In some instances, the SSAP or SSB is a host gene (an endogenous gene). Since viruses naturally infect cells, a source of SSAP or SSB could also be a cell existing in nature that has been naturally infected by a virus that encodes that SSAP or SSB.

Non-limiting examples of phages include T7 (coliphage), T3 (coliphage), K1E (K1-capsule-specific coliphage), K1F (K1-capsule-specific coliphage), K1-5 (K1- or K5-capsule-specific coliphage), SP6 (Salmonella phage), LUZ19 (Pseudomonas phage), gh-1 (Pseudomonas phage), and K11 (Klebsiella phage).

Non-limiting examples of a source of a SSAP, SSB, dominant negative mismatch repair enzyme, an exonuclease or a combination thereof include [Clostridium] methylpentosum DSM 5476, Acetobacter orientalis 21F-2, Acinetobacter radioresistens SK82, Acinetobacter sp P8-3-8, Acinetobacter sp SH024, Actinobacteria bacterium OK074, Acyrthosiphon pisum secondary endosymbiont phage 1 (BacteriophageAPSE-1), Agathobacter rectalis (strain ATCC 33656/DSM 3377/JCM 17463/KCTC5835/VPI 0990) (Eubacterium rectale), Agrobacterium rhizogenes, Ahrensia sp R2A130, Akkermansia sp KLE1798, Anaerococcus hydrogenalis ACS-025-V-Sch4, Avibacterium paragallinarum JF4211, Bacillus phage 0305phi8-36, Bacillus phage SPP1 (Bacteriophage SPP1), Bacillus sp 1NLA3E, Bacillus sp 2_A_57_CT2, Bacillus sporothermodurans, Bacillus subtilis, Bacillus subtilis subsp spizizenii (strain TU-B-10), Jeotgalibacillus marinus, Bacillus subtilis subsp spizizenii (strain TU-B-10), Jeotgalibacillus marinus, Bacillus thuringiensis Sbt003, Escherichia coli VBL21-Gold(DE3)pLysS AG\′, Enterobacteria phage HK630, Enterobacteria phage lambda (Bacteriophage lambda), Escherichia coli TA280, Escherichia coli 1-176-05_S3_C2, Escherichia coli 40967, Bacteroides caccae ATCC 43185, Bartonella schoenbuchensis (strain DSM 13525/NCTC 13165/R1), Bifidobacterium magnum, Bifidobacterium reuteri DSM 23975, Bordetella bronchiseptica (Alcaligenes bronchisepticus), Bordetella phage BPP-1, Borrelia duttonii CR2A, Bradyrhizobium sp STM 3843, Brevibacillus brevis (strain 47/JCM 6285/NBRC 100599), Burkholderia cenocepacia (strain ATCC BAA-245/DSM 16553/LMG 16656/NCTC 13227/J2315/CF5610) (Burkholderia cepacia (strain J2315)), Burkholderia cenocepacia BC7, Burkholderia phage BcepC6B, Burkholderia phage BcepGomr, Burkholderia phage BcepNazgul, Burkholderia phage BcepNY3, Campylobacter coli 80352, Candidatus Accumulibacter sp SK-12, Candidatus Cloacimonas sp SDB, Capnocytophaga sp oral taxon 338 str F0234, Caulobacter vibrioides (strain ATCC 19089/CB15) (Caulobacter crescentus), Clostridium beijerinckii (strain ATCC 51743/NCIMB 8052) (Clostridiumacetobutylicum), Clostridium botulinum (strain Eklund 17B/Type B), Clostridium botulinum C str Eklund, Clostridium phage phiC2, Peptoclostridium difficile E15, Clostridium phage phiMMP03, Peptoclostridium difficile (Clostridium difficile), Clostridium sp CAG:470, Clostridium sp FS41, Clostridium sporogenes (strain ATCC 7955/DSM 767/NBRC 16411/NCIMB 8053/NCTC 8594/PA 3679), Collinsella stercoris DSM 13279, Commensalibacter intestini A911, Coriobacteriales bacterium DNF00809, Corynebacterium striatum ATCC 6940, Cryptobacterium curtum (strain ATCC 700683/DSM 15641/12-3), Cyanophage PSS2, Dermabacter sp HFH0086, Desulfitobacterium metallireducens DSM 15288, Desulfovibrio sp FW1012B, Dialister sp CAG:486, Drosophila melanogaster (Fruit fly), Elusimicrobium minutum (strain Pei191), Endozoicomonas montiporae, Endozoicomonas montiporae CL-33, Enterobacteria phage HK022 (Bacteriophage HK022), Enterobacteria phage HK629, Salmonella phage HK620 (Bacteriophage HK620), Enterobacteria phage T1 (Bacteriophage T1), Enterococcus faecalis (strain ATCC 700802/V583), Enterococcus faecalis TX0027, Enterococcus faecalis TX0309B, Enterococcus faecalis TX0309A, Enterococcus faecalis (strain ATCC 700802/V583), Escherichia coli, Escherichia phage Rtp, Escherichia phage Tls, Faecalibacterium sp CAG:82, Flavobacterium phage 11b, Frateuria aurantia (strain ATCC 33424/DSM 6220/NBRC 3245/NCIMB13370) (Acetobacter aurantius), Fusobacterium mortiferum ATCC 9817, Fusobacterium ulcerans 12-1B, gamma proteobacterium BDW918, Gordonia soli NBRC 108243, Gramella forsetii (strain KT0803), Haemophilus influenzae, Haemophilus influenzae NT127, Haemophilus paraphrohaemolyticus HK411, Haemophilus parasuis serovar 5 (strain SH0165), Hafnia alvei ATCC 51873, Helicobacter pullorum MIT 98-5489, Helicobacter sp MIT 05-5294, Herbaspirillum sp YR522, Homo sapiens (Human), Hungatella hathewayi DSM 13479, Hydrogenobacter thermophilus (strain DSM 6534/IAM 12695/TK-6), Hydrogenovibrio marinus, Klebsiella pneumoniae subsp rhinoscleromatis ATCC 13884, Komagataeibacter oboediens, Labilithrix luteola, Lactobacillus capillatus DSM 19910, Lactobacillus phage KC5a, Lactobacillus phage phi jlb1, Lactobacillus phage Lc-Nu, Lactobacillus phage phiadh, Lactobacillus phage phigle, Lactobacillus phage phijl1, Lactobacillus prophage Lj928, Lactobacillus johnsonii (strain CNCM 1-12250/La1/NCC 533), Lactobacillus prophage Lj965, Lactobacillus johnsonii (strain CNCM 1-12250/La1/NCC 533), Lactobacillus reuteri, Lactobacillus rossiae DSM 15814, Lactobacillus ruminis SPM0211, Lactobacillus shenzhenensis LY-73, Lactococcus lactis subsp cremoris (strain MG1363), Lactococcus lactis subsp lactis by diacetylactis str TIFN2, Lactococcus lactis subsp lactis (strain IL1403) (Streptococcuslactis), Lactococcus phage bIL309, Lactococcus lactis subsp lactis by diacetylactis str TIFN2, Lactococcus lactis subsp lactis (strain IL1403) (Streptococcuslactis), Lactococcus phage bIL309, Lactococcus phage bIL286, Lactococcus lactis subsp lactis (strain IL1403) (Streptococcuslactis), Lactococcus phage c2, Lactococcus phage LL-H (Lactococcus delbrueckii bacteriophage LL-H), Lactococcus phage phi311, Lactococcus phage ul36k1t1, Lactococcus phage ul362, Lactococcus phage ul361, Lactococcus lactis, Lactococcus phage ul36k1, Lactococcus phage phi311, Lactococcus phage ul36k1t1, Lactococcus phage ul362, Lactococcus phage ul361, Lactococcus lactis, Lactococcus phage ul36k1, Lactococcus phage SK1833, Lactococcus phage SK1, Legionella pneumophila, Leifsonia xyli subsp xyli, Leifsonia xyli subsp xyli (strain CTCB07), Leifsonia xyli subsp xyli, Leifsonia xyli subsp xyli (strain CTCB07), Lentibacillus amyloliquefaciens, Leptotrichia goodfellowii F0264, Leuconostoc mesenteroides subsp mesenteroides (strain ATCC 8293/NCDO 523), Listeria monocytogenes, Listeria phage A118 (Bacteriophage A118), Listeria phage A500 (Bacteriophage A500), Listeria phage B054, Listeria monocytogenes, Listeria welshimeri serovar 6b (strain ATCC 35897/DSM 20650/SLCC5334), Listeria phage PSA, Listonella phage phiHSIC, Mameliella alba, Methylobacterium nodulans (strain LMG 21967/CNCM 1-2342/ORS 2060), Methyloversatilis universalis (strain ATCC BAA-1314/JCM 13912/FAM5), Microbacterium ginsengisoli, Microgenomates group bacterium GW2011_GWF1_44_10, Mycobacterium brisbanense, Mycobacterium marinum (strain ATCC BAA-535/M), Mycobacterium phage Che8, Mycobacterium phage Che8/Mycobacterium smegmatis, Mycobacterium phage Hamulus, Mycobacterium phage Dante, Mycobacterium phage Ardmore, Mycobacterium phage Llij, Mycobacterium phage Drago, Mycobacterium phage Phatniss, Mycobacterium phage Spartacus, Mycobacterium phage Boomer, Mycobacterium phage SiSi, Mycobacterium phage PMC, Mycobacterium phage Ovechkin, Mycobacterium phage Ramsey, Mycobacterium phage Fruitloop, Mycobacterium phage SG4, Mycobacterium phage Hamulus, Mycobacterium phage Dante, Mycobacterium phage Ardmore, Mycobacterium phage Llij, Mycobacterium phage Drago, Mycobacterium phage Phatniss, Mycobacterium phage Spartacus, Mycobacterium phage Boomer, Mycobacterium phage SiSi, Mycobacterium phage PMC, Mycobacterium phage Ovechkin, Mycobacterium phage Ramsey, Mycobacterium phage Fruitloop, Mycobacterium phage SG4, Mycobacterium phage PhatBacter, Mycobacterium phage Elph10, Mycobacterium phage 244, Mycobacterium phage Cjw1, Mycobacterium phage Phrux, Mycobacterium phage Lilac, Mycobacterium phage Phaux, Mycobacterium phage Quink, Mycobacterium phage Pumpkin, Mycobacterium phage Murphy, Mycobacterium phage PhatBacter, Mycobacterium phage Elph10, Mycobacterium phage 244, Mycobacterium phage Cjw1, Mycobacterium phage Phrux, Mycobacterium phage Lilac, Mycobacterium phage Phaux, Mycobacterium phage Quink, Mycobacterium phage Pumpkin, Mycobacterium phage Murphy, Mycobacterium phage Troll4, Mycobacterium phage Gumball, Mycobacterium phage Nova, Mycobacterium phage SirHarley, Mycobacterium phage Adjutor, Mycobacterium phage Butterscotch, Mycobacterium phage PLot, Mycobacterium phage PBI1, Mycobacterium phage Troll4, Mycobacterium phage Gumball, Mycobacterium phage Nova, Mycobacterium phage SirHarley, Mycobacterium phage Adjutor, Mycobacterium phage Butterscotch, Mycobacterium phage PLot, Mycobacterium phage PBI1, Mycobacterium phage Wildcat, Mycobacterium smegmatis, Mycobacterium virus Che9c, Neisseria lactamica Y92-1009, Nitratireductor basaltis, Nitrolancea hollandica Lb, Nocardia farcinica (strain IFM 10152), Nocardia terpenica, Oligotropha carboxidovorans (strain ATCC 49405/DSM 1227/KCTC 32145/OM5), Paenibacillus alvei DSM 29, Paenibacillus curdlanolyticus YK9, Paenibacillus dendritiformis C454, Paenibacillus elgii B69, Paenibacillus lactis 154, Paenibacillus mucilaginosus 3016, Paenibacillus polymyxa (strain E681), Paenibacillus sp FSL R7-0331, Paenibacillus sp P1XP2, Paenibacillus terrae (strain HPL-003), Paeniclostridium sordellii (Clostridium sordellii), Parasutterella excrementihominis CAG:233, Parcubacteria bacterium 32_520, Parcubacteria group bacterium GW2011_GWA2_42_14, Pediococcus acidilactici DSM 20284, Pedobacter antarcticus, Pedobacter antarcticus 4BY, Pedobacter antarcticus, Pedobacter antarcticus 4BY, Pelobacter propionicus (strain DSM 2379/NBRC 103807/OttBd1), Peptoniphilus duerdenii ATCC BAA-1640, Persephonella marina (strain DSM 14350/EX-H1), Phormidium phage Pf-WMP3, Photobacterium profundum (strain SS9), Photorhabdus luminescens subsp laumondii (strain DSM 15139/CIP105565/TT01), Pirellula sp SH-Sr6A, Prevotella sp CAG:873, Prochlorococcus phage P-SSM2, Prochlorococcus phage P-SSP7, Pseudoalteromonas lipolytica SCSIO 04301, Pseudomonas aeruginosa 39016, Pseudomonas aeruginosa, Pseudomonas aeruginosa DHS01, Pseudomonas phage LKA5, Pseudomonas phage F116, Pseudomonas aeruginosa, Pseudomonas aeruginosa DHS01, Pseudomonas phage LKA5, Pseudomonas phage F116, Pseudomonas phage vB_Pae-Kakheti25, Pseudomonas phage vB_PaeP_C1-14_Or, Pseudomonas phage vB_PaeP_p2-10_Or1, Pseudomonas phage PaP3, Rhizobium loti (strain MAFF303099) (Mesorhizobium loti), Rhizobium sp CF080, Rhodothermus phage RM378, Roseateles depolymerans, Ruminococcus sp SR1/5, Saccharomyces cerevisiae, Saccharomyces cerevisiae (strain ATCC 204508/S288c) (BakerVs yeast), Saccharomyces cerevisiae YJM1250, Saccharomyces cerevisiae YJM451, Salinicoccus halodurans, Salinisphaera hydrothermalis C41B8, Salinispora tropica (strain ATCC BAA-916/DSM 44818/CNB-440), Salmonella phage SETP3, Salmonella phage SS3e, Salmonella typhimurium, Salmonella phage ST160, Salmonella phage ST64T (Bacteriophage ST64T), Serratia odorifera DSM 4582, Simkania negevensis (strain ATCC VR-1471/Z), Sodalis glossinidius (strain morsitans), Source, Sphingopyxis sp (strain 113P3), Spiroplasma kunkelii CR2-3x, Sporosarcina newyorkensis 2681, Staphylococcus aureus (strain Mu50/ATCC 700699), Staphylococcus phage 3A, Staphylococcus phage phi7401PVL, Streptococcus pneumoniae, Staphylococcus aureus (strain NCTC 8325), Staphylococcus phage Phil2, Staphylococcus aureus, Staphylococcus phage 47, Staphylococcus phage tp310-2, Staphylococcus phage 3A, Staphylococcus phage phi7401PVL, Streptococcus pneumoniae, Staphylococcus aureus (strain NCTC 8325), Staphylococcus phage Phil2, Staphylococcus aureus, Staphylococcus phage 47, Staphylococcus phage tp310-2, Staphylococcus phage 92, Staphylococcus phage CNPH82, Staphylococcus phage phi11 (Bacteriophage phi-11), Staphylococcus phage 80, Staphylococcus phage 52A, Staphylococcus aureus (strain NCTC 8325), Staphylococcus phage Pv1108, Staphylococcus phage SA97, Staphylococcus phage phi7247PVL, Staphylococcus phage phiETA3, Staphylococcus aureus, Staphylococcus phage phi5967PVL, Stigmatella aurantiaca (strain DW4/3-1), Streptococcus gallolyticus subsp gallolyticus TX20005, Streptococcus infantis SK970, Streptococcus phage 7201, Streptococcus phage A25, Streptococcus pyogenes, Streptococcus pyogenes serotype M2 (strain MGAS10270), Streptococcus pyogenes serotype M4 (strain MGAS10750), Streptococcus pyogenes serotype M3 (strain ATCC BAA-595/MGAS315), Streptococcus pyogenes GA06023, Streptococcus pyogenes STAB902, Streptococcus phage M102, Streptococcus phage MM1 1998, Streptococcus pneumoniae, Streptococcus phage MM1, Streptococcus phage Sfi21, Streptococcus phage V22, Streptococcus pneumoniae, Streptococcus pyogenes serotype M28 (strain MGAS6180), Streptococcus pyogenes, Temperate phage phiNIH11, Streptococcus pyogenes serotype M2 (strain MGAS10270), Streptococcus pyogenes serotype M3 (strain ATCC BAA-595/MGAS315), Streptococcus pyogenes STAB902, Streptococcus pyogenes STAB902, Streptococcus pyogenes, Streptococcus pyogenes serotype M3 (strain ATCC BAA-595/MGAS315), Streptomyces albulus, Streptomyces albus, Streptomyces albus J1074, Streptomyces coelicolor (strain ATCC BAA-471/A3(2)/M145), Streptomyces cyaneogriseus, Streptomyces cyaneogriseus subsp noncyanogenus, Streptomyces longwoodensis, Streptomyces noursei, Streptomyces noursei ATCC 11455, Streptomyces phage VWB, Streptomyces rimosus, Streptomyces rimosus subsp pseudoverticillatus, Streptomyces sp HPH0547, Sulfurovum sp F506-10, Synechococcus phage Syn5, Synechococcus sp UTEX 2973, Synechocystis sp PCC 6803, Thalassomonas phage BA3, Thermaerobacter marianensis (strain ATCC 700841/DSM 12885/JCM10246/7p75a), Thermus phage phiYS40, Thiorhodovibrio sp 970, Treponema socranskii subsp socranskii VPI DR56BR1116=ATCC 35536, Ureaplasma urealyticum serovar 10 (strain ATCC 33699/Western), Ureaplasma urealyticum serovar 7 str ATCC 27819, Ureaplasma parvum serovar 3 (strain ATCC 700970), Ureaplasma urealyticum serovar 8 str ATCC 27618, Ureaplasma urealyticum serovar 4 str ATCC 27816, Ureaplasma urealyticum serovar 12 str ATCC 33696, Vibrio cholerae (strain MO10), Vibrio cholerae, Providencia alcalifaciens Ban1, Vibrio cholerae Ind4, Vibrio cholerae (strain MO10), Vibrio cholerae, Providencia alcalifaciens Ban1, Vibrio cholerae Ind4, Vibrio cholerae 1587, Vibrio natriegens NBRC 15636=ATCC 14048=DSM 759, Xanthobacter autotrophicus (strain ATCC BAA-1158/Py2), Xanthomonas phage OP2, Yersinia phage YpsP-G, Yersinia phage phiA1122, Yersinia phage YpsP-G, and Yersinia phage phiA1122. Other sources may be used.

The source, in some embodiments, is a bacterial cell. The bacterial strain may be, for example, Yersinia spp., Escherichia spp., Klebsiella spp., Agrobacterium spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Lactococcus spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are probiotic cells. In some instances, the source is an Escherichia coli (E. coli) cell, a Lactococcus lactis (L. lactis) cell, Agrobacterium tumefaciens (A. tumefaciens), or a Mycobacterium smegmatis (M. smegmatis) cell.

The source may be a gram-positive bacterial cell. Gram-positive bacterial cells stain positive in a gram stain test and often comprise a thick layer of peptidoglycan in their cell walls. Non-limiting examples of gram-positive bacterial cells include Actinomyces spp., Alicyclobacillus spp., Alicyclobacillus acidoterrestris, Alicyclobacillus aeris, Alicyclobacillus contaminans, Alicyclobacillus cycloheptanicus, Alicyclobacillus dauci, Alicyclobacillus disulfidooxidans, Alicyclobacillus fastidiosus, Alicyclobacillus ferrooxydans, Alicyclobacillus fodiniaquatilis, Alicyclobacillus herbarius, Alicyclobacillus hesperidum, Alicyclobacillus kakegawensis, Alicyclobacillus macrosporangiidus, Alicyclobacillus montanus, Alicyclobacillus pomorum, Alicyclobacillus sacchari, Alicyclobacillus sendaiensis, Alicyclobacillus shizuokensis, Alicyclobacillus tengchongensis, Alicyclobacillus tolerans, Alicyclobacillus vulcanalis, Arcanobacterium spp., Bacillus spp., Bacillus mojavensis, Bavariicoccus spp., Brachybacterium spp., Brachybacterium alimentarium, Brachybacterium aquaticum, Brachybacterium conglomeratum, Brachybacterium endophyticum, Brachybacterium faecium, Brachybacterium fresconis, Brachybacterium ginsengisoli, Brachybacterium horti, Brachybacterium huguangmaarense, Brachybacterium massiliense, Brachybacterium muris, Brachybacterium nesterenkovii, Brachybacterium paraconglomeratum, Brachybacterium phenoliresistens, Brachybacterium rhamnosum, Brachybacterium sacelli, Brachybacterium saurashtrense, Brachybacterium squillarum, Brachybacterium tyrofermentans, Brachybacterium zhongshanense, Brevibacterium linens, Collinsella stercoris, Clostridioides, Clostridioides difficile (bacteria), Clostridium spp., Clostridium acetobutylicum, Clostridium aerotolerans, Clostridium argentinense, Clostridium autoethanogenum, Clostridium baratii, Clostridium beijerinckii, Clostridium bifermentans, Clostridium botulinum, Clostridium butyricum, Clostridium cadaveris, Clostridium cellobioparum, Clostridium cellulolyticum, Clostridium cellulovorans, Clostridium chauvoei, Clostridium clostridioforme, Clostridium colicanis, Clostridium estertheticum, Clostridium fallax, Clostridium formicaceticum, Clostridium histolyticum, Clostridium innocuum, Clostridium kluyveri, Clostridium ljungdahlii, Clostridium novyi, Clostridium paradoxum, Clostridium paraputrificum, Clostridium pasteurianum, Clostridium perfringens, Clostridium phytofermentans, Clostridium piliforme, Clostridium ragsdalei, Clostridium ramosum, Clostridium saccharobutylicum, Clostridium saccharoperbutylacetonicum, Clostridium scatologenes, Clostridium septicum, Clostridium sordellii, Clostridium sporogenes, Clostridium stercorarium, Clostridium sticklandii, Clostridium straminisolvens, Clostridium tertium, Clostridium tetani, Clostridium thermosaccharolyticum, Clostridium tyrobutyricum, Clostridium uliginosum, Cnuibacter spp., Coriobacteriia spp., Corynebacterium, Corynebacterium amycolatum, Corynebacterium bovis, Corynebacterium diphtheriae, Corynebacterium efficiens, Corynebacterium glutamicum, Corynebacterium granulosum, Corynebacterium jeikeium, Corynebacterium macginleyi, Corynebacterium minutissimum, Corynebacterium renale, Corynebacterium ulcerans, Cutibacterium acnes, Deinococcus marmoris, Desulfitobacterium dehalogenans, Effusibacillus consociatus, Effusibacillus lacus, Effusibacillus pohliae, Enterococcus spp., Enterococcus faecalis, Fervidobacterium changbaicum, Fervidobacterium gondwanense, Fervidobacterium islandicum, Fodinibacter spp., Fodinibacter luteus, Gordonia soli, Georgenia ruanii, Humibacillus spp., Intrasporangium spp., Janibacter spp., Knoellia spp., Knoellia aerolata, Knoellia flava, Knoellia locipacati, Knoellia remsis, Knoellia sinensis, Knoellia subterranea, Kribbia spp., Kribbia dieselivorans, Kyrpidia spormannii, Kyrpidia tusciae, Lactobacillus spp., Lactobacillus acidophilus, Lactobacillus buchneri, Lactobacillus casei, Lactococcus lactis, Lactobacillus plantarum, Lactococcus lactis, Lapillicoccus spp., Lapillicoccus jejuensis, Listeriaceae spp., Marihabitans spp., Marihabitans asiaticum, Microbispora corallina, Mycobacterium smegmatis, Nocardia spp., Nocardia asteroides, Nocardia brasiliensis, Nocardia farcinica, Nocardia ignorata, Nonpathogenic organisms, Ornithinibacter spp., Ornithinibacter aureus, Paeniclostridium sordellii, Pasteuria spp., Phycicoccus spp., Pilibacter spp., Propionibacterium freudenreichii, Rathayibacter toxicus, Rhodococcus equi, Roseburia spp., Rothia dentocariosa, Sarcina spp., Solibacillus spp., Sporosarcina spp., Sporosarcina aquimarina, Sporulation in Bacillus subtilis, Staphylococcus, Staphylococcus aureus, Staphylococcus capitis, Staphylococcus caprae, Staphylococcus epidermidis, Staphylococcus haemolyticus, Staphylococcus hominis, Staphylococcus lugdunensis, Staphylococcus lutrae, Staphylococcus muscae, Staphylococcus nepalensis, Staphylococcus pettenkoferi, Staphylococcus pseudintermedius, Staphylococcus saprophyticus, S, Staphylococcus schleiferi, Staphylococcus succinus, Staphylococcus warneri, Staphylococcus xylosus, Streptococcus spp., Streptococcus agalactiae, Streptococcus anginosus, Streptococcus canis, Streptococcus downei, Streptococcus equi, Streptococcus bovis, Streptococcus gordonii, Streptococcus iniae, Streptococcus lactarius, Streptococcus mitis, Streptococcus mutans, Streptococcus oralis, Streptococcus parasanguinis, Streptococcus peroris, Streptococcus pneumoniae, Streptococcus pyogenes, Streptococcus ratti, Streptococcus salivarius, Streptococcus sanguinis, Streptococcus sobrinus, Streptococcus suis, Streptococcus thermophilus, Streptococcus tigurinus, Streptococcus uberis, Streptococcus vestibularis, Syntrophomonas curvata, Syntrophomonas palmitatica, Syntrophomonas sapovorans, Syntrophomonas wolfei, Syntrophomonas zehnderi, Tumebacillus algifaecis, Tumebacillus avium, Tumebacillus flagellatus, Tumebacillus ginsengisoli, Tumebacillus lipolyticus, Tumebacillus luteolus, Tumebacillus permanentifrigoris, Tumebacillus soli, and Viridans streptococci.

The source may be a gram-negative bacterial cell. Gram-negative bacterial cells do not retain the stain in a Gram staining test and often comprise a thinner peptidoglycan layer in their cell walls as compared to gram-positive bacterial cells. Non-limiting examples of gram-negative bacteria include Vibrio aerogenes, Acidaminococcus spp., Acinetobacter baumannii, Agrobacterium tumefaciens, Akkermansia glycaniphila, Akkermansia muciniphila, Anaerobiospirillum, Anaerolinea thermolimosa, Anaerolinea thermophila, Arcobacter spp., Arcobacter skirrowii, Armatimonas rosea, Azotobacter salinestris, Bacteroides spp., Bacteroides caccae, Bacteroides fragilis, Bacteroides thetaiotaomicron, Bacteroides ureolyticus, Bacteroidetes spp., Bartonella japonica, Bartonella koehlerae, Bartonella taylorii, Bdellovibrio spp., Brachyspira spp., Bradyrhizobium japonicum, Budviciaceae spp., Caldilinea aerophila, Cardiobacterium spp., Cardiobacterium hominis, Chaperone-Usher fimbriae, Chishuiella spp., Christensenella spp., Caulobacter crescentus, Chthonomonas calidirosea, Citrobacter freundii, Coxiella burnetii, Cytophaga spp., Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister spp., Dictyoglomus thermophilum, Dinoroseobacter shibae, Enterobacter spp., Enterobacter cloacae, Enterobacter cowanii, Escherichia spp., Escherichia coli, Escherichia fergusonii, Escherichia hermannii, Fimbriimonas ginsengisoli, Flavobacterium spp., Flavobacterium akiainvivens, Fusobacterium necrophorum, Fusobacterium nucleatum, Fusobacterium polymorphum, Gluconacetobacter diazotrophicus, Haemophilus felis, Haemophilus haemolyticus, Haemophilus influenzae, Haemophilus pittmaniae, Helicobacter spp., Helicobacter bizzozeronii, Helicobacter heilmannii s.s, Helicobacter heilmannii sensu lato, Helicobacter salomonis, Helicobacter suis, Helicobacter typhlonius, Kingella kingae, Klebsiella huaxiensis, Klebsiella pneumoniae, Kluyvera ascorbata, Kluyvera cryocrescens, Kozakia baliensis, Legionella spp., Legionella clemsonensis, Legionella pneumophila, Leptonema illini, Leptotrichia buccalis, Levilinea saccharolytica, Luteimonas aestuarii, Luteimonas aquatica, Luteimonas composti, Luteimonas lutimaris, Luteimonas marina, Luteimonas mephitis, Luteimonas vadosa, Mariniflexile spp., Megasphaera spp., Meiothermus spp., Meiothermus timidus, Methylobacterium fujisawaense, Morax-Axenfeld diplobacilli, Moraxella spp., Moraxella bovis, Moraxella osloensis, Morganella morganii, Mycoplasma spumans, Neisseria cinerea, Neisseria gonorrhoeae, Neisseria meningitidis, Neisseria polysaccharea, Neisseria sicca, Nitrosomonas eutropha, Nitrosomonas halophila, Nitrosomonas stercoris, Pelosinus spp., Propionispora vibrioides, Proteus mirabilis, Proteus penneri, Pseudomonas spp., Pseudomonas aeruginosa, Pseudomonas luteola, Pseudomonas teessidea, Pseudoxanthomonas broegbernensis, Pseudoxanthomonas japonensis, Rickettsia parkeri, Rickettsia rickettsii, Salinibacter ruber, Salmonella spp., Salmonella bongori, Salmonella enterica, Samsonia spp., Serratia marcescens, Shigella spp., Shimwellia spp., Solobacterium moorei, Sorangium cellulosum, Sphaerotilus natans, Sphingomonas gei, Sphingosinicella humi, Spirochaeta spp., Sporomusa spp., Stenotrophomonas spp., Stenotrophomonas nitritireducens, Thermotoga neapolitana, Thorselliaceae spp., Vampirococcus spp., Verminephrobacter spp., Vibrio spp., Vibrio adaptatus, Vibrio azasii, Vibrio campbellii, Vibrio cholerae, Victivallis vadensis, Vitreoscilla spp., Wolbachia spp., Yersinia spp., and Zymophilus paucivorans.

### Mismatch Repair Enzymes

Mismatch repair enzymes are involved in the detection of distortions in the secondary structure of DNA caused by incorrectly paired nucleotides and correction of these mismatches. Non-limiting examples of mismatch repair enzymes include MutS, MutH and MutL. Dominant negative mismatch repair enzymes disable mismatch repair. Non-limiting examples of dominant negative MutL include a dominant negative MutL protein that comprises an amino acid substitution corresponding to E32K in E. coli wild-type MutL (SEQ ID NO: 514), E33K in L. lactis wild-type MutL (SEQ ID NO: 512), or E36K in P. aeruginosa wild-type MutL (SEQ ID NO: 548). See, e.g., SEQ ID NOs: 515, 513, or 549.

Without being bound by a particular theory, a dominant negative mismatch repair enzyme may be from the same source as recombinant cell in which is being expressed.

### Variants

The proteins described herein (e.g., SSAPs, SSBs, dominant negative mismatch repair enzymes or exonucleases) may contain one or more amino acid substitutions relative to its wild-type counterpart. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

It should be understood that the present disclosure encompasses the use of any one or more of the SSAPs, SSBs, dominant negative mismatch repair enzymes, or exonucleases described herein as well as a SSAP, SSB, dominant negative mismatch repair enzyme, or exonuclease that share a certain degree of sequence identity with a reference protein. The term “identity” refers to a relationship between the sequences of two or more polypeptides or polynucleotides, as determined by comparing the sequences. Identity measures the percent of identical matches between the smaller of two or more sequences with gap alignments (if any) addressed by a particular mathematical model or computer program (e.g., “algorithms”). Identity of related molecules can be readily calculated by known methods. “Percent (%) identity” as it applies to amino acid or nucleic acid sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Identity depends on a calculation of percent identity but may differ in value due to gaps and penalties introduced in the calculation. Variants of a particular sequence may have at least 70%, 75%, 80%, 85%, 90%, 91%, 92%, 93%, 94%, 95%, 96%, 97%, 98%, 99% but less than 100% sequence identity to that particular reference sequence, as determined by sequence alignment programs and parameters described herein and known to those skilled in the art.

The comparison of sequences and determination of percent identity between two sequences can be accomplished using a mathematical algorithm. Techniques for determining identity are codified in publicly available computer programs. Exemplary computer software to determine homology between two sequences include, but are not limited to, GCG program package (Devereux, J. et al. Nucleic Acids Research, 12(1): 387, 1984), the BLAST suite (Altschul, S. F. et al. Nucleic Acids Res. 25: 3389, 1997), and FASTA (Altschul, S. F. et al. J. Molec. Biol. 215: 403, 1990). Other techniques include: the Smith-Waterman algorithm (Smith, T. F. et al. J. Mol. Biol. 147: 195, 1981; the Needleman-Wunsch algorithm (Needleman, S. B. et al. J. Mol. Biol. 48: 443, 1970; and the Fast Optimal Global Sequence Alignment Algorithm (FOGSAA) (Chakraborty, A. et al. Sci Rep. 3: 1746, 2013).

### Homologous Recombination-Mediated Genetic Engineering (Recombineering)

Aspects of the present disclosure provide methods of homologous recombination-mediated genetic engineering (recombineering) to produce modified cells. The modified cell may be gram-positive or gram-negative. Recombineering refers to integration of an exogenous nucleic acid into the genome of a cell using homologous recombination (genetic recombination in which nucleotide sequences are exchanged between two similar nucleic acid molecules). As used herein, an exogenous nucleic acid is any nucleic acid that is introduced into a cell.

The recombineering methods described herein comprise culturing a recombinant cell that comprises (1) any of the SSAPs described herein and (2) a exogenous nucleic acid comprising a sequence of interest that binds to a target locus. The exogenous nucleic acid may be single-stranded or double-stranded and may comprise ribonucleotides, deoxyribonucleotides, unnatural nucleotides, or a combination thereof. Unnatural nucleotides are nucleic acid analogues and include peptide nucleic acid (PNA), morpholine, locked nucleic acid (LNA), as well as glycol nucleic acid (GNA), threose nucleic acid (TNA). In some instances, a recombinant cell further comprises a SSB, an exonuclease or a combination thereof. For example, a recombinant cell that is capable of integrating an exogenous nucleic acid that is double-stranded may further comprise an exonuclease and SSB. The exonuclease can be used to generate 3′ overhangs of single-stranded nucleic acids for hybridization to a target locus. In some embodiments, the methods further comprise introducing a SSAP, a SSAP and a SSB, SSAP, SSB, and dominant negative mismatch repair enzyme, or a SSAP, SSB, and an exonuclease into the cell.

The exogenous nucleic acid comprising a sequence of interest for use in recombineering is capable of hybridizing to a target locus. The exogenous nucleic acid may be 100% complementary to the target locus or may comprise a nucleotide modification relative to the target locus. Nucleotide modifications include mutations, deletions, insertions, and unnatural nucleotides. In some instances, the exogenous nucleic acid comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 nucleotide modifications relative to the target locus for integration. In some instances, the exogenous nucleic acid comprises a sequence that is at least 10%, at least 20%, at least 30%, at least 40%, at least 50%, at least 60%, at least 70%, at least 80%, at least 90%, or at least 100% complementary to the target locus for integration.

In some instances, the exogenous nucleic acid comprises a contiguous stretch of nucleotides that is complementary to the target locus for integration. The contiguous stretch of nucleotides may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 in length.

In some instances, the exogenous nucleic acid comprises (1) a sequence of interest that is not complementary to the target locus for integration, and (2) flanking sequences (e.g., 10 to 500 nucleotides in length) on either side of the sequence of interest that are each complementary to the target locus for integration. In some instances, the exogenous nucleic acid does not comprise flanking sequences that are each complementary to the target locus for integration.

In some instances, the exogenous nucleic acid does not comprise a contiguous stretch of nucleotides that is complementary to the target locus for integration, but is still capable of binding to the target locus. For example, an exogenous nucleic acid may comprise a sequence that has a mutation at every other nucleotide relative to the target locus, but still binds to the target locus.

One type of recombineering is multiplex automated genomic engineering (MAGE), in which more than one locus in a cell is simultaneously targeted (e.g., targeted for modification). To carry out MAGE, more than one exogenous nucleic acid is introduced into a cell. In some instances, more than one exogenous nucleic acid targeting at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, or at least 10,000 loci in the genome of a cell are introduced into a cell. In some instances, two or more exogenous nucleic acids target the same locus in the genome of a cell. In some instances, at least two exogenous nucleic acids target different loci in the genome of a cell.

As used herein, one cycle of recombineering refers to one round of inducing integration of an exogenous nucleic acid comprising a sequence of interest in one or more cells (e.g., in a population of cells). When the SSAP, SSB, exonuclease, dominant negative mismatch repair enzyme, or a combination thereof is present on an expression vector that comprises a constitutive promoter, induction of integration of an exogenous nucleic acid may comprise introduction of one or more nucleic acids encoding a SSAP, a SSB, dominant negative mismatch repair enzyme, an exonuclease, or a combination thereof and introduction of the exogenous nucleic acid encoding a sequence of interest. When expression of SSAP, SSB, exonuclease, dominant negative mismatch repair enzyme, or a combination thereof is under the control of an inducible promoter and the recombinant cell already comprises a nucleic acid encoding an inducible promoter operably linked to the nucleic acid encoding SSAP, SSB, dominant negative mismatch repair enzyme, exonuclease, or a combination thereof, induction of integration of an exogenous nucleic acid may comprise culturing the cell in the presence of an inducing reagent and introducing the exogenous nucleic acid to the cell. As a non-limiting example, one round of recombineering in a bacteria host cell may comprise (1) growing cells that comprise at least one exogenous nucleic acid encoding an SSAP, SSAP/SSB pair, SSAP, SSB, and dominant negative mismatch repair enzyme, or SSAP, SSB and exonuclease; (2) inducing expression of proteins if expression is under the control of an inducible promoter; (3) making the cells competent (e.g., usually placing the cells on ice and washing with water, but this step may by organism); (4) introducing one or more exogenous nucleic acids comprising a sequence of interest into the cells (e.g., by electroporation); and (5) allowing the cells to rest. For MAGE, each cycle of recombineering may further comprise introducing multiple exogenous nucleic acids targeting at least two different loci in the genome of a cell. See, e.g., Wang et al., Nature. 2009 Aug. 13; 460(7257):894-898.

In some instances, the methods comprise at least 1 cycle, at least 2 cycles, at least 3 cycles, at least 4 cycles, at least 5 cycles, at least 6 cycles, at least 7 cycles, at least 8 cycles, at least 9 cycles, at least 10 cycles, at least 20 cycles, at least 30 cycles, at least 40 cycles, at least 50 cycles, at least 60 cycles, at least 70 cycles, at least 80 cycles, at least 90 cycles, at least 100 cycles, at least 200 cycles, at least 300 cycles, at least 400 cycles, at least 500 cycles, at least 600 cycles, at least 700 cycles, at least 800 cycles, at least 900 cycles, or at least 1,000 cycles of recombineering. For example, the method of recombineering could be MAGE.

The efficiency of recombineering may be measured by any suitable method that detects integration of a sequence of interest into a target locus. As a non-limiting example, the target locus of interest may be amplified in cells following introduction and/or induction of a SSAP, SSB, dominant negative mismatch repair enzyme, an exonuclease, or a combination thereof and sequenced. Polymerase chain reaction (PCR) may be used to amplify the target locus and sequencing methods include Sanger sequencing and next generation sequencing (massively parallel sequencing) technologies. The efficiency of recombineering can be calculated as the frequency of modified alleles compared to the total number of alleles detected in a cell or in a population of cells. In instances in which the target locus to be modified encodes a protein, changes in the activity level of the protein may be used to determine editing efficiency. For example, the editing efficiency of a SSAP, a SSAP and a SSB, SSAP, SSB, and dominant negative mismatch repair enzyme, or a SSAP, SSB, and an exonuclease may be measured in a bacterial cell by using an exogenous nucleic acid encoding a modification to the LacZ locus, which encodes β-galactosidase, and the efficiency of recombineering can be measured as the level of LacZ disruption. Disruption of LacZ can be measured in a β-galactosidase assay. See also, e.g., the Materials and Methods section of the Examples below.

In some instances, the efficiency of recombineering is measured as the percentage of cells comprising the integrated sequence of interest.

The efficiency of recombineering using any of the methods described herein may be at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some instances, a recombinant cell comprising a SSAP, a SSB, dominant negative mismatch repair enzyme, an exonuclease, or a combination thereof has a recombineering efficiency that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold, at least 200-fold, at least 300-fold, at least 400-fold, at least 500-fold, at least 600-fold, at least 700-fold, at least 800-fold, at least 900-fold, or at least 1,000 fold greater as compared to a control cell that is of the same type as the recombinant cell but that does not comprise the SSAP, the SSB, dominant negative mismatch repair enzyme, the exonuclease, or the combination thereof. In some instances, the control cell comprises Redβ SSAP from Enterobacteria phage λ.

As a non-limiting example, a nucleic acid sequence encoding Redβ SSAP from Enterobacteria phage λ is:

(SEQ ID NO: 473)
ATGAGTACTGCACTTGCAACATTAGCTGGCAAGTTAGCAGAGCGTGTTGG
TATGGATTCAGTCGACCCTCAGGAGCTTATAACTACCTTACGTCAAACAG
CGTTCAAGTGTGACGCCTCTGATGCACAATTTATCGCTTTGCTTATCGTA
GCTAACCAGTATGGGTTGAATCCTTGGACGAAGGAGATATACGCTTTCCC
GGATAAGCAGAACGGTATTGTTCCTGTAGTAGGTGTCGATGGATGGAGTA
GAATTATCAATGAAAATCAACAGTTCGATGGCATGGACTTCGAGCAGGAT
AATGAATCATGTACCTGCCGTATATATAGAAAAGACCGAAATCACCCAAT
TTGTGTGACTGAATGGATGGATGAGTGCAGACGTGAGCCGTTCAAGACCC
GAGAAGGCCGTGAAATCACTGGTCCGTGGCAATCACATCCAAAGAGAATG
TTGCGTCACAAGGCGATGATTCAGTGCGCCCGTTTAGCTTTTGGGTTTGC
TGGCATTTACGACAAGGACGAAGCTGAAAGAATCGTTGAAAACACTGCAT
ATACCGCTGAACGACAACCGGAGCGTGACATTACGCCAGTGAATGACGAG
ACAATGCAGGAAATTAACACGTTGTTGATTGCTTTGGACAAAACGTGGGA
CGACGACTTGTTACCACTTTGTAGCCAAATTTTTCGTCGAGACATTAGAG
CTTCATCTGAGCTTACACAAGCTGAAGCCGTCAAGGCATTGGGGTTTTTG
AAACAAAAAGCTACCGAACAGAAGGTAGCGGCATAA.

As an example, an amino acid sequence encoding Redβ SSAP from Enterobacteria phage λ is:

(SEQ ID NO: 474)
MSTALATLAGKLAERVGMDSVDPQELITTLRQTAFKCDASDAQFIALLIV
ANQYGLNPWTKEIYAFPDKQNGIVPVVGVDGWSRIINENQQFDGMDFEQD
NESCTCRIYRKDRNHPICVTEWMDECRREPFKTREGREITGPWQSHPKRM
LRHKAMIQCARLAFGFAGIYDKDEAERIVENTAYTAERQPERDITPVNDE
TMQEINTLLIALDKTWDDDLLPLCSQIFRRDIRASSELTQAEAVKALGFL
KQKATEQKVAA.

The efficiency of recombineering may be measured after at least 1 cycle, at least 2 cycles, at least 3 cycles, at least 4 cycles, at least 5 cycles, at least 6 cycles, at least 7 cycles, at least 8 cycles, at least 9 cycles, at least 10 cycles, at least 20 cycles, at least 30 cycles, at least 40 cycles, at least 50 cycles, at least 60 cycles, at least 70 cycles, at least 80 cycles, at least 90 cycles, at least 100 cycles, at least 200 cycles, at least 300 cycles, at least 400 cycles, at least 500 cycles, at least 600 cycles, at least 700 cycles, at least 800 cycles, at least 900 cycles, or at least 1,000 cycles of recombineering. For example, the method of recombineering could be MAGE.

The efficiency of recombineering may be measured after at least 1 day, at least 2 days, at least 3 days, at least 4 days, at least 5 days, at least 6 days, at least 7 days, at least 8 days, at least 9 days, at least 10 days, at least 20 days, at least 30 days, at least 40 days, at least 50 days, at least 60 days, at least 70 days, at least 80 days, at least 90 days, at least 100 days, at least 200 days, at least 300 days, at least 400 days, at least 500 days, at least 600 days, at least 700 days, at least 800 days, at least 900 days, or at least 1,000 days of recombineering. In some instances, the method of recombineering is MAGE. The recombinant cell may be of any species and may be a prokaryotic cell or a eukaryotic cell. In some instances, the recombinant cell is a bacterial cell. The bacterial strain may be, for example, Yersinia spp., Escherichia spp., Klebsiella spp., Agrobacterium spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Lactococcus spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are probiotic cells. In some instances, the recombinant cell is an Escherichia coli (E. coli) cell, a Lactococcus lactis (L. lactis) cell, Agrobacterium tumefaciens (A. tumefaciens), or a Mycobacterium smegmatis (M. smegmatis) cell.

A recombinant cell may comprise an SSAP, a SSB, dominant negative mismatch repair enzyme, an exonuclease, or a combination thereof that is not naturally expressed in the cell. When a recombinant cell comprises a SSAP and a SSB, the SSAP and SSB may be the same source or from a different source. The source may be the same or different species from that of the recombinant cell. In some instances, a recombinant cell may comprise a SSAP, a SSB, and an exonuclease that are all from different sources. In some instances, at least one protein selected from the SSAP, the SSB, and the exonuclease is from a source that is the same species as the recombinant cell. In some instances, the sources of all three proteins (the SSAP, the SSB, and the exonuclease) are of a different species as compared to the recombinant cell. In some instances, at least one protein selected from the SSAP, the SSB, the dominant negative mismatch repair enzyme, and the exonuclease is from a source that is the same species as the recombinant cell.

To make any of the proteins (e.g., SSAPs, SSBs, dominant negative mismatch repair enzyme, or exonucleases) described herein, a protein of interest can be selected and expressed in a cell using conventional methods, including recombinant technology. For example, a nucleic acid encoding a SSAP, SSB, exonuclease, dominant negative mismatch repair enzyme, or a combination thereof may be introduced into a cell. A nucleic acid, generally, is at least two nucleotides covalently linked together, and in some instances, may contain phosphodiester bonds (e.g., a phosphodiester “backbone”). A nucleic acid is considered “engineered” if it does not occur in nature. Examples of engineered nucleic acids include recombinant nucleic acids and synthetic nucleic acids. In some embodiments, an engineered nucleic acid encodes a SSAP, SSB, exonuclease, dominant negative mismatch repair enzyme, or a combination thereof. In some embodiments, a SSAP or SSB is encoded by separate nucleic acids, while in other embodiments, a single nucleic acid may encode a SSAP and a SSB (e.g., each operably linked to a different promoter, or both operably linked to the same promoter).

Nucleic acids encoding the SSAP, SSB, dominant negative mismatch repair enzyme, exonuclease, or a combination thereof described herein may be introduced into a cell using any known methods, including but not limited to chemical transfection, viral transduction (e.g. using lentiviral vectors, adenovirus vectors, sendaivirus, and adeno-associated viral vectors) and electroporation. For example, methods that do not require genomic integration include transfection of mRNA encoding one or more of the SSAPs, SSBs, or a combination thereof and introduction of episomal plasmids. In some embodiments, the nucleic acids (e.g., mRNA) are delivered to cells using an episomal vector (e.g., episomal plasmid). In other embodiments, nucleic acids encoding a SSAP, SSB, exonuclease, dominant negative mismatch repair enzyme, or a combination thereof may be integrated into the genome of the cell. Genomic integration methods are known, any of which may be used herein, including the use of the PIGGYBAC™ transposon system, sleeping beauty system, lentiviral system, adeno-associated virus system, and the CRISPR gene editing system.

In some embodiments, an engineered nucleic acid is present on an expression plasmid, which is introduced into pluripotent stem cells. In some embodiments, the expression plasmid comprises a selection marker, such as an antibiotic resistance gene (e.g., bsd, neo, hygB, pac, cat, ble, or bla) or a gene encoding a fluorescent protein (RFP, BFP, YFP, or GFP). In some embodiments, an antibiotic resistance gene encodes a puromycin resistance gene. In some embodiments, the selection marker enables selection of cells expressing a SSAP, SSB, exonuclease, dominant negative mismatch repair enzyme, or a combination thereof.

Any of the engineered nucleic acids described herein may be generated using conventional methods. For example, recombinant or synthetic technology may be used to generate nucleic acids encoding the SSAP, SSB, exonuclease, dominant negative mismatch repair enzyme, or a combination thereof described herein. Conventional cloning techniques may be used to insert a SSAP, SSB, exonuclease, dominant negative mismatch repair enzyme, or a combination thereof into an expression plasmid.

In some embodiments, an engineered nucleic acid (optionally present on an expression plasmid) comprises a nucleotide sequence encoding a SSAP, SSB, exonuclease, dominant negative mismatch repair enzyme, or a combination thereof operably linked to a promoter (promoter sequence). In some embodiments, the promoter is an inducible promoter (e.g., comprising a tetracycline-regulated sequence). Inducible promoters enable, for example, temporal and/or spatial control of SSAP, SSB, exonuclease, dominant negative mismatch repair enzyme, or a combination thereof expression.

A promoter control region of a nucleic acid sequence at which initiation and rate of transcription of the remainder of a nucleic acid sequence are controlled. A promoter may also contain sub-regions at which regulatory proteins and molecules may bind, such as RNA polymerase and transcription factors. Promoters may be constitutive, inducible, activatable, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

An inducible promoter is one that is characterized by initiating or enhancing transcriptional activity when in the presence of, influenced by or contacted by an inducing agent. An inducing agent may be endogenous or a normally exogenous condition, compound or protein that contacts an engineered nucleic acid in such a way as to be active in inducing transcriptional activity from the inducible promoter.

Inducible promoters for use in accordance with the present disclosure include any inducible promoter described herein or known to one of ordinary skill in the art. Examples of inducible promoters include, without limitation, chemically/biochemically-regulated and physically-regulated promoters such as saccharide-regulated promoters (e.g., arabinose-responsive promoter and xylose-responsive promoters) alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some instances, the promoter (e.g., for use in E. Coli) is an arabinose inducible promoter. As a non-limiting example, the arabinose inducible promoter is a rhamnose-inducible promoter or pL from lamda phage. In some instances, the inducible promoter is a nisin inducible promoter. For example, a nisin inducible promoter may be used in Lactis spp. In some instances, the inducible promoter is a tetracycline inducible promoter. As a non-limiting example, a tetracycline inducible promoter may be used in Mycobacterium spp.

In some instances, the promoter is a p23 promoter (i.e., an auto-inducible expression system comprising the srfA promoter (P_{srfA}), which could be activated by the signal molecules acting in the quorum-sensing pathway for competence). See, e.g., Guan et al., Microb Cell Fact. 2016 Apr. 25; 15:66. For example, a p23 promoter may be used in Staphylococcus aureus or in Bacillus subtillis cells.

As used herein, a native promoter refers to a promoter that is naturally operably linked to a nucleic acid encoding a protein of interest (e.g., SSAP or SSB) and a non-native promoter refers to a promoter that is not naturally operably linked to a nucleic acid encoding the protein of interest (e.g., a SSAP or SSB). For example, as long as the promoter does not naturally drive expression of a nucleic acid encoding a protein of interest, an engineered nucleic acid comprising a non-native promoter may be a promoter that naturally exists in a cell in which the engineered nucleic acid is introduced. In some instances, the non-native promoter on the engineered nucleic acid is a promoter that does not naturally exist in the cell in which the engineered nucleic acid is introduced. As a non-limiting example, a recombinant cell may comprise an engineered nucleic acid encoding a SSAP or SSB that is from a phage. The phage genome naturally comprises a promoter that naturally drives expression of the SSAP or SSB. In this case, a non-native promoter is a promoter that is not the phage promoter that normally drives expression of the SSAP or SSB. In some instances, a recombinant cell may comprise an engineered nucleic acid encoding a SSAP or SSB that is naturally encoded by the cell and the cell comprises a promoter that is operably linked to the nucleic acid encoding the SSAP or SSB. In this case, a non-native promoter is any promoter that is not the natural promoter in the cell that normally drives expression of the SSAP or SSB. In some instances, a recombinant cell may comprise an engineered nucleic acid encoding a SSAP or SSB that is naturally encoded by another cell and the other cell comprises a promoter that is operably linked to the nucleic acid encoding the SSAP or SSB. In this case, a non-native promoter is any promoter that is not the natural promoter in the other cell that normally drives expression of the SSAP or SSB.

Without being bound by a particular theory, use of a non-native promoter allows for expression of a SSAP, SSB, exonuclease, dominant negative mismatch repair enzyme, or a combination thereof above basal levels in a cell. In some instances, expression from a non-native promoter increases expression of a protein of interest (e.g., SSAP or SSB) by at least 1.5-fold, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500 fold, at least 600-fold, at least 700-fold, at least 800-fold, at least 900-fold, or at least 1,000-fold as compared to expression from the native promoter.

In some embodiments, a vector encoding a SSAP, SSB, exonuclease, dominant negative mismatch repair enzyme, or a combination thereof comprises a ribosome binding site (RBS). A RBS promotes initiation of protein translation. In some embodiments, a RBS comprises a sequence that is at least 50%, at least 60%, at least 70%, at least 80%, at least 90%, at least 91%, at least 92%, at least 93%, at least 94%, at least 95%, at least 96%, at least 97%, at least 98%, or at least 99% identical to a sequence selected from SEQ ID NOs: 505-511. In some embodiments, a RBS comprises a sequence selected from SEQ ID NOs: 505-511.

In some embodiments, a nucleic acid encoding a SSAP, SSB, exonuclease, dominant negative mismatch repair enzyme, or a combination thereof is codon-optimized for expression in a particular type of bacterial cell. In some embodiments, a nucleic acid encoding a SSAP, SSB, exonuclease, dominant negative mismatch repair enzyme, or a combination thereof is not codon-optimized.

### Additional Aspects and Embodiments of the Present Disclosure

In some aspects, the present disclosure provides a recombinant Escherichia coli (E. coli) cell comprising a single-stranded annealing protein (SSAP) selected from the group consisting of: a SSAP from a bacteriophage that can infect, or from a prophage that is stably integrated into the genome of, Thalassomonas phage, a SSAP from a bacteriophage that can infect, or from a prophage that is stably integrated into the genome of, Herbaspirillum sp., a SSAP from a bacteriophage that can infect, or from a prophage that is stably integrated into the genome of, Vibrio cholerae, a SSAP from a bacteriophage that can infect, or from a prophage that is stably integrated into the genome of, Helicobacter pullorum, and a SSAP from a bacteriophage that can infect, or from a prophage that is stably integrated into the genome of, Methyloversatilis universalis. In some embodiments, the SSAP from a bacteriophage that can infect, or from a prophage that is stably integrated into the genome of, Thalassomonas phage comprises the amino acid sequence of SEQ ID NO: 19, the SSAP from a bacteriophage that can infect, or from a prophage that is stably integrated into the genome of, Herbaspirillum sp. comprises the amino acid sequence of SEQ ID NO: 201, the SSAP from a bacteriophage that can infect, or from a prophage that is stably integrated into the genome of, Vibrio cholera comprises the amino acid sequence of SEQ ID NO: 63, the SSAP from a bacteriophage that can infect, or from a prophage that is stably integrated into the genome of, Helicobacter pullorum comprises the amino acid sequence of SEQ ID NO: 128, and the SSAP from a bacteriophage that can infect, or from a prophage that is stably integrated into the genome of, Methyloversatilis universalis comprises the amino acid sequence of SEQ ID NO: 210.

In some embodiments, the E. coli cell further comprises an exogenous nucleic acid comprising a sequence of interest. In some embodiments, the nucleic acid is integrated in the genome of the E. coli cell. In some embodiments, the nucleic acid is a single-stranded DNA. In some embodiments, the nucleic acid is a double-stranded DNA.

Also provided herein are methods comprising culturing the recombinant E. coli cell and producing a modified E. coli cell comprising the sequence of interest.

In other aspects, the present disclosure provides a recombinant Lactococcus lactis (L. lactis) cell comprising a single-stranded annealing protein (SSAP) from a bacteriophage that can infect, or from a prophage that is stably integrated into the genome of, Enterococcus faecalis and a single-stranded binding protein (SSB) selected from the group consisting of: a SSB from a bacteriophage that can infect, or from a prophage that is stably integrated into the genome of, Streptococcus sp., a SSB from a bacteriophage that can infect, or from a prophage that is stably integrated into the genome of, Lactobacillus sp., and a SSB from a bacteriophage that can infect, or from a prophage that is stably integrated into the genome of, Pseudomonas sp. In some embodiments, the SSAP from a bacteriophage that can infect, or from a prophage that is stably integrated into the genome of, Enterococcus faecalis comprises the amino acid sequence of SEQ ID NO: 5. In some embodiments, the SSB from a bacteriophage that can infect, or from a prophage that is stably integrated into the genome of, Streptococcus sp. comprises the amino acid sequence of SEQ ID NO: 366, the SSB from a bacteriophage that can infect, or from a prophage that is stably integrated into the genome of, Lactobacillus sp. comprises the amino acid sequence of SEQ ID NO: 381, and/or the SSB from a bacteriophage that can infect, or from a prophage that is stably integrated into the genome of, Pseudomonas sp. comprises the amino acid sequence of SEQ ID NO: 395.

In yet other aspects, the present disclosure provides a recombinant Lactococcus lactis (L. lactis) cell comprising a single-stranded annealing protein (SSAP) from a bacteriophage that can infect, or from a prophage that is stably integrated into the genome of, Clostridium sp. and a single-stranded binding protein (SSB) selected from the group consisting of: a SSB from a bacteriophage that can infect, or from a prophage that is stably integrated into the genome of, Escherichia coli, a SSB from a bacteriophage that can infect, or from a prophage that is stably integrated into the genome of, Haemophilus influenzae, a SSB from a bacteriophage that can infect, or from a prophage that is stably integrated into the genome of, Streptococcus sp., and a SSB from a bacteriophage that can infect, or from a prophage that is stably integrated into the genome of, Lactobacillus sp. In some embodiments, the SSAP from a bacteriophage that can infect, or from a prophage that is stably integrated into the genome of, Clostridium sp. comprises the amino acid sequence of SEQ ID NO: 143. In some embodiments, the SSB from a bacteriophage that can infect, or from a prophage that is stably integrated into the genome of, Escherichia coli comprises the amino acid sequence of SEQ ID NO: 262, the SSB from a bacteriophage that can infect, or from a prophage that is stably integrated into the genome of, Haemophilus influenza comprises the amino acid sequence of SEQ ID NO: 325, the SSB from a bacteriophage that can infect, or from a prophage that is stably integrated into the genome of, Streptococcus sp. comprises the amino acid sequence of SEQ ID NO: 366, and/or the SSB from a bacteriophage that can infect, or from a prophage that is stably integrated into the genome of, Lactobacillus sp. comprises the amino acid sequence of SEQ ID NO: 381.

In some embodiments, the L. lactis cell further comprises an exogenous nucleic acid comprising a sequence of interest. In some embodiments, the nucleic acid is integrated in the genome of the L. lactis cell. In some embodiments, the nucleic acid is a single-stranded DNA. In some embodiments, the nucleic acid is a double-stranded DNA.

Also provided herein are methods comprising culturing the recombinant L. lactis cell and producing a modified L. lactis cell comprising the sequence of interest.

In further aspects, the present disclosure provides a recombinant Mycobacterium smegmatis (M. smegmatis) cell comprising a single-stranded annealing protein (SSAP) selected from the group consisting of: a SSAP from a bacteriophage that can infect, or from a prophage that is stably integrated into the genome of, Clostridium sp., a SSAP from a bacteriophage that can infect, or from a prophage that is stably integrated into the genome of, Microbacterium ginsengisoli, a SSAP from a bacteriophage that can infect, or from a prophage that is stably integrated into the genome of, Streptomyces sp., and a SSAP from a bacteriophage that can infect, or from a prophage that is stably integrated into the genome of, Nocardia farcinica. In some embodiments, the SSAP from a bacteriophage that can infect, or from a prophage that is stably integrated into the genome of, Clostridium sp. comprises the amino acid sequence of SEQ ID NO: 143, the SSAP from a bacteriophage that can infect, or from a prophage that is stably integrated into the genome of, Microbacterium ginsengisoli comprises the amino acid sequence of SEQ ID NO: 178, the SSAP from a bacteriophage that can infect, or from a prophage that is stably integrated into the genome of, Streptomyces sp. comprises the amino acid sequence of SEQ ID NO: 140, and/or the SSAP from a bacteriophage that can infect, or from a prophage that is stably integrated into the genome of, Nocardia farcinica comprises the amino acid sequence of SEQ ID NO: 175.

In some embodiments, the M. smegmatis cell further comprises a single-stranded binding protein (SSB).

In some embodiments, the M. smegmatis cell further comprises an exogenous nucleic acid comprising a sequence of interest. In some embodiments, the nucleic acid is integrated in the genome of the M. smegmatis cell. In some embodiments, the nucleic acid is a single-stranded DNA. In some embodiments, the nucleic acid is a double-stranded DNA.

Also provided herein are methods comprising culturing the recombinant M. smegmatis cell and producing a modified M. smegmatis cell comprising the sequence of interest.

In additional aspects, the present disclosure provides a recombinant Escherichia coli (E. coli) cell comprising: a single-stranded annealing protein (SSAP) selected from a bacteriophage that can infect, or from a prophage that is stably integrated into the genome of, the group consisting of a SSAP from a bacteriophage that can infect, or from a prophage that is stably integrated into the genome of, Collinsella stercoris, a SSAP from a bacteriophage that can infect, or from a prophage that is stably integrated into the genome of, Thalassomonas sp., a SSAP from a bacteriophage that can infect, or from a prophage that is stably integrated into the genome of, Vibrio cholera, and a SSAP from a bacteriophage that can infect, or from a prophage that is stably integrated into the genome of, Helicobacter pullorum; and a single-stranded binding protein (SSB) selected from a bacteriophage that can infect, or from a prophage that is stably integrated into the genome of, the group consisting of a SSB from a bacteriophage that can infect, or from a prophage that is stably integrated into the genome of, Streptococcus pyogenes, a SSB from a bacteriophage that can infect, or from a prophage that is stably integrated into the genome of, Sodalis glossinidius, a SSB from a bacteriophage that can infect, or from a prophage that is stably integrated into the genome of, Clostridium botulinum, a SSB from a bacteriophage that can infect, or from a prophage that is stably integrated into the genome of, Salmonella sp., a SSB from a bacteriophage that can infect, or from a prophage that is stably integrated into the genome of, Gordonia soli, a SSB from a bacteriophage that can infect, or from a prophage that is stably integrated into the genome of, Paeniclostridium sordellii, and a SSB from a bacteriophage that can infect, or from a prophage that is stably integrated into the genome of, Staphylococcus aureus. In some embodiments, the SSAP from a bacteriophage that can infect, or from a prophage that is stably integrated into the genome of, Collinsella stercoris comprises the amino acid sequence of SEQ ID NO: 157, the SSAP from a bacteriophage that can infect, or from a prophage that is stably integrated into the genome of, Thalassomonas sp. comprises the amino acid sequence of SEQ ID NO: 19, the SSAP from a bacteriophage that can infect, or from a prophage that is stably integrated into the genome of, Vibrio cholera comprises the amino acid sequence of SEQ ID NO: 63, and/or the SSAP from a bacteriophage that can infect, or from a prophage that is stably integrated into the genome of, Helicobacter pullorum comprises the amino acid sequence of SEQ ID NO: 128; and/or the SSB from a bacteriophage that can infect, or from a prophage that is stably integrated into the genome of, Streptococcus pyogenes comprises the amino acid sequence of SEQ ID NO: 235, the SSB from a bacteriophage that can infect, or from a prophage that is stably integrated into the genome of, Sodalis glossinidius comprises the amino acid sequence of SEQ ID NO: 281, the SSB from a bacteriophage that can infect, or from a prophage that is stably integrated into the genome of, Clostridium botulinum comprises the amino acid sequence of SEQ ID NO: 300, the SSB from a bacteriophage that can infect, or from a prophage that is stably integrated into the genome of, Salmonella sp. comprises the amino acid sequence of SEQ ID NO: 308, the SSB from a bacteriophage that can infect, or from a prophage that is stably integrated into the genome of, Gordonia soli comprises the amino acid sequence of SEQ ID NO: 382, the SSB from a bacteriophage that can infect, or from a prophage that is stably integrated into the genome of, Paeniclostridium sordellii comprises the amino acid sequence of SEQ ID NO: 384, and/or the SSB from a bacteriophage that can infect, or from a prophage that is stably integrated into the genome of, Staphylococcus aureus comprises the amino acid sequence of SEQ ID NO: 460.

In additional aspects, the present disclosure provides a recombinant Lactococcus lactis (L. lactis) cell comprising: a single-stranded annealing protein (SSAP) selected from a bacteriophage that can infect, or from a prophage that is stably integrated into the genome of, the group consisting of a SSAP from a bacteriophage that can infect, or from a prophage that is stably integrated into the genome of, Enterococcus faecalis, a SSAP from a bacteriophage that can infect, or from a prophage that is stably integrated into the genome of, Agrobacterium rhizogenes, a SSAP from a bacteriophage that can infect, or from a prophage that is stably integrated into the genome of, Clostridium sp., and a SSAP from a bacteriophage that can infect, or from a prophage that is stably integrated into the genome of, Clostridium botulinum; and a single-stranded binding protein (SSB) selected from a bacteriophage that can infect, or from a prophage that is stably integrated into the genome of, the group consisting of a SSB from a bacteriophage that can infect, or from a prophage that is stably integrated into the genome of, Escherichia coli, a SSB from a bacteriophage that can infect, or from a prophage that is stably integrated into the genome of, Enterobacteria sp., a SSB from a bacteriophage that can infect, or from a prophage that is stably integrated into the genome of, Haemophilus influenza, a SSB from a bacteriophage that can infect, or from a prophage that is stably integrated into the genome of, Streptococcus, a SSB from a bacteriophage that can infect, or from a prophage that is stably integrated into the genome of, Desulfitobacterium metallireducens, a SSB from a bacteriophage that can infect, or from a prophage that is stably integrated into the genome of, Lactobacillus sp., and a SSB from a bacteriophage that can infect, or from a prophage that is stably integrated into the genome of, Pseudomonas sp. In some embodiments, the SSAP from a bacteriophage that can infect, or from a prophage that is stably integrated into the genome of, Enterococcus faecalis comprises the amino acid sequence of SEQ ID NO: 5, the SSAP from a bacteriophage that can infect, or from a prophage that is stably integrated into the genome of, Agrobacterium rhizogenes comprises the amino acid sequence of SEQ ID NO: 7, the SSAP from a bacteriophage that can infect, or from a prophage that is stably integrated into the genome of, Clostridium sp. comprises the amino acid sequence of SEQ ID NO: 143, and/or the SSAP from a bacteriophage that can infect, or from a prophage that is stably integrated into the genome of, Clostridium botulinum comprises the amino acid sequence of SEQ ID NO: 37; and/or the SSB from a bacteriophage that can infect, or from a prophage that is stably integrated into the genome of, Escherichia coli comprises the amino acid sequence of SEQ ID NO: 262, the SSB from a bacteriophage that can infect, or from a prophage that is stably integrated into the genome of, Enterobacteria sp. comprises the amino acid sequence of SEQ ID NO: 284, the SSB from a bacteriophage that can infect, or from a prophage that is stably integrated into the genome of, Haemophilus influenza comprises the amino acid sequence of SEQ ID NO: 325, the SSB from a bacteriophage that can infect, or from a prophage that is stably integrated into the genome of, Streptococcus comprises the amino acid sequence of SEQ ID NO: 366, the SSB from a bacteriophage that can infect, or from a prophage that is stably integrated into the genome of, Desulfitobacterium metallireducens comprises the amino acid sequence of SEQ ID NO: 368, the SSB from a bacteriophage that can infect, or from a prophage that is stably integrated into the genome of, Lactobacillus sp. comprises the amino acid sequence of SEQ ID NO: 381, and the SSB from a bacteriophage that can infect, or from a prophage that is stably integrated into the genome of, Pseudomonas sp. comprises the amino acid sequence of SEQ ID NO: 395.

### Additional Embodiments

Additional embodiments of the present disclosure are provided in the following numbered paragraphs:

Paragraph 1. A recombinant bacterial cell comprising a single-stranded annealing protein (SSAP) from a bacteriophage that can infect Collinsella stercoris, wherein the SSAP is expressed from a non-native promoter.

Paragraph 2. The recombinant bacterial cell of paragraph 1, wherein the recombinant bacterial cell is selected from the group consisting of a recombinant Escherichia coli cell, a recombinant Klebsiella pneumoniae cell, a recombinant Salmonella enterica cell, and a recombinant Citrobacter freundii cell.

Paragraph 3. A recombinant Escherichia coli (E. coli) cell comprising a single-stranded annealing protein (SSAP) from a bacteriophage that can infect Collinsella stercoris.

Paragraph 4. The recombinant E. coli cell of paragraph 3, wherein the SSAP comprises the amino acid sequence of SEQ ID NO: 157.

Paragraph 5. The recombinant E. coli cell of paragraph 3 or 4, wherein the cell further comprises a single-stranded binding protein (SSB).

Paragraph 6. The recombinant E. coli cell of paragraph 5, wherein the SSB is selected from the group consisting of: a SSB from a bacteriophage that can infect Clostridium botulinum, a SSB from a bacteriophage that can infect Gordonia soli, a SSB from a bacteriophage that can infect Paeniclostridium sordellii, and a SSB from a bacteriophage that can infect Enterococcus faecalis.

Paragraph 7. The recombinant E. coli cell of paragraph 6, wherein the SSB from a bacteriophage that can infect Clostridium botulinum comprises the amino acid sequence of SEQ ID NO: 300, the SSB from a bacteriophage that can infect Gordonia soli comprises the amino acid sequence of SEQ ID NO: 382, the SSB from a bacteriophage that can infect Paeniclostridium sordellii comprises the amino acid sequence of SEQ ID NO: 384, and/or the SSB from a bacteriophage that can infect Enterococcus faecalis comprises the amino acid sequence of SEQ ID NO: 389.

Paragraph 8. The recombinant E. coli cell of paragraph 6, wherein the SSB is from a bacteriophage that can infect Gordonia soli, optionally comprising the amino acid sequence of SEQ ID NO: 382.

Paragraph 9. The recombinant E. coli cell of paragraph 6, wherein the SSB is from a bacteriophage that can infect Paeniclostridium sordellii, optionally comprising the amino acid sequence of SEQ ID NO: 384.

Paragraph 10. A method, comprising

culturing a recombinant Escherichia coli (E. coli) cell that comprises (a) a single-stranded annealing protein (SSAP) from a bacteriophage that can infect Collinsella stercoris and (b) a nucleic acid comprising a sequence of interest that binds to a target locus of the E. coli cell genome, wherein the sequence of interest comprises a nucleotide modification relative to the target locus, and

producing a modified E. coli cell comprising the sequence of interest at the target locus.

Paragraph 11. The method of paragraph 10, wherein the modification is a mutation, insertion, and/or deletion.

Paragraph 12. A recombinant Lactococcus lactis (L. lactis) cell comprising a single-stranded annealing protein (SSAP) from a bacteriophage that can infect Enterococcus faecalis.

Paragraph 13. The recombinant L. lactis cell of paragraph 12, wherein the SSAP comprises the amino acid sequence of SEQ ID NO: 5.

Paragraph 14. A recombinant Lactococcus lactis (L. lactis) cell comprising a single-stranded annealing protein (SSAP) from a bacteriophage that can infect Clostridium sp.

Paragraph 15. The recombinant L. lactis cell of paragraph 14, wherein the SSAP comprises the amino acid sequence of SEQ ID NO: 143.

Paragraph 16. The recombinant L. lactis cell of any one of paragraphs 12-15, wherein the cell further comprises a single-stranded binding protein (SSB).

Paragraph 17. The recombinant L. lactis cell of paragraph 16, wherein the SSB is from a bacteriophage that can infect Streptococcus sp.

Paragraph 18. The L. lactis cell of paragraph 17, wherein the SSB comprises the amino acid sequence of SEQ ID NO: 366.

Paragraph 19. A method, comprising

culturing a recombinant Lactococcus lactis (L. lactis) cell comprising a single-stranded annealing protein (SSAP) from a bacteriophage that can infect Enterococcus faecalis and (b) an nucleic acid comprising a sequence of interest that binds to a target locus of the L. lactis cell genome, wherein the sequence of interest comprises a nucleotide modification relative to the target locus, and

producing a modified L. lactis cell comprising the sequence of interest at the target locus.

Paragraph 20. A method, comprising

culturing a recombinant Lactococcus lactis (L. lactis) cell comprising a single-stranded annealing protein (SSAP) from a bacteriophage that can infect Clostridium sp. and (b) an nucleic acid comprising a sequence of interest that binds to a target locus of the L. lactis cell genome, wherein the sequence of interest comprises a nucleotide modification relative to the target locus, and

producing a modified L. lactis cell comprising the sequence of interest at the target locus.

Paragraph 21. A recombinant Mycobacterium smegmatis (M. smegmatis) cell comprising a single-stranded annealing protein (SSAP) from a bacteriophage that can infect Legionella pneumophila.

Paragraph 22. The recombinant M. smegmatis cell of paragraph 21, wherein the SSAP comprises the amino acid sequence of SEQ ID NO: 44.

Paragraph 23. The recombinant M. smegmatis cell of paragraph 21 or 22, wherein the cell further comprises a single-stranded binding protein (SSB).

Paragraph 24. A method, comprising

culturing a recombinant Mycobacterium smegmatis (M. smegmatis) cell comprising a single-stranded annealing protein (SSAP) from a bacteriophage that can infect Legionella pneumophila and (b) an nucleic acid comprising a sequence of interest that binds to a target locus of the M. smegmatis cell genome, wherein the sequence of interest comprises a nucleotide modification relative to the target locus, and

producing a modified M. smegmatis cell comprising the sequence of interest at the target locus.

Paragraph 25. The recombinant cell of any one of the foregoing paragraphs, wherein the cell further comprises an exogenous nucleic acid comprising a sequence of interest that binds to a target locus of the cell genome, wherein the sequence of interest comprises a nucleotide modification relative to the target locus.

Paragraph 26. The recombinant cell of paragraph 25, wherein the nucleic acid is a single-stranded DNA.

Paragraph 27. The recombinant cell of paragraph 25, wherein the nucleic acid is a double-stranded DNA.

Paragraph 28. The recombinant cell of any one of paragraphs 25-27, wherein the nucleic acid is integrated in the genome of the cell.

Paragraph 29. A method, comprising

culturing the recombinant cell of any one of paragraphs 25-27 and producing a modified cell comprising the sequence of interest at the target locus.

Paragraph 30. A method of editing the genome of Escherichia coli (E. coli) cells, comprising

performing multiplexed automatable genome engineering (MAGE) in E. coli cells that comprise (a) a single-stranded annealing protein (SSAP) from a bacteriophage that can infect Collinsella stercoris and (b) at least two exogenous nucleic acids, each comprising a sequence of interest that binds to at least one target locus of the E. coli cell genome, wherein the sequence of interest comprises a nucleotide modification relative to the target locus, and

producing modified E. coli cells comprising the sequence of interest at the target locus.

Paragraph 31. The method of paragraph 30, wherein the SSAP comprises the amino acid sequence of SEQ ID NO: 157.

Paragraph 32. The method of paragraph 30 or 31, wherein at least 50% of the cells comprise the sequence of interest, optionally following 5-10 cycles of MAGE.

Paragraph 33. The method of paragraph 30 or 31, wherein the E. coli cells further comprise a single-stranded binding protein (SSB) from a bacteriophage that can infect Paeniclostridium sordellii.

Paragraph 34. The method of paragraph 33, wherein the SSB comprises the amino acid sequence of SEQ ID NO: 384.

Paragraph 35. The method of paragraph 33 or 34, wherein at least 50% of the cells comprise the sequence of interest, optionally following 5-10 cycles of MAGE.

Paragraph 36. The method of paragraph 35, wherein at least 75% of the cells comprise the sequence of interest, optionally following 5-10 cycles of MAGE.

Paragraph 37. A recombinant bacterial cell comprising a single-stranded annealing protein (SSAP) from a bacteriophage that can infect Pseudomonas aeruginosa, wherein the SSAP is expressed from a non-native promoter.

Paragraph 38. The recombinant bacterial cell of paragraph 37, wherein the SSAP comprises the amino acid sequence of SEQ ID NO: 24.

Paragraph 39. The recombinant bacterial cell of paragraph 37 or 38, wherein the recombinant bacterial cell is selected from the group consisting of a recombinant Klebsiella pneumoniae cell, a recombinant Salmonella enterica cell, and a recombinant Citrobacter freundii cell.

Paragraph 40. The recombinant bacterial cell of any one of paragraphs 37-39, wherein the cell further comprises a single-stranded binding protein (SSB).

Paragraph 41. The recombinant bacterial cell of any one of paragraphs 37-40, wherein the cell further comprises an exogenous nucleic acid comprising a sequence of interest that binds to a target locus of the cell genome, wherein the sequence of interest comprises a nucleotide modification relative to the target locus.

Paragraph 42. The recombinant bacterial cell of paragraph 41, wherein the nucleic acid is a single-stranded DNA.

Paragraph 43. The recombinant bacterial cell of paragraph 41, wherein the nucleic acid is a double-stranded DNA.

Paragraph 44. The recombinant bacterial cell of any one of paragraphs 41-43, wherein the nucleic acid is integrated in the genome of the cell.

Paragraph 45. A method, comprising culturing the cell of any one of paragraphs 41-43 and producing a modified cell comprising the sequence of interest at the target locus.

Paragraph 46. A recombinant bacterial cell comprising a single-stranded annealing protein (SSAP) and/or a single-stranded binding protein (SSB) of Table 1 expressed from a non-native promoter.

Paragraph 47. The recombinant bacterial cell of paragraph 46, wherein the cell further comprises an exogenous nucleic acid comprising a sequence of interest that binds to a target locus of the cell genome, wherein the sequence of interest comprises a nucleotide modification relative to the target locus.

Paragraph 48. The recombinant bacterial cell of paragraph 47, wherein the nucleic acid is a single-stranded DNA.

Paragraph 49. The recombinant bacterial cell of paragraph 47, wherein the nucleic acid is a double-stranded DNA.

Paragraph 50. The recombinant bacterial cell of any one of paragraphs 47-49, wherein the nucleic acid is integrated in the genome of the cell.

Paragraph 51. A method, comprising

culturing the recombinant bacterial cell of any one of paragraphs 47-49 and producing a modified bacterial cell comprising the sequence of interest at the target locus.

Paragraph 52. A method, comprising

(i) introducing into a recombinant cell: (a) a single-stranded annealing protein (SSAP), (b) a single-stranded binding protein (SSB), and (c) a double-stranded nucleic acid comprising a sequence of interest that binds to a genomic target locus of the recombinant cell, wherein the sequence of interest comprises a nucleotide modification relative to the target locus, and

(ii) producing a modified recombinant cell comprising the sequence of interest at the target locus, wherein the modified recombinant cell does not express an exogenous exonuclease.

Paragraph 53. The method of paragraph 52, wherein (a) and (b) are from the same species.

Paragraph 54. The method of paragraph 52, wherein (a) and (b) are from different species.

Paragraph 55. The method of any one of paragraphs 52-54, wherein the SSAP comprises SEQ ID NO: 24.

Paragraph 56. The method of any one of paragraphs 52-55, wherein the SSB comprises SEQ ID NO: 472.

Paragraph 57. The method of paragraph 36, wherein at least 95% of the cells comprise the sequence of interest following 15 cycles of MAGE.

Paragraph 58. The method of paragraph 36, wherein following 15 cycles of MAGE, the percentage of cells comprising the sequence of interest is at least four-fold greater as compared to control E. coli cells that comprise (a) a Redβ SSAP from Enterobacteria phage λ (SEQ ID NO: 474) and (b) the at least two exogenous nucleic acids, each comprising the sequence of interest that binds to a different target locus of the control E. coli cell genome, wherein the sequence of interest comprises the nucleotide modification relative to the target locus.

### EXAMPLES

### Example 1

A library of 234 SSAPs were tested both individually and co-expressed with a library of 237 SSBs (Table 1, below). In the SSAP/SSB library, SSAPs and SSBs were both individually enriched, so matrices to test all combinations of the top seven enriched SSBs against the top four enriched SSAPs in E. coli and L. lactis were constructed (FIGS. 1A-1B). The experiment was carried out in a 96-well electroporation set-up. The relative efficiencies are clearly discernable.

Top-performing SSAPs and SSAP/SSB pairs from experiments in E. coli, L. lactis, and M. smegmatis are shown in FIG. 2A, FIG. 2B and FIG. 2C, respectively. Bars in red are the proteins that had previously been reported in the literature. The proteins listed were found after ten rounds of selection for protein variants that enabled the introduction of an oligonucleotide that conferred a genomic edit that provided antibiotic resistance. Unbiased editing efficiency was tested in each case by introducing a non-coding base change at a non-essential gene and measuring the frequency of incorporation via next generation sequencing.

### Example 2

E. coli populations expressing either an efficient SSAP (SEQ ID NO: 157), an efficient SSAP/SSB pair (SEQ ID NO: 157/SEQ ID NO: 384), or the widely-used Redβ were taken through fifteen cycles of MAGE and transformed each cycle with a 10 μM pool comprising 15 unique oligos. Editing efficiency at each targeted locus was measured by NGS and averaged (FIG. 3).

The results showed the high-efficiency of SEQ ID NO: 157/SEQ ID NO: 384 for gene editing. This pair incorporated at close to 100% efficiency 15 separate mutations in one week, as compared to Redβ, which in the same time incorporated only at ˜20% efficiency.

### Example 3

The efficiency of genome-editing was tested in species that had not been tested in the previously-mentioned libraries. SSAP SEQ ID NO: 24, a high-efficiency SSAP from Pseudomonas aeruginoas (P. aeruginosa) was identified by an early experiment in E. coli. This protein displayed improved annealing kinetics in vitro (FIG. 4A). It showed improved efficiency over Redβ in many clinically relevant species of Gammaproteobacteria (FIG. 4B). In P. aeruginosa, it enabled rapid multi-drug resistance profiling (FIG. 4C). Four oligonucleotides were incorporated in one day and two cycles of MAGE, conferring resistance to three antibiotics at once.

SSAP SEQ ID NO: 24 has not previously been described, and it displayed high activity in many clinically relevant Gammaproteobacteria. Pseudomonas aeruginosa, Klebsiella pneumoniae, and Salmonella enterica were all chosen for their clinical relevance. Human infections of these bugs can acquire multi-drug resistance, becoming super-bugs. A gene-editing tool such as MAGE facilitates study of resistance trajectories.

### Example 4

Top individual SSAPs (SEQ ID NO: 157 and SEQ ID NO: 24, using Redβ as a control) were expressed in E. coli from a lambda pL promoter. The mutational profile of edits are shown in FIG. 5, including the efficiency of introducing 18-nucleotide (NT) and 30-NT mismatches. Efficiency was measured by disruption of LacZ, plating on X-gal, and counting the number of blue vs. white colonies. In contrast to FIG. 1A, a high over-performance by the SSAP (SEQ ID NO: 157) alone was observed when it was driven off of a more efficient promoter. It performed at about double the efficiency of Redβ or SSAP SEQ ID NO: 24.

Co-expression of an SSAP/SSB pair facilitated the integration of double-stranded cassettes. Erythromycin colony forming units (CFUs) were tested after expression of SSAP SEQ ID NO: 24 alone, or co-expressed with its corresponding SSB or exonuclease (FIG. 6A). The SSAP/SSB pair alone was enough for cassette insertion. EcSSAP (Redβ), performed slightly better with its associated exonuclease, but the SSAP/SSB pair alone performed nearly as well (FIG. 6B). These results show co-expression of an SSAP and SSB together can not only facilitate oligo-mediated cloning, but can improve the efficiency of double-stranded cassette integration.

The PaSSB used in this example is encoded by the following nucleic acid sequence.

(SEQ ID NO: 475)
ATGGCCCGTGGAGTGAACAAAGTAATTCTTGTCGGTAATGTGGGTGGGGA
TCCAGAGACGCGATACATGCCAAACGGGAACGCCGTGACAAATATCACCT
TAGCCACGAGCGAATCTTGGAAGGACAAACAAACAGGTCAGCAACAAGAA
CGAACCGAATGGCATAGAGTTGTATTTTTTGGCCGACTTGCTGAGATCGC
GGGTGAGTACCTTAGAAAGGGTTCTCAGGTTTATGTCGAGGGCTCATTAA
GAACACGTAAGTGGCAGGGGCAGGACGGGCAAGACCGATATACAACTGAA
ATAGTAGTGGACATAAACGGCAACATGCAACTTCTTGGTGGCAGACCGAG
TGGGGACGATTCACAGAGAGCTCCAAGAGAACCTATGCAGCGACCACAGC
AGGCTCCTCAACAGCAGTCTCGTCCGGCCCCTCAGCAGCAACCGGCTCCG
CAACCTGCACAAGATTACGATAGTTTTGATGATGATATTCCATTCTAA.

### Example 5

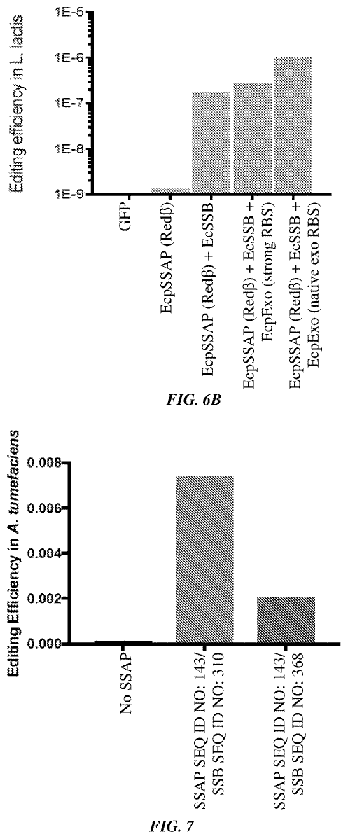

A library of the most broadly-acting three (3) SSAPs and twenty five (25) SSBs was cloned into an Agrobacterium tumefaciens (A. tumefaciens) vector (75-member library). The library was selected for efficient genome editing, and oligo-recombineering. Efficiency was measured from the two most frequent members of the library after two rounds of selection. Editing efficiency of close to 1% was measured in SSAP SEQ ID NO: 143/SSB SEQ ID NO: 310. The results demonstrate that a relatively small library of broadly acting SSAP/SSB pairs can produce active variants in a novel bacterial species. A. tumefaciens is quite distantly related to E. coli, L. lactis, and M. smegmatis (FIG. 7).

### Example 6

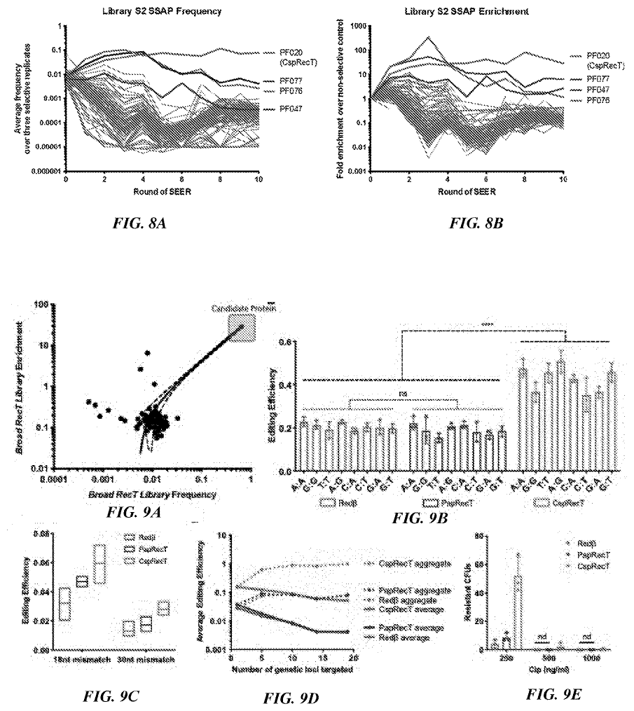

By investigating the distribution of efficient recombineering-functions across the seven principal families of phage-derived SSAPs, the initial SEER screen suggested the RecT family (Pfam family: PF03837) as the most abundant source of recombineering proteins for E. coli. Therefore, it was determined whether by screening additional RecT variants, again exploiting the increased throughput of SEER compared to previous efforts, one might discover recombineering proteins further improved over Redβ and PapRecT. To this aim a second library was constructed, identifying a maximally diverse group of 109 RecT variants, 106 of which were synthesized successfully, which was called Broad RecT Library (see Methods for more details). Next, as previously described, 10 rounds of SEER selection was performed on Broad RecT Library (FIGS. 8A-8B), and upon plotting frequency against enrichment after the final selection, a clear winner emerged (FIG. 9A). This protein, which was referred to as CspRecT (UniParc ID: UPI0001837D7F), originates from a phage of the Gram-positive bacterium Collinsella stercoris.

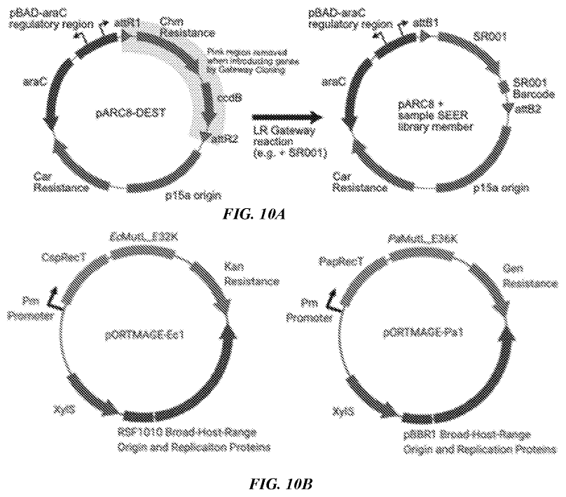

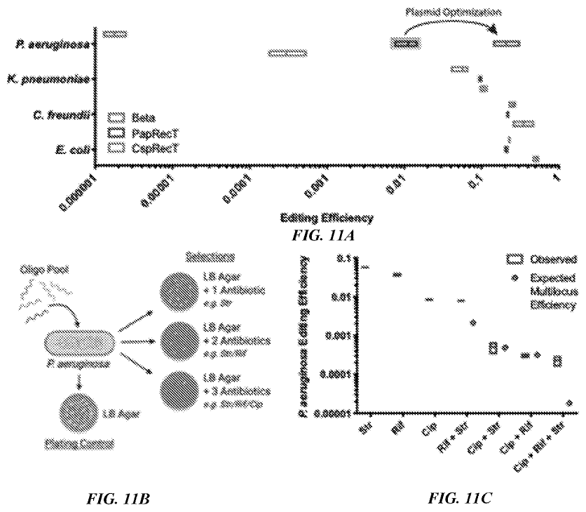

To maximize the phylogenetic reach and applicability of these new tools, CspRecT was characterized, alongside Redβ and PapRecT, subcloned into the pORTMAGE plasmid system (FIGS. 10A-10B, Addgene accession: #120418). This plasmid contains a broad-host RSF1010 origin of replication, establishes tight regulation of protein expression with an m-toluic-acid inducible expression system, and disables MMR by transient overexpression of a dominant-negative mutant of E. coli MutL (MutL E32K) (Nyerges et al., Proc. Natl. Acad. Sci. U.S.A. 113, 2502-2507 (2016)), which makes it possible to establish high-efficiency editing without modification of the host genome. Measured with a standard lacZ recombineering assay, wild-type E. coli MG1655 expressing CspRecT exhibited editing efficiency of 35-51% for various single-base mismatches, averaging 43% or more than double the efficiency of cells expressing Redβ or PapRecT off of the same plasmid system (FIG. 9B). This pORTMAGE plasmid expressing CspRecT was referred to as pORTMAGE-Ec1 (Addgene). Without being bound by a particular theory, the efficiency of CspRecT single-locus genome editing reported here is the first to significantly exceed 25%, the theoretical maximum for a single incorporation event (Pines et al., ACS Synth. Biol. 4, 1176-1185 (2015)), implying that editing occurs either at multiple forks or over successive rounds of genome replication.

CspRecT was then tested at a variety of more complex genome editing tasks. For longer strings of consecutive mismatches, which are lower efficiency events, CspRecT was again about twice as efficient as Redβ. Wild type E. coli MG1655 expressing CspRecT displayed 6% or 3% efficiency (vs. 3% or 1% for Redβ) for the insertion of oligos conferring 18-bp or 30-bp consecutive mismatches into the lacZ locus respectively (FIG. 9C). To further investigate the performance of CspRecT at complex, highly multiplexed genome editing tasks, a set of 20 oligos spaced evenly around the E. coli genome was designed, each of which incorporates a single-nucleotide synonymous mutation at a non-essential gene. Next, while expressing Redβ, PapRecT, and CspRecT separately from the corresponding pORTMAGE plasmid, a single cycle of genome editing was performed with equimolar pools of 1, 5, 10, 15, and 20 oligos and assayed editing efficiency at each locus by PCR amplification coupled to targeted next generation sequencing (NGS). NGS analysis revealed a general trend: as the number of parallel edits grew, the degree of overperformance by CspRecT also grew (FIG. 9D). For instance, when making 19 simultaneous edits (one oligo from the pool of 20 could not be read out due to inconsistencies in allelic amplification), CspRecT averaged 5.1% editing efficiency at all loci, whereas Redβ and PapRecT averaged only 0.40% and 0.43%. Importantly, despite keeping total oligo concentration fixed across all pools, aggregate editing efficiency increased as more oligos were present in each pool. For instance, when using CspRecT with a 19-oligo pool, aggregate editing efficiency was nearly 100%, implying that across the total recovered population of E. coli there averaged one edit per cell.

Finally, based on the increased integration efficiency with CspRecT in multiplexed genome editing tasks, its performance was also tested in a directed evolution with random genomic mutations (DIvERGE) experiment (Nyerges et al., Proc. Natl. Acad. Sci. U.S.A. 115, E5726-E5735 (2018)). DIvERGE uses large libraries of soft-randomized oligos that have a low basal error rate at each nucleotide position along their entire sequence to incorporate mutational diversity into a targeted genomic locus. To compare the performance of Redβ, PapRecT, and CspRecT, one round of DIvERGE mutagenesis was performed by simultaneously delivering 130 partially overlapping DIvERGE oligos designed to randomize all four protein subunits of the drug targets of ciprofloxacin (gyrA, gyrB, parC, and parE) in E. coli MG1655. Following library generation, cells were subjected to 250, 500, and 1,000 ng/mL ciprofloxacin (CIP) on LB-agar plates. Variant libraries that were generated by expressing CspRecT produced more than ten times as many colonies at low CIP concentrations (i.e., 250 ng/mL) as Redβ and PapRecT, while at 1,000 ng/mL CIP, which requires the simultaneous acquisition of at least two mutations (usually at gyrA and parC) to confer a resistant phenotype, only the use of CspRecT produced resistant variants (FIG. 9E). Because gyrA and parC mutations are usually necessary to confer high-level CIP resistance, sequence analysis of gyrA and parC from 11 randomly selected CIP-resistant colonies, many different mutations were found, in combinations of up to three (data not shown). In sum, in both MAGE and DIvERGE experiments, which require multiplex editing, CspRecT provided more than an order of magnitude improvement to editing efficiency over Redβ, the current state-of-the-art recombineering tool.

### Example 7

SSAPs frequently show host tropism (Sun et al., Appl. Microbiol. Biotechnol. 99, 5151-5162 (2015); Yin et al., iScience 14, 1-14 (2019); Ricaurte et al., Microb. Biotechnol. 11, 176-188 (2018)), but there are also indications that within bacterial clades certain SSAPs may function broadly (van Pijkeren et al., Nucleic Acids Res. 40, e76 (2012); Nyerges et al., Proc. Natl. Acad. Sci. U.S.A. 113, 2502-2507 (2016); van Kessel et al., Nat. Rev. Microbiol. 6, 851-857 (2008)). Therefore, the functionality of PapRecT and CspRecT in selected Gammaproteobacteria was investigated and their efficiency was compared to that of Redβ. Efforts were focused on two enterobacterial species: Citrobacter freundii ATCC 8090 and Klebsiella pneumoniae ATCC 10031, along with the more distantly related Pseudomonas aeruginosa PAO1. Pathogenic isolates of K. pneumoniae and P. aeruginosa are among the most concerning clinical threats due to widespread multidrug resistance (Tommasi et al., Nat. Rev. Drug Discov. 14, 529-542 (2015)). In these species, oligo-recombineering based multiplexed genome editing (i.e., MAGE and DIvERGE) holds the promise of enabling rapid analysis of genotype-to-phenotype relationships and predicting future mechanisms of antimicrobial resistance (Nyerges et al., Proc. Natl. Acad. Sci. U.S.A. 115, E5726-E5735 (2018); Szili et al., bioRxiv 495630 (2018) doi:10.1101/495630. C. freundii, by contrast, is an intriguing biomanufacturing host in which the optimization of metabolic pathways has remained challenging (Yang et al., Biochem. Eng. J. 57, 55-62 (2011); Jiang et al., Appl. Microbiol. Biotechnol. 94, 1521-1532 (2012)).

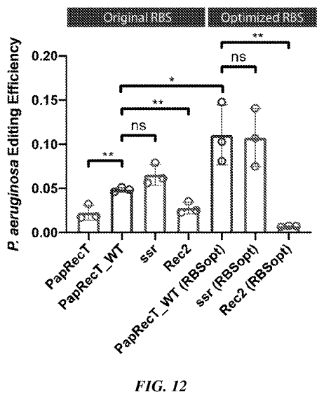

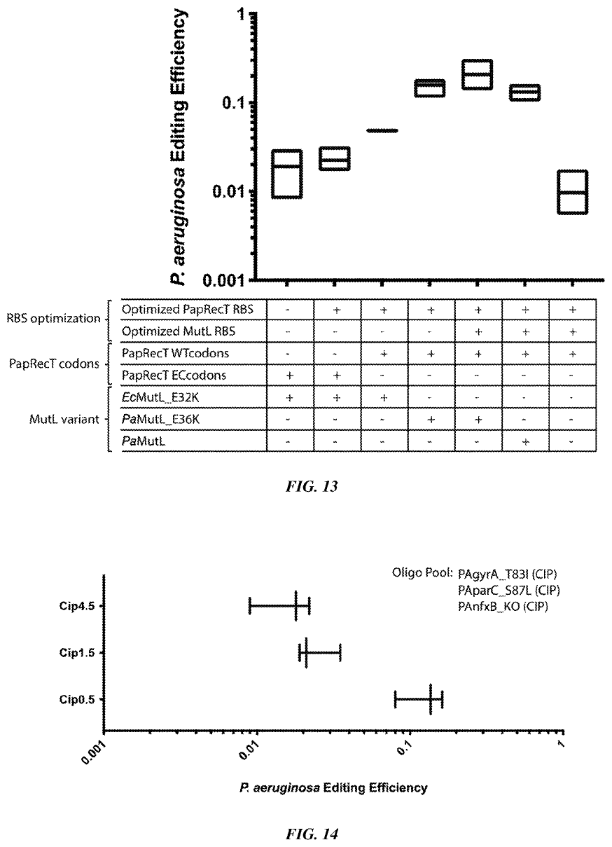

To test the activity of PapRecT and CspRecT in these three organisms, the broad-host-range pORTMAGE system (Nyerges et al., Proc. Natl. Acad. Sci. U.S.A. 113, 2502-2507 (2016)) was built on as described above. For experiments in E. coli PapRecT or CspRecT was subcloned in place of Redβ into pORTMAGE311B (Szili et al., Antimicrob. Agents Chemother. AAC.00207-19 (2019) doi:10.1128/AAC.00207-19) (FIGS. 10A-10B; Addgene accession: #120418), which transiently disrupts MMR with EcMutL_E32K, and whose RSF1010 origin of replication and m-toluic-acid-based expression system allows the plasmid to be deployed over a broad range of bacterial hosts (Honda et al., Proc. Natl. Acad. Sci. U.S.A. 88, 179-183 (1991); Gawin et al., Microb. Biotechnol. 10, 702 (2017)). These same pORTMAGE-based constructs were used for testing in C. freundii and K. pneumoniae. In P. aeruginosa the plasmid architecture remained constant, except that the origin of replication and antibiotic resistance were replaced, instead using the broad-host-range pBBR1 origin, which was shown to replicate in P. aeruginosa (Szpirer et al., J. Bacteriol. 183, 2101-2110 (2001)), and a gentamicin resistance marker (FIGS. 10A-10B). Next these constructs were tested (See methods for details), and in all three species, PapRecT and CspRecT displayed high editing efficiencies (FIG. 11A). In C. freundii and K. pneumoniae, just as in E. coli, CspRecT was found to be the optimal choice of protein, whereas in P. aeruginosa PapRecT performed the best. PapRecT was further compared to two recently reported Pseudomonas putida SSAPs (Rec2 and Ssr) (Ricaurte et al., Microb. Biotechnol. 11, 176-188 (2018); Aparicio et al., Microb. Biotechnol. 11, 176-188 (2018)), and found that PapRecT, isolated from a large E. coli screen performed equal to or better than proteins found in smaller screens run through P. putida (FIG. 12). It was found, however, that the efficiency of the plasmid construct was lower in P. aeruginosa than in the enterobacterial species that pORTMAGE was optimized for. Therefore, to increase editing efficiency in P. aeruginosa, i.) ribosomal binding sites (RBS) for PapRecT and EcMutL were optimized, ii.) EcMutL_E32K was replaced with its equivalent homologous mutant from P. aeruginosa (PaMutL_E36K), iii.) the native P. aeruginosa coding sequence for PapRecT was incorporated instead of the E. coli codon-optimized version (FIG. 13). Together these changes significantly improved the editing efficiency of the best plasmid construct featuring PapRecT in P. aeruginosa, which was called pORTMAGE-Pa1 (Addgene), to −15%.

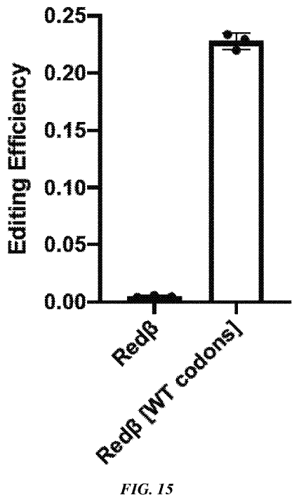

Virulent strains of P. aeruginosa are a frequent cause of acute infections in healthy individuals, as well as chronic infections in high-risk patients, such as those suffering from cystic fibrosis (Marvig et al., Nat. Genet. 47, 57-64 (2015)). The rate of antibiotic resistance in this species is growing, with strains adapting quickly to all clinically applied antibiotics (AbdulWahab et al., Lung India Off. Organ Indian Chest Soc. 34, 527-531 (2017); Tacconelli et al., Lancet Infect. Dis. 18, 318-327 (2018)). The development of multidrug resistance in P. aeruginosa requires the successive acquisition of multiple mutations, but due to the lack of efficient tools for multiplex genome engineering in P. aeruginosa (Agnello et al., J. Microbiol. Methods 98, 23-25 (2014); Chen et al., iScience 6, 222-231 (2018)), investigation of these evolutionary trajectories has remained cumbersome. Therefore, and to demonstrate the utility of pORTMAGE-Pa1-based MAGE in P. aeruginosa, a panel of genomic mutations that individually confer resistance to STR, RIF, and fluoroquinolones (i.e., CIP) were simultaneously incorporated (Cabot et al., Antimicrob. Agents Chemother. 60, 1767-1778 (2016); Jatsenko et al., Mutat. Res. 683, 106-114 (2010)). Importantly, the corresponding genes are also clinical antibiotic targets in P. aeruginosa (PEW ChariTable Trust. Antibiotics Currently in Global Clinical Development; pew.org/1YkUFkT). Following a single cycle of MAGE delivering 5 mutation-carrying oligos, a single-day experiment with pORTMAGE-Pa1, all possible combinations of five resistant mutations were able to be isolated, with more than 10⁵ cells from a 1 ml overnight recovery attaining simultaneous resistance to STR, RIF, and CIP (FIG. 11B). Interestingly, because rpsL and rpoB, the resistant loci for STR and RIF respectively, are located only ˜5 kb apart from each other on the P. aeruginosa genome, these two mutations co-segregated much more often than would be expected by independent inheritance, confirming that co-selection functions similarly in P. aeruginosa to E. coli (FIG. 11C) (Wang et al., Nat. Methods 9, 591-593 (2012)). By genotyping and characterizing resistant colonies, the Minimum Inhibitory Concentration (MIC) of CIP for various resistant genotypes could be determined (Table 2, FIG. 14). GyrA_T83I displays strong positive epistasis with ParC_S87L, and so clonal populations with mutations to parC but not gyrA were not pulled out of the antibiotic selection (Marcusson et al., PLoS Pathog. 5, e1000541 (2009)). The allure of this method is that the entire workflow took only three days to complete, in contrast with other genome engineering methods (i.e., CRISPR/Cas9 or base-editor-based strategies) that are either less effective, have biased mutational spectra, and/or would require tedious plasmid cloning and cell manipulation steps (Agnello et al., J. Microbiol. Methods 98, 23-25 (2014); Chen et al., iScience 6, 222-231 (2018)).

### Example 8

The performance of Redβ expressed off of its wild-type codons against the codon-optimized version that was included in Broad SSAP Library. This revealed significantly decreased efficiency for the codon-optimized version of Redβ (FIG. 15), which indicates that codon choice is an important consideration for library design.

### Example 9

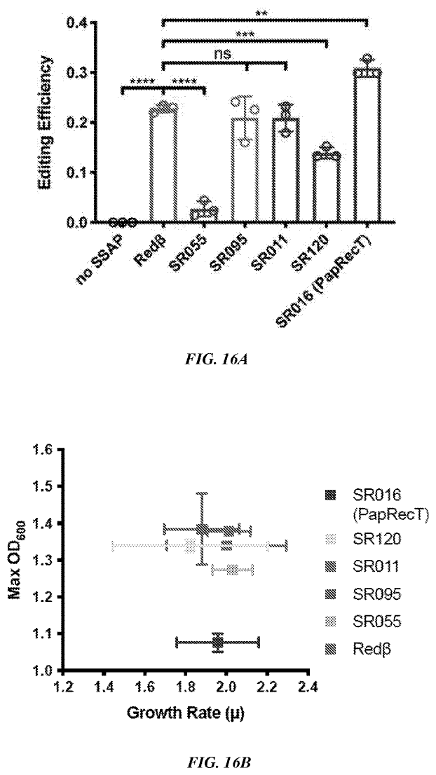

A set of five Broad SSAP library members that exhibited both high frequency and enrichment were chosen for further analysis. Their recombineering efficiency was tested against Redβ expressed off of its wild-type codons on the same plasmid system used for the SEER selections. To ensure an accurate measurement the efficiency of each SSAP was queried by NGS after performing a silent, non-coding genetic mutation at a non-essential gene, ynfF (silent mismatch MAGE oligo). Broad SSAP Library member: SR016, noted above as PapRecT (UniParc ID: UPI0001E9E6CB), demonstrated the highest efficiency of recombineering among the five Broad SSAP candidates, i.e. 31%±2% (FIG. 16A). The impact of these SSAPs on growth rate is shown in FIG. 16B.

See also, e.g., Wannier et al., bioRxiv 2020.01.14.906594 (doi.org/10.1101/2020.01.14.906594), which is incorporated by reference in its entirety.

TABLE 1
SSAP and SSB_Library

| | | | | | SEQ |
|---|---|---|---|---|---|
| | | | | | ID |
| Protein | Type | Family | Source | Amino Acid Sequence | NO: |
| N001 | SSAP | recT | Lactobacillus reuteri | MTNQVAQQQKPTKLTDLVLDRVKQMQDTQDLSPKNYNASN | 1 |
| | | | | ALNAAFLELQKVQDRNHRPALEVCSHDSIVKSLLDMTLQGLSP | |
| | | | | AKDQCYFIVYGNELQMQRSYFGTVAAVKRLDGVKKVRAEVV | |
| | | | | HEKDDFEIGANEDMELVVKRFVPKFENQDNQIIGAFAMIKTDE | |
| | | | | GTDFTVMTKKEIDQSWAQTRQKNNKVQQNESQEMAKRTVEN | |
| | | | | RAAKMFINTSDDSDLLTGAINDTTSNEYDDERRDVTPVEDEKQ | |
| | | | | STDKLLEGFQKSQEAKAKGVSNDGNSNEGKETSEEVADGQTE | |
| | | | | LFSEGTIKPADEADS | |
| N002 | SSAP | recT | Lactobacillus reuteri | MTNQVANTQQITVKQFVNMNSTKKRFEDVLGKRAPQFMSSLIS | 2 |
| | | | | IVNSDQNLQRVEAASVINSALVAAALDLPINPNFGYMYIVPYN | |
| | | | | GQAQPQMGYKGYIQLAQRSGQYRKITVAELYEDEFISWDPLM | |
| | | | | EELKYEAHREKERDEKEQPVGYFGHFELLNGFQKTVYWTRQQ | |
| | | | | VDNRRKRFSQAGGKNSDKPKGVWAKNYNAMALKTVIKDLLT | |
| | | | | KWGPMTVDMQTAYGADEEEYNENPRDVTPVQDTASAQSEEG | |
| | | | | YQTQDILNSFDQAEKEKASKEEAKPAKKATKTAKKTVKKGDE | |
| | | | | VNNANESQEELFPDGTITPHAK | |
| N003 | SSAP | recT | Escherichia coli | MTKQPPIAKADLQKTQGNRAPAAVKNSDVISFINQPSMKEQLA | 3 |
| | | | | AALPRHMTAERMIRIATTEIRKVPALGNCDTMSFVSAIVQCSQL | |
| | | | | GLEPGSALGHAYLLPFGNKNEKSGKKNVQLIIGYRGMIDLARR | |
| | | | | SGQIASLSARVVREGDEFSFEFGLDEKLIHRPGENEDAPVTHVY | |
| | | | | AVARLKDGGTQFEVMTRKQIELVRSLSKAGNNGPWVTHWEE | |
| | | | | MAKKTAIRRLFKYLPVSIEIQRAVSMDEKEPLTIDPADSSVLTGE | |
| | | | | YSVIDNSEE | |
| N004 | SSAP | recT | Enterococcus faecalis (strain | MSNDLTQITQRSLDEQVIGNLNRLQEQGLEMPPGYSPQNALKS | 4 |
| | | | ATCC 700802/V583) | AFFELTNNSGGNLLQLAANNPETKTSISNALLDMVIQGLSPAKK | |
| | | | | QCYFIKYGNKVQLMRSVFGTMAVLDRVTGGADITPVVVREGD | |
| | | | | VFEIAMDGPDLVVAKHETAFENLDNDIKAAYVVIKLANGKEVT | |
| | | | | TVMTKKQIDKSWSKAKTKNVQNDFPEEMAKRTVINRAAKYLI | |
| | | | | NTSNDNDLFVQAAKDTLENEFERKDVTPEREEQAAVLEEKLFS | |
| | | | | NNIKAVDQENENERITRVADVPEQPDIEQAKPIEKDNLTKVAD | |
| | | | | QILEEPVQETLDVMAGYETNQKESEADVSTIEEDDYPF | |
| N005 | SSAP | recT | Enterococcus faecalis TX0027 | MGNELIVSVQNRIQEMQHGEGLRLPTGYSVGNALNSAYLILSD | 5 |
| | | | | NSKGKSLLEKCHPTSVSKALLNMAIQGLSPAKNQCYFVPYGDQ | |
| | | | | CTLMRSYFGSVSILERLSNVKKVHAEVIFEGDEFEIGSEDGRTV | |
| | | | | VTNFKPSFLNRDNPIIGAFAWVEQTDGIKVYTIMTKKEIDKSWS | |
| | | | | KAKTKNVQNDYPQEMAKRTVLSRAAKMFINSSSDNDLLVKAI | |
| | | | | NETTEDEYDNNQQRKDITPNPPNIEKLEKSIFNQDENKKIAQDM | |
| | | | | IDSIDLNQADKDLQEELNIEFPDPSKNYLATGEVNGDVENEDGP | |
| | | | | YPF | |
| N006 | SSAP | recT | Bacillus subtilis | MAKNDDIRNQLANKVNSVQKEDKPKTLADYLNDMKPELQKALP | 6 |
| | | | | EHITPERITRIALTTIRSNPGLQQCSPASLLGAVMQSAQLGLEP | |
| | | | | GLVGHCYFVPFNKKIKGQNGAPDQWVKEVQFIIGYKGMIDLA | |
| | | | | RRSGHIESIYAHAVYEKDEFDYELGLHPKLVHKPSTGHRGEMT | |
| | | | | HVYAVAHFKDGGYQFDVFSKQDIENVRLRSKSKDNGPWQTDY | |
| | | | | EEMAKKTVIRRMWKYLPISIEIQKQVAQDETVRKDITSEAQSVY | |
| | | | | DDNVLDLGGNQFLSEPQQLNEKPSAQDADPFDGKPVDISEDDL | |
| | | | | PFD | |
| N007 | SSAP | recT | Agrobacterium rhizogenes | MTQTAERQPRSVLVDMSMRYGMEPAAFEATVRATCMKPDKN | 7 |
| | | | | GKVPSREEFAAFLLVAKEYNLNPLLKEIYAYPAKGGGIVPIVSV | |
| | | | | DGWVNLINSQAALDGLEFAIEHTDVGALVSITCRIYRKDRSRPI | |
| | | | | EVTEYLSECIRNTEPWAMKHRMLRHKALIQCARYAFGEAGIYD | |
| | | | | EDEGEKIAGMKDVTPPMPPAPPKPPAPPKPDETIADADGVVIEH | |
| | | | | EEPTVEDVAAANEDVIDDTTYFENLEEAMAVVSDAASLEEVW | |
| | | | | SDFDPLSRFDGKPQGEVNQGIALAIRKRAEKRIGGAA | |
| N008 | SSAP | recT | Mycobacterium virus Che9c | MAENAVTKQDSPKAPETISQVLQVLVPQLARAVPKGMDPDRIA | 8 |
| | | | | RIVQTEIRKSRNAKAAGIAKQSLDDCTQESFAGALLTSAALGLE | |
| | | | | PGVNGECYLVPYRDTRRGVVECQLIIGYQGIVKLFWQHPRASRI | |
| | | | | DAQWVGANDEFHYTMGLNPTLKHVKAKGDRGNPVYFYAIVE | |
| | | | | VTGAEPLWDVFTADEIRELRRGKVGSSGDIKDPQRWMERKTA | |
| | | | | LKQVLKLAPKTTRLDAAIRADDRPGTDLSQSQALALPSTVKPT | |
| | | | | ADYIDGEIAEPHEVDTPPKSSRAQRAQRATAPAPDVQMANPDQ | |
| | | | | LKRLGEIQKAEKYNDADWFKFLADSAGVKATRAADLTFDEAK | |
| | | | | AVIDMFDGPNA | |
| SR001 | SSAP | sak | Lactococcus phage | MSVFEQLNAINVNSKVEQKKTGKTSLSYLSWSWAWAEFKKVC | 9 |
| | | | SK1833, Lactococcus phage SK1 | PTATYEIKKFDDGKGKLVPYLVDNSLGIMVFTSVTVDDITHEM | |
| | | | | WLPVMDGANKAMKFDSYTYKTKFGEKTVEPASMFDVNKTIM | |
| | | | | RCLVKNLAMFGLGLYIVSGEDLPDLTEEQKELEAEKQRLREIQP | |
| | | | | ALNRAEELGYPNMELLKTKTKKEIFDIMKIWLAQQETEKGE | |
| SR002 | SSAP | recT | Hafnia alvei ATCC 51873 | MSNTMMLAPQTFDQAMQFANAIAASQFAPSSYRGKPNDVLIA | 10 |
| | | | | MQMGAELGFQPMQSVQGIAVINGRPSVWGDALRALILSAPDL | |
| | | | | AEFEESYDEATQTAHCKISRREQTGSIATENSSESVTDAQTAGL | |
| | | | | WGKNGPWKQYPKRMQQWRALGFCARDSYADRLKGIQLAEEVQD | |
| | | | | YEPIEKVVHTPSQDQDSAIENKITEEQSNRINEILISVDSTFDD | |
| | | | | LKKACKSMTGRDIDNQSELTSTEAAKLISSLERKLASKTGGEKD | |
| | | | | AA | |
| SR003 | SSAP | recT | Corynebacterium striatum ATCC | MSKEIARTQDHLNEMMNWSRAMSQGNLMPRQYQGNPANLM | 11 |
| | | | 6940 | FAAEYADALGISRIHVLTSIAVINGRPSPSADLMSAMVRQHGHK | |
| | | | | LRVAGDDTYAEAVLIRSDDPDFEYTARWDESKARKAGLWGN | |
| | | | | KGPWSLYPGAMLRARAISEVVRMGASDVMAGGIYTPEEVGAV | |
| | | | | VDESGHVVEQPAQHKATRQQAQPQDNSAQARLANMLDATPA | |
| | | | | NTDDPEVWADRIAEAGSEQELMELYATASTNPEWESAIKAMFT | |
| | | | | ARKQQILLDAQYADEAEALDAELVEDTTEESAA | |
| SR004 | SSAP | recT | Lactococcus lactis subsp lactis | MSNQITKTQQTLKSPEVKKKFEEVEGKKTEGFVASLLSVVGNS | 12 |
| | | | bv diacetylactis str TIFN2, | NLKNADANSVMTAAMKAATLDLPIEPSLGFAYVIPYGREAQFQ | |
| | | | Lactococcus lactis subsp lactis | IGYKGFIQLALRSGQLTGLNCGIVYESQFVSYDPLFEELELDFSQ | |
| | | | (strain IL1403) | QASGDAVGYFASMKLANGFKKVTYWSKEQVLAHKKKFVKSA | |
| | | | (Streptococcus lactis), | NGPWRDHFDAMAQKTVLKAMLTKVAPASIESKMIQTAITEDD | |
| | | | Lactococcus phage bIL309 | SERFENAKDVTPDEPVISIDESMTSEVSQNEPATESQEQLPEDEV | |
| | | | | EELFPIGKS | |
| SR005 | SSAP | recA | Mycobacterium phage Che8 | MSLSFKPATREASYARIALSGPSGSGKTYTALALGTALADKVA | 13 |
| | | | | VIDTERGSASKYVGLNGWQFDTVQPDSFSPLSLVELLGLAAGG | |
| | | | | EYGCVIVDSLSHYWMGVDGMLEQADRHAVRGNTFAGWKEV | |
| | | | | RPDERRMIDALVSYPGHVIVTMRSKTEYVIEENERGKKTPRKV | |
| | | | | GMKPEQRDGIEYEFDVVGDLDHDNTLTVVKSRIHTLAKAVVP | |
| | | | | MPGEEFAHQIRDWLSDGARVPTVAEYRKQALAAETREELKAL | |
| | | | | YDEVSGHKLTVAPTVDRDGNSTVLGDLITDLAREMKRAEA | |
| SR006 | SSAP | gp2.5 | Xanthomonas phage OP2 | MSIADHVAIFLAGSLDAPKAGKAKPDRPEFWGLFAFPPSAGAD | 14 |
| | | | | LTAACQAAAGGSLAGMRLAPKLHSRLEPDKQFAGIPQDWLIVR | |
| | | | | MGTGPDFPPALFLLDGSSVQALPINGAKIRTDLFAGQKVRVNA | |
| | | | | HGFAYPPKNGGPAGVSFSLDGVMAVGGGERRSSSSEGGEPSES | |
| | | | | VFAKYRAEVAAAPAASTAPATTGNPFQQSAAGTDNPFG | |
| SR007 | SSAP | gp2.5 | Burkholderia phage BcepNY3 | MSTIDKLAGYEAILTHHSIITPQINKLKPTKPAEFYALIALPAAA | 15 |
| | | | | QADLWAILCERATSAFGHANNEEHGIKTNATSKKPIAGVPGDA | |
| | | | | LVVRAASQYAPEIYDADGTLLNPQNPAHLQTIKAKFFAGTRVR | |
| | | | | TILTPFHWTFQGRNGVSFNLAGIMLVPSEAQRLAIGGVDTASAF | |
| | | | | KKFAQPGTGGVPATAGAPTDAAAAFAAGGNPDAAGGTLPANP | |
| | | | | NPFAQQTGSAAGAGGNPFL | |
| SR008 | SSAP | gp2.5 | Pseudomonas phage | MSKKVSQRFTFPVAKLIFPYIVTPDTEYGEVYQVTICIPTKEQAD | 16 |
| | | | vB_PaeP_C1-14_Or, Pseudomonas | ELVAKMESKDARLKGTIKYTERDGEFLFKVKQKKHVDWMQD | |
| | | | phage vB_PaeP_p2- | GERKSAVMKPIVLTSDNKPYDGPNPWGGSTGEVGILIETQKGA | |
| | | | 10_Or1, Pseudomonas phage PaP3 | RGKGTITALRLRGVRLHEIVSGGDGEDDPLFGGGFTEEEDKPED | |
| | | | | VFGEDFDDEDAPI | |
| SR009 | SSAP | recA | Paenibacillus dendritiformis | MSDRRAALEMALRQIEKQFGKGSIMKLGESNHMQVEIVPSGSL | 17 |
| | | | C454 | ALDIALGIGGLPRGRIIEVYGPESSGKTTVALHAIAEAQKVGGQ | |
| | | | | AAFIDAEHALDPTYASKLGVNIDELLLSQPDTGEQALEIAEALV | |
| | | | | RSGAVDIIVIDSVAALVPKAEIEGEMGDSHVGLQARLMSQALR | |
| | | | | KLSGAISKSKTIAIFINQLREKVGVMFGNPETTPGGRALKFYSTI | |
| | | | | RLDVRRVETIKQGNDMIGNRTRIKVVKNKVAPPFKQADIDIMY | |
| | | | | GEGISREGSIVDIGTEMDIIQKSGAWYSYEGERLGQGRENAKQF | |
| | | | | LKENGELALTIENKVREASNLSTVVRNHSEHDAEEAEDALELE | |
| | | | | LES | |
| SR010 | SSAP | recT | Neisseria lactamica Y92-1009 | MSIAQNQAVALAKQFNIQGDPQELVQTLKATAFKGNATDAQF | 18 |
| | | | | NALMIVSTQYGLNPFTKEIYAFPDKNNGITPVVGVDGWARIINS | |
| | | | | HPQFDGMEFAADAESCTCKIHRKDRNHPTIVTEYLEECRRNTQ | |
| | | | | PWNSHPRRMLRHKAMIQAARLAFGFGGIYDEDEAQRIQTTETP | |
| | | | | ETPKEVKADPELDSLLAEGEAAANKGIEEYKKWFSEIGATGRL | |
| | | | | KLGSENHERFKQIAANTIEAETAETLKPTPTEEQFAALVEAVST | |
| | | | | GVKEVAEVLEAYALTDEQAAEINAL | |
| SR011 | SSAP | ERF | Thalassomonas phage BA3 | MSKSELVTQDQSSEPVMAEPHMRLIEIAVEKGADITQLEKLMD | 19 |
| | | | | LQERYEANQAKKDFNEAMSKFQSLLPTIEKSGVVDYTTNKGRT | |
| | | | | YYDYAKLEDIAKAIRPALKETGLSYRFSQSQNQGWITVTCIVTH | |
| | | | | ASGHSEVSELTSQPDVSGGKDPLKAIASAISYLRRYTLTGSLGIV | |
| | | | | VGGEDDDGGNHQEANDETDCYSDEEFKKNFPNWEKAILAGKK | |
| | | | | TPEQIIKAGNAQGITFSQQQLETIEKVGNV | |
| SR012 | SSAP | recT | Commensalibacter intestini A911 | MVPKTFTPADIVVAVQLGTSIGLSVAQSLHNIAIINGKPSIYGDM | 20 |
| | | | | MLALCRASPECEYVKEEMEGNKKEEWVAICTVKRKGNPEVIS | |
| | | | | KFSWQDAVDAKLTGKPGPWLSYPKRMLQMRARGFALRDAFP | |
| | | | | DLLNGLISQEEAQDYPTQTIEPPPVQLQSKPVAEQEVIQEMPSIE | |
| | | | | PEKSELIKRYDWLVGQLTDIESREYLEKLTSQTKIINLRNELTEK | |
| | | | | EPKLAAVITDLIEQALASFEEQGELANAV | |
| SR013 | SSAP | sak4 | Mycobacterium phage | MVQSKIIKVADDEDYVNLLVYGDSGVGKTVFCGSDDKVLFVA | 21 |
| | | | Troll4, Mycobacterium phage | PEDNSDGLLSAKLAGTTADKWPIRDWGDLVEAYNYLDELDEIP | |
| | | | Gumball, Mycobacterium phage | YNWIVVDSLTEMQIMAMRDILDRAVEENPSRDPDIPQIQDWQK | |
| | | | Nova, Mycobacterium phage | YYEMVKRMIKCFNALPVNVLYTALSRQTEDEEGTEYLLPDLQ | |
| | | | SirHarley, Mycobacterium phage | GKKDNYAKQVVSWMTSFGCMQIKRVRVKTDDDIAKKVKEVR | |
| | | | Adjutor, Mycobacterium phage | RITWKDTGLVTGKDRTNALTPYTDIRDVTDPEDDGLTLKDIRL | |
| | | | Butterscotch, Mycobacterium | RIERKKSGSAKSATTKRPARKTASARTQKESA | |
| | | | phage PLot, Mycobacterium | | |
| | | | phage PBI1 | | |
| SR014 | SSAP | recT | Ureaplasma urealyticum serovar | MVKDDKLVPSIRLLTPNELSEQWANPNSEINQITRAVLTIQGIDL | 22 |
| | | | 12 str ATCC 33696 | KAIDLNQAAQIIYFCQANNLNPLNKEVYLIQMGNRLAPIVGIHT | |
| | | | | MTERAYMSGRLVGITQSYNDTNKSAKTTLTIRIPNLKELGVIEA | |
| | | | | EVFLSEYSTNKNLWLTKPITMLKKVSLAHALRLSGLLAFKGDT | |
| | | | | PYIYEEMQQGEAVPNKKMFTPPVAEVIEPAVENIKKVDFNEF | |
| SR015 | SSAP | ERF | Paenibacillus alvei DSM 29 | MGLKRSESITNLAAALVKFQKEVVSPKNNANNPYFSSKYAPLH | 23 |
| | | | | EVINVIREPLAKYGLSYIQSTSTDEQNVTVTTLLMHESGEFVESE | |
| | | | | PLSLPGLQVLKGGGKDFTPQGIGSAITYGRRYSLTAILGIASEDD | |
| | | | | TDGNEGTPDPRNNPSKGKNQGTGAQTGTGGVKKTIEAKYKLL | |
| | | | | HDNSLDGLTDFIKEHGANAEQVLTQMLMDR | |
| SR016 | SSAP | recT | Pseudomonas aeruginosa 39016 | MGTALTPLLTKFATRYEMGTTPEEVANTLKQTCFKGQVNDSQ | 24 |
| | | | | MVALLIVADQYKLNPFTKELYAFPDKNNGIVPVVGVDGWARII | |
| | | | | NENPQFDGMEFSMDQQGTECTCKIYRKDRSHAISATEYMAEC | |
| | | | | KRNTQPWQSHPRRMLRHKAMIQCARLAFGFAGIYDQDEAERI | |
| | | | | VERDVTPAEQYEDVSEAICLIKDSPTMEDLQAAFSNAWKAYKT | |
| | | | | KGARDQLTAAKDQRKKELLDAPIDVEFEETGDDRAA | |
| SR017 | SSAP | gp2.5 | Salmonella phage SETP3 | MGIKLNLRKVQTAWLNVFERAKDRENSDGSITKGTYNGTFILT | 25 |
| | | | | PEHPQIEELRDTVFAVVSEALGEAAAEKWMKQNYGEGKHMD | |
| | | | | KCAVRDIAERDNPFEDFPEGFYFQAKNKQQPLILTSVKGEKQV | |
| | | | | EPDFNIDGEQIEGKQVYSGCVANISIEIWFSEQYKVEGAKLNGIK | |
| | | | | FAGEGKAFGGSAVSASVDDLEDDEDETPRRERRRNR | |
| SR018 | SSAP | ERF | Paenibacillus dendritiformis | MGTEKLNIYQKLLEVRKSVSYLKKEETSQQYKYTGSAQVLAS | 26 |
| | | | C454 | VRDKINEMGLILVPRILDKSLLTETVEFIDKEKPKKTTTYFTELT | |
| | | | | LSMTWVNADNPSETVECPWYSQGVDIAGEKGVGKALTYGEK | |
| | | | | YFILKFFNIPTDKDDPDAFQKKFEQKASKEIQAKIRETWIKLGLK | |
| | | | | INLLEHQCKTIYGSGLAQLSEEAAEEFLTMLEAKMGDPDGVN | |
| SR019 | SSAP | recT | Enterococcus faecalis | MGNELIVSVQNRIQEMQHGEGLRLPTGYSVGNALNSAYLILSD | 27 |
| | | | TX0309B, Enterococcus faecalis | NSKGKSLLEKCHPTSVSKALLNMAIQGLSPAKNQCYFVPYGDQ | |
| | | | TX0309A, Enterococcus faecalis | CTLMRSYFGSVSILERLSNVKKVHAEVIFEGDEFEIGSEDGRTV | |
| | | | (strain ATCC 700802/V583) | VTNFKPSFLNRDNPIIGAFAWVEQTDGIKVYTIMTKKEIYKSWS | |
| | | | | KAKTKNVQNDYPQEMAKRTVLSRAAKMFINSSSDNDLLVKAI | |
| | | | | NETTEDEYDNNQPRKDITPNPPNIEKLEKSIFNQDENKKIAQDMI | |
| | | | | DSIDLNQADKDLQEELNIEFPDPSKNYLATGEVNGDVENEDGP | |
| | | | | YPF | |
| SR020 | SSAP | sak4 | Enterobacteria phage | MGTATLILGESGTGKSTSMRNINPEEAILIKPIGKPLPFKSKEWL | 28 |
| | | | HK629, Salmonella phage HK620 | AWDARAKKGTVVTTDKWDVIVAVIKRAHEYGKRIVIVDDFQY | |
| | | | (Bacteriophage HK620) | VMSNEFMRRSEEKSFDKFTEIGRHAWEVIKAAQDAPDDLRVYF | |
| | | | | LAHTEETPMGRVKMKTIGKMLDEKITVEGMFTIVLRTLTRDDQ | |
| | | | | FFFTTKNNGADTVKSPMGMFDSNEIDNDLSFVDATVCDYYGIN | |
| | | | | NVHQIKENAA | |
| SR021 | SSAP | recT | Klebsiella pneumoniae subsp | MAGKLASRLGMDAGTDLMNTLKNTAFKGGNVTDDQFTALLI | 29 |
| | | | rhinoscleromatis ATCC 13884 | VANQYGLNPWTKEIYAFPDKGGIVPVVGVDGWVRIINEHPQFD | |
| | | | | GMGFTYDKEEGACTCKIYRKDRTHPTIVTEYMGECKRNTQPW | |
| | | | | QSHPTRMLRHKTLIQCARLAFGFAGIFDQDEAERVIEGSAAEVH | |
| | | | | VGHESDSRRPELIAKGESAARLGTVKYQEFWVALSAEEKQVIG | |
| | | | | AVEKRRMYDMSLAVDNAEPVDAAA | |
| SR022 | SSAP | recT | Clostridium sporogenes (strain | MAENKNSAVALLEKEMVYQVGEEEVKLTGSIVKNYLAKGNK | 30 |
| | | | ATCC 7955/DSM 767/NBRC | QITNREVVVFMNLCKYRKLNPFLNEAYLVKFKDEAQIVTGKEA | |
| | | | 16411/NCIMB 8053/NCTC | FMRKAEENPNYKGHRAGIIVMREKEIVELEGCFKLKTDTLLGG | |
| | | | 8594/PA 3679) | WAEVMVEGKNCPIVAKVSLEEYNKQQSTWKSMPSTMIRKVAL | |
| | | | | VQALREAFPAEIGAMYSNEELGVDESKIVNVQHEVKEEIKEEA | |
| | | | | NKEVIDIEETELVEKETPVVEAEIVEPKDGEEETPY | |
| SR023 | SSAP | ERF | Staphylococcus phage | MAEQLNLYQKIADVKANIAGFTKDTKGYNFSYVSGSQILHRIR | 31 |
| | | | SA97, Staphylococcus phage | EKMIEHNLLLVPNTSNENWTTHTFKNKKGQEVTEFIVEMDLNY | |
| | | | phi7247PVL, Staphylococcus | TWINADKPEEQYEVSYHAYGQQNDISQAHGTALTYAERYFLM | |
| | | | phage phiETA3, Staphylococcus | KFFNIPTDEDDADAKQKQDKYSTVSQEFKDILTKEVNDFIAIAK | |
| | | | aureus, Staphylococcus phage | ESGFAEKYQEQINKLEKMNVEALNKNQINVTRQQIKKWLGGIE | |
| | | | phi5967PVL | Q | |
| SR024 | SSAP | gp2.5 | Paenibacillus lactis 154 | MAIDNQSTKVITGKVRLSYTHVFEPQENDSGDMKYSTAILIPKS | 32 |
| | | | | DKETLRKIKAAVDAAKELGKSKWGGKIPANCKTPLRDGDEER | |
| | | | | PDDEAYAGHYFLNASSKNKPGVAKPIGKDGNGKTKFQEITDST | |
| | | | | EVYSGCYAKVSLNFYPFDAKGNRGVAAGLNNIVKVQDGDFLG | |
| | | | | GRSSVNDDFANEDFDDIVDISDDDDFLN | |
| SR025 | SSAP | recT | Desulfitobacterium | MAITPNPIPAQDGSPIPSPDDIVGELARRKIYAGIPDDDVALALA | 33 |
| | | | metallireducens DSM 15288 | LCQKYGFDPLLKHLVLLATKDRDETTGQGQKHYNAYVTRDGL | |
| | | | | LHVAHTSGMLDGLETIQGKDDLGEWAEAVVYRKDMSRPFRY | |
| | | | | RVYLSEYVREAKGVWKTHPQAMLTKTAEVFALRRAFDVALTP | |
| | | | | FEEMGFDNQNIAGDTGPSPKTGFTEKAGFTGNTDFSAEASLPG | |
| | | | | KARFSTEAGLTDMTVIPPNRVTGSIPETSRLNTSAGSTGRQRRQ | |
| | | | | LF | |
| SR026 | SSAP | recT | Listeria monocytogenes | MATNDELKNQLANKQNGGQVASAQSLDLKGLLEAPTMRKKF | 34 |
| | | | | EKVLDKKAPQFLTSLLNLYNGDDYLQKTDPMTVVTSAMVAAT | |
| | | | | LDLPIDKNLGYAWIVPYKGRAQFQLGYKGYIQLALRTGQYKSI | |
| | | | | NVIEVRDGELLKWNRLTEEIELDLDNNTSEKVIGYCGYFQLING | |
| | | | | FEKTVYWTRKEIEAHKKKFSKSDFGWKKDYDAMAKKTVLRN | |
| | | | | MLSKWGILSIDMQTAVTEDEAEPRERKDVTEDESIPDIIDAPITP | |
| | | | | SDTLEAGSEVQGSMI | |
| SR027 | SSAP | recT | Bacillus phage SPP1 | MATKKQEELKNALAQQNGAVPQTPVKPQDKVKGYLERMMPA | 35 |
| | | | (Bacteriophage SPP1) | IKDVLPKHLDADRLSRIAMNVIRTNPKLLECDTASLMGAVLES | |
| | | | | AKLGVEPGLLGQAYILPYTNYKKKTVEAQFILGYKGLLDLVRR | |
| | | | | SGHVSTISAQTVYKNDTFEYEYGLDDKLVHRPAPFGTDRGEPV | |
| | | | | GYYAVAKMKDGGYNFLVMSKQDVEKHRDAFSKSKNREGVV | |
| | | | | YGPWADHFDAMAKKTVLRQLLNYLPISVEQLSGVAADERTGSE | |
| | | | | LHNQFADDDNIINVDINTGEIIDHQEKLGGETNE | |
| SR028 | SSAP | recT | Haemophilus influenzae, | MATALQTLTNKLADRFDMGDGTGLTDVLTNTAFRGQKVSQD | 36 |
| | | | Haemophilus influenzae NT127 | QMTALLVVANQYGLNPWTNEVYAFPNNGGIVPIVGVDGWARI | |
| | | | | MNEHPQYDGMDFSFSEKGDSCTCTIYRKDRSRPIIVTEYMAEC | |
| | | | | QRNTQPWKSHPKRMLRHKAMIQCARLAFGFTGIYDQDEAERI | |
| | | | | VETKDPINVTPQPTVDETQAVELITPEQIEQITQLVEVTQSNMTQ | |
| | | | | LLAAAGRAPSEEKVTKANAKHVIEKLLTKLDKQQAQDEQLGE | |
| | | | | DVPIC | |
| SR029 | SSAP | recT | Clostridium botulinum C str | MANLMEIENKFEVNGAEVKLTGSIVKNYLTRGNDAVSDQEVV | 37 |
| | | | Eklund | MFINLCKYQKLNPFLNEAYLVKFKGSPAQIITSKEAYMKKAER | |
| | | | | NTNFAGMKAGIIVQRDKEILELEGSFCLKTDILLGGWAEVYKK | |
| | | | | DREFPYKAKINLDEYDKGQSTWKKMPKTMIRKTAIVQALREAF | |
| | | | | PEDLGAMYVEEEQQYQQDMSVEIKEEIKEKGNSKPLTLNPKTT | |
| | | | | ENVQNVQEVKVEEVEIIN | |
| SR030 | SSAP | gp2.5 | Synechococcus phage Syn5 | MANRYVFNTTLEGFINVYEDSGKFNNRTFAYKFDAATLEQAE | 38 |
| | | | | KDREELLKWAKSKATGRVQEAMTPWDDEGLCKYTYGAGDGS | |
| | | | | RKGKPEPIFVDSDGEVIDRNVLKDVRRGTKVRLIVQQKPYSMG | |
| | | | | PNVGTSLRVLGVQIIELATGNGAVDSGDLSVDDVAALFGKADG | |
| | | | | YKASEPAVRKAEDTVGDGDSYDF | |
| SR031 | SSAP | recT | Peptoniphilus duerdenii ATCC | MANIVKYETSNGEVQLDKQTIKNYLVSGDADKVTDQELELFIN | 39 |
| | | | BAA-1640 | LCKYQKLNPFLRDAYLVKFGDKPANMIVGKDFFIKRASANENF | |
| | | | | KGYTAGVIVLGKTGNIEERPGSFYAKQVESLVGAWCKVEFTNG | |
| | | | | TDFYHTVAFDEYNTGKSTWASKPATMIRKVALVQALREAFPE | |
| | | | | DYQGLYDSSEMGVNEEVLPTDGVKVKAPTISKEQFNLLLKTLG | |
| | | | | EDAIVDFCKSKGYNDAAKIKVDEYEKLIAEATKKKEEDEEVIE | |
| | | | | YEDVDPDFKDLQDEDNNFEINEEELPF | |
| SR032 | SSAP | recT | Paenibacillus mucilaginosus 3016 | MAAPAKATDQKDLSKALANKAAAGNGQGKTIAQLFDEMKPAI | 40 |
| | | | | AQAIPKHLTPERLERIATTSIRTNPKLKVCTPESLLGAVMQCAQ | |
| | | | | LGLEPSILGHAYLVPYRNKKKEGNKEYFVDEAQFQIGYKGLIEL | |
| | | | | ARRTGHISSIMSQAVHEKDLFEYEYGINEKLRHVPADGDRGPV | |
| | | | | TKYVAYAKFKDGGYSFMVMSKRDIELHRDKFSKAKFGPWVD | |
| | | | | HFDEMAKKTVLKALMKYMPISVEFQKAVSMDETTKREVSDD | |
| | | | | MSEVIDVTDWSESSAEDAGGGDEQRDPDTGLLNDRPPDDQVE | |
| | | | | FE | |
| SR033 | SSAP | rad52 | Lactococcus phage | MADYEEQMLALQKPLQPDRVVWRVQQSGFSKQGKPWAMVL | 41 |
| | | | bIL286, Lactococcus lactis subsp | AYMDNRAVQERFDEVFGIAGWKNEFKTAPDGGTECGISVKFG | |
| | | | lactis (strain IL1403) | DEWVTKWDGAENTQVEAVKGGLSGSMKRAAVQWGVGRYLY | |
| | | | (Streptococcus lactis) | DLPTSFAQTSLEKTDGWNKVFDKNSKKNFWWKNPQLPSWALP | |
| | | | | QNSKVQNTKADFTEEEIPNPPKLYVVGKDKKEFDEKKLQAVV | |
| | | | | NKMAIIAGKDYGASVDEQNDWLKMPLDEAYNDIEKFVDIKKE | |
| | | | | EQND | |
| SR034 | SSAP | other | Drosophila melanogaster (Fruit | MASNNSSTTDLDSQVNVEDLPITEKVKYIGSEVARGLWGIKYT | 42 |
| | | | fly) | RRPVDIMVGVAKNLPPNKVLPNCELKVSTDGVQLEIISPKASIN | |
| | | | | HWSYPIDTISYGVQDLVYTRVFAMIVVKDESSPHPFEVHAFVC | |
| | | | | DSRAMARKLTFALAAAFQDYSRRVKEATGEEEGEATPSDTITP | |
| | | | | TRHKFAIDLRTPEEIQAGELEQETEA | |
| SR035 | SSAP | gp2.5 | Burkholderia phage BcepNazgul | MAREIKFKGKNVVVFTDGTMRIENVRASYPHLAKPYKGDDQE | 43 |
| | | | | GQEKYSLVGFIEKDLAKSVLEVMKKMRDEMLREKNDGKKIPT | |
| | | | | DKFFFRDGDASGKDEYEGCYTINASETKRPSVRGADKRLLGER | |
| | | | | EIENTIYAGCRVNILINPWWQDNKFGKRINANELAVQFVRDDE | |
| | | | | PIGEGRISEDEIDDSFDDVSDGEEALAEGYDDNGGL | |
| SR036 | SSAP | recT | Legionella pneumophila | MATQKVDKRSMMVANQKTVMGLLEQMKGEIARCEPKHLTPE | 44 |
| | | | | RMARIAMTELRKTPKLQECDPLSFIASIMQAAQEGLEPGILGSC | |
| | | | | YLIPFWNSKLGKFECTFMPGYRGFLDLARRSGQIVSLVARSVYE | |
| | | | | NDEFSYEFGLKENIIHKPAMDNKGQLIAVYAVAILKDGGHQFD | |
| | | | | VMSKEEVDTVRETSKSKDNGPWVTHYEEMAKKTVLRRLFKW | |
| | | | | LPCSVEMQKAVSLDEMQEAGMQNIKVAASEEFDIDFVIDADTG | |
| | | | | EVTEIPGNKSREDLKALIEKNKAESKNSQEKETNQDKA | |
| SR037 | SSAP | recT | Lactococcus phage | MANELGIFSVDNLNMTTIKQYLDGGGKASDAELVLLINLCKQN | 45 |
| | | | phi311, Lactococcus phage | NMNPFMKEVYFIKYGNQPAQIVVSRDFYRKRAFQNPNFVGIEV | |
| | | | ul36k1t1, Lactococcus phage | GVIVLNKDGVLEHNEGTFKTHEQELVGAWARVHLKNTEIPVY | |
| | | | ul362, Lactococcus phage | VAVSYDEYVQMKDGHPNKMWTNKPCTMLGKVAESQALRMA | |
| | | | ul361, Lactococcus lactis, | FPAEFSGTYGEEEYPEPEKEPREVNGVKEPDRAQIESFDKEDYA | |
| | | | Lactococcus phage ul36k1 | AKKIEELKEKAQPQKEVVEETGEVIDEEPLEGF | |
| SR038 | SSAP | recT | Streptococcus phage M102 | MAKNELAKGSYLTDLQKLDGNTLRDFVDPKHQASPQELQALL | 46 |
| | | | | AIVKGRNLNPFTKEVYFIKYGSAPAQIVVSKEAIMKRAEENPDF | |
| | | | | DGFEAGIVVETKDGAIERLTGTIVPKSATLRGGWCKVYRKDRS | |
| | | | | HAIEADADFAYYTTSKNLWQKMPALMIRKVAIVSAFREAFSES | |
| | | | | VGGLYTADEMQRETQAEVRARKMKEAYEEKLYELTQMEAKS | |
| | | | | YKKTKSKNENEAKKTKEAEAIETVEEPTQDGNLEW | |
| SR039 | SSAP | ERF | Streptococcus phage MM1 | MADLTFAELQRKMQIEKQTKQGVKYPFRTAEDINNKFKSLDSG | 47 |
| | | | 1998, Streptococcus pneumoniae, | WSVSDPEDDIIQKGDKLYYKAVAVVKRESDGTIEKAIGWAREE | |
| | | | Streptococcus phage MM1 | DVPIFHTQKGDVKQMQDPQWTGAVGSVARKYALQGLFAIGGE | |
| | | | | DVDEYPVEESQEQGQNNQQQKPNNQQAQGQNQVRYIDNTQY | |
| | | | | QEINDLINDIAKIKGMPFDTLANYVLSEKLKGLQDFHRVQVGD | |
| | | | | YEVLKNYLTEQLAKAKAKAKRGN | |
| SR040 | SSAP | recT | Paenibacillus elgii B69 | MAQNKAIQTIDTQAVVGSFTQSELDTLKNTIAKGTTNEQFSLEV | 48 |
| | | | | QTCARSGLNPFLNQIYCIVYNTRDHGPTMSIQIAVEGIVALAKR | |
| | | | | HPQYKGFIASEVKENDEFQIDVVSGEPVHKIKTLQRGKTIGAYC | |
| | | | | VAYREGAPNISVIVTLDQIEHLLKGRNATMWKDYEDDMIVKH | |
| | | | | AIKRAFKRQFGIEVAEDEYIPSGPNIDNIPEYQPQARRDITHEAE | |
| | | | | LLAAAGRAPSLEKVTKANAKHVIEKLLTKLDKQQAQDEQLGE | |
| | | | | DVPIC | |
| SR029 | SSAP | recT | Clostridium botulinum C str | MANLMEIENKFEVNGAEVKLTGSIVKNYLTRGNDAVSDQEVV | 37 |
| | | | Eklund | MFINLCKYQKLNPFLNEAYLVKFKGSPAQIITSKEAYMKKAER | |
| | | | | NTNFAGMKAGIIVQRDKEILELEGSFCLKTDILLGGWAEVYKK | |
| | | | | DREFPYKAKINLDEYDKGQSTWKKMPKTMIRKTAIVQALREAF | |
| | | | | PEDLGAMYVEEEQQYQQDMSVEIKEEIKEKGNSKPLTLNPKTT | |
| | | | | ENVQNVQEVKVEEVEIIN | |
| SR030 | SSAP | gp2.5 | Syncchococcus phage Syn5 | MANRYVFNTTLEGFINVYEDSGKFNNRTFAYKFDAATLEQAE | 38 |
| | | | | KDREELLKWAKSKATGRVQEAMTPWDDEGLCKYTYGAGDGS | |
| | | | | RKGKPEPIFVDSDGEVIDRNVLKDVRRGTKVRLIVQQKPYSMG | |
| | | | | PNVGTSLRVLGVQIIELATGNGAVDSGDLSVDDVAALFGKADG | |
| | | | | YKASEPAVRKAEDTVGDGDSYDF | |
| SR031 | SSAP | recT | Peptouiphilus duerdenii ATCC | MANIVKYETSNGEVQLDKQTIKNYLVSGDADKVTDQELELFIN | 39 |
| | | | BAA-1640 | LCKYQKLNPFLRDAYLVKFGDKPANMIVGKDFFIKRASANENF | |
| | | | | KGYTAGVIVLGKTGNIEERPGSFYAKQVESLVGAWCKVEFTNG | |
| | | | | TDFYHTVAFDEYNTGKSTWASKPATMIRKVALVQALREAFPE | |
| | | | | DYQGLYDSSEMGVNEEVLPTDGVKVKAPTISKEQFNLLLKTLG | |
| | | | | EDAIVDFCKSKGYNDAAKIKVDEYEKLIAEATKKKLEDEEVIE | |
| | | | | YEDVDPDFKDLQDEDNNFEINEEELPF | |
| SR032 | SSAP | recT | Paenibacillus mucilaginosus 3016 | MAAPAKATDQKDLSKALANKAAAGNGQGKTIAQLFDEMKPAI | 40 |
| | | | | AQAIPKHLTPERLLRIATTSIRTNPKLKVCTPESLLGAVMQCAQ | |
| | | | | LGLEPSLLGHAYLVPYRNKKKEGNKEYFVDEAQFQIGYKGLIEL | |
| | | | | ARRTGHISSIMSQAVHEKDLFEYEYGINEKLRHVPADGDRGPV | |
| | | | | TKYYAYAKFKDGGYSFMVMSKRDIELHRDKFSKAKFGPWVD | |
| | | | | HFDEMAKKTVLKALMKYMPISVEFQKAVSMDETTKREVSDD | |
| | | | | MSEVIDVTDWSESSAEDAGGGDEQRDPDTGLLNDRPPDDQVE | |
| | | | | FE | |
| SR033 | SSAP | rad52 | Lactococcus phage | MADYEEQMLALQKPLQPDRVVWRVQQSGFSKQGKPWAMVL | 41 |
| | | | bIL286, Lactococcus lactis subsp | AYMDNRAVQERFDEVFGIAGWKNEFKTAPDGGTLCGISVKFG | |
| | | | lactis (strain IL1403) | DEWVTKWDGAENTQVEAVKGGLSGSMKRAAVQWGVGRYLY | |
| | | | (Streptococcus lactis) | DLPTSFAQTSLEKTDGWNKVFDKNSKKNFWWKNPQLPSWALP | |
| | | | | QNSKVQNTKADFTEEEIPNPPKLYVVGKDKKEFDEKKLQAVV | |
| | | | | NKMAIIAGKDYGASVDEQNDWLKMPLDEAYNDIEKFVDIKKE | |
| | | | | EQND | |
| SR034 | SSAP | other | Drosophila melanogaster (Fruit | MASNNSSTTDLDSQVNVEDLPITFKVKYIGSEVARGLWGIKYT | 42 |
| | | | fly) | RRPVDIMVGVAKNLPPNKVLPNCELKVSTDGVQLEIISPKASIN | |
| | | | | HWSYPIDTISYGVQDLVYIRVFAMIVVKDESSPHPFEVHAFVC | |
| | | | | DSRAMARKLTFALAAAFQDYSRRVKEATGEEEGEATPSDTITP | |
| | | | | TRHKFAIDLRTPEEIQAGELEQETEA | |
| SR035 | SSAP | gp2.5 | Burkholderia phage BcepNazgul | MAREIKFKGKNVVVFTDGTMRIENVRASYPHLAKPYKGDDQE | 43 |
| | | | | GQEKYSLVGFIEKDLAKSVLEVMKKMRDEMLREKNDGKKIPT | |
| | | | | DKFFFRDGDASGKDEYEGCYTLNASETKRPSVRGADKRLLGER | |
| | | | | EIENTIYAGCRVNILINPWWQDNKFGKRINANLLAVQFVRDDE | |
| | | | | PIGEGRISEDEIDDSFDDVSDGEEALAEGYDDNGGL | |
| SR036 | SSAP | recT | Legionella pneumophila | MATQKVDKRSMMVANQKTVMGLLEQMKGEIARCLPKHLTPE | 44 |
| | | | | RMARIAMTELRKTPKLQECDPLSFIASLMQAAQLGLEPGILGSC | |
| | | | | YLIPFWNSKLGKFECTFMPGYRGFLDLARRSGQIVSLVARSVYE | |
| | | | | NDEFSYEFGLKENIIHKPAMDNKGQLIAVYAVAILKDGGHQFD | |
| | | | | VMSKEEVDTVRETSKSKDNGPWVTHYEEMAKKTVLRRLFKW | |
| | | | | LPCSVEMQKAVSLDEMQEAGMQNIKVAASEEFDIDFVIDADTG | |
| | | | | EVTEIPGNKSREDLKALIEKNKAESKNSQEKETNQDKA | |
| SR037 | SSAP | recT | Lactococcus phage | MANELGIFSVDNLNMTTIKQYLDGGGKASDAELVLLLNLCKQN | 45 |
| | | | phi311, Lactococcus phage | NMNPFMKEVYFIKYGNQPAQIVVSRDFYRKRAFQNPNFVGIEV | |
| | | | ul36k1t1, Lactococcus phage | GVIVLNKDGVLEHNEGTFKTHEQELVGAWARVHLKNTEIPVY | |
| | | | ul362, Lactococcus phage | VAVSYDEYVQMKDGHPNKMWTNKPCTMLGKVAESQALRMA | |
| | | | ul361, Lactococcus lactis, | FPAEFSGTYGEEEYPEPEKEPREVNGVKEPDRAQIESFDKEDYA | |
| | | | Lactococcus phage ul36k1 | AKKIEELKEKAQPQKEVVEETGEVIDEEPLEGF | |
| SR038 | SSAP | recT | Streptococcus phage M102 | MAKNELAKGSYLTDLQKLDGNTLRDFVDPKHQASPQELQALL | 46 |
| | | | | AIVKGRNLNPFTKEVYFIKYGSAPAQIVVSKEAIMKRAEENPDF | |
| | | | | DGFEAGIVVETKDGAIERLTGTIVPKSATLRGGWCKVYRKDRS | |
| | | | | HAIEADADFAYYTTSKNLWQKMPALMIRKVAIVSAFREAFSES | |
| | | | | VGGLYTADEMQRETQAEVRARKMKEAYEEKLYLLTQMEAKS | |
| | | | | YKKTKSKNENEAKKTKEAEAIETVEEPITQDGNLEW | |
| SR039 | SSAP | ERF | Streptococcus phage MM1 | MADLTFAELQRKMQIEKQTKQGVKYPFRTAEDINNKFKSLDSG | 47 |
| | | | 1998, Streptococcus pneumoniae, | WSVSFPEDDIIQKGDKLYYKAVAVVKRESDGTIEKAIGWAREE | |
| | | | Streptococcus phage MM1 | DVPIFHTQKGDVKQMQDPQWTGAVGSYARKYALQGLFAIGGE | |
| | | | | DVDEYPVEESQEQGQNNQQQKPNNQQAQGQNQVRYIDNTQY | |
| | | | | QEINDLLNDIAKIKGMPFDTLANYVLSEKLKGLQDFHRVQVGD | |
| | | | | YEVLKNYLTEQLAKAKAKAKRGN | |
| SR040 | SSAP | recT | Paenibacillus elgii B69 | MAQNKAIQTIDTQAVVGSFTQSELDTLKNTIAKGTTNEQFSLFV | 48 |
| | | | | QTCARSGLNPFLNQIYCIVYNTRDHGPTMSIQIAVEGIVALAKR | |
| | | | | HPQYKGFIASEVKENDEFQIDVVSGEPVHKIKTLQRGKTIGAYC | |
| | | | | VAYREGAPNISVIVTLDQIEHLLKGRNATMWKDYLDDMIVKH | |
| | | | | AIKRAFKRQFGIEVAEDEYIPSGPNIDNIPEYQPQARRDITHEAE | |
| | | | | QMNNEKEQPASPQPSEDDEAAKIKKARTEMKKKFAQLGITDPE | |
| | | | | EISAYIAKNAKIKGEKPTLAEYTGLLKIMDMEIERRKAEQANDD | |
| | | | | DLLMD | |
| SR041 | SSAP | gp2.5 | Burkholderia phage BcepC6B | MAKIKLTNVRIAFINNLRTPAEFEAGDGKFRYSATFLVEKGSAN | 49 |
| | | | | DKAIEAAIKSVAVEGWAKKADAMLESFRSNSNKFCYQNGDLK | |
| | | | | DFDGFEGNMYIAAHRKRDDGRPLLLDNVADPETGKPARLVDA | |
| | | | | NGEWLAGKEGRIYAGCYVNATIDIYAQTKTNPGIRCGLMGVQ | |
| | | | | YHGPGDSFSGASRANEDDFEATAPETEDELD | |
| SR042 | SSAP | gp2.5 | Yersinia phage YpsP-G, Yersinia | MAKKIFTSALGTAEPYAYIAKPDYGNEERGFGNPRGVYKVDLT | 50 |
| | | | phage phiA1122 | IPNKDPRCQRMVDEIVKCHEEAYAAAVEEYEANPPAVARGKK | |
| | | | | PLKPYEGDMPFFDNGDGTTTFKFKCYASFQDKKTKETKHLNLV | |
| | | | | VVDSKGKKMEDVPIIGGGSKLKVKYSLVPYKWNTAVGASVKL | |
| | | | | QLESVMLVELATFGGGEDDWADEVEENGYVASGSAKASKPRD | |
| | | | | EESWDEDDGDSYEEDSDGDF | |
| SR043 | SSAP | recA | Prochlorococcus phage P-SSM2 | MDFLKEIVKEIGDEYTQVAADIQENERFIDTGSYIFNGLVSGSIF | 51 |
| | | | | GGVSSSRITAIAGESSTGKTYFSLAVVKNFLDNNPDGYCLYFDT | |
| | | | | EAAVNKGLLESRGIDMNRLVVVNVVTIEEFRSKALRAVDIYLK | |
| | | | | TSEEERKPCMFVLDSLGMLSTEKEIRDALDDKQVRDMTKSQLV | |
| | | | | KGAFRMLTLKLGQANIPLIVTNHTYDVIGSYVPTKEMGGGSGL | |
| | | | | KYAASTIIYLSKKKEKDQKEVIGNLIKAKTHKSRLSKENKEVQI | |
| | | | | RLYYDERGLDRYYGLLELGEIGGMWKNVAGRYEMNGKKIYA | |
| | | | | KEILKNPTEYFTDDIMEQLDNIAKEIIFSYGTN | |
| SR044 | SSAP | rad52 | Salmonella typhimurium, | MDLNKFDAPFNPEDIEWRIQQSGKTRDGKVWAMVLAYVTNR | 52 |
| | | | Salmonella phage | AIMKRLDDVCGKEGWRNEYRDIPNNGGVECGISIKIDSEWVTK | |
| | | | ST160, Salmonella phage ST64T | WDAAENTQVEAVKGGRSGAMKRAAVQWGIGRYLYNLEEGFA | |
| | | | (Bacteriophage ST64T) | QISSDKKQGWHRAKLKDGTGFYWLPPSLPDWAMPASCNQPSP | |
| | | | | ENTNQKSPSVDCEQILKDFSDYAATETDKKKLIERYQHDWQLL | |
| | | | | AGHDDAQTRCVQVMNIRINELKQVA | |
| SR045 | SSAP | gp2.5 | Salmonella phage SS3c | MDKCAVRDIAERDNPFEDFPEGFYFQAKNKQQPLILTSVKGEK | 53 |
| | | | | QVEPDFNIDGEQIEGEQVYSGCVANISIEIWFSEQYKVLGAKLN | |
| | | | | GIKFAGEGKAFGGSAVSASVDDLEDDEDETPRRERRRNR | |
| SRQ46 | SSAP | recT | Bacteroides caccae ATCC 43185 | MEENKLTKQENDALAIFGKGKTIYQVAGNDVALSFDIVRNYLT | 54 |
| | | | | KGNGQVSDQDIVQFISICKFNQLNPFLNEAFLVKFGQQPAQMIV | |
| | | | | SKEAFFKRADASEKYEGFKAGIIIIRDNKIVEVEGCFYNEKTDIL | |
| | | | | VGGWCEVYRSDRRFPIIAKVNLAEYDKKQSIWNEKKSTMISKI | |
| | | | | AKVQALREAFPAQLGAMYTQEEQEVKFAEYEDVTDKESKANK | |
| | | | | LAEIALKNAGVEEQPKTEQPVNQPQNNTNDKPAQKTLL | |
| SRQ47 | SSAP | ERF | Lactobacillus prophage | MEIYGKEEDRAKWAMHYAQVKANIKQPEKTHKVTVSGKTKQ | 55 |
| | | | Lj928, Lactobacillus johnsonii | GTPYSYDYNYADLNDIDAAVMDGIKKVTDKDGNVVFSYFFDI | |
| | | | (strain CNCM I-12250/La1/ | RTENNTVEVQTILVDSSGFTLKTNKVVFQNNKAWDAQATASLI | |
| | | | NCC 533) | SYAKRYSLSGAFGIAADNDDDAQDQKTIYEPKILTKQELEDYK | |
| | | | | VYYNGIQANLYDLYQEAKDGIKDAQDWLKEPHTPQDAQAVH | |
| | | | | QIAEMFKQNHSVKQETKEKQAAIDKIRQTVKKDPIEDKKVESK | |
| | | | | DPEVDKLF | |
| SR048 | SSAP | sak4 | Burkholderia phage BcepGomr | MECEMPLNWTTTANAARNGVKVLCYAPAGYGKTVLGATAPR | 56 |
| | | | | PIILSSEFGNLSIRKHNIPVLEIRNGLDMRDAYDWIMKSNEFKQH | |
| | | | | FDTLVWDGASESAETFLRVAKASGGDGRAHYGTVADMIADYF | |
| | | | | TKFRALPEKHVYITAKMGFVQDSVTGGVKYGPDFPGKQLGQQ | |
| | | | | SPYWLDEVFTLRIGTLPDGTGTYRYLQTQPDPQYDAKDRSGSL | |
| | | | | DAMSSRT | |
| SR049 | SSAP | FRF | Lactococcus phage c2 | MESKVLKLINEIKVPKSQYNSFGKYNFRNNEDIQTALKPLLLQF | 57 |
| | | | | GLMEKATTEMLEMNNELMLHVHIDIFDPDNPNDIASGDGWAV | |
| | | | | IDINKKGMDKAQATGASQSYASKYAYGQALKLDDTKDADSTN | |
| | | | | KGPNNATQMKSRPKSNYQYNLSDLKKKVANKEISSDQANELC | |
| | | | | KQGKVNMNA | |
| SR050 | SSAP | recT | Fusobacterium ulcerans 12-1B | MEVKNNLVKTEEKKNVVNFTVDGMDVKLSPAIIRSYLVNGNG | 58 |
| | | | | AVTEQEINYFIQLCKARQLNPHVKDAYLIKYGSQPATMVVSKD | |
| | | | | AIERRAIKHPQYNGKKVGIYVLDKNNELVKREHSILLEDLKLV | |
| | | | | GAWCEVYRKDWEFPAKADVNYEEYVGRKSDGTVNSQWANK | |
| | | | | PVTMLTKVAKAQALREAFIEELGGMYDSSEMSKDIPNIEEIIEAP | |
| | | | | KQDLEMKNFIEDNKEEIKDKQEKILKEAEKVEEDELIQEIFGKK | |
| SR051 | SSAP | ERF | Streptococcus phage 7201 | MEEMTFTELQQKIQLEKKKEGTAKYASRHVEDIYNVFKNLKSN | 59 |
| | | | | WNVVVNYELVEFSGKTFIKAIATASNKDEKVQAQAFAELSPVP | |
| | | | | ILKTRNGELKQMNEPQWVGAVQSYAGKYALQALFAIGEEDVD | |
| | | | | HFEVAEQSMRPNQNHNQMQSHQQANYINQQQHNQINQLIDEL | |
| | | | | AKVTGQPVETVARYYLGKYKLNNFSELLTTGFDILANDIQDKI | |
| | | | | KQRKG | |
| SR052 | SSAP | ERF | Lactobacillus prophage | MELLNQPTPPISSPQAVGLMNIQTIVGMDAKQSLDAKLSLLNDF | 60 |
| | | | Lj965, Lactobacillus johnsonii | VEMKRVLAQPSKSKDGYGYKYADLNDVLSVIQQAIGDLDLSFI | |
| | | | (strain CNCM I-12250/La1/ | QQPINKTAKTGVENYVFNSKGAILDFGSYMLDITKPQAQQYGS | |
| | | | NCC 533) | ALTYCRRYSISSIFGIASEEDTDAKALPQYMSPEEIDRLTLPYKG | |
| | | | | KQVSLAKLFSLGLAGDSKAKAKLLDRENNNVTKLAVKSMTD | |
| | | | | MWDFSKDIEAMKINEQTEIKASKDQEEKAKQAALNKVQKGKK | |
| | | | | DPFEDKKVESDSDPEVDKLF | |
| SR053 | SSAP | ERF | Clostridium phage | METNNVYIKLVNIQSTLKAPKSQFNSFGKYNYRSCEDILEGLKP | 61 |
| | | | phiC2, Peptoclostridium | ILKEEKALVILDDNIVQIGNRFYVEATATLIDAETGEKVSTKAL | |
| | | | difficile E15, Clostridium | AREDETKKGMDLAQVTGSVSSYARKYALNGLFCIDDTKDSDA | |
| | | | phage phiMMP03, | TNKHGNEQKKKEVNESELNTLYSLGESIEKDKNRVDSEVYKKF | |
| | | | Peptoclostridium difficile | GKLAVDETKQEYEKVENGYKSILEKQKQE | |
| | | | (Clostridium difficile) | | |
| SR054 | SSAP | recT | [Clostridium] methylpentosum | MEKGKQVLAEKKPEENIRLDENNIFTGFREFQVAQRMATALAS | 62 |
| | | | DSM 5476 | STIVPKDYQNNPGNCLIALEMANRLKTSPMMVMQNLYVVNGR | |
| | | | | PAWSSQYIVAMINSSRKYKTELQYEMKGSRTDGSLECTAWVE | |
| | | | | DYNGHRVTGPTVTMKMAQEEGWIGRNGSKWKTMPEVMIRYR | |
| | | | | AASFFGRLNCPDMIMGIYSEEEAIELEPSQFEFVDQVKAAEQEIK | |
| | | | | DNVGRQDIDVDVHTGEVITKPTAQTQVEEESGEPDLETQMSIEE | |
| | | | | PDF | |
| SR055 | SSAP | recT | Vibrio cholerae (strain | MEKPKLIQRFAERFSVDPNKLFDTLKATAFKQRDGSAPTNEQM | 63 |
| | | | MO10), Vibrio cholerae, | MALLVVADQVGLNPFTKEIFAFPDKQAGIIPVVGVDGWSRIINQ | |
| | | | Providencia alcalifaciens | HDQFDGMEFKTSENKVSLDGAKECPEWMECIIYRRDRSHPVKI | |
| | | | Ban1, Vibrio cholerae Ind4 | TEYLDEVYRPPFEGNGKNGPYRVDGPWQTHTKRMERHKSMIQCS | |
| | | | | RIAFGFVGIFDQDEAERIIEGQATHIVEPSVIPPEQVDDRTRG | |
| | | | | LVYKLIERAEASNAWNSALEYANEHFQGVELTFAKQEIFNAQQ | |
| | | | | QAAKALTQPLAS | |
| SR056 | SSAP | sak4 | Listeria welshimeri serovar 6b | MLEFIQSEEMKRSEVFNIMIYAKPGAGKTTTIKYLKGKTLMLD | 64 |
| | | | (strain ATCC 35897/DSM 20650/ | CDGTSKVLSGLPDITIATLNPRNPVQDMADFYGYAKTHADEYD | |
| | | | SLCC5334), Listeria phage PSA | NVVIDNLSHYQKLWLMFNGRNTKSGQPELQHVGIFDTHLIDMI | |
| | | | | SVFNNLANTNIVYTAWENTRQIQLESGQLYNQFLPDIREKVVN | |
| | | | | HVMGIVPVVARLIRNPETGQRGFLLTENNGNFAKNQLDNREFA | |
| | | | | LQEDLFKIGDIDAKA | |
| SR057 | SSAP | recT | Hydrogenobacter thermophilus | MLSKANTNTIVREEDYKKVLQMLRVALPSLAKVDDETLITAVA | 65 |
| | | | (strain DSM 6534/IAM 12695/ | YARHLGLDPLRKEVHFVPYYNKELNKTIIQPIVSYTEYLKRAER | |
| | | | TK-6) | SGKLKGWSCKFRKEGDELVAVVEIKRVDWEIPFVWEVPLSEV | |
| | | | | KRDTPLWQSHPLFMLKKTAIAQAFRMCFPEETSHLPYEEAEFW | |
| | | | | EEPSIPEQTQPTKTDVDTDTESDYITEAQRKRFFAIVKKELGYRE | |
| | | | | DAIKEILKERFGIESTAEIPKDKYEEIIDYFRSLSKPIEAEAQS | |
| SR058 | SSAP | other | Paenibacillus polymyxa (strain | MLFRRRAASAFVRKVLYFAIICGLLWLLWLGLHGVMSTGEDD | 66 |
| | | | E681) | QAVEALNEFYTMEQSGNFGGSWEFFHSQMKKKFEKSTYVQQR | |
| | | | | ARFYAGYGDGYVHV | |
| SR059 | SSAP | sak4 | Streptococcus phage Sfi21 | MRIIRAKDIQRTKNWRILIYGKAGLGKTSLIKNMPGKTLVLSLD | 67 |
| | | | | NSSKVLAGTENVDIIDFDREHPTEFITEFLTQADNLIKNYENLVI | |
| | | | | DNISSFQSDWFIEQGRKSKNGISNELQHYSQWTNYFLRVLTVIY | |
| | | | | SKPINIYVTAWEDTHELNLETGQILTQYVPQIRASVENQLLGLT | |
| | | | | DVVGRIVVNAKTGARGLILEGSEGTYAKNRLDNRTACKIEDLF | |
| | | | | KFGDLDGTKELPE | |
| SR060 | SSAP | sak4 | Lactobacillus phage phiadh | MPAFKWDEDKGTKYRWLVYGVPGVGKTTLSKYLKGKTYLLS | 68 |
| | | | | LDESFHRIDFWKGKNDIWSIDPEKPIEDLADFVKAFKPDKYDNL | |
| | | | | IIDNVSNLQKLFFIEKARETRTGLDNKMSDYNEWTTLITRFIAKC | |
| | | | | FSWNINILVTAWEAQNKVTDPSGQEFMQVGPDIRPNPRDYILG | |
| | | | | NCDVVARMVQKPQTGERGLIMQGSIDTYAKNRLDGRKGCKVE | |
| | | | | DFFEVQDK | |
| SR061 | SSAP | ERF | Mycobacterium phage PhatBacter, | MPDEAQTGPLAEDPAPHTPTVFEAWSRVMSDVQAISKDSRNEQ | 69 |
| | | | Mycobacterium phage | QHFNFRGIDAVMNVVGPALRAHGVTVIPRAVEEYSERVETQPR | |
| | | | Elph10, Mycobacterium phage | GNRPGTPMINREVRVEFTVEGPRGDWFAGTTYGEAADSGDKA | |
| | | | 244, Mycobacterium phage | MSKAHSVAYRTFLLQALTIPTDEPDPDEDVHERAPRQERRREP | |
| | | | Cjw1, Mycobacterium phage | KDEPPPLSDENAEGRAELREFCEENNLDAKVVAGKFATDNPGQ | |
| | | | Phrux, Mycobacterium phage | SIRTADNETIRAYIATMKAGLVKADV | |
| | | | Lilac, Mycobacterium phage | | |
| | | | Phaux, Mycobacterium phage | | |
| | | | Quink, Mycobacterium phage | | |
| | | | Pumpkin, Mycobacterium phage | | |
| | | | Murphy | | |
| SR062 | SSAP | recT | Cryptobacterium curtum (strain | MPQEIAKVEYTAADGQEVRLTPGVIAKYIVSGNGLASEKDIVSF | 70 |
| | | | ATCC 700683/DSM 15641/ | MARCQARGLNPLAGDAYMTVYQGKDGNTSSSVIVSKDYFVRT | |
| | | | 12-3) | ATAQDSFDGMEAGVTVLNGQGQIQKREGCEFFPSLGEKLLGG | |
| | | | | WAKVHVKDREHPSKAAVTMDEYDQHRSLWKSKPATMIRKVA | |
| | | | | IVQALREAYPGQFGGVYDRDEMPPSQEPQQVPVEVYEAPEAVE | |
| | | | | TPDNQNRATEEF | |
| SR063 | SSAP | ERF | Enterobacteria phage T1 | MHLIHQSGEVKMQLSPETNEILPALFNARNKFAKAKKDAKNN | 71 |
| | | | (Bacteriophage T1) | HLKNSVATLDAMMAAVSPALTDNDIMILQSMLDTSTETTFHLE | |
| | | | | TMLIHKSGQWAKFFMMMPIAKRDPQGVGSAMTYARRYSLAA | |
| | | | | ALGISQSDDDAQLAVKSVKDWKKELDACEDIESLKDVWANAY | |
| | | | | RQTDTASKSIIQDHYNALKAKFEIGKARGIRPAQPEQKKQVEAT | |
| | | | | SAKPVQSQSITNFE | |
| SR064 | SSAP | recT | Acinetobacter sp SH024 | MQDVDPAELANTLVNTVFKKATNDEFLSLLIVANQYKLNPFTK | 72 |
| | | | | EIYAFPAKGGGITPVVGIDGWARIINDNPVCDGIQFEQDDESCT | |
| | | | | CKIFRKDRNHPTVVTEVLSECQGNSEPWKKYPKRMERHKALIQ | |
| | | | | CARVAFGFSGIYDEDEARRIDDCHIPTVQTVSSDLPQGYEAYEQ | |
| | | | | QHLDNMRALAMEGTEALQTGYAELPQGDCKKYFWTKHSASL | |
| | | | | KEAAQHADQPQGQVYEHSPA | |
| SR065 | SSAP | other | Paenibacillus alvei DSM 29 | MQRKKLKRVMKSTNMGEDALLKTFDNFDLSDLDQRVINSYEL | 73 |
| | | | | AREYSDGLIYRIKNAKSLKGVPWFGILGTSGSGKTHLVTAAVA | |
| | | | | PLIKYDVYPLFFNWVQSFTEWFSYYNTDESYMVEEIRQRIYNCE | |
| | | | | LLVVDDICKESQKDTWIKEFYGIVDYRYRKQLPIVYTSEYFAEL | |
| | | | | IGFLSKATAGRLFERTVSPKGKKFLGEMLLNDGEDPLALDYRF | |
| | | | | KELFR | |
| SR066 | SSAP | sak4 | Lactobacillus phage Lc-Nu | MQPIKHASAIDRTKNWRVLIYGKPGVGKTSAIRNLNGKTLVLD | 74 |
| | | | | LDDSSKVLSGAPNIDVQPFDRSKPSEEWKEFLKNLAERVSGYD | |
| | | | | NLVIDNVSAFEKDWFVERGRASKNGIGNEIQDYGQWANYFARI | |
| | | | | MTMIFMDAPVNVLVTAWENTRDITSETGQSFSQYAPAIRDSVR | |
| | | | | DGLLGLTDVVGRVVVNPKTDGRGVILEGTDAIFAKNRLDNRK | |
| | | | | LVPINELFKFGNQEKSVKQED | |
| SR067 | SSAP | sak4 | Pseudomonas phage vB_Pae- | MQMSQLKPASQLARRYGVKSVVFGAPGSGKTPLINTAPRPVLL | 75 |
| | | | Kakheti25 | VTEPGMLSMRGSNVPAWEAYTPALMVEFFEWFMKSREAANF | |
| | | | | DTLGIDSISNIAEIILADELGKVKHGMKAYGNMSERVMKIANDL | |
| | | | | YYMPQKHIVMIAKQALVENGRQTILQNGEVTYEPIMQKRPFFP | |
| | | | | GKDLNVKVPHLFDNVMHLGEASVPGMPKPVRALRTKEIPEVF | |
| | | | | ARDRLGNLNELEQPDLTALFAKAMQ | |
| SR068 | SSAP | ERF | Escherichia phage Rtp | MQISELCKSILKALHTAKSLFAKAEKSKQNSHLKNKYATLEDV | 76 |
| | | | | LAAVEPGLYECGLVMFQSVLDDEQTNRMKVETKLFHVESGEW | |
| | | | | VSFLMIVPISKNDAQGYGSALTYARRYGITAALGLSQADDDGN | |
| | | | | LAAKGVKDFKRELEKCNTLDELRNVWKEAKQSLDAAGWKVF | |
| | | | | EPHIIERKAELEANAMTGFNPATPKKVAEKSSPEEKIKVESQQID | |
| | | | | TF | |
| SR069 | SSAP | recT | Streptomyces phage VWB | MIQTIAFDVGETLVSDDRYWASWADWLGAPRHTVSALVGAV | 77 |
| | | | | VAQGRDNADALRLVRPGLDVAAEYAAREAAGRGEHLDDTDL | |
| | | | | YDDVRPALAALQALGVRVIIAGNQTIRAGELLRGLDLPVDVIAT | |
| | | | | SGEWGCAKPTREFFDRVVDVAGTPRQSILYVGDHPANDTIPAA | |
| | | | | AAGLRTAHLRRGPWGHLWADDPQVRDTADWVIGSLHDLIDIV | |
| | | | | AGT | |
| SR070 | SSAP | rad52 | Paenibacillus alvei DSM 29 | MINGKTVEQVMAELAEPFPPEDIEWRVGSTNGGKTKGIALAYV | 78 |
| | | | | TNRAIMNRLDSVVGAFNWQNNYREWKGHSQLCGIGIRFGEDW | |
| | | | | IWKWDGADDSQTEAVKGGLSDSMKRAGYQWGIGRYLYKLEN | |
| | | | | VWVPIEPIGKSYRLAQTPKLPQWALPTGYTGQQTNTGNKTQRS | |
| | | | | TQKTQGKQENNSPSNNDGGSQQSSNDTGTQKRQMTEKQKQR | |
| | | | | WEVVRLLKAGGLDDAQAQAWIDKQKEQKRDYKTMLDICQRA | |
| | | | | SKR | |
| SR071 | SSAP | recT | Listeria phage B054, Listeria | MMAKENYSDPNGKLLNSITTFEVNGEEVKLSGNIIRDYLVSGN | 79 |
| | | | monocytogenes | AEVTDQEIIMFLQLCKYQKLNPFLNEAYLVKFKNTKGPDKPAQ | |
| | | | | IIVSKEAFMKRAETHEQYDGFEAGVIVERGGEIIELEGAVSLASD | |
| | | | | KLLGGWAKVFRKDRNRPVSVRISEKEFNKRQSTWNTMPLTMM | |
| | | | | RKTAVVNAMREAFPDNLGAMYTEEEQGSLQNTETSVQQEIKQ | |
| | | | | NANAEVLDIPSQQNEVPDFKEVREPEHVEMPPIYGEQQSTPPAR | |
| | | | | PY | |
| SR072 | SSAP | ERF | Mycobacterium phage Wildcat | MMRSSDNCADLLTALAKARTEIGPVEKSAQNPFFNSSYMTLDD | 80 |
| | | | | IMDAVQPVLAKNGLSVSQWPDQSVNGEPALTTLLFHEPSGQYV | |
| | | | | QATATLVLGKKDPQAEGSAITYLRRYAYVSILGIKGVEPDDDG | |
| | | | | NYASQSDKKVTRTGKVADESNASAEVTRLRKELEVAAKAAGL | |
| | | | | TSAALAKGYHDRYGADYKRDNDETHLAAALTEMQLRVKSE | |
| SR073 | SSAP | sak | Rhodothermus phage RM378 | MMNPEMKEILKKLMKPFHPDRHSYRVTGTFRTREGRNMGVV | 81 |
| | | | | AFYISSRDVMDRLDAVVGPENWRDEYEVPAPGVMKCVLYLRI | |
| | | | | GGEWVGKSDVGTGNIENPESGWKGAASDALKRAAVKWGIGR | |
| | | | | YLYALPKCYVEVDDRKRIVNEEAVKSFLHKHVTELLKNYQ | |
| SR074 | SSAP | other | Paenibacillus terrae | MIQGTLFDNEHDRPLPNRVRPQSLEDFIGQKHLLGPGKVLQDM | 82 |
| | | | (strain HPL-003) | IKNDQVSSMIFWGPPGVGKTTLAKIIANQTKSKFIDFSAVTSGIK | |
| | | | | DIRNVMKEAEGNRQLGEKTLLFIDEIHRFNKAQQDAFLPYVEKG | |
| | | | | SIILIGATTENPSFEVNSALLSRSKVFVLHQLSSEDIVELLEKAI | |
| | | | | LDPKGYGDQKIGFEDGVLSAIAEYSNGDARVALNTLEMAVLN | |
| | | | | GEKQGEAIEISKEGLLQIIHRKSLLYDKDGEEHYNIISALHKSMR | |
| | | | | NSDVNASIYWLSRMLESGEDPLFIARRLVRFASEDVGLADNRA | |
| | | | | LEITVSVFQACQFIGMPECDVHLTQAVIYLTLAPKSNSAYLAYR | |
| | | | | YAKKDALHSMSEPVPLQLRNAPTKLMKELNYGKGYQYAHDT | |
| | | | | EEKLTRMQTMPDSLIGREYYHPTTQGSESKIKQRMEEIAAWYK | |
| | | | | QNNNQK | |
| SR075 | SSAP | sak4 | Listonella phage phiHSIC | MSVLSSITKPVDRPVIMTVLGEAGLGKTSLAATFPKPIFIRTEDG | 83 |
| | | | | LQAIPEASRPDAFPMADTVESLMEQLGALVHEEHDYKTLVIDS | |
| | | | | VTQLETMFTQHVIDNDPKKPKSIQQANGGYGSGLSAVAALHG | |
| | | | | RVRKAAKMLNDKGMHIVFIAHADTETIELPDQDPYMRYSLRL | |
| | | | | GKKSMSHYVDNVDLVGLVKLQMFLKGDDDKRKKAISTGQRVI | |
| | | | | TATTSASSVTKNRYGITQDLPCELGVNPFINHIPSLTE | |
| SR076 | SSAP | sak4 | Staphylococcus phage Pv1108 | MSEEQDILQELGIEEINEDTQNYYSIMVYGKSGTGKTTLATREN | 84 |
| | | | | NAFIIDIHEDGTQVTRQGFVKRVDNYIAFRNTITSIESIVNTARQ | |
| | | | | RGKLLDVVVIETAQKLRDITLTHVMNTHQVKKARIQDYGETSK | |
| | | | | LIVNSIRHLLKVKDKLGFHVVLTGHEGLNSEDKDENGKIINPRIS | |
| | | | | IEVQPAIHNNLVTQFDIIGHTFIEDHTDENGNATHNYVESVEPSN | |
| | | | | LYTTKVRHNPQITINNPGIKNASISKIIDMAQNGN | |
| SR077 | SSAP | sak | Mycobacterium phage Hamulus, | MSEPDVEGLAKLREPFPPNQIGKLPKGGITLDFLGHGYLTARFL | 85 |
| | | | Mycobacterium phage Dante, | DVDPLWTWEPFAVGDNGLPLLDEHGGLWIRLTLCGVTRIGYG | |
| | | | Mycobacterium phage | DAGGKKGPNAVKEAIGDALRNAGMRFGAALDLWCKGDPDAP | |
| | | | Ardmore, Mycobacterium phage | APPDPAVAERNALLHELGDACAALTLDEKTVAAQFYGKYKVT | |
| | | | Llij, Mycobacterium phage Drago, | ARNAKPAQLREFIDDLMENGAPA | |
| | | | Mycobacterium phage Phatniss, | | |
| | | | Mycobacterium phage Spartacus, | | |
| | | | Mycobacterium phage Boomer, | | |
| | | | Mycobacterium phage SiSi, | | |
| | | | Mycobacterium phage PMC, | | |
| | | | Mycobacterium phage Ovechkin, | | |
| | | | Mycobacterium phage Ramsey, | | |
| | | | Mycobacterium phage Fruitloop, | | |
| | | | Mycobacterium phage SG4 | | |
| SR078 | SSAP | recT | Bacillus subtilis subsp | MSEQNELMTKSVEFSVHGEPVKLTGKTVKNFLVRGNSDVTDQ | 86 |
| | | | spizizenii (strain TU-B-10), | EAAMFINLCKYQKLNPFLNEAYLVKFKGSPAQMIVGKEAFMK | |
| | | | Jeotgalibacillus marinus | RAENNEQFQGFKAGIIVEREGKMVDLEGAIKLSNDKLIGGWAE | |
| | | | | VYRADRQQPISVRISLEEFSKGQSTWKSMPLNMIRKTAIVNALR | |
| | | | | EAFPNSLGNLYTEEEEQANDDILAEDRVRREVNENANTEIIDVN | |
| | | | | PILEPEPETTQAGQPEPQPIKRESQDKPSIFESGPDF | |
| SR079 | SSAP | recT | Paenibacillus lactis 154 | MSSQQLALVKRDTVDVVADKVRQFQERGEIHEPANYSPENAM | 87 |
| | | | | KSAWLLLQTVQTKDYKPALEVCSRDSIANALLDMVVQGLNPA | |
| | | | | KKQGYFIVYGKTLTFQRSYFGTMAVTKRVTGAEDIDAQIIYEG | |
| | | | | DEFEFEIVRGRKKITKHKPSFSNMDDSKIAGAYCTIYWPDGREY | |
| | | | | TEVMTMKEIRQAWKKSKQNPDKENSTHNEFPGEMAKRTVINR | |
| | | | | TCKTYMNTSDDGSLIMKHFKRQDEVLAEAEVEAEIAANANGE | |
| | | | | VIDITPPATTEAEPEPEQPKETTRSSKSNVELAGQGEMDFEIHPD | |
| | | | | DIPPFGTEGPDE | |
| SR080 | SSAP | recT | Pediococcus acidilactici DSM | MSNELMTKAVTYEVNNEEVKLSGQIVKQYLTSGQAVTDQEVT | 88 |
| | | | 20284 | MFIQLCRYQHLNPFLNEAYLVKENGKPAQIITSKEAFMKRAESN | |
| | | | | PNYAGLKAGCIVERNGELIYTEGAFTLKTDNILGAWADVIRKD | |
| | | | | RREPTHVEISMDEFSKSQATWKSMPATMIRKTAIVNALREAFP | |
| | | | | QDLGALYTEDDKNPNEATQTTYKQEPEVNTTKTADVLAKKFS | |
| | | | | GAPQIKSVENVQESEEESNNASNHGEATEPVNNVEEPTATAEV | |
| | | | | EQGQLL | |
| SR081 | SSAP | ERF | Enterobacteria phage HK022 | MSKEFYARLAAIQENLNAPKNQYNSFGKYKYRSCEDILEGVKP | 89 |
| | | | (Bacteriophage HK022) | LLNGLFLSISDEVVLIGDRYYVKATATITDGENSHTATALAREE | |
| | | | | ESKKGMDSAQVTGATSSYARKYCLNGLFGIDDAKDADTDEHK | |
| | | | | HQQNAAAKQSNPSPTPEQVLKAFTDAAMQKNTVEELKQAFAK | |
| | | | | AWKMLEGTPEQHKAQDVYNIRRDELEGAAA | |
| SR082 | SSAP | rad52 | Homo sapiens (Human) | MSGTEEAILGGRDSHPAAGGGSVLCFGQCQYTAEEYQAIQKAL | 90 |
| | | | | RQRLGPEYISSRMAGGGQKVCYIEGHRVINLANEMFGYNGWA | |
| | | | | HSITQQNVDFVDLNNGKFYVGVCAFVRVQLKDGSYHEDVGYG | |
| | | | | VSEGLKSKALSLEKARKEAVTDGLKRALRSFGNALGNCILDKD | |
| | | | | YLRSLNKLPRQLPLEVDLTKAKRQDLEPSVEEARYNSCRPNMA | |
| | | | | LGHPQLQQVTSPSRPSHAVIPADQDCSSRSLSSSAVESEATHQR | |
| | | | | KLRQKQLQQQFRERMEKQQVRVSTPSAEKSEAAPPAPPVTHST | |
| | | | | PVTVSEPLLEKDFLAGVTQELIKTLEDNSEKWAVTPDAGDGVV | |
| | | | | KPSSRADPAQTSDTLALNNQMVTQNRTPHSVCHQKPQAKSGS | |
| | | | | WDLQTYSADQRTTGNWESHRKSQDMKKRKYDPS | |
| SR083 | SSAP | recT | Burkholderia cenocepacia (strain | MSDVITTNQDTAPGAFDLSPRSLEQAMQLANILADSSIVPKDFI | 91 |
| | | | ATCC BAA-245/DSM 16553/ | GKPGNVLVAIQWGMELGLKPMQAMQNIAVINGRPSLWGDAL | |
| | | | LMG 16656/NCTC 13227/ | LALVLASPVCEYVHEWEENGTAFIKVKRRGKPEDVQSFGDED | |
| | | | J2315/CF5610) (Burkholderia | AKKAGLIGKQGPWAQYPQRMKKMRARAFALRDNFADVLKGI | |
| | | | cepacia (strain | AVAEEVMDIEPVERDITPRATAAQIAHSAADSSRPARTERHDEI | |
| | | | J2315)), Burkholderia cenocepacia | VKKLEDVARNLGFEPFKEEWTKLSRDDRAALGLRERDRIAAIA | |
| | | | BC7 | GAPVVHQQTDGAPQDDGHGAGQREPGGDDE | |
| SR084 | SSAP | recT | Photorhabdus luminescens subsp | MSTAVQKVYEIINPLKTEFEQICSEPGIVFKRESEFAMQIFANND | 92 |
| | | | laumondii (strain DSM 15139/ | FLATTALNNPVSARSAVMNIAAIGISLNPAQKLAYLVPRNKSVC | |
| | | | CIP105565/TT01) | LDISYMGLMHIAQQSGAIKWCQSAVVRKNDIFKRTSIDTAPIHE | |
| | | | | YNEFDTAQSRGDIVGAYTVIKTDDCDYITHTMRASAIFDIRDRS | |
| | | | | SAWIAYKTKGKSCPWVTDEEQMILKTVVKQAAKYWPRRERL | |
| | | | | DKAIDYVNTEAGEGIDFSKGQNSVIDVTPAADSTIESITNLLTTM | |
| | | | | NKTWDDDLLPLCSKIFRRQILSPAELTESEAIKASDFLRKKAS | |
| SR085 | SSAP | recT | Bacillus thuringiensis | MSTALATLAGKLAERVGMDSVDPQELITTLRQTAFKGDASDA | 93 |
| | | | Sbt003, Escherichia coli\′BL21- | QFIALLIVANQYGLNPWTKEIYAFPDKQNGIVPVVGVDGWSRII | |
| | | | Gold(DE3)pLysS | NENQQFDGMDFEQDNESCTCRIYRKDRNHPICVTEWMDECRR | |
| | | | AG\′, Enterobacteria phage | EPFKTREGREITGPWQSHPKRMLRHKAMIQCARLAFGFAGIYD | |
| | | | HK630, Enterobacteria phage | KDEAERIVENTAYTAERQPERDITPVNDETMQEINTLLIALDKT | |
| | | | lambda (Bacteriophage | WDDDLLPLCSQIFRRDIRASSELTQAEAVKALGFLKQKAAEQK | |
| | | | lambda), Escherichia coli | VAA | |
| | | | TA280, Escherichia coli 1-176- | | |
| | | | 05_S3_C2, Escherichia coli 40967 | | |
| SR086 | SSAP | recT | Listeria phage A118 | MSTNDELKNKLANKQNGGQVASAQSLGLKGLLEAPTMRKKFE | 94 |
| | | | (Bacteriophage A118) | SVLDKKAPQFLTSLLNLYNGDDYLQKTDPMTVVTSAMVAATL | |
| | | | | DLPIDKNLGYAWIVPYKGRAQFQLGYKGYIQLALRTGQYKSIN | |
| | | | | VIEVRDGELLKWNRLTEEIELDLDNNTSEKVIGYCGYFQLINGF | |
| | | | | EKTVYWTRKEIEAHKKKFSKSDFGWKKDYDAMAKKTVLRNM | |
| | | | | LSKWGILSIDMQTAVTEDEAEPRERKDVTEDESIPDIIDAPITP | |
| | | | | SDTLEAGSEVQGSMI | |
| SR087 | SSAP | ERF | Streptococcus pyogenes serotype | MRKSESITEYAKAFCKAQLEVKQPLKDKDNPFFKSKYVPLENV | 95 |
| | | | M28 (strain | TEAITKAFANNGISFSQDPTTNAENGYIDVATLVMHTSGEWVE | |
| | | | MGAS6180), Streptococcus | YGPLSVKPTKNDVQGAGSAITYAKRYALSAIFGITSDQDDDGN | |
| | | | pyogenes, Temperate phage | EASKPNKTNQSQKPTNKTSKGASFQTPKISNIQVETYKSDLSDI | |
| | | | phiNIH11, Streptococcus pyogenes | AKATNQNVEELTKWLTDTLKVKTLEDLRTEQIVSTDNLINKLK | |
| | | | serotype M2 (strain | KKAEQKND | |
| | | | MGAS10270), Streptococcus | | |
| | | | pyogenes serotype M3 (strain | | |
| | | | ATCC BAA-595/ | | |
| | | | MGAS315), Streptococcus | | |
| | | | pyogenes STAB902 | | |
| SR088 | SSAP | sak4 | Staphylococcus phage phill | MTEKTNQDVDILTQLGVKDISKQNANKFYKFAIYGKFGTGKTT | 96 |
| | | | (Bacteriophage phi- | FLTKDNNALVLDINEDGTTVTEDGAVVQIKNYKHFSAVIKMLP | |
| | | | 11), Staphylococcus phage | KIIEQLRENGKQIDVVVIETIQKLRDITMDDIMDGKSKKPTFND | |
| | | | 80, Staphylococcus phage | WGECATRIVSIYRYISKLQEHYQFHLAISGHEGINKDKDDEGSTI | |
| | | | 52A, Staphylococcus aureus | NPTITIEAQDQIKKAVISQSDVLARMTIEEHEQDGEKTYQYVLN | |
| | | | (strain NCTC 8325) | AEPSNLFETKIRHSSNIKINNKRFINPSINDVVQAIRNGN | |
| SR089 | SSAP | rad52 | Pseudomonas aeruginosa, | ATGACTAACATGAATTCAAATATGGCTATCTGGGATCAAGT | 97 |
| | | | Pseudomonas aeruginosa | GAAAGAGACAGATACCAGATATACCAGACAAGCTAAGCTCA | |
| | | | DHS0, Pseudomonas phage | ATGGCCAGGACATGACATCCATCAACGGACTCTATATAGTT | |
| | | | LKA5, Pseudomonas phage F116 | CGCCGCGCTACAGAATTATTTGGTCCAGTTGGCAAAGGTTG | |
| | | | | GGGTTGGAAAGTCCTCGTAGAACGTTTTGACGAAGGGGCCC | |
| | | | | CGCATTTAGACAAGAATGGTGCGGTTATTTGTCACGATAAA | |
| | | | | ACACATACCTTGTATATAGAATTGTGGTACCGTCATGATGGA | |
| | | | | ACAATTAACCATGCTAGGCAGTATGGGCATACCCCGTACGT | |
| | | | | CTATAAAACTGAGTGGGGTTTCAAAACGGATCACGATTATG | |
| | | | | GGAAAAAATCGCTCACAGATGCGATTAAAAAGTGCCTGAGT | |
| | | | | TTGCTCGGTTTTTCTGCGGATATCCACATGGGTATGTTCGAC | |
| | | | | GATACAACGTATGTTGAGGGTCTGAAATTAAAAGAGAGATT | |
| | | | | AGCTGATGCTGGTGATCCGGAGACTGCTTTAGATGAAGCCA | |
| | | | | AAGACGAGTTTAAAACTTGGCTTCGTGCCCAGTTAGATGCC | |
| | | | | ATTGCTGCTGCTCCGAATAGCCGTGCTTTAGAACTAATGCGC | |
| | | | | AAACAGGTGGCCGAAAAAGCTAGAGCCAAAGCCCCTGTAGT | |
| | | | | TAATTTCAATCCGAGCGAAATTGAGCTGAGAGTGAATGAGG | |
| | | | | CGGCGGATGAGAGGCTAAGACAGCTATCTCCCGCCCCGACG | |
| | | | | AGCCCGGAAGAGTAA | |
| SR090 | SSAP | sak | Staphylococcus phage CNPH82 | MTEETLFNQLNQKDVNDHVEKKNGLTYLAWSVAHQELKKIDS | 98 |
| | | | | NYSIKTHEFVHPDVPQDNYFVPYLATPEGYFVQVSVTVKGQTE | |
| | | | | TEWLPVLDFRNKSLAKGSATTFDINKAQKRCFVKAAALHGLG | |
| | | | | LYIYNGEEVPSANDNDITELEERINQFVTLSQEKGRDATLDKTM | |
| | | | | RWLGIQNINKVTKKDIANAHQKLDAGLKQLDKENSND | |
| SR091 | SSAP | gp2.5 | Streptococcus pyogenes | MTTTPNTTKVVTGKVRLSYVALLEPKAFEGQEAKYSTVILIPKT | 99 |
| | | | STAB902, Streptococcus | DKVTIKKIKDAQKAAYEAAKDNKLKGVKWERVKTTLRDGDE | |
| | | | pyogenes, Streptococcus | EMDTEEHPEYTGHMFMSVSSKTKPQIIDKYKNFVDSAEEVYSG | |
| | | | pyogenes serotype M3 (strain | VYARVSLNAYAYNTAGNKGISCGLNNVQIVAKGDYLGGRSSA | |
| | | | ATCC BAA-595/MGAS315) | DADFDEWNEEEDEDDIL | |
| SR092 | SSAP | other | Paenibacillus curdlanolyticus | MTNMQHMMKQVKKMQEQMLKAQEELGTKTIEGSAGGGVVN | 100 |
| | | | YK9 | VKVNGHKQVLSITIKPEVVDPEDIEMLQDLVITAVNDALAKAE | |
| | | | | EMANEDMGKYTGGMKIPGLF | |
| SR093 | SSAP | recT | Streptococcus phage | MTNNQLATQTKRNITTDPSLLTGADIKKYFDPQNLLTEKQVGQ | 101 |
| | | | V22, Streptococcus pneumoniae | ALALCKGRNLNPFANEVYIVAYTNRNGGKEFSLIVSKEAFLKR | |
| | | | | AAQCKDYEGFEAGVVVVDSEGVMHERKGAIMLPEDTLIGGW | |
| | | | | ARVHRKNFKVPVEIFVSREEYDKKQSTWNTMPATMIRKVALV | |
| | | | | NALREAFPEDLGNMYTEDDGGETFDRIKDVTPQESREDVVARK | |
| | | | | MAEIEQFNKEQEANHADPEPAQNEDPIQGELLDGELEY | |
| SR094 | SSAP | recT | Cyanophage PSS2 | MTEQHAQRWDERTKKIVFATVAKDVRSEADKAHFEHLCTSKG | 102 |
| | | | | LDPLAKEIWCVARGGKPTFMLSIDGFLKLANSTGMLDGIDIEFF | |
| | | | | DADGKGSEVWVSSKPPAACVARAYRKNCSRPFSASCRFDAYA | |
| | | | | QNSPLWKKLPEVMLSKVATTLALRRGFSDVLSGLHSPEEMDQ | |
| | | | | AGMAPAEALAPSAPAAPAPVVNPPAEKPRKRMAPVEPVHEVID | |
| | | | | RPTDDLHHGGVTEVQKNVAPKAVAVMEKPVEEGLPARDDLK | |
| | | | | GAISKLYEVARAKGLTVKGWESLGLQMGGLTPGSAAKFLTPM | |
| | | | | SSISEEKVGYLNSGKSTTGEALR | |
| SR095 | SSAP | recT | Vibrio cholerae 1587 | MTQVIPFEQQYPLVAQRGIDEATWSALQNSVYPGAKPESILMV | 103 |
| | | | | VDYCRARSLDPILKPVHIVPMSVKNSQTNQNEWRDVVMPGIG | |
| | | | | MYRIQADRSKTYAGSTEPEFGPPITMEFHGEGNAKETITEPEWC | |
| | | | | KITVYKLINGSPVAFSAKEMWLENYATAGRNSQIPNAMWKKR | |
| | | | | QYAQLAKCTEAQALRKAWPEIGQQATAEEMEGKELIIEHDITP | |
| | | | | AKKSAPQIQQYPQDDFDKNFPAWEKKIKLGTNTPERIIAMVQS | |
| | | | | RGQLTDEMKQRLFDASAINGELSHANSN | |
| SR096 | SSAP | recT | Haemophilus | MTTALNTLTQKLAERFEMGSSENLPQTLMATAFKGQNVTPDQ | 104 |
| | | | paraphrohaemolyticus HK411 | MVALLVVANQYELNPWTNEIYAFPNKEGIIPIVGVDGWVAIIN | |
| | | | | KHPQFDGIEFEQDAESCTCRIYRKDRQRPTVVTEYLDECKRETD | |
| | | | | PWRKYPKRMLRHKAAIQCARVAFSLSGIYEPDEAERIQESDKTS | |
| | | | | QNITPEKSATTGNPTHPEFEALVFTLEEEAKKGTSHLETYWKTT | |
| | | | | LTKEQRQIVGVNEITRIKEKAKQYDNIQEAEYEEA | |
| SR097 | SSAP | recT | Bartonella schoenbuchensis | MTTSIVATVAQKYGLSEQEFCKKIIKNCINFNISKEDFEDFVYL | 105 |
| | | | (strain DSM 13525/NCTC 13165/R1) | ADGYKLNPLRKEIYAVPKRGGGIDAVVGIKGWYKLIRSQDDYD | |
| | | | | GIDIIYQHDNNGKLHAAQCIIYLKSKKYPIKFTEFLEECRRDTE | |
| | | | | HWRKSSSRMLCYKAITQCARLAFGFDDIYDEDEVDCIDEAFISE | |
| | | | | VNHNPQNERVSDEVLAQIRELMEQTKTEEKKVLSFAKVASLTEM | |
| | | | | SHETGQIILEGLKERQRFQMEEEQQTLPSPKQPHTPTQQDLPGV | |
| SR098 | SSAP | ERF | Phormidium phage Pf-WMP3 | MTNNITPDFIKAFVKVQSELPAIPKSSTNPHFKSKFASLDTFNE | 106 |
| | | | | LVLPVLHTYGFTLMQPCSVENDNAVIDTYLMHESGGYVVSRYLI | |
| | | | | GFDDNPQKQGARVTYGRRYAAFAILGVVGDEDDDGNSAVGLS | |
| | | | | GTKQTSAEPVATLKRSGGERKLQ | |
| SR099 | SSAP | recT | Simkania negevensis (strain | MTVQLVQPRNSDEYDFDQTKLDLIKRTICKGATNDELQLFIHA | 107 |
| | | | ATCC VR-1471/Z) | CKRTGLDPFMRQIFAVKRWDSSTKKEIMTIQTGIDGYRLIADRT | |
| | | | | GKYAPGKDTEFGYDNKGNIRWAKAYIKKMTPDGQWHEISAIA | |
| | | | | FWEEYVQTTREGKSTLFWLKKSHIMLSKCTEALAERKTFPAER | |
| | | | | SGIYTKEEMAQEFSPLEEHLVERIAASRNDQGRS | |
| SR100 | SSAP | sak | Lactobacillus phage | MTDKKKSVYETLAKVDVKPLLEKKGNLNYLSWAKAWGLVKS | 108 |
| | | | KC5a, Lactobacillus | LYPDATYQIKEEPEYVLTKESWLATGRNVDYRQTIAGTEVEVT | |
| | | | phage phi jlb1 | VTIEDQSYSSKLYVMDYRNKVIAKPTYFEINKTQMRCLVKALA | |
| | | | | FAGLGLDVYAGEDLPEQPQRKQTVPKPAQKPARRQATLSKPK | |
| | | | | KMTKDQLYGYQADYNGEKKFLVTIYTECDKQKKMGLDAKTS | |
| | | | | VPIEWWHEKLKENSADGEAVRQFTEMAIAANKKKQANGIPDY | |
| | | | | AKDPEVEKAIEDAIS | |
| SR101 | SSAP | rad52 | Thermus phage phiYS40 | MTEKEIRIELMRPFPDQAILFRVDKKLKNGSYLIVPYLDVRVII | 109 |
| | | | | HRLNTMIPGDWELKTEITPITVETTDKSGFLIVGHMAKAELTIM | |
| | | | | GKTMTGTGSSYLVFDNDLEKLKKQFIKADPKSAETDAIRRAAAN | |
| | | | | HSIGLYIWFFKNQIFATEEELKNRNSKPIQEALANERIIGDKLY | |
| | | | | RQAKEYALGKRGGAE | |
| SR102 | SSAP | recT | Streptococcus gallolyticus | MTTNEITQQKGGYLTDLQQLDGATLRNFVDPKHQASPQELQTL | 110 |
| | | | subsp gallolyticus TX20005 | LAIVKNRNLNPFTKEVYFIKYGNNPAQIVVSKDAFMKRAEQNP | |
| | | | | NYDGFESGVIYENQTGELKSKKGVILPKNCKEVGGWCEVYRK | |
| | | | | DRSRPVYREVELSAYNTGKNWWGKAPGQMIEKVAIVAAVRD | |
| | | | | TFSEDVGGLYTTDEMEQAAPIDVTPQETQEDVIARKQAQIENW | |
| | | | | QNQKAQQEEAQAEPAQEEQTELLDENGELVY | |
| SR103 | SSAP | recT | Acinetobacter radioresistens | MNAVTQVENQLGLSLKDYDVDQAMWSALTSSIFPGAKPESIV | 111 |
| | | | SK82 | MAVEYCKARNLDIMKKPCHIVPMSVKDAKTGNSDWRDVIMPSI | |
| | | | | AEHRITASRSHSYAGIDAPVFGPMVNISFGGVSHTVPEFCTVT | |
| | | | | VYRIIHGEKVAFAHTEYFEEACATVKGGGLNSMWTKRKRGQLA | |
| | | | | KCAEAGALRKAFPEEIGDGYTKEEMEGKIITVGGTEHIQEEAKA | |
| | | | | IEDIRPTLTEKQIETAIKKIKANQGSLDALKAHYNITPEQENYV | |
| | | | | RAQTEVLEHDPV | |
| SR104 | SSAP | recT | Acinetobacter sp P8-3-8 | MNAPIQHSTIVTAQITQMADLLGLGNVDPVELKNTLIATAFNN | 112 |
| | | | | GDKEISDAQMASLLIVAGQYKLNPWTKEIYAFPDKNKGIIPVVG | |
| | | | | VDGWSRIVNSSPNFDGLEFKYSENMVTMEGAKVAAPEWVDCII | |
| | | | | YRKDRERPTVVREYLAECYRPPFKGKYGDVVGPWQSHPSRFL | |
| | | | | RHKATIQCARLAFGFVGIYDQDEAERIQDGGTVRTVQGETGDL | |
| | | | | IPDGYQEFEEAHLNNMRELAFEGMEALETGYAELPKGKCRSHF | |
| | | | | WTMHKESLKAAAQKADQPQGEVYEHSPAE | |
| SR105 | SSAP | recT | Lactobacillus ruminis SPM0211 | MNQLQKQQTRSITFKANGDDVTLSPSIVRDYLVRGNSKEVTGQ | 113 |
| | | | | EIAMFLNLCKFQHLNPFLNEAFIVKFGDKPAQLITSKEAFMKRA | |
| | | | | ESHPQYNGLKAGVIVVNNNGVEFRNGAFTVPDFDQLVGGWCE | |
| | | | | VYRKDRDIPVRVEISLSEFSKGQSTWKTMPATMIRKTAIVNALR | |
| | | | | EAFPETLGALYTEDDDGQMQMQQTKKQVQATENSKAKNKAD | |
| | | | | ALIAQAMDPEHVQQQETEEFQREPRPVDLFNPAEEYSKGE | |
| SR106 | SSAP | sak | Bacillus phage 0305phi8-36 | MNINECFQVSHYEVDKNLRKLIDEPVNEDLIDKNKYANNSEEV | 114 |
| | | | | PDSIYKRVLNKATNYQWSFVPYDISTVEGKYVQFIGLLIVPGYG | |
| | | | | VHTGIGTQKLQKTDNSNALSAAKTYAFKNACKEMGIAPNVGN | |
| | | | | DDFDEALFENFDDDEIEVEEKPKKKPVKKEEKKPAKKPAKKEA | |
| | | | | KPLKERIEEIRQAYELDDDDEVAFIQIWDENIIDLKDMDDKKWK | |
| | | | | AFLQDVEENPEEYEDF | |
| SR107 | SSAP | ERF | Staphylococcus phage 92 | MNKSETVVEINKAMVAFRKEVKQPEKDKNNPFFKSKYVPLEN | 115 |
| | | | | VVEAIDEAATPHGLSYTQWALNDVDGRVGVATMLMHESGEYI | |
| | | | | EYDPVFMNAEKNTPQGAGSLISYLKRYSLSAIFGITSDQDDDGN | |
| | | | | EASGKNNNPKQQTRTQWASSETIGILRKEVIDFTKLIKGTDKEA | |
| | | | | PQNIVEQKFDINNYKLTEKQAAEAIQKIRNNTKTVTGGKQ | |
| SR108 | SSAP | sak4 | Lactobacillus phage phig1e | MKFYEDGNIPEIPNMYFIYGDGGTGKTSVVKQFVGHKLLFSFD | 116 |
| | | | | MSSNVLIGDKDVDVIMFEHRDMPNIQAMVEQYVMQGIQDVKY | |
| | | | | RIIVLDNITALQNLVLENIDDAAKDNRQNYQKLQLWFRDLGTIL | |
| | | | | KESGKTVYATAHQLDNGSSGLSGEGRYQADMNEKTFNAFTSM | |
| | | | | FDLVGRIYLTGGERMIDLDPEKGNHAKNRIDGRKLIKANELIQT | |
| | | | | SKGAK | |
| SR109 | SSAP | recT | Bacillus phage 0305phi8-36 | MKSRELTPEFKKEFIMKIGGPKGKEAILYNGLLALAHEDPRFGH | 117 |
| | | | | IEAYVTQYPSSENKFTCYARAEIFDKEGKRIGMEEADASVNNC | |
| | | | | GKMTAASFPRMALTRAKGRAFRDFLNVGMVTADELQAYEPD | |
| | | | | LASVDSIGKIKRLGKKLGFSRTQLEDFLYDNIGTDKMNELTEPE | |
| | | | | AQEIIDAMNAELADQEPDEEVEEKPARKKRPAKKKSASVDDED | |
| | | | | F | |
| SR110 | SSAP | gp2.5 | Staphylococcus phage | MKAKVLNKTKVITGKVRASYAHIFEPHSMQEGQEAKYSISLIIP | 118 |
| | | | 3A, Staphylococcus phage | KSDTSTIKAIEQAIEAAKEEGKVSKFGGKVPANLKLPLRDGDTE | |
| | | | phi7401PVL, Streptococcus | REDDVNYQDAYFINASSKQAPGIIDQNKIRLTDSGTIVSGDYIRA | |
| | | | pneumoniae, Staphylococcus | SINLFPFNTNGNKGIAVGLNNIQLVEKGEPLGGASAAEDDFDEL | |
| | | | aureus (strain NCTC | DTDDEDFL | |
| | | | 8325), Staphylococcus phage | | |
| | | | Phi12, Staphylococcus aureus, | | |
| | | | Staphylococcus phage 47, | | |
| | | | Staphylococcus phage tp310-2 | | |
| SR111 | SSAP | ERF | Flavobacterium phage 11b | MKEIATALVKAQKEMTTPKKGSVNPFFKNKYADLNDVLAAIV | 119 |
| | | | | PALNNNGIVLLQPLVNIEGKNFVKTVLMHESGEVFESLAEIFCN | |
| | | | | KNNDAQAYGSGVSYARRYSLSSICGIGSEDDDAHTAVNTKPKA | |
| | | | | VPVVLTLTDAVIDSVIAKGIDTIKSCLESIKNGSRIATQVQILK | |
| | | | | LENGSK | |
| SR112 | SSAP | gp2.5 | Acyrthosiphon pisum secondary | MKIKLNNVRLAFPELFEPTQVSGQGAFKYRANFLIPKSRTDLIE | 120 |
| | | | endosymbiont phage 1 | EIKAGIKHVIGEKWGNKDIEKIYNSICNNPNRFCLRDGDSKEYD | |
| | | | (BacteriophageAPSE-1) | GYAGNLYLSASNKSRPLVIDRNTSPLTAQDGRPYSGCYVNATV | |
| | | | | EFYGYDNNGKGVSASLRGVQFFRDGDAFTGGGVASVEEFDDL | |
| | | | | SMAEEEELLAS | |
| SR113 | SSAP | sak4 | Streptococcus phage | MKITKATEIKNNDSCYLIYGNPGFGKTSTAKYLPGKTIVINIDK | 121 |
| | | | A25, Streptococcus pyogenes, | SAKVLRGNENIDIADIDTHKIWGEWLDTVKELLNGAANDYDNIV | |
| | | | Streptococcus pyogenes serotype | IDNVSELFRACLANLGREGKNHRVPSQADYQRVDFTILDSLRA | |
| | | | M2 (strain | LLQLNKRIVFLAWETSDQWTDENGMIYNRAMPDIRTKILNNFL | |
| | | | MGAS10270), Streptococcus | GLTDVVARLVKKTTDDGEEVRGFILQPSASVYAKNRLDDRKG | |
| | | | pyogenes serotype M4 (strain | CKVEELFETT | |
| | | | MGAS10750), Streptococcus | | |
| | | | pyogenes serotype M3 (strain | | |
| | | | ATCC BAA-595/ | | |
| | | | MGAS315), Streptococcus | | |
| | | | pyogenes GA06023, Streptococcus | | |
| | | | pyogenes STAB902 | | |
| SR114 | SSAP | gp2.5 | Prochlorococcus phage P-55P7 | MKNIHVTKEPVTLEGYQAILKPSKFGYSLKAVVGSDIVDALETE | 122 |
| | | | | RADCLKWAESKLKNPKRATLKPTPWEEVEEGKFIVKFSWAED | |
| | | | | KRPPVVDTEGTPITNEDVPVYEGSKVKIGFHQKPYILRDGVTYG | |
| | | | | TSLKLSGIQVVSIQSGAGVDTGDEDQDGVAELFGKTQGFKADD | |
| | | | | PNVTPAEEAPVPDDDF | |
| SR115 | SSAP | sak4 | Lactobacillus phage phij11 | MKKIVNFTDGANIFVVLGQVGSGKTHLTLGHKGKKLVISFDGS | 123 |
| | | | | YSTLEGHEDEMTVVEPEITDYGNPDKLVSEIDDLAKGCDLVVF | |
| | | | | DNISAVETSLVDAITDGKLGNNTDGRAAYGVVQKLMAKFARW | |
| | | | | AIHFNGDVLFTLWSEVTEEGKEKPAMNAKAFNSVAGYAKLVS | |
| | | | | RTETGEDGYTVVVNPDNRGVIKNRLADKIKKQSIKNDDYWKA | |
| | | | | VEFAKGSKHENA | |
| SR116 | SSAP | recT | Komagataeibacter oboediens | MNALTVQGHTPAIQITSFEQLVKFADIASRSGMVPSAYSGKPQA | 124 |
| | | | | VLIAVQMGSELGLAPMQSLQNIAVINGRPSVWGDALLGLVKAS | |
| | | | | AVCDDVVETMEGEGDALTAICVAKRKGKSPVEARFSVEDAKA | |
| | | | | AGLWNKQGPWKQYPKRMLQMRARGFALRDAFPDVLRGLITA | |
| | | | | EEAGDIPQDDFRSSVSGQRPVDVTPQVRHVEQERAPNYIAYFES | |
| | | | | KLADCRNTTSVDALWKQWNDRIEKAHSAGRPIAQETIERVQE | |
| | | | | MMGERMGEVEQAERQQTEELAAAPVEEPVA | |
| SR117 | SSAP | rad52 | Saccharomyces cerevisiae (strain | MNEIMDMDEKKPVFGNHSEDIQTKLDKKLGPEYISKRVGFGTS | 125 |
| | | | ATCC 204508/S288c) (Baker\s | RIAYIEGWRVINLANQIFGYNGWSTEVKSVVIDFLDERQGKESI | |
| | | | yeast), Saccharomyces cerevisiae | GCTAIVRVTLTSGTYREDIGYGTVENERRKPAAFERAKKSAVT | |
| | | | YJM1250, Saccharomyces | DALKRSLRGFGNALGNCLYDKDFLAKIDKVKFDPPDFDENNLF | |
| | | | cerevisiae YJM451 | RPTDEISESSRTNTLHENQEQQQYPNKRRQLTKVTNTNPDSTKN | |
| | | | | LVKIENTVSRGTPMMAAPAEANSKNSSNKDTDLKSLDASKQD | |
| | | | | QDDLLDDSLMFSDDFQDDDLINMGNTNSNVLTTEKDPVVAKQ | |
| | | | | SPTASSNPEAEQITFVTAKAATSVQNERYIGEESIFDPKYQAQSI | |
| | | | | RHTVDQTTSKHIPASVLKDKTMTTARDSVYEKFAPKGKQLSM | |
| | | | | KNNDKELGPHMLEGAGNQVPRETTPIKTNATAFPPAAAPRFAP | |
| | | | | PSKVVHPNGNGAVPAVPQQRSTRREVGRPKINPLHARKPT | |
| SR118 | SSAP | recT | Clostridium botulinum (strain | MNENKELTEQQGANIANDLDTFGSIKGFENTCRMAKALASSTI | 126 |
| | | | Eklund 17B/Type B) | VPKEYHDNIGNCLIALDVANRVGLSPIMVMQNLYVVNGRPAW | |
| | | | | GSQSISALINTSKKYAKPLQYKIDGSGDELSCYAYTFDHEENEIK | |
| | | | | GPVITIKMAKEEGWINKSGSKWKTMPEVMIRYRAASFFGRLHC | |
| | | | | SELLLGIYSADEAVELEPATVIEVTHEEVKQEIKENANQEIIDI | |
| | | | | QIEEVKEDDKEKSKNENPTVKQTVVKTEPF | |
| SR119 | SSAP | recT | Campylobacter coli 80352 | MNTKITNTQNQAVSTSFTQEKIELIRKHFFPVNAKTVEMEYCLN | 127 |
| | | | | IANKYNLDPFLKQIFFVPRRSQVEVNGKKEWVDKIDPLVGRDG | |
| | | | | FLAIAHKTGKFGGIRSYSEIKQFPRLNDNGKWEYIQDLVAVCEV | |
| | | | | HRTDSDKPFVVEVAYNEYVQKKASGEATSFWTTKPDTMLKKV | |
| | | | | AESQALRKAFNLSGLYSAEEMGVGMT | |
| SR120 | SSAP | recT | Helicobacter pullorum MIT 98- | MNKEIINKENNQVALADAENLAFIKKQFFPVNATEKDIEFCLKV | 128 |
| | | | 5489 | AQAYSLNPVLREIFFVERMANVNGAWVVKVEPLVSRDGLLSIA | |
| | | | | HKSGKLSGIKSESFLKETPVLINGEWEIKKDLCAVANVYRTDTK | |
| | | | | EVFSAEVFYSEYAQKTKEGKITKFWAEKPHTMLKKVAESQAL | |
| | | | | RKAFNINGIYTPEELDGIKSVGGDVKTLSGVDLEVEADIDEPEIF | |
| | | | | YELDSTPAEADSEKKAVEALGLSVEEKNGYLKIMGNTYKKEA | |
| | | | | HIKALGYTLHKASSGENIWIKKLA | |
| SR121 | SSAP | ERF | Escherichia phage Tls | MKFSEQNANVIKALFEARQLFTKVKKDKQNTHLKNKYATLDS | 129 |
| | | | | VLDAIMPGLTDKGLFLTQDQKVGEDLKSMTVLTRFIHVESNEW | |
| | | | | VEYSFTLPMQKLDPQGGGSTNSYARRYALCTALGLATADDDA | |
| | | | | NLATKNAQDWKKDLDACDNLTDLQETFKTAYKQSDAANRRII | |
| | | | | KEHYDKLKAKMEIGKARGFNPAEPAANVAKNEKVEKEPQQEV | |
| | | | | KSQSITDFE | |
| SR122 | SSAP | gp2.5 | Bordetella bronchiseptica | MKLKLNNVRLAFPVLFEAKTVNGEGKPAFSASFLIDPSDAQVK | 130 |
| | | | (Alcaligenes bronchisepticus) | ALNQAIEQVAKEKWGAKADAVLKQMRAQDKVCLHDGDLKA | |
| | | | | NYDGFPGNLYVSARSATRPLVIDKDRSPLAEADGKPYAGCYVN | |
| | | | | ASIELWAQDNNYGKRVNAGLRGVQFLRDGDAFAGGGVASED | |
| | | | | EFDDITEGAAAADLV | |
| SR123 | SSAP | ERF | Lactococcus phage LL-H | MKMQHSDSIKEIFGALSKFRAQVKQPAKTANNPVFNSKYVTLE | 131 |
| | | | (Lactococcus delbrueckii | GVMQAIDAALPGTGLAYSQLVENGDNGASVSTLITHSSGEWMI | |
| | | | bacteriophage LL-H) | VGPLTLNPTKRDPQGQGSAITYAKRYQLASAFGISSDIDDDGNA | |
| | | | | GTFGEDSRWQSGYQRSRPKTTHTEAKTLTKAITLASSLAHHSK | |
| | | | | PKSGP | |
| SR124 | SSAP | ERF | Listeria phage A500 | MKMSESVLELSVALSKFQEKVEQPAKTANNPFFKSSYVPLENV | 132 |
| | | | (Bacteriophage A500) | ISAVKKHAPDLGLSYIQIPLTEENKVGVKTILMHSSGEFVEFDPF | |
| | | | | MLPLDKNTAQGAGSALTYARRYTLSSAFGIASDEDDDGNGAS | |
| | | | | GNTKANKSSKNYQQTKQTQPTQQSDNLASPAQRKAIFAKASV | |
| | | | | VGDPFGHDAKYVLESYKITDTKSMSKGEASALIKKEDAEIEAQ | |
| | | | | KQVN | |
| SR125 | SSAP | recT | Acinetobacter radioresistens | TTKEWKWNERARKSLPEEKVHTVKLRNITCKAWAIETATGER | 133 |
| | | | SK82 | LESAEISMEMAVKEGWYQKNGSKWQTMPEQMLRYRAASFFG | |
| | | | | RIYAPEVLMGIRTQEEEQDAIIDVTPEPVQQTSPVTTSDLKANV | |
| | | | | VKEAPVEQQAKQQPVQEEKKPRARKQPKVIETENVQNSTENE | |
| | | | | VVDAELTVEDLKRLQQEAENLIQQNKTSETAQVDVNKFAEIKK | |
| | | | | SYMKTLTSTVQASSINGLKSQIEKE | |
| Strep01 | SSAP | recT | Streptomyces longwoodensis | MSLTTLKERVQQRAAAASSDGIPPGQADEDTPAGRDHDAEQFR | 134 |
| | | | | ADIAAALPAHVSVDRFLAVFRPVLPGLRKCTPASVRQAVITAA | |
| | | | | RFGLLPDGEEAVITADDGIATFLATYHGYIELMWRSGMVKSIV | |
| | | | | AKVVYDGDEWDYVPTAPVGQDFTHRPKLGSPKDRTPLFAYTF | |
| | | | | AWLDGGARSDVAIVTVEEAEAIRDEFSKAYQRAEANGQKNSL | |
| | | | | WHTRFPDMLLKTAIRRAAKLLPKSAELRALVAVERAADEGHT | |
| | | | | QILAALTPEALALEAEARAAARAAEASQDVPPPRLPVKRGRAR | |
| | | | | PRRRHRDKNKATRR | |
| Strep02 | SSAP | recT | Streptomyces albus | MSQISNALATRDQGPAAQIEQYRDEYAALVPSHVNADQWVRL | 135 |
| | | | | AVGAVRGDEKLMEAAQNDIGLFLREMKTAARLGLEPGTEQFY | |
| | | | | LTPRKSKPHGGRKVIKGIVGYQGIVELIYRAGAASTVIVEAVRE | |
| | | | | NDTFRYVPGRDDRPVHEIDWFANDRGPLVGVYAYAVMKDGA | |
| | | | | VSKVVVLNRSRVMEFKAKSDSKHSEYSPWNTNEEAMWLKSA | |
| | | | | VRQLAKWVPTSAEYRRDQLLAHTETADSVVASVSTAPLPPQPS | |
| | | | | ALDDADPDDDGPIDAELVD | |
| Strep03 | SSAP | recT | Streptomyces noursei ATCC | MTSPIRAAVARRAGDPAALISQYTADFAAVEPSHIKPATFVRLA | 136 |
| | | | 11455 | QGILRRDEKLAQAAANDPGQFMSVLLDAARLGLEPGTEAYYL | |
| | | | | VPFKGRVQGIVGWQGEIELMYRAGAISSVIVETVREDDVFVWT | |
| | | | | PGLVDRETPPRWEGPMSYPFHEVEWAGDRGPLRLVYAYAVM | |
| | | | | KGGATSKVVVLNAQDIERAKKTSQGADSPSSPWRQHEAAMWS | |
| | | | | KTAVHRLAKYVPTSAEYITAQVRAVRQADALSAPPVEEVVDV | |
| | | | | ELVGDGQEQEARR | |
| Strep04 | SSAP | recT | Streptomyces albulus | MNNPSEEPSVDGAPTAQRPGGENASPDQAVTPSAIILRAEQTEF | 137 |
| | | | | DHRQRMALALISPELKKASRPELAVFFHHCVRSGLDPFARQIY | |
| | | | | MIGRKNKGRDREENPITWTIQTGIDGFRTIAHRAAERTGERISYE | |
| | | | | DTVYYAADGTTSDVWLADEPPAAVKVTVLRGTSRFPLVARW | |
| | | | | AEFAPEHWDNKQGKYVVAAMWKQMPAHMLRKCAEAGSLR | |
| | | | | MAAPQDLSGVYADEEMEQADAEVVAGEAKEASSRLRAAAGL | |
| | | | | DDAHSEAESNAEVEEPEAPTPKKSRPKPATEASTQSEGSKRPRK | |
| | | | | RTAGKRAANKAADSGAGG | |
| Strep05 | SSAP | recT | Streptomyces rimosus subsp | MSQISNAIATRDTGPAAQIEQYRDEYAALVPSHINADQWIRLAV | 138 |
| | | | pseudoverticillatus | GAIRGNKDLEKAARTDVGIFLRELKTAARLGLEPGTEQFYLTPR | |
| | | | | KSKAHGVALIIKGIVGYQGIVELIYRAGAVSSVIVETVREHDTFS | |
| | | | | YVPGRDERPVHEIDWFGADRGALVGVYAYAVMKDGATSKVV | |
| | | | | VLNRARVMEIKAKSDSKDSEYSPWKTSEEAMWLKSAVRQLAK | |
| | | | | WVPTSAEYMREQLRAQAEVAAETPSVASAPLPPQPSALDDYDP | |
| | | | | ADDGPIEGELVD | |
| Strep06 | SSAP | recT | Streptomyces cyaneogriseus | MSQIGNAIEKRDQGPAAQIEQYRDEYAALVPSHVNADQWIRLA | 139 |
| | | | subsp noncyanogenus | VGAIRGNKDLEQAARNDVGVFLRELKTAARLGLEPGTEQFYLT | |
| | | | | PRKSKAHGYKLIIKGIVGYQGIVELIYRAGAVSTVIVKAVRERD | |
| | | | | TERYVPGRDDRPIHEIDWFGTDRGPLVGVYAYAVMKDGAVSK | |
| | | | | VVVLNRTRVMEIRAKSDSKDSEYSPWQTNEEAMWLKSAVRQL | |
| | | | | AKWVPTSAEYMREQLRAQAEVAGELAAPSVMGAPAMPQPSV | |
| | | | | LDDTDPDDEPIDGELVD | |
| Strep07 | SSAP | recT | Streptomyces sp HPH0547 | MSSQISNAIATRDNGPTAQLKQYRDEYAALVPSHINLDQWIRL | 140 |
| | | | | ATGALRGDEKLMEAAQNDVGVYLREMKTAARLGLEPGTEQF | |
| | | | | YLTPRKSKAHDGRPIIKGIVGYQGIIELIYRAGAVSSVVVEAVRR | |
| | | | | ADTFHYVPGRDERPIHEIDWFNDDRGPLVGVYAVAVMKDGAT | |
| | | | | SKVVVLNKRQVMEAKAKSDSRNSQYSPWQTNEEAMWLKTAV | |
| | | | | RRLAKWVPTSAEYMREQLRAAKEVAAEPTPDAPSLPPVQAAP | |
| | | | | LDDVDPDTGEVLEGELLDEPSTT | |
| PF001 | SSAP | recT | Hungatella hathewayi DSM 13479 | MDELTLNQPVTSLSSGVFSSAESFQELFNIGKMFSASTLVPQAY | 141 |
| | | | | QGKPMDCTIAVDMANRMGVSPMMVMQNLYVVKGKPSWSGQ | |
| | | | | ACMSMIRASKEFKNVRLVYTGDKGTDSWGCYVQAEHRETGEP | |
| | | | | VKGTEVTIKMAKEEDWYGKTGSKWKTMPEQMLAYRAAAFFA | |
| | | | | RVYIPNSLMGLHVEGEAEDITNESEIPEIPDIFGEKEKEAQV | |
| PF002 | SSAP | recT | Ureaplasma urealyticum serovar | MVKDDKLVPSIRLLTPNELSEQWANPNSEINQITRAVLTIQGIDL | 142 |
| | | | 10 (strain ATCC 33699/ | KAIDLNQAAQIIYFCQANNLNPLNKEVYLIQMGNRLAPIVGIHT | |
| | | | Western), Ureaplasma | MTERAYRTERLVGIVQSYNDVNKSAKTILTIRSPGLKGLGTVEA | |
| | | | urealyticum serovar 7 str ATCC | EVFLSEYSTNKNLWLTKPITMLKKVSLAHALRLSGLLAFKGDT | |
| | | | 27819, Ureaplasma parvum | PYIYEEMQQGEAVPNKKMFTPPVAEVIEPAVENIKKVDFNEF | |
| | | | serovar 3 (strain ATCC | | |
| | | | 700970), Ureaplasma urealyticum | | |
| | | | serovar 8 str ATCC | | |
| | | | 27618, Ureaplasma urealyticum | | |
| | | | serovar 4 str ATCC 27816 | | |
| PF003 | SSAP | recT | Clostridium sp FS41 | MAENEKQALLQEENKSENVVSTVKRTALATNPFSDTDQFNNIF | 143 |
| | | | | KMAQLISQSDMIPATYKGKPMNCVIALEQANRMGVSPLMVMQ | |
| | | | | NLYVVKGVPSWSGQGCMMIIQGCGKFRDVDYVYSGEKGTDSR | |
| | | | | SCKVVATRISDGKRIEGTEITMQMVKSEGWISNTKWKNMPEQ | |
| | | | | MLGYRAATFFARMYCPNELNGFATEGEAEDMNHKPQRIEAIN | |
| | | | | VLGDTAHE | |
| PF005 | SSAP | recT | Clostridium sp CAG:470 | MSNEVQKNNELMVKFDIDGNEIKLTPSIVQEYIVGTDAKITNQE | 144 |
| | | | | FKLFTELCKVRKLNPFLREAYLIKYKAGVPAQLVVGKDAILKR | |
| | | | | AVLNPNYDGMESGIIVQKEDGSVEERQGTFRLGNEQLVGGWA | |
| | | | | RVFRKDWTHPTYSSVSFNEVAQKTGQGQLNSNWGSKGATMV | |
| | | | | EKVAKVRALRETFVEDLAGMYEAEEMQQEISQQEPIEVQAEIE | |
| | | | | EQTENTKEVSMNEL | |
| PF006 | SSAP | recT | Coriobacteriales bacterium | MASKNEAIEVSPAEIASVKEKPASIVKAEKAKKEPCALVKYEDAE | 145 |
| | | | DNF00809 | GREVVLTREDIINTISSNPRITDKEIKEFIELARAQKLNPFTREI | |
| | | | | FITKYGDYPATFIVGKDVFTKRAQSNPLFKGMQAGIIVQRGNA | |
| | | | | VDQREGSATFGDEMLIGGWCKVYVQGYDVPIYDSVSFNEYAA | |
| | | | | RKTDGTLNAMWASKPATMIRKVAIVHALREAFPSDEQGLYDQ | |
| | | | | SEMGLSGQGGE | |
| PF007 | SSAP | recT | Paeniclostridium sordellii | MSKLKSALQSKEAKGDGLTVSKSYAMKQLTIKMKNDIKEALP | 146 |
| | | | (Clostridium sordellii) | SHFCIDNFQKSAINTYNLDKSLQECEATTFISAMIECAKEGLEPN | |
| | | | | NILGQAYLVPVCVDGVNKVEFQIGYKGLIELAYRSGKIKSLYA | |
| | | | | NEVFEKDEFHIDYGLDQKLIHKPFLGGDRGEVIGYVAVYQMDN | |
| | | | | RGASFVFMTRDEILGHSKKYSRSFGCDLWESEFDAMAKKTVIK | |
| | | | | KLLKYAPLSIELQKSVSIDESVKGVGCI | |
| PF008 | SSAP | recT | Streptococcus infantis SK970 | MTNNQLSTQQTKRDISVNALDWTFEDIKRYFDPQNLLTEKQVG | 147 |
| | | | | QALSLIKGRNLNPLANEVYIVAYKNRNGGTEFSLIVSKEAFLKR | |
| | | | | AAQSKNYEGFEAGVVAVDKDGVMHERKGALMLPGDTLVGG | |
| | | | | WARVYRKNFKVPVEIQVSLEEYNKKQSTWNSMPATMIRKTAL | |
| | | | | VNALREAFPEDLGNMYTEDDGGETFDRIKDVTPQESREDVVAR | |
| | | | | KMAQIEQFNKEQAHTDPEPTQNEEPIQGELLDGELEY | |
| PF009 | SSAP | recT | Leptotrichia goodfellowii F0264 | MGRLTQNEANDKLIIFKVGNDEVKLSNNIVKRYLVTGQGNVT | 148 |
| | | | | DEEIMYFMKLCKARNLNPFVRDAYLIKYSDKDAATIVVAKDAI | |
| | | | | EKRAIQHPKYNGKEVGLYIIKKETGDLEKRNGTIYLKEKEEIAG | |
| | | | | AWCTVYRKDWDNPVTVEVNFDEYVGRKKDGTANINWANRP | |
| | | | | VTMITKVAKAQALREAFIEEISGMYEAEEAGINVNDLDDTPIEQ | |
| | | | | KDMQNMPDVTNTVQETEIDDEDLKKELFDNENKKNPLE | |
| PF010 | SSAP | recT | Lactobacillus rossiae DSM 15814 | MAVVKRLKGVKSIEAYVVRQGNDFDVASEGEQVVVTKWQM | 149 |
| | | | | HVADLDKPIAYAVCVIEKEDGTKSFTIMTKKQIDVSWSHAKTT | |
| | | | | KVQREYPDEMAKRTVINRAAKLFINTSDDSDLFVQAVNDTTSD | |
| | | | | EYDNDSRKDVTPSAESKGTQDLLDGFQSSNQSQQPSATSQSSQ | |
| | | | | PVSTATSAEKSANPSAESAVTSKTAKPSSTAEDIINGYEQSQSSK | |
| | | | | GADTVDEDGHNVVIDHEDEEPDVHQGDIFNQHDNPQS | |
| PF011 | SSAP | recT | Paenibacillus sp FSL R7-0331 | MTIAIETQVQETLDRILDSKHDALPSDFNKKRFSENCKVYVFDD | 150 |
| | | | | KDLHKYTADEIAANLFKGAVLGLDFLAKECHLISGGVDLKFQT | |
| | | | | DYKGEMKLTKKYSIRPLLDVYAKNVREGDEFREEVIEGRQVIH | |
| | | | | FAPRPFNTSKIIGSFAVAFFHDGGMVCESIPAGEIEEIRKNYGKA | |
| | | | | LGDAWDKSQGEMYKRTVLRRLCKTIETDFDAEQRLVYDAGG | |
| | | | | AFEFTKQPARSRQQSPFNPPEESEVIQNDRAAETDQG | |
| PF012 | SSAP | recT | Nocardia terpenica | MSEISKAVATQQNPLAVVARYKRELGTVLPTVLRQDPDRWLM | 151 |
| | | | | AAENAARKNPDIMAVTKADQGASYMRALVECARLGHEPGSK | |
| | | | | DEHFIKRGNAISGEESYRGIIKRVLNSGFYRSVVARTVFSNDTYS | |
| | | | | FDPLTDIVPNHVPAQGDRGKPLSAYAFAVHWDGTPSTVAEATP | |
| | | | | ERIATAKAKSFASDKPTSPWQLPTGVMYRKTAIRELEPYVHVA | |
| | | | | PEPQPRRHLDGTVGGIPATDFDVDDGDVLDITADQLAEAGEIV | |
| PF014 | SSAP | recT | Dermabacter sp HFH0086 | MSNALTITQDQTEFTPKQLSVLENLGVQGAAPQEVAMFFDYCQ | 152 |
| | | | | RTGLSPWARQIYMIGRWDRNLGRKKYAVQVSIDGQRLVAERS | |
| | | | | GVYEGQTAPQWCGPDGQWVDVWLANEPPQAARVGVWRKSF | |
| | | | | REPAYGVARLSSYMPVTRDGKPQGLWGTMPDVMLAKCAESL | |
| | | | | ALRKAFPLELSGLYTSEEMQQADAPRTEPAPVDEDVVDAEIVD | |
| | | | | DEERMQWVEAIQAAETTDVLRKMWADIKTCPDALQAELRELI | |
| | | | | PARAKELAA | |
| PF015 | SSAP | recT | Leifsonia xyli subsp xyli, | MAVKKNPTIEDYLIKVEPEFQRALGASMDAAKFAQDALTAIKQ | 153 |
| | | | Leifsonia xyli subsp xyli | NPKIGHSDPRSLFGALFLAAQLKLPVGGPLAQFHLTTRTVKGNL | |
| | | | (strain CTCB07) | TVVPIVGYGGYVQLIMNTGLYSRVSAFLIHAGDYFVTGANSER | |
| | | | | GEFYDFRRADSDRGEVKGVIAYAKVKGHNESSWVYIDAETMR | |
| | | | | AKHRPKYWESTPWADDAGEMFKKTGIRVLQKYLPKSVESLNV | |
| | | | | ALAASADQAIVRKVDGVPDLDIQHDRDTETVAVPEQPVSVPQP | |
| | | | | GDET | |
| PF017 | SSAP | recT | Lactobacillus shenzhenensis | MSAVSESKDLQHVDQLSVLNSAMTAASLNLPINQNLGFFYLVP | 154 |
| | | | LY-73 | YKGIAQAQMGYKGYIQLAQRSGQYQRLNAIPVYADEFGSWNP | |
| | | | | LTEELDYTPHFEDRKASDKPVGYVGFFKLANGFEKTVYWSRK | |
| | | | | QIEAHRDRFSKSSKSSASPWNTDFDAMALKTVLRNLITKWGPM | |
| | | | | TTDIQRANDADEGDYKNDLSTDTSEPKDVTPGASLEQFLGETD | |
| | | | | QQQKPATKPAPKKKAEEAKPNDLKPDVTHDPNEHTEQTSLSD | |
| | | | | DDLPFD | |
| PF018 | SSAP | recT | Pseudoalteromonas lipolytica | MSLSLQEYQNLLYGKLTACKGQFDAYLSENGYKLDFNTELNY | 155 |
| | | | SCSIO 04301 | VYQIVMSGLNVEYSFPYTPVESVISSFLKAAKIGLSLCPTEQLCE | |
| | | | | LKTEYSESSGQYVTQLGLGYKGILKLAYRSGKVKQINANVFYE | |
| | | | | KDTFQYNGVNSKVTHTTTVLSKAMRGQLAGGYCQTELIDGSF | |
| | | | | KTTVMPPEEILAIEEQGKVMGNEAWLSVHVDQMREKTLIKRH | |
| | | | | WKTLCPCIVRDSVMNDPMLFDDQDCQHSSNQQAYEEQFESAY | |
| | | | | SREAY | |
| PF019 | SSAP | recT | Acetobacter orientalis 21F-2 | MSNAVATHNPVLQPNNFQELIGFAKMAAASDLMPKDYKGKPE | 156 |
| | | | | NIMIAVQMGSELGLAPMQAIQNIAVINGRPSVWGDAMLALVR | |
| | | | | GSGKCSSVKEIFEGDGDNLAAVCVVRRVDGDEVRGEFSVADA | |
| | | | | KRANLWSKQGPWQQYPRRMLQMRARGFALRDAFPDVLRGLI | |
| | | | | GAEEAQDIPADPIDVTPRHRHVEPPKTIDHKQIFSDRLEGCPDAL | |
| | | | | SVDNQWTVWESTIQKAFDAGRPIPEAVQDAVRDMIAAKRAEF | |
| | | | | DRQATEAPIEEPVA | |
| PF020 | SSAP | recT | Collinsella stercoris DSM 13279 | MNQIVKFTDDSGLAVQVTPDDVRRYICENATEKEVGLFLQLCQ | 157 |
| | | | | TQRLNPFVKDAYLVKYGGAPASMITSYQVFNRRACRDANYDG | |
| | | | | IKSGVVVLRDGDVVHKRGAACYKKAGEELIGGWAEVRFKDG | |
| | | | | RETAYAEVALDDYSTGKSNWAKMPGVMIEKCAKAAAWRLAF | |
| | | | | PDTFQGMYAAEEMDQAQQPEQVRAQAEQPVDLQPIRELFKPY | |
| | | | | CEHFGITPAEGMTAVCGAVGAEGMHSMTEQQARRARAWMEE | |
| | | | | EMAAPAVEAEYEVVDEGEVF | |
| PF021 | SSAP | recT | Lactobacillus capillatus DSM | MANEVAEKNEVVYLAGNEEVKLKPSTVRDFLTSGNGNITSQE | 158 |
| | | | 19910 | AFMFIKLCEYQHLNPFLNEAYLIKFGNKPAQIITSKEAFMKRAE | |
| | | | | SHPAYNGVKAGLIVLRNSDVKYTKGAFKLPTDKILGAWAEVN | |
| | | | | RKDRDEPHHIEISMEEFSKQQSTWKSMPATMIRKTAIVNALREA | |
| | | | | FPETLGGMYTEDDKNPNEAVKESEPVDDKAESVVDDLVKDIKP | |
| | | | | KNEPAEKEGEPKESEQYEEQETTFEEVNSKEAEKDGDSNSEEQ | |
| | | | | TSLFKGATIQPNV | |
| PF022 | SSAP | recT | Treponema socranskii subsp | MNEIIKKEATEIITPVDEKTIMEYLDTTGLTKSLLPKEKAMFVN | 159 |
| | | | socranskii VPI DR56BR1116 = | MARLYGLNPFKREIYCTVYGEGQYRQCSIVTGYEVYLKRAERI | |
| | | | ATCC 35536 | GKLDGWQAQITGSLQDGTLAATVTIWRKDWTHPFTHTAFYTE | |
| | | | | CVQTSKKTGEPNAIWRKMPSFMVRKVAIAQAFRLCFSDEFGG | |
| | | | | MPYTNDEMGVDAPKERDITHEATATIADEAEAPSAEVKNEPKP | |
| | | | | ADVVQQLETLLMKYESQLSGKPYELAEEALRTGSDAEVIAMY | |
| | | | | ERVVSYLKRKGIQVGK | |
| PF023 | SSAP | recT | Bifidobacterium magnum | MTSSTLVPSTLEEQKTYAELVAQSTGMVPAAYQGKPANIFVAI | 160 |
| | | | | QVGSSLGLEPMASLQGINVIQGKPTLSAQAMLGLLKSQHFKVH | |
| | | | | ITKDDENQRVTVEVIDPDDPDYSTVSVWDMAKARKAGLGGDQ | |
| | | | | YWTKQPMTMLKWRAVSEAAREAAPHLFLGLGGAYTKEELEE | |
| | | | | SVTVENVEETPRPRSPYSSYTRKPEPVEPEVVEAEPVEPEPINL | |
| | | | | IIQTMRENGVDTAQKARLVLTQIVGVENVNDIPADKLETLCANL | |
| | | | | DGFAHDIQQTLGDNK | |
| PF024 | SSAP | recT | Frateuria aurantia (strain ATCC | MSEISVARAQNLAQIERVNALLPTSIGEAMQLAEFMAKSDLLPP | 161 |
| | | | 33424/DSM 6220/NBRC 3245/ | HLKGRQGDCLLVVMQAQRWGMDALSVAQCTSVVHGRLCYE | |
| | | | NCIMB13370) (Acetobacter | GKLVAAALYSQKAIDGRLHYEISGHGQDASIVVTGTPRGTGQT | |
| | | | aurantius) | QSVSGSVRKWRTITMKKQDGAPPKRVDNAWDTIPEDMLVYRG | |
| | | | | TRQWARRYAPEVMLGVQTPDEVDDTPMQTTVIHSTAASSPAIE | |
| | | | | PLIPYPEEEFSKNFDTWLGRIQCGRNSAEEVIAKLQTKYTLTPG | |
| | | | | QLGAIRDLETTEAEVVE | |
| PF025 | SSAP | recT | Parcubacteria bacterium 32_520 | MKKETTQNKAKKITKKASSKKKETKKIVVKEQQITDPALLTKA | 162 |
| | | | | KIDLIKRTVAKGATDDELQMFLTVAARAGLDPFTKQIHLVKRH | |
| | | | | STAEGRDIATIQTGIDGYRAIAEKTGKYGGSDDATFTFADGQDK | |
| | | | | IPTSATVKVYKIIGDKVIEIKATAYWDEFAPTNEKLAFMWRKM | |
| | | | | PKLMLAKCAEALALRKAFPNVLSGIYTHEEMEQLDEEKNVND | |
| | | | | KKATLANSKAYIVSAKSLDELKGFRERIEKSKIDKATKEKLLKY | |
| | | | | CQEKEVELSAIEAEIESV | |
| PF026 | SSAP | recT | Helicobacter sp MIT 05-5294 | MNEVAKLNESRASNQKMIATILESDSSHKKLNDFFAGDSAKVQRF | 163 |
| | | | | KSSLINIAGNSILSSCSPASIIRSAFSLAEIGLDINQTLKQSYVL | |
| | | | | KYGIEAEPVISYKGWQSILEKVGKKSRAFCVFKCDTFNIDFSTFE | |
| | | | | GNLTFVPNYAQRDETNRKWFNDNLLGVVVLIKDKDASEIVNF | |
| | | | | VSAKKIDKIKGCSPSVKKGRNSPYDEWFEEMYLAKAIKYVLSK | |
| | | | | QALTEKEETIARAIDIENEVEAKIQKEASNYEIKDLEELMQDKE | |
| | | | | VMAMTLDAEEGIPQI | |
| PF027 | SSAP | recT | Mycobacterium brisbanense | MTETAVAKPEQRPTTISQVLQVMIPELTRAIPKGMDADRLARIV | 164 |
| | | | | QTEIRKSRNAKAAGITRQSLDDCTQESFAGALLTASALGLEPGI | |
| | | | | NGECYLVPYRDTRRRVVECQLIIGYQGIVKLFWQHPRARGIKT | |
| | | | | DWVGANDHFEYEDGENTVLVHRKAADRGEPIAFWAVVKVAD | |
| | | | | ADPLVTVLTADEVKELRKGKVGSSGDIKDVQHWMERKTVLK | |
| | | | | QGLKLAPKTTRLDAAIVNDDRAGTDLSRSQALMLPPGVQSTAD | |
| | | | | YIDGTVDEEPQGYEEIAETEAQVQ | |
| PF028 | SSAP | recT | Capnocytophaga sp oral taxon 338 | MSAITQTTDKRLGNFLNQANTADFLTKTLGARKSEFVSNLLAIS | 165 |
| | | | str F0234 | DADKNLSQCDPSEVMKCAMNATALNLPLSKNLGYAVVIPYKD | |
| | | | | WKTQEVHPQFQIGYKGFVQLAIRSGQYRTINTCEVREGEIKRN | |
| | | | | KFTGHTEFLGENPEGKVIGYLAYIELQNGFQQSLYMTLEQVQE | |
| | | | | HVSKYSKSGIDKDTKEFKGIWRNEFDTMAKKTVLKELLNRFGV | |
| | | | | LSVEMQQAIEKDQADSEGRYIDNPQGGRYVQDAVIIEQSEPTEV | |
| | | | | VSQEEPSPAPTKGIKQVDFKQM | |
| PF029 | SSAP | recT | Salinisphaera hydrothermalis | MTNGASNQGRSGGQKKLIERIGDKYGVDANKMLSTLTATAFR | 166 |
| | | | C41B8 | QKNDQPITNEQMMALLVVADQYNLNPFTKEIYAFPDKNAGIVP | |
| | | | | IVGVDGWSRIINEHDSEDGMDFEASDEFIELDKHHKKCPEYITC | |
| | | | | RMYRKDRSRPVEITEYLDEVYRPPFMKNGNPMIGPWQSHTKRF | |
| | | | | LRHKAMIQCARLAFGFVGIFDQDEAERIVQGEVVSSERNGAAP | |
| | | | | PARQTEAPAAAERTLNDQQQNQVQAAIEESGADKAGLLKQLG | |
| | | | | VASIDQIPVTRFSLVMDRINAQTEDA | |
| PF030 | SSAP | recT | Candidatus Cloacimonas sp SDB | MQSLAVAKNESLMPLSFQEKLSMASVFIKSNLMPRGYDTPEKV | 167 |
| | | | | VIALEMGHELGLPPLVAIMNIAIINGTPTLKADMMVALALRSGK | |
| | | | | IEDIKIQYSGKENMNDNQFKCKVTIRRRGVETPFKASFSRQDAK | |
| | | | | VAGLDYKDNWKKYERRMLKHRAMAFCIRDALPEIFAGVYLPE | |
| | | | | ELEGIESYGGNRNTVILPAAQIEKTASNIEDNTADESLPSKVTDS | |
| | | | | QIKALKELSERLFNEGIVTSYIYDQYAVNRIEDLDQGKAGKLIL | |
| | | | | LLQNEPGKISEWIEDTYQKTA | |
| PF031 | SSAP | recT | Elusimicrobium minutum (strain | MTTENIQRTPALGILLEQAPFYSKAQNLEQKKIEDMMVSFAVIA | 168 |
| | | | Pei191) | HKVIKRHQDKGRGEVDVQSIKEAFKCSMDTGIPVDNRRLAYLT | |
| | | | | IVKNNSTGKYEIQYEPGYMGFVHKERQIKPGAVVQTILLWEKD | |
| | | | | VFTYKSTTGVAEYSYVPEKPMRSDFNHIIGGFCVISYFEHGREY | |
| | | | | SFVTPMTKAELDLARGKAKTQDVWGVWPGEMYKKVIIKRAG | |
| | | | | KVEFIGEPEMEKLNEIDDRSYTGFERKQPAKVDYSAVKPLPQEI | |
| | | | | TETEALPAPGGDDTVQDVFSMEEPQ | |
| PF033 | SSAP | recT | Spiroplasma kunkelii CR2-3x | MNNKIELIENNSKVNNWINEKLTNQKEINLFRKNILTIYNNNPN | 169 |
| | | | | LFDKEKINLMSVLSACVKAMLLDLPLDPNLGYAYIVPYNGKGQ | |
| | | | | LQIGYKGYIQLAIRTGKYLTINAIEVKQGELLNFDELEQEYNFK | |
| | | | | WITNENERTKKETIGYVAFFKLLNGYKKTLYWSKEKCENHFKT | |
| | | | | YSKNYQTYGKFTVGSYEGMALKTVETQLLRKWGIMSVEMQE | |
| | | | | VYQHDQAIIINNSKEFSDNPNIKNKEDKNVIELNNKKDELNLNL | |
| | | | | EQEFSINEINDDEEIAKTVDEMFSD | |
| PF034 | SSAP | recT | Avibacterium paragallinarum | MLKTAKPESIFNAACMAATLNLPLQNGLGFAYLVPFRTKRKVP | 170 |
| | | | JF4211 | LVDTEGNVILDRDGKPRTKEEYVIEAQFQIGYKGFIQLAQRSGQ | |
| | | | | FKRLVALPVYKKQLVKKDFINGFEFDWEQEPEEGELPIGYYAY | |
| | | | | FKLVNDFSAELYMTHEEIEKHAKKYSQTYRTYLEKKAKGQWA | |
| | | | | SSVWADNFESMALKTVMKLLLSKQAPLSVEMQNAVLADQSVI | |
| | | | | KDSENGEFDYPDNNIDDAEIVTMNVSQETFEQCKQNILNRETTL | |
| | | | | QALCDSGFKFSPEQYAQLEALENNRE | |
| PF035 | SSAP | recT | Endozoicomonas montiporae, | MPKATAKKSPATQVVDTAEKPATIKDLLRNNKNTIAQALENTP | 171 |
| | | | Endozoicomonas montiporae CL- | LTPERLLSVCMTEIRKTPKLRECSQASLVGSIIQSAQLGLEPGSA | |
| | | | 33 | LGHCYLIPFWNNKERSLECQFMLGYRGMLALARRSGSIVSIDA | |
| | | | | RVVYAADEFSLLYGTITEIIHKPKETGEKGGVVGVYAVAQLQG | |
| | | | | GGTQVDFMPVEDILKIRDSSKGVQSGKDTPWKTHFEEMAKKT | |
| | | | | VVRRLFKFLPVSVEALTAVGLDEKAEAGQSQCNEALIQEGDGG | |
| | | | | VLVDMDTGEIIEGKVTSAADLNRAL | |
| PF036 | SSAP | recT | Roseateles depolymerans | MSEIAPVNQTAIAANTDLGNPSAFLFDSERMTALMEFSNLMSR | 172 |
| | | | | GSVTVPKHLQGKPADCMAITLQAMRWRMDPYIVGSKTHLVN | |
| | | | | GNLGYEAQLVVAVLKNSGAVKGRPHYEFRGEGNNLECRAGFI | |
| | | | | PAGEDAVLWTEWESISGIKVKNSPLWQTNPKQQFGYLQARNW | |
| | | | | ARLYAPDALLGVYTEDELQVMPSQPSVRDMGQAEEVARPALP | |
| | | | | AYPQADFDKNLPGWRNVVESGRKSAPDLLAMLQTKATFSAEQ | |
| | | | | QEAILALGVIEQDAAEVDDPFVRDMEAAEGEQQ | |
| PF037 | SSAP | recT | Haemophilus parasuis serovar 5 | MTTNLPSNIQTALSERGIDHAVWSTLQNSIFPGAKDESILLAIDY | 173 |
| | | | (strain SH0165) | CKARKMDILKKPCHIVPMNVTNAKTAEKEWRDVIMPGIVEQRI | |
| | | | | TAFRTGQMAGQDDPVFGDTIEYLGVNAPEWCKVTVYRFVNGE | |
| | | | | RCAFSHTEYFTEACAITEIWKDKQRTGKYKVNSMWTKRPRGQ | |
| | | | | LAKCAEAGALRKAFPDELGGVITADEITEEPINPASPQQTVIDV | |
| | | | | DGVVRIADEQRQQLDQLIQITQTDVAKAIAVYGVNSLDQLPKD | |
| | | | | KAEHLIKILNERVDKLQAQSENLGENIPL | |
| PF038 | SSAP | recT | Sodalis glossinidius (strain | MANELNTTSAILAEKGVDFATWSALKNSIYPGAKDESVMMAL | 174 |
| | | | morsitans) | DYCRARQLDPLMKPVHLVPMYITDAKTGKGQQRDIVMPGVEL | |
| | | | | YRIQADRSGNYAGAKEPEFGPDETKIFGADETKNFKGIEVTFPQ | |
| | | | | WCKYTVCKMMPSGQIVEYSAKEYWLENYATAGRDSSAPNAM | |
| | | | | WKKRPYGQIAKCAEAQALRKAWPEIGQQPTAEEMEGKTEDVI | |
| | | | | DARDVTPSASHSTSHKSATTETLQAINDLLLSLDKTWDEDFLPL | |
| | | | | CSTVFKRQVSKAAELTDQEALKALDFLRKRATKDAA | |
| PF039 | SSAP | recT | Nocardia farcinica (strain IFM | MARNLVDRIEQNAPAKNDDLKAAIQKMEKAFALAMPKGAEA | 175 |
| | | | 10152) | TQLIRDAFTAIQRTPKLAQCEQLSVLGAFMTCAQLGLRPGVLG | |
| | | | | QAYVLPFWDRKDRMYKAQFVAGYRGLVDLAYRSDRVLSVSA | |
| | | | | RIVHANDYFELEYNAVEDRLVHRPYLDGARGEARLYVAAGRT | |
| | | | | RGGGSAITDPVTVADMLKYRDRYATAKNKEGKVFGPWVDEF | |
| | | | | DGMAKKTMIRQLSKMLPMSTELTLAVENDGSVRFDLGKDAIE | |
| | | | | SPQRLEPDVIDAEAVATSDVQDAPADYVEDVPPAAEGGMFAPE | |
| | | | | GE | |
| PF040 | SSAP | recT | Parasutterella excrementihominis | MENIEIKAQTVSLVARLAAKFGVEPGKLMACLMNQVFKQSDG | 176 |
| | | | CAG:233 | VAPSNEELMVLLLVCENFGLNPFNREIFAFRGKGGDVIPVVSLD | |
| | | | | GWCKIVRNQKDFNGMSFKFSESTIKLNCYGGELPEYVECSIKLK | |
| | | | | GVEDPVTIQEYMVECFNEKSSVWRKWPRRMLRTRGFIQCARL | |
| | | | | AFSLTGIYDEGDTFGEDSENGSGTTSDLKSQIPLVQQIPARPSLE | |
| | | | | KSRLDALIGKLIEHAKNRDDGWKRAFQWIEEKFSQEDCAYARE | |
| | | | | VLTAKRKELSLSSLPEGNEILPEETSPATLQGVSNEDR | |
| PF041 | SSAP | recT | Salinispora tropica (strain ATCC | MTQTVSQAVATRDNSPAGLITQYSESFAQVLPSHIKPATWVRL | 177 |
| | | | BAA-916/DSM 44818/CNB- | AQGALKRGKRGDGGRFELEIAASNNPGVFLAALEDAARQGLE | |
| | | | 440) | PGTEQYYLTPRKVKGRLEIEGITGYQGHIELMYRAGAVASVVA | |
| | | | | EIVREHDEYRYQRGIDDVPVHRYKPFARDAERGALIGVYAYAR | |
| | | | | MKDGAVSRVVELSRDDIDRIKASSQGANSEYSPWQKHEAAMW | |
| | | | | LKSAVRQLQKWVPTSAEFRREQLRAAAEAHRVASAADAPDGA | |
| | | | | TAPQGDVLDGEVLDEAPTEPARSDDAGGHVEQEWPDAARPGG | |
| | | | | AQ | |
| PF042 | SSAP | recT | Microbacterium ginsengisoli | MSEVALPSSVRPDTWNADTAAMMEFAGLTWIEGVGDAAHRV | 178 |
| | | | | FAPSGVMAAFIAACARTGLDPTAKQIYAAQMGGKWTVLVGID | |
| | | | | GMRVVAQRTGQYDGQDPIEWLAAEDGQWTTVPPKAPYAARV | |
| | | | | AIYRKGVSRPLVQTVTLAEFGGRGGNWSQRPSHMLGIRAESHA | |
| | | | | FRRAFPMELAGLYTPEDFESDDVDTSDAPWEEREDWGALIASA | |
| | | | | TTTGDLARVGARIAESGQGNDDIRAAYRARAAVLATEANTVD | |
| | | | | ADVVDDQTPAEGEDASPTPPASPSAGTPDDYEAAASAEFDAAV | |
| | | | | ERGEI | |
| PF043 | SSAP | recT | Labilithrix luteola | MASTEMTRSAGAAPLARRGGGDLKEFLGSDAVRKKLAEAAG | 179 |
| | | | | KVMRPEDLIRFALVAASRTPDLAKCSKESVLRSLLDSAALNIMP | |
| | | | | GGLMGRGYLVPRKNTKNNTTECHFDPGWRGLIDIARRGGKVR | |
| | | | | RIEAHVVHAADVFSVERSPLTTLHHVPSELDDPGEIRAAYAVAE | |
| | | | | FVDGGIQIEVVTRRDLNKIRAMGAKNGPWSTWGEEMARKTAV | |
| | | | | RRLCKYLPYDPLLERAMHASDESDVNAFDEVVEVTAPKKRRR | |
| | | | | SLDEKVDEVAASMLPPAEPDSQQDLPVDADFDEADDGTSDAA | |
| | | | | EPTN | |
| PF044 | SSAP | recT | Agathobacter rectalis (strain | MAEQNAVATQQGTQLSVAAQVKSMISQDAVKKKFTEVLGQK | 180 |
| | | | ATCC 33656/DSM 3377/JCM | APQFLASITNVVAGSAQLKKCPATTIMSAAFVAATYDLPIDSNL | |
| | | | 17463/KCTC5835/VPI 0990) | GFAAIVPYNNNKYNPQTRQWEKHPEAQFQMMYKGFIQLAIRS | |
| | | | (Eubacterium rectale) | GYYEKMNCSVVYKDELVSYNPITGEVEFVTDFSKCTQRAEGKS | |
| | | | | ENIAGYYAWFKLLTGFRKELFMTTAEVENHARKYSTAYRYDL | |
| | | | | ENNKKGSKWTTDFEAMALKTVIKMLLSKWGILSVDMQRAIQD | |
| | | | | DQKVYDEDGEGSYGDNQPDIVEAQDPFGNIEQKEEEQQIGGLD | |
| | | | | LEEVE | |
| PF045 | SSAP | recT | Spiroplasma kunkelii CR2-3x | MITELQQVIKGAIIKYELKVNDKYLKENILALEELKMKNGQSYM | 181 |
| | | | | QLVNTADNTKITALGIIKLSNKGLIFGKDFNIIPFKNKLTTVIDS | |
| | | | | KVYCKRIEESGYSPRKAIIFKGEKFEWDSENSCPKIHEINFNANT | |
| | | | | SDYNEIIGAYAFAKDKNGNYQGILLRKADIERLKNSSPSGNSEY | |
| | | | | SPWNKWPKEMVEAKLYRKLALEMGIDISDIDLDEKEIKEDGNF | |
| | | | | EYISFKDINVAKNKGQISDEPLSSAFLIKILDIVFSLILAFSIVC | |
| | | | | KISLYLFPSPKNEVILFIKFCSFLWYFFKPNSSSKG | |
| PF046 | SSAP | recT | Methylobacterium nodulans | MTAGALTLSDAERRVAERVDPAATAALSVSAESGGVAFANAG | 182 |
| | | | (strain LMG 21967/CNCM I- | QLMEFAKLMAVSSAGVRKHLRGNPGACLAICTQAVEWGMSP | |
| | | | 2342/ORS 2060) | YAVANNSYFVNDQIAFESKLVQAVILKRAPIKGRIRFEYTGDGD | |
| | | | | ERRCRAWARLADEPDEVVDYLSPPLGKITPKNSPLWKQDPDQ | |
| | | | | QLAYFSGRALCRRHFPDVLLGVYADDEIEVAPRGPDQARDVTP | |
| | | | | RGLAGRLDALAAAPPPTASAAVDAFTDTEAPGNGPATAPAEDE | |
| | | | | PETGAQGADVGESGDWHDPALGGAADDFPGNDALSDMRTGR | |
| | | | | RSSRWEA | |
| PF047 | SSAP | recT | Anaerococcus hydrogenalis ACS- | MTNAKNALKKNAQNKAPAKKQNTTVRGLLMAMKGEIQNALP | 183 |
| | | | 025-V-Sch4 | SYLPTDKFIRTALTAINTTPKLAECTQDSLLAAIMNSAQLGLEEN | |
| | | | | TPLGESYLIPYENKKLGITTVNFQIGYQGLLKLAYNTGQFKRIT | |
| | | | | AREVRENEDFVINYGTGEVKHEPCLTGDSGDVIGYYAIYQTKD | |
| | | | | GGQDVFYMSKADAEKYGRTYSKSYNFSSSPWQSNFDAMAKK | |
| | | | | SCLIQVLKYAPKAIESQKLVEATKTDNANFKSYKKEDDGSINLD | |
| | | | | VDYEVEVEEDKKEAPKNVDKETGEIKSEDIAQTGFFEDDFEPVS | |
| | | | | E | |
| PF048 | SSAP | recT | Sporosarcina newyorkensis 2681 | MTNQLQPQSNTPAEKKNELVTKVADKVQRMVENNQINIPQNY | 184 |
| | | | | SIVNAVQAAYFKLTEVDFKKKTSLMDTATPDSVAFSLQDMAIQ | |
| | | | | ALSVAKNQGYFIVYGDKMQFVRSYHGTQAVLKRLNGVKDVW | |
| | | | | ANVIWKGETFDVEYNDRGQLAFKSHTVDWQAATGKKEDIQG | |
| | | | | AYCIIEREDGVQFLTVMTMAEIKTSWSQSSTTAVQDKYPQEMA | |
| | | | | KRTVINRASKAFINTSDDSDLFIGAVNRTTENEFEDDRPIKEINP | |
| | | | | QHEIDQNANKEVLDFTEPEQSPQPVQEPELVQEPVRQPEPASSG | |
| | | | | GPGF | |
| PF050 | SSAP | recT | Clostridium beijerinckii (strain | MAETGLVLSKEDAFNNVMAKIAALEKNNGIKLPKNYSAENAIN | 185 |
| | | | ATCC 51743/NCIMB 8052) | SAWLMLQEVVDKEKKQALEVCTKTSIVEALYNMVLQGLSPSK | |
| | | | (Clostridium acetobutylicum) | RQCYFIVHGSKLTLMKSYMGSIVATKRLSGIKDVKAFVIYEGD | |
| | | | | VFETVFNNETYTIEFNYQPKEENINSNKIKGAFALIIGDDNKLLH | |
| | | | | TEIMTIDQIRKSWGMGIAYKSGKSNTHNDFGEEMAKKSVINRA | |
| | | | | CKRFYNTSDDSDVLIESLNNTDEDYDEADIIENAKEQVHEEIKA | |
| | | | | NANQEVIDVDPKQVTEIDNNNNENPIQKDLNNKKTEKAEQQM | |
| | | | | MCEF | |
| PF051 | SSAP | recT | Paenibacillus alvei DSM 29 | MSTAVQNINTQAVVGSFTQSELDTLKSTIAKGTSNEQFALFVQT | 186 |
| | | | | CARSGLNPFLNQIYCIVYNGKNGPVMSIQIAVEGIVALAKKHPQ | |
| | | | | YKGFIAAEIRQNDHFKAKIHTGEVEHEPDVMNPGETIGAYCVA | |
| | | | | YRENAPNVLVIVRRDQVEHLLKGRNSDMWRDYFDDMIVKHAI | |
| | | | | KRAFKRQFGIEVSEDEYVQPNSIDNTASYETRKDITADVESGTP | |
| | | | | QLQQPLQNSQNGENGEIKKVRRDISAAFKKLGITTEKAMTEYT | |
| | | | | MARMKQKGDKPTLQELTGLLKVMQLEIEEKEVFADGASEAGL | |
| | | | | EPLE | |
| PF052 | SSAP | recT | Bacillus sporothermodurans | MANNQLAVYQDLTFGELTQQDIVTVRETIGKDCNESQFKLFMS | 187 |
| | | | | IAKNSGANPILNEIYPAVRGGQLTVQFGIDFYVRKAKETEGYQG | |
| | | | | YDVQLVHENDGFKMHQEKDEDGRYVIVIDEHSWGFPRGKVIG | |
| | | | | GYAIAYKEGLKPFTVIMEVDEVEHFKKSNIGMQKTMWTNYFN | |
| | | | | DMFKKHMTRRALKAAFNLNFDDDEVGEGSGSDGIPEYKPQTR | |
| | | | | KDITPNQEIIDAPSKQEVHEEDPQLAQAKKDMKTKFKKLGITTK | |
| | | | | KGMQDYIKQHAPQIGDNPTYQQLVGLLDIMDMHIDMNEVQAS | |
| | | | | DADDVLE | |
| PF053 | SSAP | recT | Bacillus sp 2_A_57_CT2 | MAKNNELATQSAELNELHQIGGFGVNELMTMKETVGKDLSIP | 188 |
| | | | | QFNLFMYQCNRMGLDPSLRHAFPIVYGGKMDIRVSYEGLKSLA | |
| | | | | QKSDGYQGVFTQVVCENEIDDFDVLLNDEGEMVGVKHKPRFP | |
| | | | | RGKVLGAYAVAKREGRSNYVVFMDVSEVQKWMKINGKFWK | |
| | | | | QDNGDVDPDMFKKHVGTRAIKGQFDIADVVVDGMEAVADSN | |
| | | | | PIPEYKPGERKDITPDNNVIEPPKESEEEKHKKAERSSINQAFKK | |
| | | | | LGITGKKETAEYIEKHAPSFTNENPSEADLNGLLKLLEMNLEMI | |
| | | | | EMQSGGDELE | |
| PF054 | SSAP | recT | Thermaerobacter marianensis | MAVQTDQVRNKLARRAQENGAPAPSQQPKTIEQWLRDERFRA | 189 |
| | | | (strain ATCC 700841/DSM | EIERALPRHLSADRKKRITLTVLRTTPELRRCTVPSLLAAVLQCA | |
| | | | 12885/JCM10246/7p75a) | QLGLEPGVLGHVYLVPFKNGKTGEYEVQVIIGYKGWVELARR | |
| | | | | SGQIQSLTARVVYQNDEFELSFGIEDNERHVPWYMRPNVQDGG | |
| | | | | PIRGAYSVARFKDGGYHLHYMPIQQIEARRKRSRAADSGPWKT | |
| | | | | DYEAMVLKTVVRDASKWWPLSPEIARGLAQDESIKRTVDDVD | |
| | | | | SDAPYFGEDVIDVQGEDVGEGGEDAAHEEGGDASAGGDGSAQ | |
| | | | | FGLFGGGGQ | |
| PF055 | SSAP | recT | Salinicoccus halodurans | MTKNEVLVKNQKMGDNVLARVKELEGQGNLNFPANYAPENA | 190 |
| | | | | MKSAMLILQDLKGSKKDGYKPALEFANPNSVANALMDMVVQ | |
| | | | | GLNPQKSQGYFIMYGDKVQFQRSYLGTMSVTKRVTGAKEINA | |
| | | | | EVIFEGDDVEYETINGKITNLKHKTKFGNRDTKNIIGAVATIVFE | |
| | | | | DESKNYTEIMTTDEIETAWKQSQMVYNGEFKADGTHRKYPQE | |
| | | | | MSKKTVINRACKKLLNSSDDSSLLKQQVMNSDDRQRKEVFDT | |
| | | | | QVSENHATEDLDFPDDVVDGEFKEADQEPEPMQKPVDYDEET | |
| | | | | GEVKEDDKDDNPF | |
| PF056 | SSAP | recT | Dialister sp CAG:486 | MDARKGIVATRNEMTAQQQERPSIPKLLNNTLDNSGYKRRFDE | 191 |
| | | | | LLGKRAPQFVSSLAALINSTPQVLSIFQNNPVAIIQSALKAAAVD | |
| | | | | LPIEPSEGYAYILPFGQTATFVLGYKGMVQLAERTGLYMRLNA | |
| | | | | VDVREGELISYDRLTEDIEFRWEADNAKRAKIPVIGYAAFYRLK | |
| | | | | NGMEKTLFMTKAEIDAHELANRKGRSQNPVWRNHYDEMAKK | |
| | | | | TVLRRLLSKWGIMSINYLDASPADQEVMRNMAEGTLDDTEMP | |
| | | | | QERTIANSPNLAPVSPADPAEAITIPWEQGNAQEGQNQAVAGE | |
| | | | | AMNGGEGK | |
| PF057 | SSAP | recT | Leuconostoc mesenteroides subsp | MANEVAQVQKIINSDKMQKHFEEILQDNAAGFLSGLSTVVALN | 192 |
| | | | mesenteroides (strain ATCC | PDLAKTNMNDLTNAAMRAAILDLSVLPDLGEAYVIPYGKRAK | |
| | | | 8293/NCDO 523) | VDGKWVTKDVKAQFQLGYRGIIKLVQNTGRVGRLGGSVVYE | |
| | | | | ANKPHYNYVFDEFTMENENYDPYVDGESPVAGYLAFYYLDGE | |
| | | | | RIVKYWPIQRVINHATKFSQTYKGPDHKDRYGKTPQTPWYTDF | |
| | | | | DAMAIKTVMKDLLKFAPKTTKVAQAIAEDDKNEREARDVTPE | |
| | | | | TEEITPEEQNVEPEIIDNQPAEKSDNPFSGVDTGDAPNPFAEKNE | |
| | | | | TSEDVKWVSQKQ | |
| PF058 | SSAP | recT | gamma proteobacterium BDW918 | MSDQNPLVICKDQIAKSAVKFGSSELSYESEEIFAMQQLMKND | 193 |
| | | | | YLLKTAVNDPDSLRLAMYNVASTGISLNPARHEAYLVPRADK | |
| | | | | KGQPAKIKLDISYRGLIALAQSEGIIANAVCELVYEQDTYLFKG | |
| | | | | PTRIPEHAANVFSTTRGAVIGGYCITELTNGKVQIHNMSKADM | |
| | | | | DQIRDSSQAFGSGSGPWVEWESQMQLKSIVKRAAKWWPASTP | |
| | | | | KMAKVIEFLNVENGEGIATIEHQPSNRIVAPAKPEDIDSRTLGFI | |
| | | | | NQALTRAVQSQSFEACGELIRERVKDPAALAYGLEKLKELQTQ | |
| | | | | HGDYRHAG | |
| PF059 | SSAP | recT | Paenibacillus sp P1XP2 | MANNTQIILTPEIKEAFPAEVLDVIRTSLCPTATDPEFLLFAHKA | 194 |
| | | | | ASYRLDPFKNEIFFIKYGSQARIQFAAEAYLAKAREKEGFQPPD | |
| | | | | TQTVCANDTFKARKFKNDKGEDEWEVIEHEVTFPRGPIVGAYS | |
| | | | | IAYRDGYRPVTVFVDKEHIAHMYTGQNKDNWNKWEPDMIGK | |
| | | | | HAEQRALKKQYGLDFGNEELEHPPAPAASSFERKDITNEVNQA | |
| | | | | TAAANAQSTPSSDQPNGSQAEEDEEAKINALKAQMKQNFAKL | |
| | | | | GLVNKADRDAHMAKHFKIKGEQPTIAEIMAYLKIMDLQIQEKQ | |
| | | | | ASLNDELPL | |
| PF061 | SSAP | recT | Gramella forsetii (strain KT0803) | MSSETSKQLVKQKKQDISTRVLAKVNEFEQTGELRIPKNYSVE | 195 |
| | | | | NSLKSAYLIELSETKDKNKKPVLESCSVTSLSEALLKMVVWGLN | |
| | | | | PMKKQCYFVPYGGKIECIPDYTGKIAMAKRYAGLKDIKAHAVF | |
| | | | | KDDTFEFEVDPSTGRKKVTKHTQTLESMGSNEFKGAYAIMEM | |
| | | | | NDGTFDVEIMSKPQIVAAWQQGHANGTSPAHKKFPDRMARKS | |
| | | | | VINRACDALIRSSDDSVLYEDEDERKIIDVPSEDLKHEVKTKAN | |
| | | | | KRNFDTSDIEDADYEEDEEPTNEAENEDDENERLYEEAMAEEE | |
| | | | | GQSSMANSPGFGA | |
| PF062 | SSAP | recT | Brevibacillus brevis (strain 47/ | MSEAKTVNQSALAGQLAQRTMTKAENFNAVIKKELADNFQAI | 196 |
| | | | JCM 6285/NBRC 100599) | KSLVPKHMTPERLARITLTAISRTPALIDCTPASIVGAVMNCATL | |
| | | | | GLEPNLIGHAYLVPFKNNRTKQMECQFQIGYKGQIDLIRRTGD | |
| | | | | VSKIYAETVYENDLFIYIKGEDKRLVHVPFDMLHHLENFTPSKD | |
| | | | | DFMDIMMAQAIGAIKSRGANDQGKPVRYYSAYRLKDGAFDFV | |
| | | | | TMTAEQCQKHAMTHSAAKKDGKLVGPWKDHFESMCKKTCIK | |
| | | | | EMAKYMPISIEVQEKLSTDEAVLKLRKDNGIESDNIFDVDYKIV | |
| | | | | EEGQAEPDEEAAE | |
| PF063 | SSAP | recT | Prevotella sp CAG:873 | MSKVNFSAQYIASLKPMQIVKDNLVRQRFIDLYGALWGEANA | 197 |
| | | | | EATYEREVIHFNRLLADNANLKACTPVSIFIAFIDLAVCGLSVEP | |
| | | | | GVRALAYLQPRGYKTGQKDATDKDIYEQRCTLTISGYGELIQR | |
| | | | | TRAGQIRHADNPVIVYEGDGFSFTDHNGTKSVEYTLNINHNPER | |
| | | | | PVACFMRITRADGSIDYAVMLEEDWRRLAGYSGKANKKWDY | |
| | | | | DTRSYVEVPNSLYSSGEGNRIDSGFLMAKCIKHAFKTYPKLRIG | |
| | | | | KGTTYEADEAPRQEDDFYGMGDNEQPTAQPEESFAPAPDTSAG | |
| | | | | VTVDPSQSDDNDETF | |
| PF064 | SSAP | recT | Bacillus sp 1NLA3E | MSTQNELAVKTSSERFLNNIQAQFAAEAGSPVAFSDYEKALAQ | 198 |
| | | | | HLFLKLDSVLQDFEAKRINSGKTQQAPYNWHNINMRKLSLDA | |
| | | | | VHTVQLGLDALIANHIHPVPYFNGKEKKYDLDLRVGYIGKAYY | |
| | | | | RMEAAVEKPKDVVIELVYSTDHFKPMKKSFSNSIESYEFEIQKP | |
| | | | | FDRGEIVGGFGYLVYDDPAKNKLILVSEADFDKSRKAAKGDTF | |
| | | | | WSKHPAEMRFKTLVHRVTEKLQIDPKKVNASYLYVENKESED | |
| | | | | EVRKEISENANKDVIDVEFSETPDEPVNKEPKVENHSEHFEEAP | |
| | | | | PQQEQMEFAPTGTGGPGF | |
| PF065 | SSAP | recT | Faecalibacterium sp CAG:82 | MAKAMQPQKLYFSQAMQTEKYKKLINNTLGDPVRAARFAANI | 199 |
| | | | | TSAVAVNPTLQECDAGTILAGALLGESELLQPSPQLGQFYLVPF | |
| | | | | KSKAKRDRQGNVIEPACLKAQFVLGYKGYTQLALRTGQYKRL | |
| | | | | NVLEVKSGELGGWDPFEEREHEMHFIEDFEKRAAMPTVGYIAH | |
| | | | | FEYINGFEKTLYWTADRMMAHADKVSPAFSATAYKKLLNGEI | |
| | | | | PQEDMWKYSSFWYRDFDGMAKKTMLRQLISKWGIMTVEMTT | |
| | | | | AYERDGRVMVPNSADDGLLPETPDFADAGQNGLSEQDPPKIER | |
| | | | | TAKTMDLPEPEADEVKAAVDLATL | |
| PF066 | SSAP | recT | Pelobacter propionicus (strain | MSNALVKLEFDKEQMAVIETQLEPSGTSKAEQQVCLSVARELC | 200 |
| | | | DSM 2379/NBRC 103807/ | LNPITKEIFFVKRRQKIDDKWVTKVEPMVGRDGFLSIAHRSKQF | |
| | | | OttBd1) | AGIETTAGIREVPQLEGGQWGFKNQLVTECIVWRKDSPKPFTV | |
| | | | | QVAYNEYCQRNSEGNPTKFWAEKPETMLKKVAESQALRKAFN | |
| | | | | IHGVYCPEELGAGFELASGDIVIQAIEEERPGNETDKSHLSVVKP | |
| | | | | PQAETQATKTPKHQKSSTATTSQSAPINEEVQTSPPSPGQVIDEA | |
| | | | | ALEVIELLDGKHIPYDIAINGEDGIISAKSFNEKELEKSSGFRWS | |
| | | | | ADQKRWIYKFRNEPF | |
| PF067 | SSAP | recT | Herbaspirillum sp YR522 | MNQVALSPAGTLNQFLKQHKNQIEMALPKHITPDRMMRLTLT | 201 |
| | | | | AFSQNRSLQDCTPQSIFASVIVASQLGLEIGVGGQGYLVPYKGT | |
| | | | | CTFVPGWQGLVDLVSRAGRATVWTGAVYRGDKFDWALGDRP | |
| | | | | FVKHQPEGDGDDWHDITHVYAIGRVNGSDHPVIEVWTMDRIV | |
| | | | | RHLNKFNKVGGKHYALTNNGQNMEMYARKVALLQVLKYMP | |
| | | | | KSVEVMRAMDVANAVDSGKNFTFDGDVVVIDDRDIDESPGDS | |
| | | | | SPGATGVQQQSGGRPEIPECTDEEFKAKTPRWRKQLLEDGVSE | |
| | | | | ADLVKMIETRTKLTEDQKTTINSWTHEND | |
| PF068 | SSAP | recT | Desulfovibrio sp FW1012B | MSNGNLPQESGGASVAALIQQQIPAIAMAVSGGTKEERQKRAE | 202 |
| | | | | RFARVALTTIRNNDKLAQCRVESLLGALMTSASLNLEIDPRGLA | |
| | | | | YLIPYGREAQLQIGYKGIKELAYRAGGIKAIYAEVVYKPEVEAG | |
| | | | | MFTIEIGLSRSLTHKLDPERPELRNGDLVLAYAVAEMEDGRRH | |
| | | | | FAYCLRDEVEKRRKTSKMNTASPDSTWGKWAEEMWRKTAVK | |
| | | | | KLCKDLPQSTEDAMAKAVALDDQAEAGVPQTFDLPKDFIDVT | |
| | | | | PEPKTASDRAAQVLGKTEAEAAPPVDAPTSVPCPNRPNDETGG | |
| | | | | FWDVKATVCEGCMDRGGCPSWVAA | |
| PF069 | SSAP | recT | Nitrolancea hollandica Lb | MADNNHALAIVPDSNEVKSLAKRLVPQFPSDCNAEQAADVAR | 203 |
| | | | | VAVAYGLDPFLGELIPYKGKPYLTFDGRIRIADNHPAYDGYDH | |
| | | | | GPVLGDERTAFMPQNGEVIWKCTVYRRDRSRPTVAYGRAGGD | |
| | | | | KETNPIAKKDPVTMAQKRAIHRALRAAFPVPIPGGDDDPVTPA | |
| | | | | QLKAIHATDSEQGITVTERHEVLEATFGVGSSKDLTAAQAGAY | |
| | | | | LDERAAQKPAMEQPAIEVTARPATPPQEPTDPPVGALLSARQQ | |
| | | | | RRIYELRDELGKQTAELNAAIQELYKVDSIEGLSEAQASRFIRSL | |
| | | | | QRSAEKARQQQADDVIDAELADIPF | |
| PF070 | SSAP | recT | Fusobacterium mortiferum ATCC | MAETKRATNSLVKGSNGAVTEKKPNKTIYEIIKAGEKQFAAAL | 204 |
| | | | 9817 | PKHLNSERFTRIAITTIRQNPKLAECNAESLLGSLMTIAQLGLEP | |
| | | | | GVLGQCYLIPFKNNKLGTIECQFQLGYKGMIELLRRTGQLSDIY | |
| | | | | AYTVYSNDEFDIEYGLDRTLKHKPAFTNPEGRGEIVGFYSVAIL | |
| | | | | KDGTRAFEYMTKKEVIEHEEKYRKGNFKNEIWNKNFEEMSLK | |
| | | | | TVTKKMLKWLPISVEMIENLRKDEQIHKLDEKTNEVTSEYIDEN | |
| | | | | IINYDEDGVIVEEKPTTEDMQTEMTISQASGIDITKKAKELYGVS | |
| | | | | TLDDINMEQYNELKEIAING | |
| PF071 | SSAP | recT | Akkermansia sp KLE1798 | MALTPGLVTLNHAKIMSNESTDKLDLPQAPVPKKTLYEIVMSE | 205 |
| | | | | DVKTHITQFVEGMMTPERCISIFWNCCQKTPLLQQCAPITLISSL | |
| | | | | KNLLLMRCEPDGIHGYLVPFWVNDKRTGNSILTCAPVPSARGL | |
| | | | | MRMARSNGVTNLNIGIVREGEPFSWREDDGKFTMGHIPGWGD | |
| | | | | NEDPIRGFYCTWTDKDSYLHGERMSLKAVEEIKGRSKSKNKKG | |
| | | | | EIVGPWVTDFGQMGLKTVIKRASKQWDLPLVIQAAMQAADDQ | |
| | | | | EFEGNMRNVTPEKTDGPAEGETPWNNAPAPEAFQNDQPEALPE | |
| | | | | PKPEGQDDLIPGLKMPAPKETATVNMEDY | |
| PF072 | SSAP | recT | Thiorhodovibrio sp 970 | MTRSMQAVPKADAPRIPMPAVDPALNLTPSTWKVLTDSIEFPAA | 206 |
| | | | | KTAEGILLAVHYCAARNLDVIKRPVHVVPMWSKALGQEIETV | |
| | | | | WPGIAEVQTTAARTGQWAGIDPPRFGPVIERTFSGKVKRNGAW | |
| | | | | QDLDYAVSFPEWCEVTVYRLVGGQRCPFTEPVFWLEAYARQG | |
| | | | | GAYSELPTEMWVKRPRGQLMKCAKAASLRAAFPEEASYTAEE | |
| | | | | MEGKVIEADTSIPVAASLDATGTVPAKADTAPSGVAEAASDAA | |
| | | | | PSKASETPTDSEPRAEPITLDPSLQARIDKVVARAAEKSAWKQA | |
| | | | | EQYLRARCKGAELKQALEALDQAQQTAEQRLAA | |
| PF073 | SSAP | recT | Pedobacter antarcticus, | MSNQIQVTKDYIDRLHPLSVVKDAAIGDHFINKFVAMYRVPRE | 207 |
| | | | Pedobacter antarcticus 4BY | QAVAFHEREKDNFIKRITDSEDLSACTPMSIFLAYMQVGGWQL | |
| | | | | SFEGGPQSDVYLIPGNRNVAPKGQPDKWIKEVVAQPTPYGEKK | |
| | | | | IRIQNGQIKDAAKPIIVYECDDYEEFTDDIGNVRVTWKKGNRGD | |
| | | | | KPVIVGSFIRIEKPDGSFEIKTFDMGDVAKWKASSENKNSKWD | |
| | | | | AAAGRKLPGKANALYTSNSGQIDKLFFEGKTLKHAFKLYPKVV | |
| | | | | NSPKLPDAFVPVASDAIRQGFDVSEFTEAEYVPEEDLSQSEQSD | |
| | | | | FDVALEEAHNTEPIQTKTFAGIDTSDEPEF | |
| PF074 | SSAP | recT | Rhizobium loti (strain | MMNAITTYTHSPRQLALIQKTVAKDCNTDEFNLFVEVARAKGL | 208 |
| | | | MAFF303099) (Mesorhizobium | DPFLGQIIPMIFSKGDSNKRKMTIIISRDGQRVIAQRCGDYRPAS | |
| | | | loti) | KPPSYEFDAELKSETNPQGIVSATVYLWKQDAKTAAWFEVAG | |
| | | | | QSYWDEFAPISYPYDAYKMVDTGETWEDSGKPKKKRVLRDG | |
| | | | | ATPQLDDSGNWCRMPRLMIAKCAEMQALRAGWPEQFTGLYD | |
| | | | | EAEMDRAKVLEMAASEIVAHEQEENRLRLVAGNDAITMSWGD | |
| | | | | GWALENVPAGEFMDRCLAFIRESDHQTVVKWNSANRAGLQLF | |
| | | | | WAKHPGDALELKKAIEKASRAPVEIDHIADAARREIAQHPVSA | |
| | | | | G | |
| PF075 | SSAP | recT | Stigmatella aurantiaca (strain | MDGHNKAETTAAQQAWGRERVELIKRTICPKGIGDDEFSLFIE | 209 |
| | | | DW4/3-1) | QCKRSGLDPLLKEAFCVGRRQNAGSRERPTWVTRYEFQPSEAG | |
| | | | | MLARAERFPDFKGIQASAVYAEDEIIVDQGKGEVVHRFNPAKR | |
| | | | | KGALVGAWARVVREGKEPVVVWLDFSGYVQQTPLWAKIPTT | |
| | | | | MIEKCARVAALRKAYPEAFGGLYVREEMPAEEYEPSSAAEEPA | |
| | | | | PTTGTGTYEVLGARPGPVKASFPPLPAAQLSMEVQPPVAAPVA | |
| | | | | EPPPAAETAPRPRSSATVVAFGPYKGKTASELSDDELSETIDLA | |
| | | | | HEKLMEQPKAKWAKAMRENLVALDAETELRCRVPASGKKEA | |
| | | | | SAEA | |
| PF076 | SSAP | recT | Methyloversatilis universalis | MTGPELAAAAKQTAQQAGSATVKKFFEANRGTLEALLPRHFD | 210 |
| | | | (strain ATCC BAA-1314/JCM | SERMLKLALGALRTTPKLANASLSSLLGSVVTCAQLGLEPNTPL | |
| | | | 13912/FAM5) | GHAYLLPFDKREKQNGQWVTVETQVQVIIGYKGMLDLARRSG | |
| | | | | QIVSIAAHEVCQNDEFVFAYGLNEELVHRPAMKDRGPVIGFYA | |
| | | | | VAKLTGGGYSFEFMSVDEVNHIRDKAAEKNRAKRDAAGNPIIT | |
| | | | | GPWADNYVEMGRKTVLRRLFKYLPISIESLAFASAVDGQAIREP | |
| | | | | APLEQVAFESSEPAEEPTSYDQQDEDLRALEQSQPARVPPVQVE | |
| | | | | QPAEAWQPSAEEAAELERQQAAEAAADQQRASAPRGRGQRS | |
| | | | | MSLE | |
| PF077 | SSAP | recT | Hydrogenovibrio marinus | MYSQQAYPEQNQYPAATNNVVSVFDNSENKSAIKKLLPRHISI | 211 |
| | | | | DKEMQIANTAAMQFPDLQECTPESLFVAFSRCAQDGLIPDGRE | |
| | | | | AAIVSYNKKQGNTWIKVAQYQPMVEGVLKRLRMSSQVKNVIA | |
| | | | | KVVYENENFNHWIDIDGEHLMHQPVFDDKRGELKLVYALAKL | |
| | | | | DSGEKVVEVMLKSEVEEIMMNSKSAVDKNGVLKPYSVWATH | |
| | | | | FPRMALKTVIHRITRRLPNASEVAEMLEREIDYKEVSEDRQPQT | |
| | | | | IEHNQNQGSQDREIIPLEQQMELKKLVEQTGSDENNMFNWISN | |
| | | | | KTEILVSSYDQLDFNQFTRLKTRLENAIAKQVAAQRQQAMEQS | |
| | | | | GEFVSA | |
| PF078 | SSAP | recT | Photobacterium profundum (strain | MNELQRTNNQAPVNHQVNAVPTSSISILDTNKLDVMIRAAEFM | 212 |
| | | | SS9) | SKAVVTVPEHLRGNSSDCLAIVMQAEQWGMNPFTVAQKTHLV | |
| | | | | SGTLGYEAQLVNAVISSSRAIVGRFHYRFSGGWERLVGKVGYE | |
| | | | | KQQRTNFKTKAKWEVTVATKKWLPEDEAGLWVECGAVVAG | |
| | | | | ESEITWGPKLYLASILVRNSELWVTKPQQQIAYASLKDWSRLY | |
| | | | | TPAVMQGVYDREELAQPSFENIRDITPEIKPIKQLNNEPSIDSLM | |
| | | | | GGDAVEAETAGSNEPNLNALVSGDIEMMQNDDTPSVFETTRD | |
| | | | | LITDISDIQECVNYRRDIETMKNNQTITANEFSILKKMIKTVHNT | |
| | | | | FNVQG | |
| PF079 | SSAP | recT | Pirellula sp SH-Sr6A | MTAAAEERTEHVSGASPKRSEAMILLEDLQSDDTSRKLLQILPS | 213 |
| | | | | HMKPEFFVRVVINQLNKNPKLALCTRQSFIGSVLDLAAIGLEPD | |
| | | | | GRRAHLIPYGRTCTYQLDYKGIAELVLRSGKVSHVAADVIRRG | |
| | | | | DIFVWNKGQLKEHVPHFLRTDAKAPKEAGEVFAAYSLVVFKD | |
| | | | | GTERAEVMSRADILAIRDGSPGWQAFVAKKSNDTPWDPRFPH | |
| | | | | KEFEMWKKTTFKRLSKWLPISAEAQAAIEKEDRNDYGDRNSN | |
| | | | | VINSPSVRTVRSADDLKEELKRRNAESTSTEVIHDTEFVAPEGIS | |
| | | | | KQAELLELKAATDPLTIIEIERDLEHVRAEDRDEVLAAIASAKA | |
| | | | | RL | |
| PF080 | SSAP | recT | Ahrensia sp R2A130 | MNAIARLESGIDRSISNEIAVGSSGLTFQNAGQIMEYAKMMAVS | 214 |
| | | | | GSAVPKHLRGNAGACLGIVDDALRFQMSPYALARKSYFVNDN | |
| | | | | LGYEAQVLAAIVISRSPLRERPNVNFEGEGQDRVCIVSATFNDG | |
| | | | | AEREVRSPAFKAITPKNSPLWKSDPDQQHSYYTLRAFARRHCP | |
| | | | | DVLLGMYDPEELSSMAARDVTPPRAPTAQERLKSIAAEKPAEP | |
| | | | | VLHNEGFGTGNMLEDAPASAVSEPEQSQADTGEIVDQQEQEPA | |
| | | | | PAVETRIEPERTTLNQDDKDVLTDFIIPFWKAESEEARKMVRM | |
| | | | | KHDPMIEARGKLATTAARKMVEAVIAGKPAEAGEVIGLTMQD | |
| | | | | MEELS | |
| PF081 | SSAP | recT | Borrelia duttonii CR2A | MTKNNILKNQSTNTVDVIIEKMNSSNIAEVWETYKIMHNLKKI | 215 |
| | | | | DAYSEREILTLLQVNKLNPFKKEAYIIPFNGRYTVVVAYQTLLI | |
| | | | | RAYEAGYNKYDLDFEEKLVKSLKIDSKGNKMIQEDWQCTAFF | |
| | | | | KSDDGIRYSFSVLLSEYFKNTPIWREKPVFMLRKCAVSCLCRTL | |
| | | | | PGSGLESMPYIREELEDMGTVLQQELQGFEEVNNSTPEATIEIQS | |
| | | | | VNHNSGDDINKSIPTKYYCYQNLLIAARNMYNFVSDKPFGSLS | |
| | | | | EINTYLESVKSGDDSKLLEYFNTNKMLKSIEYWCNLLKEYFTK | |
| | | | | SSRDLSRLEKFNIFMSFDLDKVGNSPLKLFSQLSITKEFQCLFSL | |
| | | | | T | |
| PF082 | SSAP | recT | Candidatus Accumulibacter sp | MSKTLRNLQKSRQGTPANAVSFPVMLEQFKGEIARALPRHLSP | 216 |
| | | | SK-12 | ERIVRVALTAFRLNPKLADVDPRSIFAAVIQSSQLGLEVGLMGE | |
| | | | | AHLVPFGSQCQLIPAYQGLMKLARNSGLVQDIYGHEVRINDRF | |
| | | | | DIVLGLHRSLMHEPLKQNGFPAADDERGAIVGFYVVAVFKDGT | |
| | | | | RTFYALSREQVEQVRDHSRGYQMARKLRKESPWDTHFVSMGL | |
| | | | | KTVIRRVCNLLPKSPELAMALAMDELNERGETQNLGVTEAIDG | |
| | | | | SWAPVLDEQPDDPGQASDPARASTRKPLADLLAGIGQALSLEA | |
| | | | | LDEVYAQAEDAVAGDDLERLLRAYRSRKAALTPPLSPSSPIRG | |
| | | | | VVNNASA | |
| PF083 | SSAP | recT | Bifidobacterium reuteri DSM | MGQLARSIQNRQLQAMPNDQERKQQFMTALEKDWPRIVASMP | 217 |
| | | | 23975 | KHMTPDRIFQMYQSTLSREPKLRECTLNSVLSCFMKCAQLGLE | |
| | | | | LSNADGLGKAYIIPFFNNKNNEMEATFLIGYRGMLQLARNSGEI | |
| | | | | KSMQAKAVYDGDEFHYQFGLHEDLVHIPAEKRPRNAKLTHVY | |
| | | | | FIALFKDGGHQLDVMTRDEVDAVRSRSKSKNNGPWVTDYEA | |
| | | | | MAIKSVIRRAFKMLPNSADVKEEVQQHVDAYTPDYSGILPSSPE | |
| | | | | GLPDSDADAPEEPSDIDGVLADGGTEQPAEEPDPVEVKRRDVIR | |
| | | | | RFQQLGVTSDAEACGTISKVLGREVKETGGLPEADEDVVLAQL | |
| | | | | KASVREGE | |
| PF084 | SSAP | recT | Serratia odorifera DSM 4582 | MSTEIIEQKKNGIIDNVSILTNGDLFDRLMKISEVMAKSGAMVP | 218 |
| | | | | QHFRDQPDACMAITMQAARWGMDPFVVAQKTHLVNGTLGYE | |
| | | | | AQLVNAIINSMAPTKDRIHFEWFGPWENILGCFVEKTGKSGNK | |
| | | | | YIAPGWSLADEKGVGVRAWATLKGEDEPRELVLYLSQAQVRN | |
| | | | | STLWASDPRQQLAYLSVKRWARLYCPDVILGIYSDDELLEPTP | |
| | | | | RAEKDITPVASAVTELDSPPTSTEPDAGKAAELAEALSAALDEA | |
| | | | | STQEEAATIEQRIAKHKDALGSSLLFSLRGKAQKKRSGFRGVSE | |
| | | | | IDAAFSALDGTDNQKFQQLETLVANWKNALPPADVERFTLAL | |
| | | | | DDLRPEYQQ | |
| PF085 | SSAP | recT | Persephonella marina (strain DSM | METKTLAPPTALMGNVDYDKLMQQAGQLILRDREGKPYTNEQ | 219 |
| | | | 14350/EX-H1) | RALIVLAAQQLGLNPILGHLTIIQDRLYITNAGLLHIAHNSGKLQ | |
| | | | | GIKTRPATEEERKAYFLGNNPKDIHLWRAEVYLKGQKEPYIGW | |
| | | | | GKASTNEKSWAVKSNPQEMAETRAVNRALRKAFNVAGYTSV | |
| | | | | EEIDEEPQFINEEDDEEPITQEQVKILHAIANLISKDFYDHEFKN | |
| | | | | WLREKTGKESSKELTKSEASDIADRLLTVLEDKLKFLADEAGIN | |
| | | | | YYKLLNDKYSIGTLSETDNYYIWKEVRDYIEEAYKRKGLLKSIL | |
| | | | | EVAQEKNISNEEIKQIIFKEFGKESSKQLTIDELERLLNIVENI | |
| | | | | SFEPSDEDVPF | |
| PF086 | SSAP | recT | Parcubacteria group bacterium | MSNFQNAVAIIAKQESKFMELVKAASTDVVFKQEMLYASQAM | 220 |
| | | | GW2011_GWA2_42_14 | MNNDYLCKTAINNPLSLRNAFESQVAACGLTLNPSRGLCYLVPR | |
| | | | | DGQVILDVSYKGMIKTAVNDGAIRDCIVELVYSNDKFVYKGKR | |
| | | | | HSPVHEFDPFDLKEQRGEFRGVYVEVTLPDGRVHVEAVTAVDI | |
| | | | | YKARNASDLWTRKKKGPWVDFEDSMRKKSGIKIAKKYWPQV | |
| | | | | GEKLDSVIHYLNTDAGEGFASQDVPVSVVERYMGQVEQVEPA | |
| | | | | EPLPTSNETVPVVASVAVAEQAAATQSQPTPESEAKAPLQGTV | |
| | | | | DPNVEAAESALPERTLNKVEELVKRARNSGAWKAAHEYISTW | |
| | | | | PEIARDYATKKLKAAEYQLAASGE | |
| PF087 | SSAP | recT | Xanthobacter autotrophicus | MNALAPINVQSSAIRAMVPQTMDEVFHLADAVHRSGLAPTGL | 221 |
| | | | (strain ATCC BAA-1158/Py2) | KNVQAVAIAIMTGLELGVPPMTALQRIAVINGRPTIWGDLAIAL | |
| | | | | VRASGLAETIKEEILGEGDDRVAICTVKRKGDPQQVVGSFSVA | |
| | | | | DAKRARLWDPREKVQRRRQDGNGTYEALNDAPWHRFPERM | |
| | | | | MKMRARAFALRDGFADVLGGLYLREELEEEQVDIRDVTPPTAP | |
| | | | | PPTITEEKKAPAPPAPAPAALAPPDPHVDLPSPLDRVTTPEVRD | |
| | | | | MVLNSPSAPAGLAQKAPAPPPPSVASVPAREERAPSPGEGAPRP | |
| | | | | DRAPSPAPFPNMAPHLVSLWAELGKCQNPTALENTWAFEEDDI | |
| | | | | NTWSMADRESAAALYERRLAELCRKG | |
| PF088 | SSAP | recT | Ruminococcus sp SR1/5 | MANEMTVQKTESLSNSEAFTNKVLKEFGSNVAGNIQVTDYQR | 222 |
| | | | | QLIQGYFIATDRALKMAEEKRVSKNENNKDHKWDNLDPINWN | |
| | | | | TVDLNALALDVVHYARMGLDMMQDNHLSAIPFKDNNRLSRT | |
| | | | | GTKMYVVNLMPGYNGIQYIAEKYALEKPVSVTVELVYSTDTF | |
| | | | | KPLKKNRENRVESYDFEINNAFDRGEIVGGFGYIEYTEPTKNKL | |
| | | | | IIMTIKDILKRKPDKASGEFWGGKKTAWEKGQKVEVETEGWFE | |
| | | | | EMCLKTVKREVYSAKNMPRDPKKIDDAYEYMRMQEIRLAQM | |
| | | | | ETQEVIDAEANQVVIDTEAQETPQKPAQPAFLTDDGGQQALDL | |
| | | | | GSPAKPQAQPQPARNTTAARSTATRAAGPTF | |
| PF089 | SSAP | recT | Sulfurovum sp ES06-10 | MSKNEVTHVSQGQGAMIKSPFSAYALTEEQSNIIKTQIAPGITD | 223 |
| | | | | GDLMYCLEIAKQAQLNPIIKEIYFVPRRSKIDNQWVTKHEPMIG | |
| | | | | RKGARSIARRKGMIVPPTTGYTLKQFPFLENGEWKEKRDLIGW | |
| | | | | AELEITGQKVRKEAAYSVFVQKTSEGAVTKFWSTMPTVMIEKV | |
| | | | | AEFQLLDAVYGLDGLMYMDAGYIEDESSSTQEMSSLADLGKIE | |
| | | | | RELKSLNLEYHLVGDEIRIQDKGAFQFAAALKKEGFVYENSTK | |
| | | | | FWKIRVAMVENADIEAPKELPKPEPEQIDPKKELANAKKALMK | |
| | | | | VLLGNGLTKEEAGEFAQTLDVSNVDSINKLLPGNGGHDELIAKI | |
| | | | | NVFLTPQSSQTHQDNLFDGDEEENPFG | |
| PF090 | SSAP | recT | Microgenomates group bacterium | MEVKNELAIQFDKTKAVMRSEQAIERFAEALGSGQQAKRFIGS | 224 |
| | | | GW2011_GWF1_44_10 | VLLVVSQSDRLMECTPQSIMVSGVRAATLKLSVDPSLGHAVIV | |
| | | | | PYKNKGIPTATFIIGYKGLKQLAYRTGQYAFINEKIVYDGQTIEE | |
| | | | | DDFSGIQRVRGIPTTYGKDGWKPIGYWMGFELKNGFRQTFYM | |
| | | | | TIEEVEAHAKRYSKTYDKVKKQFYPDSLWHTDFDVMAIKTVIR | |
| | | | | LGLSKYGYFDEDSLLAMAESSEFDDDLVVDGELIEDALEQEAE | |
| | | | | REQAREAEHAGKSTEQLSSLLGFEDPPDTSKKEPVKKAAAKEA | |
| | | | | VTKVKPKEYEVAASFNSKRLNKLYGEMSVEELQGEIMALTQY | |
| | | | | ETKTQEEKVQVNDRIQAAKELIRYIESNVL | |
| PF091 | SSAP | recT | Mycobacterium marinum (strain | MTEIATIDETTTDLIRGQVGFNSTQRAMLAQLGLSDAPEGDLIL | 225 |
| | | | ATCC BAA-535/M) | FSHVCQKSGLDPFRREIFMIGRNTQVTRYEKVDPDDPESNQRK | |
| | | | | VTRWETVYTIQTGIQGFRKRARELADEKGDRLGFDGPYWCGE | |
| | | | | DGNWKEIWPDTDKPVAAKYIVFRNGEPVPAVTHYSEYVQTTK | |
| | | | | VDGVAQPNSMWSKMPRNQLAKCAEALALQRAYPDELSGIVLE | |
| | | | | DAAQVIDSDGQIINETQRPPARARGAAALRDRAKAEAKPDDPE | |
| | | | | AVNADVVEPRHEDVASQPRPMSDGSRRKWLNRMFQLLGESDC | |
| | | | | TEQESQLTVIAKLANAPGIEHRDQLDDGQLKGVVNQLNQWEK | |
| | | | | SGQLVDKVADILDQAAIDEAQAAEQDAQQTIDGGN | |
| PF092 | SSAP | recT | Mameliella alba | MNTQIAKIPLRQVQDVKTLLHNDQARQQLAAVAAKHMSPERL | 226 |
| | | | | MRVTANAIRTTPKLQEADPLSFLGALMQCAALGLEANTVLGH | |
| | | | | AYLVPFKNNRKGITEVQVIIGYKGFIDLARRSGHITSISAGIHYS | |
| | | | | DDELWEHEEGTEARLRHRPGPQEGEKLHAYAIAKFTDGGHAY | |
| | | | | VVLPWARVMKIRDGSQNWQSAVKFGKTKQSPWYTHEDAMAS | |
| | | | | KTAIRALAKYLPLSVEMADAITIDHDEGTRVDYAAFAQSPEDG | |
| | | | | PQVEEGEEIDGEATEVDEGDRHQQDADPKGATGETEEKPNQKT | |
| | | | | KERKPEAKKEQDKPKDDDAGIPPASDPKFVKLFQDIEAELTDG | |
| | | | | APARGILRFHEAALAEVEKEDPDLHGQIMSMIREAEAND | |
| PF093 | SSAP | recT | Gordonia soli NBRC 108243 | MSSTEIATTTGAVATQPTSDLAIQPNQTEFTSVQRAALAQLGIE | 227 |
| | | | | EATDEDVQVFFHQAKRTGLDPFARQIYMIGRRTKVKEWDPNQ | |
| | | | | RKQIEKWVMKQTIQIGIDGYRLGGRRIASALGIKLEKDGPHWH | |
| | | | | DGNGWVDVWLDPARPPAAARYSITRDGETFTATAMYSEYVQT | |
| | | | | YNTQQGPQPNSMWSKMPANQLAKCAEAAAWRQAFPDQFSGV | |
| | | | | IFEDAAQHTVIDADVIEEEPKTKQGSRGTAGLAAALGVDESTA | |
| | | | | DEPEPQDGPVGTIESGLISAEPSETPEDEADSSHEPRPEPKKPTQ | |
| | | | | KQIHALNALLSQAGLTKAEKKGRQIVVSSFLPNREDPAAALTA | |
| | | | | DETEHVTTQLSALVENQGEQALIDTVEALIQQHDQQGAE | |
| PF094 | SSAP | recT | Sphingopyxis sp (strain 113P3) | MNAQTQIATRADNPLAILKTQIDERAKEFQAALPSHISPEKFQR | 228 |
| | | | | TILTAVQADPELLKASRKSLILACMKAANDGLLPDRREAALIVE | |
| | | | | KRNYKDAQGAWQQALEVQYLPMVFGLRKKILQSKEVTDIKPN | |
| | | | | VVYRREVEEGHFIYEEGTEAMLRHKPILDLTDEESSDDNIVVAY | |
| | | | | SIATYKDGTKSYEVMRRFEINKVQNCSQTGALIDKRGKPRTPSG | |
| | | | | PWVDWYPEQAKKTVMRRHSKTLPQSGDLVDVEGSEIDQQRA | |
| | | | | ALSAMGALGAGEPIDATPVAPALPQADDLPDHDADTGEIIDTV | |
| | | | | TPDNPNAAEGRADEQHGDQHDGTEEPQGDEDQPYAATVSELIE | |
| | | | | RAAAAGTVIDLNAVEKDWQKHMAALPDVENARIDTAVKERR | |
| | | | | DQLTAK | |
| PF095 | SSAP | recT | Actinobacteria bacterium OK074 | MTVDTPVTWLAIREDQKAFEDQQLRALRAAFPDLVDASPAQL | 229 |
| | | | | GIFFHYCKASGLDPFGRQIYMIKRKSRGEVRWTIQTGIDGYRLI | |
| | | | | ARRAADRAGQSIAYEDEFVWYDAEGGEHAVWLRDEPPVACRV | |
| | | | | VIWRGEARFPAVAHWREYAPKVWDYEAQEYKLGGLWPQMP | |
| | | | | ASQFGKVAEALSLRRACPADLSGLHVDEEMHAADAAESRERV | |
| | | | | KEAAARLRSPDEQQANGGSPAQPTSDVVDAEIVESQTANTDAA | |
| | | | | APQEEQRADKAAEPPASGRTDEVDRVRERMSALDQERSRLEE | |
| | | | | ARARIRETADRLALGFDTVNDRCFDAFGTSFQDASAEQLSELN | |
| | | | | DSLTPAAGTSDEPATAGRRNGSARRTRTPAKKAAASAKKKTA | |
| | | | | RKATDGTTSPTRVSK | |
| PF096 | SSAP | recT | Rhizobium sp CF080 | MNSLTTVEAGRLIDGLRVELEPLVLDSGKSFDRLRSVFMIAVQ | 230 |
| | | | | QNPDILKCSENSIKREISKCAADGLVPDNKEAAMIPYKGELQYQ | |
| | | | | PMVQGIIKRMKELGGVFNIVCNLVFEKDVFTLDESDPDSLSHVS | |
| | | | | DRFATDRGKVVGGYVVFRDEHKRVMHLETMSIVDFENVRKAS | |
| | | | | KAPDSPAWKNWTNEMYKKAVLRRGAKYISVNNDKIRALLER | |
| | | | | QDELFDFTPNRVVERTNPFTGEVIEGNATPSIENKQQPPMQQPR | |
| | | | | ETQPAGSQQEPRQQRINNTGDGRASRKQERQPENKDAGNTQQ | |
| | | | | TKTETKKDLVPPAVPEVDVFPADKAKAAEAAEKLLGVALLPD | |
| | | | | LDPRGRRGVLKQAAVDWKEATPAYTHPLLKACIDMSDWAIQQ | |
| | | | | QVKEEAWAGEHAMFVHKIKSLLDVEKLNVGKYP | |
| PF097 | SSAP | recT | Bradyrhizobium sp STM 3843 | MNSARGWWLDKNLVKLARRTAFKETNEDEFDQAVAFCREKN | 231 |
| | | | | LSPMSGQLYAFVFNKDDAKKRNMVIVTSIMGYRAIANRSGDY | |
| | | | | MPGPTKAFFDPGAKNSLINPRGLVRAEGGADRFIHGGWKNVTE | |
| | | | | EALWESFAPIIKSGSDDDAYEWVDTGEVWADSGKPKKKRRLR | |
| | | | | QGAEVQEILDPKKEGWHRMPDVMLKKCAEAAALRRGWPEDL | |
| | | | | SGLYVEEEVHRSQVIDADYVDLTPSEMVAKAETDARIEKIGGPS | |
| | | | | IFAAFDAAGTLERVEIGKFFDRVDAHTRNLKPEDVASFAVRNR | |
| | | | | EALREFWGRAKNDALTLKTILETRSSAATPASQANGHDGAAGT | |
| | | | | AAPRSESQTDSGPDTGAADPSLSSDAAAKLKSALLADVAQLRT | |
| | | | | RSDFDSWERDAKAHLEKLPEQMRSEVQAELDHRRADIR | |
| PF098 | SSAP | recT | Nitratireductor basaltis | MNQIATSTQRELAEKDKFRQQMKGQSEAFCEALIGSNIPPEKFQ | 232 |
| | | | | RVVATAVMTDTNILFADRKSLMEATMRAAQDGLLPDKREGAF | |
| | | | | VLFKNRVQWMPMIGGIIKKIHQSGDISLITAKVVYGGDTYRTW | |
| | | | | VDDEGEHVLYEPAEEPDHNVIRQVFAMAKTKDGTLYVEALTT | |
| | | | | RDIEKIRSVSRSGEKGPWKDWWEEMAKKSAIRRLAKRLPLSSD | |
| | | | | IHDLIQRDNEMYDESRAPEPRQSVMARFRASATPQIEMDRGEG | |
| | | | | FDIDHITRETGTLTSEDAEQVSSASPSLADEGDGADPAPETPSTV | |
| | | | | ATESEPAEAADQAEEASTEQASTPEASHGDEAGASSAVNPQTY | |
| | | | | AAMTECIDRLLGAATDTSGGDVEKRKEKVEKFAAAWCNELPD | |
| | | | | HTAFVDKCKDTALRIAEKPAERAKAEEYLKAKLPEVEA | |
| PF099 | SSAP | recT | Lentibacillus amyloliquefaciens | MEKAIQFSNSEKSLIWKRFIEPAKGTQEEAEHFLEVCENFGLNP | 233 |
| | | | | LLGDIVFQRYETKRGAKTQFITTRDGLELRVATSQPGYVGPPNA | |
| | | | | NVVKEGDHFEFLPSEGTVRHKFGTKRGQILGAYAIMQHKKHNP | |
| | | | | VAVFVDFEEYELANSGRQNSRYGNPNVWDTLPSAMIIKIAETF | |
| | | | | VLRRQFPLGGLYTQEEMGLDDNLQTEDAKETASPDKQHSAQT | |
| | | | | KPAAKEPEKEVSQPGDDVIHQEMVVKSYDIKTSSSKKQVGVLS | |
| | | | | VQSKTSNQAVQVLIRDKSLMKSLQHVSAGEILNLELYEENSFVF | |
| | | | | LKDVAERASVDNQQKDEKEENQKGESSKETDGPAKAPQENEQ | |
| | | | | SEAGYPVEVMIQNVKFGEKASEKFAKITGVIDGQTQLMLARGE | |
| | | | | QAVQKADGLEQGDNVTLSLKKENGFLFLVDLVEETQQRAG | |
| PF100 | SSAP | recT | Oligotropha carboxidovorans | MAKTETRERTQADHRSAEPAPNVNVPAQKSNHPVAVFREYAM | 234 |
| | | | (strain ATCC 49405/DSM 1227/ | QRISTLQELPHIDPQQLLSVALTAIQRKPDLMRCTPQSLWNACV | |
| | | | KCTC 32145/OM5) | LAAQDGLLPDGREGAIVPYGENADGKRVAEIATWMPMVEGLR | |
| | | | | KKVRNSGQIKDWYVELVYAGDFFRYRKGDDPRLEHEPVPPSQ | |
| | | | | RTPNTPFHGIVAAYSIAVFTDGSKSAPEVMWIEEIEKVRTKSKA | |
| | | | | KNGPWQDSAFYPEMCKKVVARRHYKQLPHSAGMDKLIQRDD | |
| | | | | DDYDFDRQDEALVQQRQHRRLVSTTSAFDEFARNGQTIDHRAS | |
| | | | | DPVHQDGDDEFAEDEPSHDETGADETDRTSANSKPETGGAAD | |
| | | | | QREEAAVNSKDEQQQHAEPQQQTQAEQTTANDGKPAAKFDPH | |
| | | | | VGEEVRRWPPGAVPSDPDEYEFYVETKLSDYTRETADKIPDW | |
| | | | | WKSAEEKKLREACGISKQRHDELRNKAASRKTELLKG | |
| SSB_01 | SSB | Viral- | Streptococcus pyogenes serotype | MINNVVLVGRMTKDAELRYTPSQVAVATFTLAVNRTFKSQNG | 235 |
| | | SSB | M28 (strain | EREADFINCVIWRQPAENLANWAKKGALIGVTGRIQTRNYENQ | |
| | | | MGAS6180), Streptococcus | QGQRVYVTEVVADNFQMLESRATREGGSTGSFNGGFNNNTSS | |
| | | | pyogenes, Temperate phage | SNSYSAPAQQTPNFGRDDSPFGNSNPMDISDDDLPF | |
| | | | phiNIH11, Streptococcus pyogenes | | |
| | | | serotype M2 (strain | | |
| | | | MGAS10270), Streptococcus | | |
| | | | pyogenes serotype M3 (strain | | |
| | | | ATCC BAA-595/ | | |
| | | | MGAS315), Streptococcus | | |
| | | | pyogenes STAB902 | | |
| SSB_02 | SSB | Bacterial- | Streptococcus pyogenes | MINNVVLVGRMTKDAELRYTASQVAVATFTLAVNRRFKEQNG | 236 |
| | | SSB | STAB902, Streptococcus | EREADFINCVIWRQSAENLANWAKKGALIGVTGRIQTRNYENQ | |
| | | | pyogenes, Streptococcus pyogenes | QGQRVYVTEVVADNFQMLESRNQQSGQGNSSQNDNSQPFGNS | |
| | | | serotype M3 (strain ATCC BAA- | NPMDISDDDLPF | |
| | | | 595/MGAS315) | | |
| SSB_03 | SSB | Bacterial- | Bacillus subtilis | MLNRVVLVGRLTKDPELRYTPNGAAVATFTLAVNRTFTNQSG | 237 |
| | | SSB | | EREADFINCVTWRRQAENVANFLKKGSLAGVDGRLQTRNYEN | |
| | | | | QQGQRVFVTEVQAESVQFLEPKNGGGSGSGGYNEGNSGGGQY | |
| | | | | FGGGQNDNPFGGNQNNQRRNQGNSFNDDPFANDGKPIDISDD | |
| | | | | DLPF | |
| SSB_04 | SSB | Bacterial- | Saccharomyces cerevisiae (strain | MSSVQLSRGDFHSIFTNKQRYDNPTGGVYQVYNTRKSDGANS | 238 |
| | | SSB | ATCC 204508/S288c) (Baker\′s | NRKNLIMISDGIYHMKALLRNQAASKFQSMELQRGDIIRVIIAEP | |
| | | | yeast), Saccharomyces cerevisiae | AIVRERKKYVLLVDDFELVQSRADMVNQTSTFLDNYFSEHPNE | |
| | | | YJM1250, Saccharomyces | TLKDEDITDSGNVANQTNASNAGVPDMLHSNSNLNANERKFA | |
| | | | cerevisiae YJM451 | NENPNSQKTRPIFAIEQLSPYQNVWTIKARVSYKGEIKTWHNQR | |
| | | | | GDGKLFNVNFLDTSGEIRATAFNDFATKFNEILQEGKVYVVSK | |
| | | | | AKLQPAKPQFTNLTHPYELNLDRDTVIEECFDESNVPKTHFNFI | |
| | | | | KLDAIQNQEVNSNVDVLGIIQTINPHFELTSRAGKKFDRRDITIV | |
| | | | | DDSGFSISVGLWNQQALDFNLPEGSVAAIKGVRVTDEFGGKSLS | |
| | | | | MGFSSTLIPNPEIPEAYALKGWYDSKGRNANFITLKQEPGMGG | |
| | | | | QSAASLTKFIAQRITIARAQAENLGRSEKGDFFSVKAAISFLKVD | |
| | | | | NFAYPACSNENCNKKVLEQPDGTWRCEKCDTNNARPNWRYIL | |
| | | | | TISIIDETNQLWLTLFDDQAKQLLGVDANTLMSLKEEDPNEFTK | |
| | | | | ITQSIQMNEYDFRIRAREDTYNDQSRIRYTVANLHSLNYRAEAD | |
| | | | | YLADELSKALLA | |
| SSB_05 | SSB | Bacterial- | Saccharomyces cerevisiae | MATYQPYNEYSSVTGGGFENSESRPGSGESETNTRVNTLTPVTI | 239 |
| | | SSB | | KQILESKQDIQDGPFVSHNQELHHVCFVGVVRNITDHTANIFLTI | |
| | | | | EDGTGQIEVRKWSEDANDLAAGNDDSSGKGYGSQVAQQFEIG | |
| | | | | GYVKVFGALKEFGGKKNIQYAVIKPIDSFNEVLTHHLEVIKCHS | |
| | | | | IASGMMKQPLESASNNNGQSLFVKDDNDTSSGSSPLQRILEFCK | |
| | | | | KQCEGKDANSFAVPIPLISQSLNLDETTVRNCCTTLTDQGFIYPT | |
| | | | | FDDNNFFAL | |
| SSB_06 | SSB | Bacterial- | Saccharomyces cerevisiae | MASETPRVDPTEISNVNAPVERIIAQIKSQPTESQLILQSPTISS | 240 |
| | | SSB | | KNGSEVEMITENNIRVSMNKTFEIDSWYEFVCRNNDDGELGFLI | |
| | | | | LDAVLCKFKENEDLSLNGVVALQRLCKKYPEIY | |
| SSB_07 | SSB | Viral- | Staphylococcus phage phi11 | MKITGRTQYIQETNQEAFMKGGDFLGAGEFTVKVANVEFNDR | 241 |
| | | SSB | (Bacteriophage phi11), | ENRYFTIVFENNEGKQYKHNQFVPPFQQDYQEKQYIELLSREGI | |
| | | | Staphylococcus phage | KLNLPDLTFDTDQLINKIGTIVLKNKFNEEQGKVFVRLSYVKV | |
| | | | 80, Staphylococcus phage | WNKDDEVVNKPEPKTDEMKQKEQQANGKQTPMSQQSNPFAN | |
| | | | 52A, Staphylococcus aureus | ANGPIEINDDDLPF | |
| | | | (strain NCTC 8325) | | |
| SSB_08 | SSB | Viral- | Salmonella typhimurium, | MASRGVNKVILVGNLGQDPEVRYMPSGGAVANLTLATSESWR | 242 |
| | | SSB | Salmonella phage | DKQTGEMKEQTEWHRVVMFGKLAEVAGEYLRKGSQVYIEGQ | |
| | | | ST160, Salmonella phage ST64T | LRTRKWTDQSGQERYTTEINVPQIGGVMQMLGGRQGGGAPAG | |
| | | | (Bacteriophage ST64T) | GQQQGGWGQPQQPQQPQGGNQFSGGAQSRPQQSAPAPSNEPP | |
| | | | MDFDDDIPF | | |
| SSB_09 | SSB | Bacterial- | Enterococcus faecalis | MINNVVLVGRLTKDPDLRYTASGSAVATFTLAVNRNFTNQNG | 243 |
| | | SSB | TX0309B, Enterococcus faecalis | DREADFINCVIWRKPAETMANYARKGTLLGVVGRIQTRNYEN | |
| | | | TX0309A, Enterococcus faecalis | QQGQRVYVTEVVCENFQLLESRSASEQRGTGGGSFNNNENGY | |
| | | | (strain ATCC 700802/V583) | QSQNRSFGNNNASSGFNNNNNSFNPSSSQSQNNNGMPDFDKDS | |
| | | | | DPFGGSGSSIDISDDDLPF | |
| SSB_10 | SSB | Viral- | Acyrthosiphon pisum secondary | MASRGVNKVILIGHLGQDPEVRYMPNGNAVVNMTLATSENW | 244 |
| | | SSB | endosymbiont phage 1 | KDKNTGENKEKTEWHRIVLFGKLAEIAGEYLRKGSQVYIEGSL | |
| | | | (Bacteriophage APSE-1) | QTRKWQDQNGLERYTTEIIVNIGGTMQMLGNRNSNLQAMTVNDK | |
| | | | | NSTGIKIKKTDAVEFDKKIETHESNKDLTHSPSEIDEDDEIPF | |
| SSB_11 | SSB | Viral- | Bacillus phage SPP1 | MNSVNLVGRLAADPELRHTNNGTAVVNFIMAVRRNRKDPTTG | 245 |
| | | SSB | (Bacteriophage SPP1) | QYEADFIRCQAWRGIAEVIANNFGTGRMIGVSGSWRTGAFEGQ | |
| | | | | DGKRVYTNDCVVENITFVDPNKSDSSSPDNSQGSSNTNTFGGS | |
| | | | | QNGSGGQGGYNNDPFANDGKTIDINESDLPF | |
| SSB_12 | SSB | Viral- | Lactococcus phage LL-H | MAGINNVVLVGRLTKDVNLRSTQSGTMVGTFTLAVDRTTKDQ | 246 |
| | | SSB | (Lactococcus delbrueckii | NGNRQADFIKCVVWNNKYSKMAENLATYAHKGSLIGVQGRIQ | |
| | | | bacteriophage LL-H) | TRNYDNKDGQRVDVTEVRVDNFSLLESRNSAQREYTGQQGGY | |
| | | | | SQQQGNQPQGNYQASQAANFGQQGQFTAPAGQSDTIDVSNDD | |
| | | | | LPF | |
| SSB_13 | SSB | Viral- | Escherichia phage Rtp | MAQRGVNKVILIGTLGQDPEIRYIPNGGAVGRLSIATNESWRDK | 247 |
| | | SSB | | QTGQQKEQTEWHKVVLFGKLAEIASEYLRKGSQVYIEGKLKTR | |
| | | | | KWTDDAGVERYTTEIIVSQGGTMQMIGARRDDSQSSNGWGQS | |
| | | | | NQPQNHQQYSGGGKPQSNANNEPPMDFDDDIPF | |
| SSB_14 | SSB | Viral- | Streptococcus phage 7201 | MINNTVLVGRLTKDPEFKYTGSNIAVASFSLAVNRNFKDANGE | 248 |
| | | SSB | | READFINCVIWRQQAENLANWAKKGALIGITGRIQTRSYENQQ | |
| | | | | GQRVYVTEVVAENFQMLESRAAREGGNANNSYSQQQVPNFA | |
| | | | | RKNTEYSNKQPLDISSDDLPF | |
| SSB_15 | SSB | Viral- | Bacillus phage 0305phi8-36 | MSNELKQVEQTEEAVVVSETKDYIKVYENGKYRRKAKYQQLN | 249 |
| | | SSB | | SMSHRELTDEEEINIFNLLNGAEGSAVEMKRAVGSKVTIVDFIT | |
| | | | | VPYTKIDEDTGVEENGVLTYLINENGEAIATSSKAVYFTLNRLL | |
| | | | | IQCGKHADGTWKRPIVEIISVKQTNGDGMDLKLVGFDKKK | |
| SSB_16 | SSB | Viral- | Listeria phage A118 | MMNRVVLVGRLTKDPDLRYTPAGAAVATFTLAVNRMFTNQN | 250 |
| | | SSB | (Bacteriophage A118) | GEREADFINCVVWRKPAENVANFLKKGSMAGVDGRVQTRNY | |
| | | | | EDNDGKRVFVTEVVAESVQFLEPKNNNVEGATSNNYQNKANY | |
| | | | | SNNNQTSSYRADTSQKSDSFASEGKPIDINEDDLPF | |
| SSB_17 | SSB | Viral- | Lactococcus phage | MINNVTLVGRITKEPELRYTPQNKAVATFTLAVNRAFKNANGE | 251 |
| | | SSB | bIL286, Lactococcus lactis | READFINCVIWGKSAENLANWTHKGQLIGVTGSIQTRNYENQQ | |
| | | | subsp lactis (strain IL1403) | GQRVYVTEVIANNFQVLEKSNQANGERVSNPAAKPQNNDSFG | |
| | | | (Streptococcus lactis) | SDPMEISDDDLPF | |
| SSB_18 | SSB | Viral- | Lactococcus phage | MAIITVTAQANEKNTRTVSTAKGDKKIISVPLFEKEKGSSVKVA | 252 |
| | | SSB | SK1833, Lactococcus phage SK1 | YGSAFLPDFIQLGDTVTVSGRVQAKESGEYVNYNFVFPTVEKV | |
| | | | | FITNDNSSQSQAKQDLFGGSEPIEVNSEDLPF | |
| SSB_19 | SSB | Viral- | Mycobacterium phage Che8/ | MAGDTTITVVGNLTADPELRFTPSGAAVANFTVASTPRMFDRQ | 253 |
| | | SSB | Mycobacterium smegmatis | SGEWKDGEALFLRCNIWREAAENVAESLTRGSRVIVTGRLKQR | |
| | | | | SFETREGEKRTVVEVEVDEIGPSLRYATAKVNKASRSGGGGGG | |
| | | | | FGSGGGGSRQSEPKDDPWGSAPASGSFSGADDEPPF | |
| SSB_20 | SSB | Bacterial- | Ureaplasma urealyticum serovar | MNKVILIGNLVRDPEARQIPSGRLVTNFTIAVNDNTPNANANFI | 254 |
| | | SSB | 10 (strain ATCC 33699/Western), | RCVAWNNQANFLTTYLKKGDAIAIEGRIVSRSYVDNNGKTNY | |
| | | | Ureaplasma urealyticum | VTEVYADQVQSLSRRNQSPSDNNKVNVDTMMESYTGINTDAA | |
| | | | serovar 7 str ATCC | FSSNKPQTTLSSTTSNLNKNNDEEDEITSWINLDDDLE | |
| | | | 27819, Ureaplasma parvum serovar | | |
| | | | 3 (strain ATCC | | |
| | | | 700970), Ureaplasma urealyticum | | |
| | | | serovar 8 str ATCC | | |
| | | | 27618, Ureaplasma urealyticum | | |
| | | | serovar 4 str ATCC 27816 | | |
| SSB_21 | SSB | Viral- | Lactococcus lactis subsp lactis | MINNVVLVGRITRDPELRYTPQNQAVATFSLAVNRQFKNANGE | 255 |
| | | SSB | bv diacetylactis str | READFINCVIWRQQAENLANWAKKGALIGVTGRIQTRNYENQ | |
| | | | TIFN2, Lactococcus lactis subsp | QGQRVYVTEVVADSFQMLESRSAREGMGGGTSAGSYSAPSQS | |
| | | | lactis (strain IL1403) | TNNTPRPQTNNNNATPNFGRDADPFGSSPMEISDDDLPF | |
| | | | (Streptococcus lactis), | | |
| | | | Lactococcus phage bIL309 | | |
| SSB_22 | SSB | Bacterial- | Rhizobium loti (strain | MAGSVNKVILVGNLGADPEIRRLNSGEPVVNIRIATSESWRDK | 256 |
| | | SSB | MAFF303099) (Mesorhizobium | NSGERKEKTEWHNVVIFNEGIAKVAEQYLKKGMKVYVEGQLQ | |
| | | | loti) | TRKWQDQTGADKYTTEVVLQRFRGELQMLDGRQGEGGQVGG | |
| | | | | YSGGGSSRGSDFGQSGPNESFNRGGGAPRGGGGGGSSRELDDE | |
| | | | | IPF | |
| SSB_23 | SSB | Bacterial- | Homo sapiens (Human) | MVDMMDLPRSRINAGMLAQFIDKPVCFVGRLEKIHPTGKMFIL | 257 |
| | | SSB | | SDGEGKNGTIELMEPLDEEISGIVEVVGRVTAKATILCTSYVQF | |
| | | | | KEDSHPFDLGLYNEAVKIIHDFPQFYPLGIVQHD | |
| SSB_24 | SSB | Bacterial- | Homo sapiens (Human) | MVGQLSEGAIAAIMQKGDTNIKPILQVINIRPITTGNSPPRYRL | 258 |
| | | SSB | | LMSDGLNTLSSFMLATQLNPLVEEEQESSNCVCQIHRFIVNTLK | |
| | | | | DGRRVVILMELEVLKSAEAVGVKIGNPVPYNEGLGQPQVAPPAP | |
| | | | | AASPAASSRPQPQNGSSGMGSTVSKAYGASKTFGKAAGPSLSHT | |
| | | | | SGGTQSKVVPIASLTPYQSKWTICARVTNKSQIRTWSNSRGEGK | |
| | | | | LFSLELVDESGEIRATAFNEQVDKFFPLIEVNKVYYFSKGTLKI | |
| | | | | ANKQFTAVKNDYEMTFNNETSVMPCEDDHHLPTVQFDEFTGID | |
| | | | | DLENKSKDSLVDIIGICKSYEDATKITVRSNNREVAKRNIVLMD | |
| | | | | TSGKVVTATLWGEDADKFDGSRQPVLAIKGARVSDFGGRSLS | |
| | | | | VLSSSTIIANPDIPEAYKLRGWFDAEGQALDGVSISDEKSGGVG | |
| | | | | GSNTNWKTLYEVKSENLGQGDKPDYFSSVATVVVLRKENCM | |
| | | | | YQACPTQDCNKKVIDQQNGLYRCEKCDTEFPNFKYRMILSVNI | |
| | | | | ADFQENQWVTCFQESAEAILGQNAAYLGELKDKNEQAFEEVF | |
| | | | | QNANFRSFIFRVRVKVETYNDESRIKATVMDVKPVDYREYGRR | |
| | | | | LVMSIRRSALM | |
| SSB_25 | SSB | Bacterial- | Homo sapiens (Human) | MWNSGFESYGSSSYGGAGGYTQSPGGFGSPAPSQAEKKSRARAQ | 259 |
| | | SSB | | HIVPCTISQLLSATLVDEVFRIGNVEISQVTIVGIIRHAEKAPT | |
| | | | | NIVYKIDDMTAAPMDVRQWVDTDDTSSENTVVPPETYVKVAG | |
| | | | | HLRSFQNKKSLVAFKIMPLEDMNEFTTHILEVINAHMVLSKAN | |
| | | | | SQPSAGRAPISNPGMSEAGNFGGNSFMPANGLTVAQNQVLNLI | |
| | | | | KACPRPEGLNFQDLKNQLKHMSVSSIKQAVDFLSNEGHIYSTV | |
| | | | | DDDHFKSTDAE | |
| SSB_26 | SSB | Bacterial- | Homo sapiens (Human) | MFRRPVLQVLRQFVRHESETTTSLVLERSLNRVHLLGRVGQDP | 260 |
| | | SSB | | VLRQVEGKNPVTIFSLATNEMWRSGDSEVYQLGDVSQKTTWH | |
| | | | | RISVFRPGLRDVAYQYVKKGSRIYLEGKIDYGEYMDKNNVRR | |
| | | | | QATTIIADNIIFLSDQTKEKE | |
| SSB_27 | SSB | Viral- | Enterobacteria phage T1 | MAKKIFTSALGTAEPYAYIAKPDYGNEERGFGNPRGVYKVDLT | 261 |
| | | SSB | (Bacteriophage T1) | IPNKDPRCQRMVDEIVKCHEEAYAAAVEEYEANPPAVARGKK | |
| | | | | PLKPYEGDMPFFDNGDGTTTFKFKCYASFQDKKTKETKHINLV | |
| | | | | VVDSKGKKMEDVPIIGGGSKLKVKYSLVPYKWNTAVGASVKL | |
| | | | | QLESVMLVELATFGGGEDDWADEVEENGYVASGSAKASKPRD | |
| | | | | EESWDEDDEESEEADEDGDF | |
| SSB_28 | SSB | Bacterial- | Escherichia coli | MASRGVNKVILVGNLGQDPEVRYMPNGGAVANITLATSESWR | 262 |
| | | SSB | | DKATGEMKEQTEWHRVVLFGKLAEVASEYLRKGSQVYIEGQL | |
| | | | | RTRKWTDQSGQDRYTTEVVVNVGGTMQMLGGRQGGGAPAG | |
| | | | | GNIGGGQPQGGWGQPQQPQGGNQFSGGAQSRPQQSAPAAPSN | |
| | | | | EPPMDFDDDIPF | |
| SSB_29 | SSB | Viral- | Mycobacterium phage PhatBacter, | MSKDLQVHLVGTLTADPELRFTQGGQAVANFTVVSNERRRDA | 263 |
| | | SSB | Mycobacterium phage Elph10, | QGNWVDGDATFLRCTIWRDAAENVAESLQKGQRVIVHGYLK | |
| | | | Mycobacterium phage 244, | QRSFETKEGDKRTVIEVEVDEIGPSLRWATARVSKVGGNSNGG | |
| | | | Mycobacterium phage Cjw1, | GSSSFKEADANKWGDDDVPF | |
| | | | Mycobacterium phage Phrux, | | |
| | | | Mycobacterium phage Lilac, | | |
| | | | Mycobacterium phage Phaux, | | |
| | | | Mycobacterium phage Quink, | | |
| | | | Mycobacterium phage Pumpkin, | | |
| | | | Mycobacterium phage Murphy | | |
| SSB_30 | SSB | Viral- | Bacillus thuringiensis | MMNRVILVGRLTKDPDLRYTPNGVAVATFTLAVNRAFANQQG | 264 |
| | | SSB | Sbt003, Escherichia coli\′BL21- | EREADFINCVIWRKQAENVANYLKKGSLAGVDGRLQTRNYEG | |
| | | | Gold(DE3)pLysS | QDGKRVYVTEVLAESVQFLEPRNGGGEQRGSFNQQPSGAGFG | |
| | | | AG\′, Enterobacteria phage | NQGSNPFGQSSNSGNQGNSGFTKNDDPFSNVGQPIDISDDDLPF | |
| | | | HK630, Enterobacteria phage | | |
| | | | lambda (Bacteriophage | | |
| | | | lambda), Escherichia coli | | |
| | | | TA280, Escherichia coli 1-176- | | |
| | | | 05_S3_C2, Escherichia coli 40967 | | |
| SSB_31 | SSB | Viral- | Bordetella phage BPP-1 | MASVNKVILVGNLGRDPEVRYSPDGAAICNVSIATTSQWKDKA | 265 |
| | | SSB | | SGERREETEWHRVVMYNRLAEIAGEYLKKGRSVYIEGRLKTR | |
| | | | | KWQDKDTGADRYSTEIVADQMQMLGGRDSGGDSGGGYGGG | |
| | | | | YDDAPRQQRAPAQRPAAAPQRPAPQAAPAANLADMDDDIPF | |
| SSB_32 | SSB | Viral- | Burkholderia phage BcepNazgul | MASVNKVILVGNLGADPEVRYLPSGDAVANIRLATTDRYKDK | 266 |
| | | SSB | | ASGDIKEATEWHRVSFFGRLAEIVDEHLRKGASVYIEGRIKTRK | |
| | | | | WQDQSGQDRYTTEIVADRMQMLGKPGGSRDDSGDQQQRQHG | |
| | | | | GQQQRGGGRNGYADATGRAQPQRTAEQRGASGFDDMDDSIPF | |
| SSB_33 | SSB | Bacterial- | Photorhabdus luminescens subsp | MASRGINKVILIGNLGQDPEVRYMPNGGAVTNITLATSESWRD | 267 |
| | | SSB | laumondii (strain DSM 15139/ | KQTGEMKEKTEWHRVVLFGKLAEVAGEYLRKGSQVYIEGSLQ | |
| | | | CIP105565/TT01) | TRKWQDQNGQERYTTEVVVNMGGTMQMLGGRAGGGSFQDS | |
| | | | | QQSQGGGWGQPQQPQPQQQSQQFSGGGAPSRPAQSSAPQSNE | |
| | | | | PPMDFDDDIPF | |
| SSB_34 | SSB | Bacterial- | Photobacterium profundum (strain | MELSMASRGVNKVILVGNLGNDPEIRYMPSGSAVANITIATSES | 268 |
| | | SSB | SS9) | WRDKATGEQREKTEWHRVALFGKLAEVAGEYLRKGSQVYIE | |
| | | | | GQLQTRKWQNQQGQDQYTTEVVVQGFNGVMQMLGGRQGGG | |
| | | | | QQQQQQQQQGNWGKPQQPAAAPQQSVAPQQQQQAPQQPQQ | |
| | | | | APQQPQQQYNEPPMDFDDDIPF | |
| SSB_35 | SSB | Viral- | Lactobacillus phage Lc-Nu | MAITMDYSQAAEGNGDIQDGVYECVINRFGFDNYKDREFIKED | 269 |
| | | SSB | | LIVRNDVPQKYQNKHIFDNFYPKKDTGEYAMGYLFMIGKNAGI | |
| | | | | PDHKAWSDLAAMLADFTGHAVKVTVKNEEYNGKTYPHIKKW | |
| | | | | EPTAFPQIQHRWKDSKDESSSNSNPSFGTPAQTSQTNTSDPFAN | |
| | | | | SGQPIDVSDDDLPF | |
| SSB_36 | SSB | Bacterial- | Leuconostoc mesenteroides subsp | MINRVVLIGRLTRDVELRYTQSGVAVGTFSLAVNRQFTNASGE | 270 |
| | | SSB | mesenteroides (strain ATCC 8293/ | READFINAVIWRKAAENFANFTGKGALVAVEGRLQTRNYENN | |
| | | | NCDO 523) | AGQRVYVTEVVVDNFSLLESRAESEKRRSQNGSSASNNGADNF | |
| | | | | SGSNDHSFGGNDNSFNGVDPFASASSNSNTQSSASSNSAPNPFA | |
| | | | | ASGNTEIDISDDDLPF | |
| SSB_37 | SSB | Viral- | Staphylococcus phage | MLNRTILVGRLTRDPELRTTQSGVNVASFTLAVNRTFTNAQGE | 271 |
| | | SSB | 3A, Staphylococcus phage | READFINIIVFKKQAENVNKYLSKGSLAGVDGRLQTRNYENKE | |
| | | | phi7401PVL, Streptococcus | GQRVYVTEVVADSIQFLEPKNSNDTQQDLYQQQVQQTRGQSQ | |
| | | | pneumoniae, Staphylococcus | YSNNKPVKDNPFANANGPIEENDDDLPF | |
| | | | aureus (strain NCTC | | |
| | | | 8325), Staphylococcus phage | | |
| | | | Phi12, Staphylococcus | | |
| | | | aureus, Staphylococcus phage | | |
| | | | 47, Staphylococcus phage tp310-2 | | |
| SSB_38 | SSB | Viral- | Escherichia phage Tls | MAVRGINKVILVGRLGKDPEVRYIPNGGAVANLQVATSETWR | 272 |
| | | SSB | | DKQTGEMKEQTEWHRVVLFGKLAEVAGEYLRKGAQVYIEGQ | |
| | | | | LRTRSWEDNGITRYVTEILVKTTGTMQMLGSAPQQNAQVQPQ | |
| | | | | PQQNGQSQSADATKKGSAKTKGRGRKAAQPEPQPQPPEGEDY | |
| | | | | GFSDDIPF | |
| SSB_39 | SSB | Bacterial- | Leifsonia xyli subsp xyli, | MAGETVITVVGNLTSDPELRYTQNGLAVANFTIASTPRTFDRQ | 273 |
| | | SSB | Leifsonia xyli subsp xyli | ANEWKDGEALFLRASVWRDFAEHVAGSLTKGSRVIAQGRLKQ | |
| | | | (strain CTCB07) | RSYETKEGEKRTSIELEIDEIGPSLRYATAQVTRAQSSRGPGG | |
| | | | | PGGFGGGAPAVEEPWAATVPADPSAGTDVWNTPGAYNDETPF | |
| SSB_40 | SSB | Bacterial- | Legionella pneumophila | MISMARGINKVILVGNVGADPDVRYLPNGNAVTTLSVATSET | 274 |
| | | SSB | | WKDKTTGEKQDRTEWHRVVCFNRLGEIAGEYIRKGSKLYVEG | |
| | | | | SLRTRKWQDQQGQDRYTTEIVASDIQMLDSKGSSATNYDDMP | |
| | | | | SFQGTSTPQQASTKNQATPTSTAQDAFDELDDDIPF | |
| SSB_41 | SSB | Bacterial- | Nocardia farcinica (strain IFM | MAGDTVITVIGNLTADPELRFTPAGQAVANFTVASTPRVFDRN | 275 |
| | | SSB | 10152) | TNEWKDGEALFLRCNIWREAAENVAESLTRGARVIVSGRLKQ | |
| | | | | RSYETREGEKRTVVELEVDEVGPSLRYATAKVNKASRGGGGG | |
| | | | | GGFGGGGGGGYASDRSGGGGSRSGAAEDDPWGSAPAAGSFG | |
| | | | | GGRMDDEPPF | |
| SSB_42 | SSB | Viral- | Streptococcus phage Sfi21 | MINNVVLVGRMTRDAELRYTPSNVAVATFSLAVNRNFKGANG | 276 |
| | | SSB | | ERETDFINCVIWRQQAENLANWAKKGALVGITGRIQTRNYENQ | |
| | | | | QGQRVYVTEVVADNFQMLESRAAREGHSGGSYNVGGFDNSN | |
| | | | | SFGGGASTGGSFGESQPAQSTPNFGRDESPFGNSNPMDISDDDL | |
| | | | | PF | |
| SSB_43 | SSB | Bacterial- | Campylobacter coli 80352 | MFNKVVEVGNLTRDIEMRYGQNGNAIGASAIAVTRRFTTNGER | 277 |
| | | SSB | | REETCFIDISFYGRTAEVANQYLSKGSKVLIEGRERFEQWNDQN | |
| | | | | GQMRSKHSVQVENMEMLGNNPQQGGNFNNGGNNYGANNNY | |
| | | | | SNYENQSYDPYMSENNFKKAPQQAQTKTQNPNQNQEKIKEID | |
| | | | | VDAYDSDDTDLPF | |
| SSB_44 | SSB | Viral- | Staphylococcus phage 92 | MLNRVVLVGRLTKDPEYRTAPNGVSVTTFTIAVNRTFTNAQGE | 278 |
| | | SSB | | READFINCVTFRKQAENVKNYLSKGSLAGVDGRLQTRNYENK | |
| | | | | DGQRVYVTEVVADSVQFLEPKNSNQQNNQQHNEQTQTGNNPF | |
| | | | | DNTTAITDDDLPF | |
| SSB_45 | SSB | Bacterial- | Pelobacter propionicus (strain | MASLNKVMLIGNLGRDPEVRYTASGQAVASFNLATTEKFKNR | 279 |
| | | SSB | DSM 2379/NBRC 103807/ | NGEWEERTEWHRVTLWARLAEIAGEYESKGKTVYIEGREQTR | |
| | | | OttBd1) | EYEKDGIKRYTTEIVGEKMQMLSPKGERRSSGDSYSPAPAGTS | |
| | | | | GGGYEPPPFQDDDIPF | |
| SSB_46 | SSB | Bacterial- | Clostridium beijerinckii (strain | MNKVVLIGRLTKDPELRFTPGSGAAVTTLTLAVDKYNTKTGQR | 280 |
| | | SSB | ATCC 51743/NCIMB 8052) | EADFVPVVVWGKQAESTANYMSKGSQVAISGRIQTRSYDAKD | |
| | | | (Clostridium acetobutylicum) | GTKRYVTEVVADQFGGVEFLGSKGSNSSGNSFGNSNEYSAPAN | |
| | | | | DAFSGGFEEDITPVDDGDMPF | |
| SSB_47 | SSB | Bacterial- | Sodalis glossinidius (strain | MASRGVNKVILVGNLGQDPEVRHMPNGGAVANITLATSESWR | 281 |
| | | SSB | morsitans) | DKQTGETKEKTEWHRVVLFGKLAEVAGEYLRKGSQVYIEGSL | |
| | | | | QTRKWQDQSGQDRYTTEVVVNVGGTMQMLGGRQGGAAASG | |
| | | | | GQQSGWGLPQQPQGNNAQFSGGNAPASRRAQNSAPAPSNEPP | |
| | | | | MDFDDDIPF | |
| SSB_48 | SSB | Bacterial- | Xanthobacter autotrophicus | MAGSVNKVILVGNLGRDPEIKSEQNGGRVCNLSVATSENWRD | 282 |
| | | SSB | (strain ATCC BAA-1158/Py2) | KASGERRERTEWHRVVIFNENLAGVAERFLKKGSKVYIEGQLE | |
| | | | | TRKYEKDGRETYTTEIVLRPYRGELTLLDGRGEGAGAGGDDY | |
| | | | | AAGSDFGSASPMGGGYGGGSGGSRRMSGAPAGSSGGVAGGG | |
| | | | | RPAADLDDEIPF | |
| SSB_49 | SSB | Viral- | Clostridium phage phiC2, | MNTITLVGRLVADAELKYLPNSGTPKITFSMAVDRRFKDKNGN | 283 |
| | | SSB | Peptoclostridium difficile | KITDFIQCEQLGKHVENLVQYLVKGKPIYAVGELNIYNYKDEN | |
| | | | E15, Clostridium phage | GCWKSITKVNVNALELLSSKNDNNAKQEYVPPGLDPQGFQAID | |
| | | | phiMMP03, Peptoclostridium | DDDIPF | |
| | | | difficile (Clostridium | | |
| | | | difficile) | | |
| SSB_50 | SSB | Viral- | Enterobacteria phage HK022 | MAQRGVNKVILIGTLGQDPEIRYIPNGGTVGRLSIATNESWRDK | 284 |
| | | SSB | (Bacteriophage HK022) | QTGQQKEQTEWHRVVLEGKLAEIASEYLRKGSQVYIEGKLKTR | |
| | | | | KWTDDAGVERYTTEIIVSQGGTMQMIGARRDDSQFSNGWGQS | |
| | | | | NQPQNHQQYSGGSKPQSNANSEPPMDFEDDIPF | |
| SSB_51 | SSB | Viral- | Mycobacterium phage Wildcat | MSKDLQVHLVGTLTADPELRFTQGGQAVANFTVVSNERRRDA | 285 |
| | | SSB | | QGNWVDGDATFLRCTIWRDAAENVAESLQKGQRVIVHGYLK | |
| | | | | QRSFETKEGDKRTVIEVEVDEIGPSLRWATARVSKVGGNSNGG | |
| | | | | GSSSFKEADANKWGDDEPPF | |
| SSB_52 | SSB | Viral- | Streptococcus phage MM1 | MINNVVLVGRLTRDAELRYTQSNIAVATFTLAVNRPFKNEAGE | 286 |
| | | SSB | 1998, Streptococcus pneumoniae, | READFINCVIWRQLAENLANWAKKGSLIGVTGVIQTRSYDNQQ | |
| | | | Streptococcus phage MM1 | GQRVYVTEVVASNFQLLESRNSQQNNQGHQDHHGGYQQQGY | |
| | | | | SNQGSSFQNGNSYGQQGSFVEGNTTNLVPDFTRDNNPFGRPTN | |
| | | | | PLDISDDDLPF | |
| SSB_53 | SSB | Viral- | Lactococcus phage c2 | MSINNVTLVGRLVRDPELKNTAQGIANVSFTLAVNRNYKNDQ | 287 |
| | | SSB | | GQREADFINVVIWRKQAELLAQYATKGALIGITGRIQTRNYEN | |
| | | | | QQGQRIYVTEVVADNFQLLESRGAQQGGQQQQQNQGYGQQQ | |
| | | | | NQRPQGNYQSNPRNNQQRQNQPDPFRGSPMEISDDDLPF | |
| SSB_54 | SSB | Bacterial- | Salinispora tropica (strain | MAGDTIITVIGNLTDDPELRFTPSGAAVAKFRVASTPRFFDKSS | 288 |
| | | SSB | ATCC BAA-916/DSM 44818/CNB- | SEWKDGEPLFLSCTVWRQAAEHVAESLQRGTRVIVSGRLRQRS | |
| | | | 440) | YETREGEKRTVIELEVDEIGPSERYATAKVQKMSRSGGGGFGG | |
| | | | | GGGQGGGGNFDDPWASAAPAPAPSRGGSGGGNFDEEPPF | |
| SSB_55 | SSB | Bacterial- | Stigmatella aurantiaca (strain | MAGGVNKVILIGNLGADPEVRFTPGGQAVANFRIATSDSWTDK | 289 |
| | | SSB | DW4/3-1) | NGQKQERTEWHRIVVWGKLAELCGEYLKKGRQCFVEGRLQT | |
| | | | | REWTDKENRKNYTTEVVASSVTFLGGRDAGEGSGMGSRRGG | |
| | | | | GASSRGGEPDYGAPPPGMDDGMNQGGSGDDDIPF | |
| SSB_56 | SSB | Viral- | Listeria welshimeri serovar 6b | MMNRVVLVGRLTKDPELRYTPAGVAVATFTLAVNRTFTNQQG | 290 |
| | | SSB | (strain ATCC 35897/DSM 20650/ | EREADFINCVVWRKPAENVANFLKKGSMAGVDGRVQTRNYE | |
| | | | SLCC5334), Listeria phage PSA | GNDGKRVYVTEIVAESVQFLEPRNSNGGGGNNYQGGNNNNY | |
| | | | | NNGGSNSGQAPTNNGGFGQDQQQSQNQNYQSTNNDPFASDG | |
| | | | | KPIDISDDDLPF | |
| SSB_57 | SSB | Bacterial- | Gramella forsetii (strain | MTGTLNKVMLIGHTGDDVKMHYFEGGGSIGRFPLATNESYTN | 291 |
| | | SSB | KT0803) | RTTGERVTNTEWHNVVVRNKAAEVCEKYLKKGDKVYIEGRIK | |
| | | | | TRKWQDDAGNEKYSTEVHCTEFTELTPKNESNAEPSGKSQASG | |
| | | | | NTSAKPKSDNFANKNEFYSQDEEEDDLPF | |
| SSB_58 | SSB | Viral- | Staphylococcus phage CNPH82 | MTNLTILTGRITKDLELKQAGQTQVTNFSMAVDNPFKKDDTSF | 292 |
| | | SSB | | EDIVAFGKTAQLLNDYCGKGSKVLIEGNEKQDRFQDKEGNNRS | |
| | | | | VVRVIANRIEFLDSKGSNQSNNQSPKRGQAPAGNNPFANGTDIE | |
| | | | | NSELPF | |
| SSB_59 | SSB | Viral- | Staphylococcus phage Pvl108 | MKITGQAQFTKETNQEKFYNGSAGFQAGEFTVKVKNIEFNDRE | 293 |
| | | SSB | | NRYFTIVFENDEGKQYKHNQFVPPYKYDFQEKQLIELVTRLGIK | |
| | | | | LNLPSLDFDTNDLIGKFCHLVLKWKFNKDEGKYFTDFSFIKPYK | |
| | | | | KGDDVVNKPIPKTDKQKAEENNGAQQQTSMSQQSNPFESSGQ | |
| | | | | EGYDDQDLAF | |
| SSB_60 | SSB | Viral- | Prochlorococcus phage P-SSP7 | MNHCVLEVEVIQAPTIRYTQDNQTPIAEMEVRFDALRVDTAPG | 294 |
| | | SSB | | QLKVVGWGNLAQDLQNRVQVGQRLVIEGRLRMNTVPRQDGT | |
| | | | | KEKRAEFTLAREHSLSGQAGQPASSPERPTSTTTNQTSRPIPIA | |
| | | | | ETPTPKPATTAEPEAASWNSAPLVPDTDDIPF | |
| SSB_61 | SSB | Bacterial- | Vibrio cholerae 1587 | MASRGVNKVILIGNLGQDPEVRYMPSGGAVANITIATSETWRD | 295 |
| | | SSB | | KATGEQKEKTEWHRVTLYGKLAEVAGEYLRKGSQVYIEGQLQ | |
| | | | | TRKWQDQSGQDRYSTEVVVQGYNGIMQMLGGRTQQGGMPA | |
| | | | | QGGMNAPAQQGSWGQPQQPAKQHQPMQQSAPQQYSQPQYNE | |
| | | | | PPMDFDDDIPF | |
| SSB_62 | SSB | Bacterial- | Bacteroides caccae ATCC 43185 | MSVNRVILIGNVGQDPRVKYFDTGSAVATFPLATTDRGYTLQN | 296 |
| | | SSB | | GTQIPERTEWHNIVASNRLAEIVDKYVHKGDKLYLEGKIRTRS | |
| | | | | YSDQSGAMRYITEIYVDNMEMLTPKGAGQGAGTSAQQGAATP | |
| | | | | SQQPQQMQQNQQPQQSQAQPVQDNPADDLPF | |
| SSB_63 | SSB | Bacterial- | Elusimicrobium minutum (strain | MASQIRLPEQNLVLLTGRLTRDANTAFTQKGAAVSRFDIAVNR | 297 |
| | | SSB | Pei191) | RYMDANGSWQDETTFVPVTLWGPAAERSKDRLKKGVPVHVE | |
| | | | | GRLVLNEYTDKNGVAHKNEQVNCRRIQILQSAFSESASGSGAS | |
| | | | | FNDTPVDNEAIDDDVPF | |
| SSB_64 | SSB | Bacterial- | Methylobacterium nodulans | MAGSVNKVILVGNLGRDPETRRETSGDPVVNLRLATSESWKD | 298 |
| | | SSB | (strain LMG 21967/CNCM I- | KATGERKEKTEWHSVVIYNENLARVAEQYLRKGSKVYVEGQL | |
| | | | 2342/ORS 2060) | QTRKWQDQSGVEKYTTEVVLQRFRGELTILDGRGGGETGAMD | |
| | | | | EMGGGQISRGGDFGGDRSSGGERRPAPAGGSQKRYDDLDDDIP | |
| | | | | F | |
| SSB_65 | SSB | Viral- | Yersinia phage YpsP-G, Yersinia | MAVNKIILLGNVGNYPEVNYTGSGTAVAKFSVATTEKWKDQS | 299 |
| | | SSB | phage phiA1122 | GEKKEKTNWHRCVVFGKRAEVVGEYVRKGSQLYIEGSMEYGS | |
| | | | | YDKDGVTMYTADVHVKDFQFIGGKREDGGNAGGGQSGGWG | |
| | | | | QPQQTQQQRQPASQPTNTATLKPEGQPKQRQMSQAERMLAQQ | |
| | | | | AAQAQQQSSEPPMDFDDDIPF | |
| SSB_66 | SSB | Bacterial- | Clostridium botulinum C str | MNRVVLVGRLTKDPELRFTPGAGKAVATFTLAVNRRFKSQGQ | 300 |
| | | SSB | Eklund | PDADFLPIVVWGKQAENTANYVGKGSQVGVSGAIHTRSYEAK | |
| | | | | DGTRRYVTEIVADEVQFLDSRNAVSRTEPRSTGMNEFDSSNND | |
| | | | | NDFNQSYDEEITPIDDGDIPF | |
| SSB_67 | SSB | Bacterial- | Ureaplasma urealyticum serovar | MNKVILIGNLVRDPEARQIPSGRLVTNFTVAVNDNIPNANANFI | 301 |
| | | SSB | 12 str ATCC 33696 | RCVAWNNQANFLTTYLKKGDAIAIEGRIVSRSYVDNNGKTNY | |
| | | | | VTEVYADQVQSLSRRNQNANDHNNDKVNVDTMMGAYASINT | |
| | | | | DAAFSSNQPQTNFQSTTSNSNKNDDEEDEITSWINLDDDLE | |
| SSB_68 | SSB | Bacterial- | Mycobacterium marinum (strain | MAGDTTITVVGNLTADPELRFTPSGAAVANFTVASTPRIYDRQS | 302 |
| | | SSB | ATCC BAA-535/M) | GEWKDGEALFLRCNIWREAAENVAESLTRGARVIVSGRLKQRS | |
| | | | | FETREGEKRTVVEVEVEEIGPSERYATAKVNKASRGGGGGGFG | |
| | | | | GGGGGGGGGGARQAPAQASSPAGGDDPWGSAPASGSFGGDD | |
| | | | | EPPF | |
| SSB_69 | SSB | Bacterial- | Oligotropha carboxidovorans | MAGSVNKVILVGNLGADPDVRRTQDGRPIVNESIATSDTWRDK | 303 |
| | | SSB | (strain ATCC 49405/DSM 1227/ | ATGERKEKTEWHRVVIFNEGLCRVAEQYLKKGAKVYIEGALQ | |
| | | | KCTC 32145/OM5) | TRKWQDKDGKDKYSTEVVLQGFNSTLTMLDGRSGGGGGSFA | |
| | | | | GDDAGSSFGSSGSSQRRSLPPSGGRDDMNDDIPF | |
| SSB_70 | SSB | Bacterial- | Clostridium botulinum (strain | MNKVVLIGRLTKDPELRFTPGAGTAVTTLTLAVDKYNSKSGQK | 304 |
| | | SSB | Eklund 17B/Type B) | EADFVPVVVWGKQAESTANYMSKGSQMAISGRIQTRNYEAKD | |
| | | | | GTKRYVTEVVATEVQFLSKSNASGNVGTNYGNNTEYSSSNNPF | |
| | | | | DGMNFEEDITPVDDGDMPF | |
| SSB_71 | SSB | Bacterial- | Collinsella stercoris DSM 13279 | MSINRVNISGNLTRDPELRATSSGTQVLSFGVAVNDRRRNPQT | 305 |
| | | SSB | | GDWEDYPNFVDCTMFGTRAEAVKRYLSKGSKVAIEGKLRYSS | |
| | | | | WERDGQRRSKLEVIVDEIEFMSRGQQGEGGGYAPAPSYGQQG | |
| | | | | GYAPAPAPQQAPAPMAAPVPPAVDVYDEDIPF | |
| SSB_72 | SSB | Bacterial- | Haemophilus parasuis serovar 5 | MAGVNKVIIVGNLGNDPDMRTMPNGDAVATLSVATSESWND | 306 |
| | | SSB | (strain SH0165) | KMTGERREVTEWHRIVFFRRQAEVAGQYLRKGSKVYVEGKLK | |
| | | | | TRKWQDQNGQDRYTTEIQGDVLQMLDSRSGDQGGGWNQTQT | |
| | | | | NYNQDGYTDSYAQNNNFNGGNATRPQPAQKPAAQAEPPMDN | |
| | | | | FDDDIPF | |
| SSB_73 | SSB | Bacterial- | Agrobacterium rhizogenes | MAGSVNKVILVGNEGADPEIRRTQDGRPIANLNIATSETWRDR | 307 |
| | | SSB | | NSGERKEKTEWHRVVIFNEGLCKVAEQYLKKGAKVYIEGALQ | |
| | | | | TRKWQDQNGQDKYSTEIVLQGFNSTLTMLDGRGEGGGGGNR | |
| | | | | GGGGGDFGGGDYGGDDYGQPAPSSGGGRSAGASRGAPASSGG | |
| | | | | GSNFSRDLDDDIPF | |
| SSB_74 | SSB | Viral- | Salmonella phage 553e | MASRGVNKVIILGRVGQDPEVRYSPSGTAFANLTVATSEQWRD | 308 |
| | | SSB | | KQTGEQKEQTEWHRVAVVGKLAEVVGQYVKKGDQVYFEGM | |
| | | | | LRTRKWQDQTGQDRYTTEINVGINGVMQMLGGTGDSKQQAA | |
| | | | | DRQSQKPQQQPSPTQHNEPPMDFDDDIPFAPVTLPFPRHAIHAI | |
| SSB_75 | SSB | Bacterial- | [Clostridium] methylpentosum | MLNKVILMGRLTADPEVRQTPNGISVLSFSIAVDRNYSKAEKK | 309 |
| | | SSB | DSM 5476 | TDFINLVAWRQTAEFIGRFFTKGQMIAVEGSIQTRNYEDKTGA | |
| | | | | KRTAFEVVVDQAHFTGGKSENPVRTTQPSYQQPPKFEEPAQNG | |
| | | | | ESFSVGDFNDFDGFEEIGTDDGDLPF | |
| SSB_76 | SSB | Bacterial- | Helicobacter pullorum MIT 98- | MFNKVILVGNLTRDVELRYLPSGAALARLNLATNRRYKKQDG | 310 |
| | | SSB | 5489 | TQAEEVCFIDVNLFGRTAEVANQYLKKGSQVLIEGRLVLESWT | |
| | | | | DNTGAKRTKHSITAESMQMLGQRQSTQEENHDYGAGDYNNY | |
| | | | | QEYEKPAYTASAAPKAQPVQKEPELPVIDINDDEIPF | |
| SSB_77 | SSB | Bacterial- | Persephonella marina (strain | MLNKVFLIGRLTRDPEIRELPSGSQVTSFSVAVNRSYRVNNEWK | 311 |
| | | SSB | DSM 14350/EX-H1) | EETYFFDVEAFGYLAERLGKQLNKGTQILIEGQLRQDRWETAG | |
| | | | | GDKRTKVKIVADKVSILSPKGEKAEKSEEEPELDIENSIEDFSS | |
| | | | | DEDVPF | |
| SSB_78 | SSB | Bacterial- | Brevibacillus brevis (strain 47/ | MNKVILIGNLTKDPELRYTPNGVAVATFTVAVNRPRTNQAGER | 312 |
| | | SSB | JCM 6285/ NBRC 100599) | ETDFINIVAWQKLADLCASYLRKGRQAAIEGRMQTRSYDNKE | |
| | | | | GKKVYVTEVVAENVQFLGGRGNESGGDNPGYDPGPGMGGGN | |
| | | | | KPSGQRNNDYDPFGDPFASAGKPINISDDDLPF | |
| SSB_79 | SSB | Bacterial- | Corynebacterium striatum ATCC | MAIDIITITGGLPRDAELRFTKSGKAVTSFTLANSDNKFDQEQN | 313 |
| | | SSB | 6940 | QWVKTRSMYLDVTIWDESTERKQNPVQWARLASELKQGDQV | |
| | | | | AVKGKLTTRTWETDGGEKRSKMEFLATSFYRMPTTQGAPNGQ | |
| | | | | QAQQNITQGLGAQAAGDPWNGAPQGGFSGADANPPF | |
| SSB_80 | SSB | Bacterial- | Cryptobacterium curtum (strain | MSSINRVFITGNLSRDPELRTTASGSAVLSFGVAVVDSVKNQQT | 314 |
| | | SSB | ATCC 700683/ DSM 15641/12- | GEWEDRPNWVECTLFGSRAQAISGYLHKGSKVAIDGRLHWSQ | |
| | | | 3) | WERNGEKRSKLEVIINDIQFLSPREGAQGAPQQPQYQQPAPTYQ | |
| | | | | QPAPTYQQTAQQPPQQPQTAPQYASASVYDEDIPF | |
| SSB_81 | SSB | Viral- | Listeria phage A500 | MRGGWFCMMNRVVLVGRLTKDPELRYTPSGVAVATFTLAVN | 315 |
| | | SSB | (Bacteriophage A500) | RTFTNQQGEREADFINCVVWRKPAENVANYLKKGSLAGVDGR | |
| | | | | VQTRNYEGQDGKRVYVTEIVAESVQFLEPRNNSGQHQDNNNN | |
| | | | | SYNQAPANNGFNQNQQQSQSYQTTNNDPFANDGKPIDISDDDL | |
| | | | | PF | |
| SSB_82 | SSB | Viral- | Lactobacillus prophage | MINNVVLVGRLTRDPDLRTTGSGISVATFTLAVDRQYTNSQGE | 316 |
| | | SSB | Lj928, Lactobacillus johnsonii | RGADFINCVIWRKAAENFANETSKGSEVGIQGRIQTRTYDDKD | |
| | | | (strain CNCM 1-12250/La1/ | GKRVYVTEVIVDNFSLLESRRDRENRQTNGGNFAPQGGNAPST | |
| | | | NCC 533) | NNFGGSSAPSMNNAPASGESNKPQDPFADSGSTIDISDDDLPF | |
| SSB_83 | SSB | Bacterial- | Vibrio cholerae (strain | MITEQNMASRGVNKVILIGNLGQDPEVRYMPSGGAVANITIAT | 317 |
| | | SSB | MO10), Vibrio | SETWRDKATGEQKEKTEWHRVTLYGKLAEVAGEYLRKGSQV | |
| | | | cholerae, Providencia | YIEGQLQTRKWQDQSGQDRYSTEVVVQGYNGIMQMLGGRAQ | |
| | | | alcalifaciens | QGGMPAQGGMNVPAQQGSWGQPQQPAKQHQPMQQSAPQQY | |
| | | | Ban1, Vibrio cholerae Ind4 | SQPQYNEPPMDFDDDIPF | |
| SSB_84 | SSB | Bacterial- | Fusobacterium mortiferum ATCC | MNVVVLVGRLTRDPELKFGQSGKAYSRFSLAVDRPFSKGEADF | 318 |
| | | SSB | 9817 | INCVAFGKTAELIGEYLRKGRKVGVNGRLQMNRFEMNGEKRT | |
| | | | | SYDVLVEAIEFLESKGSGDSMGGYEPEYSSPTPKSSAKEVEEI | |
| | | | | PYEDDDEFPF | |
| SSB_85 | SSB | Viral- | Burkholderia phage BcepNY3 | MASVNKVILVGNLGADPETRYLPSGDAISNIRLATTDRYKDKA | 319 |
| | | SSB | | SGEFEKEVTEWHRVAFFGRLAEIVDEHLRKGASVYIEGRIKTRK | |
| | | | | WQDQSGQDRYTTEIVADRMQMLGKPSGSRDDGGGERQQRAP | |
| | | | | QQQQQQRGQRNGYADATGRGQPQRDAQQRPPAGGGFDEMD | |
| | | | | DDIPF | |
| SSB_86 | SSB | Bacterial- | Agathobacter rectalis (strain | MNKVILMGRLTRDAEIRYAQGDNSLAIARFSLAVDRRYSKNAE | 320 |
| | | SSB | ATCC 33656/DSM 3377/JCM | EQSTDFINCVAFGKIAEFFERFGRKGTKFVVEGRIQTGSYTNKD | |
| | | | 17463/KCTC5835/VPI 0990) | GQKVYTTDVVVENAEFAESKSAASGNAGGFAPADRPAPSQAA | |
| | | | (Eubacterium rectale) | GDGFMNIPDGIDEELPFN | |
| SSB_87 | SSB | Viral- | Cyanophage PSS2 | MATINILGREGKDPEVKFFDSGNCVAKFTIGDVAGRKDDPTNW | 321 |
| | | SSB | | FDCEIWGKRAQLLGDTVSKGQRLMVSGDIKTETWTAKDGGNR | |
| | | | | SKQVVRVSDFQYIESRGEAAGGGQATSEEIPF | |
| SSB_88 | SSB | Bacterial- | Acinetobacter radioresistens | MRGVNKVILVGTLGRDPETKTFPNGGSLTQFSIATSESWTDKST | 322 |
| | | SSB | SK82 | GERKEQTEWHRIVLHNREGEIAQQYLRKGSKVYIEGSLRTRQW | |
| | | | | TDQNGQERYTTEIRGEQMQMLDSGRQQGDQAGAGFGGDQGY | |
| | | | | GQPRFNNNQGSQGGYGNGNQQGGGFNNNNNQGGGYGNNNP | |
| | | | | GGFAPKAPQSAPASQVPADLDDDLPF | |
| SSB_89 | SSB | Bacterial- | Enterococcus faecalis TX0027 | MINQVVLVGRETKDIDERYTASGSAVGSFTLAVNRNEKNQNG | 323 |
| | | SSB | | DREADFINCVIWRKPAETMANYARKGTLLGVVGRIQTRNYDN | |
| | | | | QQGQRVYVTEVICESFQLLEPKSANENSNSIQTSQNDGTSVQNN | |
| | | | | FEGNYATNQNKGENQQNNSQQMSFGGDVDPFAGAGNSIDISD | |
| | | | | DDLPF | |
| SSB_90 | SSB | Bacterial- | Klebsiella pneumoniae subsp | MKVISRGQVQQVPAKRQNRGPGNSTGDGFKRQNNGLRRFIMA | 324 |
| | | SSB | rhinoscleromatis ATCC 13884 | ARGVNKVILVGYLGQDPEVRYMPNGGAVANLTLATSETWRD | |
| | | | | KQTGEMRENTEWHRVVMFGKLAEVAGEYLRKGAQVYIEGQL | |
| | | | | RTRNWQDDAGVTRYVTEVLVGQNGTMQMLGGRRESGVPESA | |
| | | | | AQPQNPATPAQPAQAAAKSPKAKGGKKGRQDAAPSQQPPQPL | |
| | | | | PDDFPPMDDDAPF | |
| SSB_91 | SSB | Bacterial- | Haemophilus influenzae, | MRFFMAGINKVIIVGHLGNDPEIRTMPNGDAVANISVATSESW | 325 |
| | | SSB | Haemophilus influenzae NT127 | NDRNTGERREVTEWHRIVFYRRQAEICGEYERKGSQVYVEGRL | |
| | | | | KTRKWQDQNGQDRYTTEIQGDVMQMLGGRNQNAGYGNDMG | |
| | | | | GAPQPSYQARQTNNGGSYQSSRPAPQQSAPQAEPPMDGFDDDI | |
| | | | | PF | |
| SSB_92 | SSB | Bacterial- | Leptotrichia goodfellowii | MNQVLLIGRETKDPELKYSQSGKAFCRFSIAVTKEFNRNETDFF | 326 |
| | | SSB | F0264 | DCVAWNKTAEIIAEYMRKGKKIAIQGRLETGSYEKEGRNIKTY | |
| | | | | SIIVDKFEFVDSAGGQGQQQSSSYSQGTQPKETFADNDNDEIMD | |
| | | | | DDDFPF | |
| SSB_93 | SSB | Viral- | Lactobacillus phage phij11 | MINNVVLVGRLTRDPDLRTTGSGISVATFTLAVDRQYTNSQGE | 327 |
| | | SSB | | RGADFINCVIWRKAAENFANFTSKGSLVGIQGRIQTRTYDDKD | |
| | | | | GKRVYVTEVIVDNFSLLESRRDRENRQANGGNFAPQGGNAPST | |
| | | | | NNFGGSSAPSMNNAPASGESNKPQDPFADSGSTIDISDDDLPF | |
| SSB_94 | SSB | Bacterial- | Fusobacterium ulcerans 12-1B | MNLVVLTGRLTRDPELKFGQSGKAYSRFSLAVDRPFQKGEADF | 328 |
| | | SSB | | INCVAFGKTAELIGEYERKGRKVGVNGRLQMNRYEANGEKRT | |
| | | | | SYDVLVENIEFLEAKGSGDSAGYEPHDYAAAAPASAPKPSVKE | |
| | | | | AEDVPFDDDDEFPF | |
| SSB_95 | SSB | Bacterial- | Pediococcus acidilactici DSM | MINRAVLVGRLTRDPELRYTSSGAAVVSFTVAVNRQFTNSQGE | 329 |
| | | SSB | 20284 | READFINCVMWRKAAENFANFTRKGSLVGIDGRIQTRSYENQQ | |
| | | | | GQRVYVTEVVADNFSLLESRSASERRQENEGFNNGQSAPSQSS | |
| | | | | AGNPFDSGQANNNGAASQPNNSNPNDPFANGGQSIDISDDDLP | |
| | | | | F | |
| SSB_96 | SSB | Bacterial- | Desulfovibrio sp FW1012B | MAGSINKVILVGRLGQDPKLTYLASGSPVAEFSVATDESYKDR | 330 |
| | | SSB | | EGNKQEKTEWHRVKVFGRSAEFCNNYLTKGRLVYIEGTLRTRS | |
| | | | | WEDQQGQKRYTTEVVVTGPGHTVQGLDSRGQASEAPMGEEG | |
| | | | | GFQPRRAPQQGGGGGGQGGAPRGNYGGQGQSGGSRQQPYPD | |
| | | | | EDQGPAFPSEASGMDDVPF | |
| SSB_97 | SSB | Bacterial- | Hungatella hathewayi DSM 13479 | MNRVILMGRLTRDPEVRYSQGERAMAIARYTLAVDRRGRRNQ | 331 |
| | | SSB | | DGNEQTADFINCVAFDRAGEFAEKYFRQGMRVLISGRIQTGSY | |
| | | | | TNKDGIKVYTTDIIVDDQEFADSKGAASGEGGGYQPTSRPAPSS | |
| | | | | AIGDGFMNIPDGVEDEGLPFN | |
| SSB_98 | SSB | Bacterial- | Streptococcus gallolyticus | MINNVVLVGRMTRDAELRYTPSNQAVATFTLAVNRNFKNQNG | 332 |
| | | SSB | subsp gallolyticus TX20005 | EREADFINCVIWRQQAENLSNWAKKGTLIGVTGRIQTRNYENQ | |
| | | | | QGQRVYVTEIVADNFQILESRATREGQSGGSYNGGFNNNNSSF | |
| | | | | GGSSNDGGFSSQPSQSQTPNFGRDESPFGNSNPMDISDDDLPF | |
| SSB_99 | SSB | Bacterial- | Hydrogenobacter thermophilus | MLNKVLIIGRLTKDPSVRYLPSGNQITEFSIAYNRRYKVGDDW | 333 |
| | | SSB | (strain DSM 6534/IAM 12695/ | KEESHFFDVKAYGKLAESESTRISKGYTVVVEGRLTQDRWTDK | |
| | | | TK-6) | EGKAQSKVRIVADAVRIINKPKEDEAPEEEVIPDTYEQEAEEKL | |
| | | | | WNSQDDEIPF | |
| SSB_100 | SSB | Bacterial- | Ruminococcus sp SR1/5 | MNKVILMGRLTRDPEVRYSAGENALAIARYTLAVDRRFRRDG | 334 |
| | | SSB | | EASADFISCVSFGRTAEFAEKYFRQGLKIAVTGRIQTGSYTNRE | |
| | | | | GQKVYTTEVVVEDQEFAESKASSDSYAAAHPRTEAAPATSMPS | |
| | | | | PSAASADGFMNIPDGIDEELPFN | |
| SSB_101 | SSB | Bacterial- | Serratia odorifera DSM 4582 | MVLFGKLAEVAGEYLRKGSQVYIEGALQTRKWTDQAGVEKY | 335 |
| | | SSB | | TTEVVVNVGGTMQMLGGRQGGGAPAGGGQAAGGQGNWGQP | |
| | | | | QQPQGGNQFSGGQQSRPAQNSNAPAASSNEPPMDFDDDIPF | |
| SSB_102 | SSB | Bacterial- | Acinetobacter sp SH024 | MRGVNKVILVGTLGRDPETKTFPNGGSLTQFSIATSEAWTDKN | 336 |
| | | SSB | | TGERKEQTEWHRIVLHNRLGEIAQQFLRKGSKVYIEGSLRTRQ | |
| | | | | WTDQNGQERYTTEIRGDQMQMLDARQQGEQGFAGGNDFNQP | |
| | | | | RFNAPQQGGNGYQNNNNQGGGYGQNSGGYGSQGGFGNGGSN | |
| | | | | PQAGGFAPKAPQQPASAPADLDDDLPF | |
| SSB_103 | SSB | Viral- | Burkholderia phage BcepGomr | MASVNKVILVGNLGADPEVRYLPSGDAVANIRLATTDRYKDK | 337 |
| | | SSB | | ASGEMKEATEWHRVSFFGRLAEIVSEYLKKGSSVYLEGRIRTR | |
| | | | | KWQAQDGTDRYSTEIVAEQMQMLGGRGGSMGGGGDEGGYS | |
| | | | | RGEPSERSGGGGGGRAASGGGSRGGSGGGAGGGASRPSAPAG | |
| | | | | GGFDEMDDDIPF | |
| SSB_104 | SSB | Viral- | Burkholderia phage BcepC6B | MASVNKVILVGNLGADPEVRYLPSGDAVANIRVATTDRYKDK | 338 |
| | | SSB | | ESGELKEVTEWHRVSFFGRLAEIVSEYLKKGSSVYIEGRLRTRK | |
| | | | | WQQDGVDRYSTEIVADQMQMLGGNGKGRTGEGEGDSESGAA | |
| | | | | PETAAGAPAEGSSSKTRSRRAAPQRRASATAGNEMDDDEPFA | |
| SSB_105 | SSB | Bacterial- | Komagataeibacter oboediens | MAGSVNKVILVGNEGKDPEIRNSQNGAKIVSLTLATSETWNDR | 339 |
| | | SSB | | ASGERRERTEWHRVVIFNERIGDVAERFLRKGRKVYLEGTLQT | |
| | | | | RKWTDQSGMERYTTEVVIDRFRGELVELDSNRGGEGGEGGGY | |
| | | | | GGGPGGGGGYGGGAPRPAQAPRSTPPAGGGGGWDAPSGGSDL | |
| | | | | DDEIPF | |
| SSB_106 | SSB | BacterialV | Peptoniphilus duerdenii ATCC | MNVVTLIGRLTRDPELRYSPSGMANVRITVAVDRGYNQQKRQ | 340 |
| | | SSB | BAA-1640 | EAESQNQPTADFISCVAFGKTAELIANYFNKGNRIGLEGRIQTG | |
| | | | | SYDKPDGTRVYTTDVVVNRVHFIESRSESQTYQRRPQEGAGGF | |
| | | | | SAPSQNPGGFNKPPVNSSYDQFSTSEDEGEAFFPVDNEDIPF | |
| SSB_107 | SSB | Bacterial- | Paenibacillus curdlanolyticus | MLNRVILIGRLTRDPELRYTPAGVAVTQFTLAVDRPFTSGGGER | 341 |
| | | SSB | YK9 | EADEIPVVTWRQLAETCANYLRKGRLAAVEGRIQVRNYENNE | |
| | | | | GKRVYVTEVIADNVRFLESNREGGAPREEGSYGGGNSGGGFG | |
| | | | | GGSNAGGGSYGGNSGGGSRSGQNQRNDNRDPFSDDGRPIDISE | |
| | | | | DDLPF | |
| SSB_108 | SSB | Bacterial- | Ahrensia sp R2A130 | MAGSVNKVILVGNLGRDPEIRRTQDGKAIANFSIATSETWRDR | 342 |
| | | SSB | | NSGERREKTEWHRIACFNEGLNKVIEQYVKKGSKVYIEGQLQT | |
| | | | | RKWTDNAGVEKYTTEIVLQNFTGVLTMLDSRNSGGDGGGDSF | |
| | | | | GGGGGGGQIGGSGGGNYGGGGSGGGNQGGGFPSGGDMDDDI | |
| | | | | PF | |
| SSB_109 | SSB | Bacterial- | Parasutterella | MASVNKVIILGNLGRDPETRFSGNNLQITSMSVATTSYRRSAET | 343 |
| | | SSB | excrementihominis | QERVEETEWHRVVLFGRQAEIAQQYLKKGSRVYLEGRLRTRK | |
| | | | CAG:233 | WEKDGQTHYSTEILADTLQLIDRKSDVVGGGQSVAPQSSGDGF | |
| | | | | ESPAAPRRSEFTSGAPAARPAAPAQPAAPVPSAAPTDDFEADEI | |
| | | | | PF | |
| SSB_110 | SSB | Viral- | Mycobacterium phage | MASVDIQQVGNLTADPELRFLPSGVAVAQFSVASTPRVKKGDE | 344 |
| | | SSB | Troll4, Mycobacterium phage | WVDGETVFLRCTVWRELAEGAAETLRKGDQVVVLGKLKQRS | |
| | | | Gumball, Mycobacterium phage | FETKEGEKRTVFEVDGEFVGKSVRARKSRDGGYSAAATEEPPF | |
| | | | Nova, Mycobacterium phage | | |
| | | | SirHarley, Mycobacterium phage | | |
| | | | Adjutor, Mycobacterium phage | | |
| | | | Butterscotch, Mycobacterium | | |
| | | | phage PLot, Mycobacterium | | |
| | | | phage PBI1 | | |
| SSB_111 | SSB | Bacterial- | Paenibacillus polymyxa (strain | MLNRVILIGRLTKDPELRYTPSGVAVTQFTLAVDRPFTSQGGER | 345 |
| | | SSB | E681) | EADFLPIVTWRQLAETCANYLRKGRLTAVEGRVQVRNYENNE | |
| | | | | GKRVYVTEIVADNVRFLESNRDGGNGGGNSGGAAREESPFGG | |
| | | | | GNSNSGRGNNNSRNNQDPFSDDGKPIDISDDDLPF | |
| SSB_112 | SSB | Bacterial- | Neisseria lactamica Y92-1009 | MSLNKVILIGRLGRDPEVRYMPNGEAVCNFSVATSETWNDRN | 346 |
| | | SSB | | GQRVERTEWHNITMYRKLAEIAGQYLKKGGLVYLEGRIQSRK | |
| | | | | YQGKDGIERTAYDIVANEMKMLGGRNENSGGAPYEEGYGQSQ | |
| | | | | EAYQRPAQQNRQPAPDAPSHPQEAPAAPRRQPVPAAAPVEDID | |
| | | | | DDIPF | |
| SSB_113 | SSB | Bacterial- | Hafnia alvei ATCC 51873 | MASRGVNKVILVGNLGQDPEVRYMPNGGAVANITLATSETWR | 347 |
| | | SSB | | DKQTGEQKEKTEWHRVVLFGKLAEVAGEYLRKGSQVYIEGAL | |
| | | | | QTRKWTDQAGVEKYTTEIVVNVGGTMQMLGGRQGGGAPMG | |
| | | | | GGQAQGQQGGWGQPQQPQGNQFSGGSQPAARPQSAPAAQPQ | |
| | | | | SNEPPMDFDDDIPF | |
| SSB_114 | SSB | Bacterial- | Thermaerobacter marianensis | MLNVVVLIGRLVRDPELRYTPSGVAVGGFTLAVDRPFTNQQGE | 348 |
| | | SSB | (strain ATCC 700841/DSM | READFIDIVVWRKLAETCANHLSKGRLVAVRGRLQVRSYETQ | |
| | | | 12885/JCM10246/7p75a) | DGQRRRVAEVVADDVRFLDRGPGADRGAAPGSPAGDELADFG | |
| | | | | DVTGLPDDDIPF | |
| SSB_115 | SSB | Bacterial- | Bacillus sp 2_A_57_CT2 | MMNRVVLVGRLTKDPDLRYTPNGVPVASFTLAVNRTFTNQQG | 349 |
| | | SSB | | EREADFINCVVWRKPAENVANFLKKGSLAGVDGRIQSRSYEGQ | |
| | | | | DGKRVYVTEVQAESVQFLEPKNSSGGQGGNPNYGGPRDQDFP | |
| | | | | FGNNSNQNQRQDNRNQGGYTRVDQDPFANDGQIDISDDDLPF | |
| SSB_116 | SSB | Bacterial- | Bartonella schoenbuchensis | MAGSLNKVILIGNLGADPEIRRLNSGDQVANLRIATSESWRDR | 350 |
| | | SSB | (strain DSM 13525/NCTC | NTNERKERTEWHSIVIFNENLVKIAEQYLKKGNKIYIEGQLQTR | |
| | | | 13165/R1) | KWQDQNGNDRYTTEIVLQKYRGELQMLEGRGAMDGGERMQ | |
| | | | | DTSPLGGGDFGDSSFDRKEDFSQNSNYLEGNFSHQLDDDVPF | |
| SSB_117 | SSB | Viral- | Streptococcus phage | MINNVTLVGRLVAPPDLRKTPNNVSSLQGTLAVNRNFKNENGE | 351 |
| | | SSB | V22, Streptococcus pneumoniae | READFINFQAWRGTADVIAQVCSKGSLIGLTGRLQVRSYEKDG | |
| | | | | QRRYVTEVIAESVALLESRNSQHGQGNSFQNGNSSPFTDPNPFD | |
| | | | | LPNDGLPF | |
| SSB_118 | SSB | Bacterial- | Anaerococcus hydrogenalis ACS- | MNKVFLIGRLTKDPDLRYTQQGQAVVSFSLAVDRGLSKQKRQ | 352 |
| | | SSB | 025-V-Sch4 | EMESMNRPTADFPRITVWGVQAENVSRYLKKGNQCAIDGRIQT | |
| | | | | GSYQDKDGKMVYTTDVVADRVEFLESRSEGQYQNNNNPMNQ | |
| | | | | DNGFGDMNQDRSYNNSNNFQQNNNQNMNSNNDDFFDDDFTE | |
| | | | | IEDDGRIPF | |
| SSB_119 | SSB | Bacterial- | Capnocytophaga sp oral taxon | MNIVGRLTENAQVQTLSNGKQVVNFSVAVNDNYRNKAGENV | 353 |
| | | SSB | 338 str F0234 | QQTSFFDCAYWLSTGVAPYLTKGTLVELEGRVSARAWLNREG | |
| | | | | EPQAGLNFHTSKIKFHFSKKAEVTPNTSASEANNANAITPLPKP | |
| | | | | QVTTGQVAEEDEDDLPF | |
| SSB_120 | SSB | Viral- | Enterobacteria phage | MASKGVNKVILVGNLGQDPEVRYMPNGGAVANLSLATSDTW | 354 |
| | | SSB | HK629, Salmonella phage HK620 | TDKQTGDKKERTEWHRVVLYGKLAEIASEYLRKGSQVYIEGA | |
| | | | (Bacteriophage HK620) | LRTRKWTDQSGVEKYTTEVVVSQSGTMQMLGGRSNAGNGQQ | |
| | | | | QGGWGQPQQPAAPSHSGTPPQQHPASEPPMDFDDDIPFAPFGH | |
| | | | | SVSRHALYALS | |
| SSB_121 | SSB | Bacterial- | Methyloversatilis universalis | MASVNKVIIVGNLGRDPETRYAPNGDAICNITVATTDSWKDKQ | 355 |
| | | SSB | (strain ATCC BAA-1314/JCM | TGERKEQTEWHRVSFYGRLAEIAGQYLRKGSPVYVEGSLRTRK | |
| | | | 13912/FAM5) | WQDKEGQDRYTTEIRAEQMQMLGSRQGAGGEGGGSGGGYGG | |
| | | | | GGGGYGGGDDFDQAPPQRQSAPRQAPSRPQSAPSAPPASSGGG | |
| | | | | FGGMDDDIPF | |
| SSB_122 | SSB | Bacterial- | Streptococcus infantis SK970 | MINNVVLVGRMVRDAELRYTPSNVAVATFTLAVNRTFKSQNG | 356 |
| | | SSB | | EREADFINVVMWRQQAENLANWAKKGSLIGVTGRIQTRSYDN | |
| | | | | QQGQRVYVTEVVAENFQMLESRSVREGQTGGAHSAPSSNYSA | |
| | | | | PTQSVPDFSRDENPFGTTNPLDISDDDLPF | |
| SSB_123 | SSB | Viral- | Listonella phage phiHSIC | MASRGVNKVILVGNLGNDPEIRYMPGGAAVANITIATSDSWRD | 357 |
| | | SSB | | KATGEQREKTEWHRVALFGKLAEVAGEYLRKGSQVYIEGQLQ | |
| | | | | TRKWQDQSGQDRYTTEVVVQGFNGVMQMLGGRAQGGAPAQ | |
| | | | | GGMPQQQQQGGGWGQPQQPAMQKQPQQQQSAPQQAQPQYN | |
| | | | | EPPMDFDDDIPF | |
| SSB_124 | SSB | Bacterial- | Lactobacillus ruminis SPM0211 | MINRVVLVGRLTRDPDLRYTNSGTSVASFTVAVDRNFTNQQG | 358 |
| | | SSB | | NREADFINCVVWGKSAENFANFTHKGSLVGIEGRIQTRSYENQ | |
| | | | | QGNRVYVTEVVTENFSLLESKAESDRYRAQHGGSASSAPRQQS | |
| | | | | QSSFVGNPYGAPANNQGSYQQDNGYGNVNNDAMQDPFAGNG | |
| | | | | SKTDVSEDDLPF | |
| SSB_125 | SSB | Bacterial- | Paenibacillus mucilaginosus | MLNRVILIGRLTRDPELRYTPAGVAVTQFTLAVDRPFSSNQGQR | 359 |
| | | SSB | 3016 | EADFIPVVTWRQLAETCANYLRKGRLAAVEGRIQVRNYDNNE | |
| | | | | GRRVYVTEVIADNVRFLESPNSGNREDGSGMGGSSGGGSSSGG | |
| | | | | GNRGSYGGGREQQDPFQDDGRPIDISDDDLPF | |
| SSB_126 | SSB | Bacterial- | Simkania negevensis (strain | MNQLTIMGHLGADPEVRFTSSGQKVTTLRVAENQKRGGKDET | 360 |
| | | SSB | ATCC VR-1471/Z) | LWWRITIWGDQFDKLVSYLKKGSAIIVTGEMSKPEIYNDRDGK | |
| | | | | PQISLNMTAYHIAFSPFGRTEKQPQEEPAMAGQSSGMSGFGGD | |
| | | | | QGQQHHYHKGGYDQSHMSQGQGPTSYNEPSDDEIPF | |
| SSB_127 | SSB | Bacterial- | Sporosarcina newyorkensis 2681 | MINRVVLVGRLTKDPELKYTQSGIAVTRFTLAVNRAFSNQQGE | 361 |
| | | SSB | | READFINCVAWRKQAENIANYLRKGSLAGVDGRIQTGSFEGQD | |
| | | | | GKRVYTTEVVADSTQFLEPRSANQERPQTPSYGGAPSYNNAPS | |
| | | | | QDQGYNQQSYQPNQQNMTRVDNDPFQPGGGPIEVTDDDLPF | |
| SSB_128 | SSB | Bacterial- | Lactobacillus reuteri | MLNRAVLTGRLTRDPELRYTTSGTAVVSFTLAVDRQFRNQNG | 362 |
| | | SSB | | DRDADFINCVIWRKSAENFSNFTHKGSLVGIEGRIQTRNYENQQ | |
| | | | | GNRVYVTEVVVDNFALLEPRQNGGMNQSGMQQPFNNNQQSF | |
| | | | | GAQAPQYGSQPQPGNNAPQSNPSPSMDNGFDPNQNAGNQFPG | |
| | | | | SSDDGGQSIDLADDELPF | |
| SSB_129 | SSB | Bacterial- | Bacillus subtilis subsp | MLNRVVLVGRLTKDPELRYTPNGAAVATFTLAVNRTFTNQSG | 363 |
| | | SSB | spizizenii (strain TU-B-10), | EREADFINCVTWRRQAENVANFLKKGSLAGVDGRLQTRNYEN | |
| | | | Jeotgalibacillus marinus | QQGQRVFVTEVQAESVQFLEPKNGGGSGSGGYNEGNSGGGQY | |
| | | | | FGGGQNDNPFGGNQNNQKRNQGNSFNDDPFANDGKPIDISDD | |
| | | | | DLPF | |
| SSB_130 | SSB | Bacterial- | Thiorhodovibrio sp 970 | MASRGVNKVILIGNLGNDPEIRYFPNGDAVTNLSIATSESWKDR | 364 |
| | | SSB | | NTGEPQERTEWHRIVIRGKLAEIAKQYLRKGSKVFIEGKLRTRK | |
| | | | | WQGQDGQDRYTTEVIVDMTGSMQMLDSRPSGGDYGADNSGG | |
| | | | | SWTSDPAPGIGTAAATTYAQAPYPDQQSPQQQAPAPQNSGQYP | |
| | | | | SQYPNQQPAQSPAPPPDQSGPGLDEPFDDDIPF | |
| SSB_131 | SSB | Bacterial- | Paenibacillus lactis 154 | MLNRVILIGRLTRDPELRYTPSGVAVTQFTLAVDRPFTGQGGER | 365 |
| | | SSB | | EADFIPVVTWRQLAETCANYLRKGRLTAVEGRIQVRNYENNE | |
| | | | | GKRVYVTEVIADNVRFLESNREGGGGGNREESSFGGGSGSGNR | |
| | | | | GGNNFSRNNQDPFSDEGKPIDISDDDLPF | |
| SSB_132 | SSB | Viral- | Streptococcus phage M102 | MINNVVLVGRTTKEIELKYTSNNLAYANFTLAVNRNFKNQNG | 366 |
| | | SSB | | EREADFINIVIWRQQAENLANWAKKGTLLGITGRIQTRNYENQ | |
| | | | | QGQRVYVTEVVADNFQILERREVQANTQKPAQQEAFSDVDDI | |
| | | | | DLPF | |
| SSB_133 | SSB | Bacterial- | Commensalibacter intestini A911 | MAGSVNKVILVGNLGKDPDVRTTQMGTKVVNLTLATSDTWN | 367 |
| | | SSB | | DRQTGERKENTEWHRVVIFNERLADVAEKYLRKGRKVFIEGQ | |
| | | | | LKTRKWTDQQGMERYTTEVVVDRFRGELVLLDSNRSGGDDM | |
| | | | | GYGDDYGASAPMAPASRPAMSAPSAPSKSAGGGWDSSMPGH | |
| | | | | NDLDDEIPF | |
| SSB_134 | SSB | Bacterial- | Desulfitobacterium | MLNRVVLIGRLTKDPELRYTPSGVAVATFTLAVDRNEKNSNGE | 368 |
| | | SSB | metallireducens DSM 15288 | RDTDFIPCVVYRQLAELCANFLSKGRLASVDGRLQVRSFEGQD | |
| | | | | GQRRWVTEVIAENVQFLSPKENGNSGNGTSGNASNVGTYGHE | |
| | | | | VSLDDDIPF | |
| SSB_135 | SSB | Bacterial- | Paenibacillus terrae (strain | MLNRVILIGRLTKDPELRYTPSGVAVTQFTLAVDRPFTSQGGER | 369 |
| | | SSB | HPL-003) | EADFLPIVTWRQLAETCANYLRKGRLTAVEGRVQVRNYENNE | |
| | | | | GKRVYVTEIVADNVRFLESNRDGGNGGGNSGGAAREESPFGG | |
| | | | | GNSNSGRGNNNSRNNHDPFSDDGKPIDISDDDLPF | |
| SSB_136 | SSB | Bacterial- | Bradyrhizobium sp STM 3843 | MAGSVNKVILVGNLGKDPEIRRTQDGRPIANLSIATSETWRDK | 370 |
| | | SSB | | ATGERKEKTEWHRVVIFNEGLCKVAEQYEKKGAKVYIEGALQ | |
| | | | | TRKWTDQSGVEKYSTEVVLQGFNSTLTMLDGRGGGGGSFADE | |
| | | | | PGGDFGSSGPSMAPPRRAVASAGGGRNSDMDDDIPF | |
| SSB_137 | SSB | Bacterial- | Frateuria aurantia (strain ATCC | MARGINKVILVGNLGGDPEERYTGGGTAVCQLRVATAETWND | 371 |
| | | SSB | 33424/DSM 6220/NBRC 3245/ | KQSGQRQERTEWHRVVLFGKEGEIAQEYLRKGRQVYIEGSLRT | |
| | | | NCIMB13370) (Acetobacter | KEYTDKEGIKRFTTEVIATDMQMLSGDGGSSGNRQQPGNSRGR | |
| | | | aurantius) | GQQANQRGHAQQHEPPPDQGAPPFDDDDIPF | |
| SSB_138 | SSB | Bacterial- | Bacillus sp 1NLA3E | MMNRVVLVGRLTKDPDLRYTPNGVAVATFTLAVNRSFSNQQG | 372 |
| | | SSB | | EREADFINCVVWRRPAENVANFLKKGSLAGVDGHIQTRHYEG | |
| | | | | QDGKRVYVTEVVAESVQFLEPKSSASGDRGGSGTYNEPREQQ | |
| | | | | GSPFGNSNNNQNQNQRQNNNNKGYTRVDEDPFAGDGQIDISD | |
| | | | | DDLPF | |
| SSB_139 | SSB | Bacterial- | Paenibacillus dendritiformis | MLNRVILIGRLTRDPELRYTPSGVAVTQFTLAVDRPFSNQSGER | 373 |
| | | SSB | C454 | EADFIPVVTWRQLAETCANYLRKGRLTAVEGRIQVRNYDNNE | |
| | | | | GKRVYVTEVIADNVRFLESNRDSGGSRDDMGGGYGGGQPNN | |
| | | | | NSRPYGGGGSNQSRGPAADPFSDDGRPIDISDDDLPF | |
| SSB_140 | SSB | Bacterial- | gamma proteobacterium BDW918 | MARGINKVILVGNLGQDPETRYMPSGGAVTNVTIATSETWKD | 374 |
| | | SSB | | KQSGQPQERTEWHRVVFFNRLAEIAGEYLKKGSKVYVEGSLRT | |
| | | | | RKWQNKEGVDQYTTEIVAAEMQMLDGRGGAGGGASGGASN | |
| | | | | YDDGGYGQQQAPQAQAAAPAPRRAPPPQQNRAPAAPAQNPPA | |
| | | | | GFDDFDDDIPF | |
| SSB_141 | SSB | Bacterial- | Haemophilus | MAGVNKVIIVGNLGNDPEMRTMPNGEAVANISVATSESWTDK | 375 |
| | | SSB | paraphrohaemolyticus HK411 | NTGERREATEWHRIVFYRRQAEVAGQYLRKGSQVYVEGRLKT | |
| | | | | RKWQDQNGQDRYTTEIQGDVLQMLGSRNQGGEMGGQGGWS | |
| | | | | QSNNQGGNWNQAPASNNYNQGNSYNNNYSQTASKPVAKPAQ | |
| | | | | AEPPMDNFDDDIPF | |
| SSB_142 | SSB | Viral- | Xanthomonas phage OP2 | MASRGVNKVILVGNLGNDPEIRYMPNGGAVANITVATSESWN | 376 |
| | | SSB | | DKQTGEKKEVTEWHRVVLFGKVAEIAGEYLKKGSQVYIEGQL | |
| | | | | KTRKWEKDGVERYTTEIVVNVGGTMQMIGKAPEGGSGGGNRS | |
| | | | | ASNTGWGQPQQPQHSGTPNNKPQNRPSANHQGAAQNGEPPM | |
| | | | | NFDDDIPF | |
| SSB_143 | SSB | Bacterial- | Nitrolancea hollandica Lb | MARRDLNKIQIIGNLGRDPEMRYTPGGTPVTEFSVAVNRPPRR | 377 |
| | | SSB | | GQDGQATEETDWFRVVCWDKLAEITDQYLKKGSRVYIEGRLQ | |
| | | | | IRKYTGNDGVDRTSVEIIARDMLMLSGREEGGYAGREEGGTRR | |
| | | | | EPASSRSGDSGEEDFDDLPF | |
| SSB_144 | SSB | Bacterial- | Herbaspirillum sp YR522 | MASVNKVIIVGNLGRDPETRYMPNGEAVTNIAVATTESWKDK | 378 |
| | | SSB | | NSGEKKELTEWHRITFYRKLAEIAGQYLKKGSQIYVEGRLQTR | |
| | | | | KWTDKDGGERYTTEIIADTMQMLGSRQGGGGGGGSMDDGGS | |
| | | | | YGGGGGGYGGGAPRQAGGGAGGGGGASAPRQQPARQPASNN | |
| | | | | FQDMDDDIPF | |
| SSB_145 | SSB | Bacterial- | Rhizobium sp CF080 | MAGSVNKVILVGNVGADPEIRRTQDGRPIANLRIATSETWRDR | 379 |
| | | SSB | | NNGERREKTEWHTVVVFNEGLCKVVEQYVKKGAKLYIEGAL | |
| | | | | QTRKWQDQTGNDRYSTEIVLQGFNSTLTMLDGRGEGGGDRGG | |
| | | | | AGGNRVGNDFGGNDFGGGDDYERRPAAGGASRGGQSSGGQP | |
| | | | | AGGNFSRDLDDDIPF | |
| SSB_146 | SSB | Bacterial- | Paenibacillus alvei DSM 29 | MLNRVILIGRLTRDPELRYTSSGVAVTQFTLAVDRPFSSQGGER | 380 |
| | | SSB | | EADFIPVVTWRQLAETCANYLRKGRLTAVEGRIQVRNYDNNE | |
| | | | | GKRVYVTEIIADNVRFLESNRDNGGTRDDMGSNYGAPAPQYN | |
| | | | | APARGGNSNSRGQAAADPFSDDGRPIDISDDDLPF | |
| SSB_147 | SSB | Viral- | Lactobacillus phage | MINRVVLAGRPTRNLELKSIKSGNSVCSFTLAVDRNFKSKSGER | 381 |
| | | SSB | KC5a, Lactobacillus phage | EADFINCVAWGKTAEVMSQYVKKGSAIGVDGRIQTRSYDNRD | |
| | | | phi jlb1 | GQRVYVTEVVVENFSFLSDPPKNSQVSKNNQSLNQSNDPFDSN | |
| | | | | GQVDIADDDLPF | |
| SSB_148 | SSB | Bacterial- | Gordonia soli NBRC 108243 | MAGDTVITVIGNLTADPELRFTPSGAAVANFTVASTPRTFDRQT | 382 |
| | | SSB | | NEWKDGEALFLRCNIWRDAAENVTESLTKGSRVIVQGRLKQR | |
| | | | | SFETREGEKRTVVELEVDEIGPSLRYATAKVNKASRGGGGGGG | |
| | | | | FGSSGGGSRGGGSNQQVADDPWGSAPASSGSFGGGDDEPPF | |
| SSB_149 | SSB | Bacterial- | Streptomyces rimosus | MSFGETPVTIIGNLTADPELKYTTGGQALARFTVASTPRTFDRE | 383 |
| | | SSB | | ANQWKDGTSTFFRCATWRALAEHVAESLTKGSRVVLSGRIRQ | |
| | | | | HNWQTEQGENRSMLAVEVDEIGASLRFTTVTIEGKRTNGTAPA | |
| | | | | DDPWNTAGNPAKTDEEPPF | |
| SSB_150 | SSB | Bacterial- | Paeniclostridium sordellii | MNHVVLVGRLTKDPELRYIPGTGTAVATFTIAIDRDYAKKDGS | 384 |
| | | SSB | (Clostridium sordellii) | RETDFIPVEVMGKSAEFCANYISKGREVALQGSIRVDNYQNQS | |
| | | | | GERRTFTKVSARNIQALDSNKNRSDSPYPSQNQSFEPSFEPSFEP | |
| | | | | TGLDPQGFQAIDDDDIPF | |
| SSB_151 | SSB | Viral- | Pseudomonas | MATPVFWEGNIGSAPEHRSFPNGNNPPRQLLRLNVMFDNSIPD | 385 |
| | | SSB | aeruginosa , Pseudomonas | GQGGYKDRGGFWCSVEWWHQDAQRFAELFAKGMRVKVEGR | |
| | | | aeruginosa DHS01, Pseudomonas | AIMDRWPDKESGEEVQALKVEASRISILPHRLAEVTLLPSTNGQ | |
| | | | phage LKA5, Pseudomonas phage | ATQHQQTRQVSQQDYDSAFDDDIPM | |
| | | | F116 | | |
| SSB_152 | SSB- | Bacterial- | Lactobacillus rossiae DSM | MINRAVLTGRLTRDPEVRYTQSGAAVGSFTLAVDRQFTNQQG | 386 |
| | | SSB | 15814 | QREADFINCVIWRKSAENFANFTHKGSLVGIEGRIQTRNYENQQ | |
| | | | | GQRVYVTEVVVENFALLESRSQSDQRTSGNTNDNGGYNNNAP | |
| | | | | QSGSANPFGGTGNNGGNNAGNTAPSSQSSQTPADPFAGNGESI | |
| | | | | DISDDDLPF | |
| SSB_153 | SSB | Bacterial- | Nocardia terpenica | MFGAITPTVIGNLTADPELFFSKKGEAGVHFTVACNDRYYDKD | 387 |
| | | SSB | | AERYKDLPAVFMRCTAWGALAEHISDSLSRGMTVIAYGRLKQ | |
| | | | | SSYTVKDTGEKRTVMEMTIDVLGPSLQYATAVVTKASRAATVI | |
| | | | | GEPIDDPWGDAAEQTPVSVGAGEQSSPEGDDDKPPF | |
| SSB_154 | SSB | Bacterial- | Spiroplasma kunkelii CR2-3x | MNQFTAIGRTTKDIEIKKTNNGKEYAIFQLAVARPHSNKKETDF | 388 |
| | | SSB | | IPCQVWNKQASVLQQYCQKGSQIAIKGILQSFKDKDNKIHWMV | |
| | | | | RVYSYEFLQTKNNLNNDTYNDIQSITPNITDQTQLIPRNIRLEEV | |
| | | | | ETKELFENQDDTILWD | |
| SSB_155 | SSB | Bacterial- | Enterococcus faecalis (strain | MINNLTLVGRLTKDVDLRYTKSGTAVGQFILAVNRNFTNQNG | 389 |
| | | SSB | ATCC 700802/V583) | DREADFINCVIWRKAAESLANYARKGTLIGLTGRIQTRNYENQ | |
| | | | | QGQRVYVTEVVIENFQLLESKEVNEQRRGQSTGAGQATFDKQP | |
| | | | | TDKPDPLDPFEQGNSPIEISDDDLPF | |
| SSB_156 | SSB | Viral- | Mycobacterium phage Hamulus, | MALTLPVVTIEGTLTADPTLNFTQGGTAVSNITIACNTRRKNPQ | 390 |
| | | SSB | Mycobacterium phage Dante, | TDQWEDGDTTFMRGTIWKQLAENVSDSLRKGDRVLAHGVLK | |
| | | | Mycobacterium phage Ardmore, | QKDYEKDGVKRTAYELDIEGIGPSLRFATASVTKSSGGSGGGA | |
| | | | Mycobacterium phage Llij, | RQQPKEDPWGGSSDDWGGDF | |
| | | | Mycobacterium phage Drago, | | |
| | | | Mycobacterium phage Phatniss, | | |
| | | | Mycobacterium phage Spartacus, | | |
| | | | Mycobacterium phage Boomer, | | |
| | | | Mycobacterium phage SiSi, | | |
| | | | Mycobacterium phage PMC, | | |
| | | | Mycobacterium phage Ovechkin, | | |
| | | | Mycobacterium phage Ramsey, | | |
| | | | Mycobacterium phage Fruitloop, | | |
| | | | Mycobacterium phage SG4 | | |
| SSB_157 | SSB | Bacterial- | Dialister sp CAG:486 | MNSVQVMGNLARDPVVRATKTGRAMASFTVAVNRSFTTPQG | 391 |
| | | SSB | | EQREITDWINVVAWGNLAEAVGNQLKKGSRVFVEGRFTTRSY | |
| | | | | DTPDGQRRWVSEVTANFVALPIGGFSHSQQQPSGNAFGGNNG | |
| | | | | YTGNNGYGGNNGGFNNGASPFGQSQPQGQPKSSFGQFGQASK | |
| | | | | DEDIPF | |
| SSB_158 | SSB | Bacterial- | Faecalibacterium sp CAG:82 | MLNVVAIMGRLVADPELRTTQSGINVVSFRIACDRNFARQGEQ | 392 |
| | | SSB | | RQADFIDIVAWRQQAEFVSKYFQKGSLIAIEGSLQTRQYQDKN | |
| | | | | GNNRTAVEVVANNISFAGPKSNNQGGGSYQNAAPSYQNQAPA | |
| | | | | RPAAVEAAPSYSSGSADDFAVIDDSDDLPF | |
| SSB_159 | SSB | Bacterial- | Clostridium sp CAG:470 | MNKVILMGRLTRDPEVRYTQTNNTLVASFSLAVNRRFARQGE | 393 |
| | | SSB | | ERQADFINIVAWSKLGEFCSKYFKKGQQVGIIGRIQTRTWDDD | |
| | | | | QGVKHYVTEVVAEEAYFADSKREGDMGAGTFENTFGNTMPG | |
| | | | | SMGSDFETSSTDDLPF | |
| SSB_160 | SSB | Bacterial- | Prevotella sp CAG:873 | MNKVMLIGNVGKDPDVKYYDADQAVAQFSLATTERGYQLPN | 394 |
| | | SSB | | GTRVPDRTDWHNIVMWGNLAKIVERYVHKGDRLYVEGKMRY | |
| | | | | RYYDDKKGQRRFIAEVYADNMELLTPRSAANTAADSTSQNTN | |
| | | | | AAANNANQNAASSSNASIASTEDDDQLPF | |
| SSB_161 | SSB | Viral- | Pseudomonas phage vB_Pae- | MARGVNKVILVGTCGQDPEVRYLPNGNAVTNLSLATSEQWTD | 395 |
| | | SSB | Kakheti25 | KQSGQKVERTEWHRVSLFGKVAEIAGEYLRKGSQVYIEGKLQT | |
| | | | | REWEKDGIKRYTTEIIVDMQGTMQLLGGRPQNQQGGGDQYNQ | |
| | | | | GGGNNYNQGGQQQQYNQAPQRQQAPRPQQQRPAPQQPAPQP | |
| | | | | AADFDSFDDDIPF | |
| SSB_162 | SSB | Bacterial- | Avibacterium paragallinarum | MAGVNKVIIVGNLGNDPEIRTMPNGEAVANISVATSESWMDK | 396 |
| | | SSB | JF4211 | NTGERREITEWHRIVFYRRQAEVAGEYERKGSKVYVEGRLRTR | |
| | | | | KWQDQNGQDRYTTEIQGDVLQMLDSRADRGQGGYSAQGGYA | |
| | | | | QQGSNQYAQPAQPSYQAPQQQAAARPSSPPTPMVDDNFDDDN | |
| | | | | IPF | |
| SSB_163 | SSB | Bacterial- | Streptomyces sp HPH0547 | MAMGDTPVTVVGNLVADPELKYTQSGTALARFTVASTPRSYD | 397 |
| | | SSB | | RESGQYKDGTAMFMRCSAWRGLAENIASSLAKGNRVVVTGRL | |
| | | | | RQHNWQTPEGENRSMLALEVDEIGPSLRFATAQPAKADNETK | |
| | | | | KTAPPADDPWNTTPAPAGGDEPPF | |
| SSB_164 | SSB | Bacterial- | Dermabacter sp HFH0086 | MANDTVITVVGNLTADPELRFTNSGIPVASFTVASTPRTFDRQS | 398 |
| | | SSB | | GEWKDGEALFLRCSIWRDAAENVAESLTKGTRVIVQGRLQQR | |
| | | | | SYTDREGNNRTSIELQVDEIGPSLRYATAKPTRTQRGGGGNFG | |
| | | | | GGFNGGNSGGGNYGGGQGGYSNQGGYGGNRGGAQGGPQGG | |
| | | | | QNPADVDPWSNGGAEEPPPF | |
| SSB_165 | SSB | Bacterial- | Treponema socranskii subsp | MTDINHVLVIGRLTRDFGADPRTFFYTTGGTACAKVSIAVNRS | 399 |
| | | SSB | socranskii VPI DR56BR1116 = | VKQSDGQWTDEVSFFDVTIWGKTAENLKPYLVKGKQIAVDGY | |
| | | | ATCC 35536 | LKQDRWQKDGQNFSKVNIVANSVQLLGGGTSAPESAPAPQNY | |
| | | | | GRVQETYRDNPSQVPPRMQGNYMQPQQPAYQTTAPQQQFGG | |
| | | | | GDDFPEDLPF | |
| SSB_166 | SSB | Bacterial- | Lactobacillus shenzhenensis | MINRVTLVGRLTQDVEVKHTESGIAVANFTVAVERHFKNAEGE | 400 |
| | | SSB | LY-73 | KQADFVTCKMWRKSAENFANFTCKGSLVGILGEVRTHTYEKD | |
| | | | | GQKVYRTDIEADTFALLEPKAVTEARRAGTLKSGSGGGSDNVF | |
| | | | | AAAGANGEKIDITDDDLPF | |
| SSB_167 | SSB | Bacterial- | Bifidobacterium magnum | MIQVTFTGNAGQDPETKTFNNGGSITQVNVGIGQGYKDRASGQ | 401 |
| | | SSB | | WIDKGTAWVTVKANTSQTKETLQHVHKGTHLLVTGSLTVRTY | |
| | | | | QKQDGTQGTALDVNATAIAIIPRKQQQMQQPQQPMQQPVQQP | |
| | | | | QQQNTWASMPTGTDPWSQGSFNQEPEF | |
| SSB_168 | SSB | Bacterial- | Borrelia duttonii CR2A | MISKEWKSGVGFMMMGVLMSDINNITLSGRLVKDSLLSYSSTN | 402 |
| | | SSB | | LAILNFSIANNIKVKREGEWEDNAQFFNCVLFGKRAETLFHFLS | |
| | | | | KGKQVVVNGSMRHEYYKNKHSEVNKIKSIIFVEQLRLFGADSK | |
| | | | | HHNPKVDIPIPVPPPVPDSACEFNEDIPF | |
| SSB_169 | SSB | Viral- | Pseudomonas phage | MRGVNKVILVGNVGGDPETRYMPNGNAVTNITLATSESWKDK | 403 |
| | | SSB | vB_PaeP_C1-14_Or, Pseudomonas | QTGQQQERTEWHRVVFFGKLAEIVGQHVKKGQQLYVEGSLRT | |
| | | | phage vB_PaeP_p2- | RKWQAQDGQDRYTTEIIVDMHGQMQMFGGKPGNEQAAQSRS | |
| | | | 10_Or1, Pseudomonas phage PaP3 | STQQQSAPQQRSAQDEFDDDIPL | |
| SSB_170 | SSB | Bacterial- | Mycobacterium brisbanense | MAGDTTITVIGNLTADPELRFTPSGAAVANFTVASTPRTFDRQT | 404 |
| | | SSB | | NEWKDGEALFLRCNIWREAAENVAESLTRGSRVIVQGRLKQRS | |
| | | | | FETREGEKRTVVELEVDEIGPSLRYATAKVNKASRSGGGGGGF | |
| | | | | GGGGGGFSGGGGGSRQSEPKDDPWGSAPASGSFSGADDEPPF | |
| SSB_171 | SSB | Bacterial- | Pseudoalteromonas lipolytica | MARGVNKVILVGNLGQDPEVRYMPNGNGVANISIATTDSWKD | 405 |
| | | SSB | SCSIO 04301 | KNTGQMQERTEWHRVVLFGKLAEVAGEYLRKGSQVYIEGRLQ | |
| | | | | TRKWTDQSGQEKFTTEIVVDMGGQMQMLGGRGGDQQGGGY | |
| | | | | QGGQSQGGYQGGQQQGGGYGGGSQQAQSNSYAPQQQSAPAP | |
| | | | | AQQQRPQQQPAPAPAPQQNNNQYGGYGQQQSSAPQQGGFAP | |
| | | | | KPQNAPQGGASNPMEPPIDFDDDIPF | |
| SSB_172 | SSB | Bacterial- | Candidatus Accumulibacter sp | MASVNKVILVGNLGADPETRYLPSGDAVCNIRMATTDRSRDK | 406 |
| | | SSB | SK-12 | VSGELKEYTEWHRVVFFGKLAETAGQYLKKGRQVYVEGRIRT | |
| | | | | NKWQDKEGNERYTTEIVANEMKMLGSREGMGAPAGEAEYGG | |
| | | | | SMPSAAGQAAGAARNAPARKTPGFEDMDDDIPF | |
| SSB_173 | SSB | Viral- | Listeria phage B054, Listeria | MMNRVVLVGRLTKDPELRYTPAGVAVATFTLAVNRPFKNAQ | 407 |
| | | SSB | monocytogenes | GEQEADFINCVVWRKPAENAANFLKKGSMAGVDGRVQTRNY | |
| | | | | EDSDGKRVFVTEVVAETVQFLEPKNINAEGATSNNYQNQANY | |
| | | | | SNNNKTSSYRADTSQKSDSFADEGKAIDINEDDLPF | |
| SSB_174 | SSB | Bacterial- | Hydrogenovibrio marinus | MRGVNKVILVGTLGRDPEMKYAANGNAIANLSLATSENWTDK | 408 |
| | | SSB | | NSGQKQEKTEWHRVVIFGKLAEIAGQYLTKGSQIYLEGKLQTR | |
| | | | | KWQDQNTGQDRYSTEVVIDFNGQMQMLGGGNKPQGQGAAP | |
| | | | | QGQGFAGQQPPQGQPMANQQQQAPQQNQQPQAGNQAAMQN | |
| | | | | QAPPMQQAPAYPENDFGDDDVPF | |
| SSB_175 | SSB | Viral- | Lactobacillus phage phig1e | MSINSVVLTGRLTKDVDLRATNSGKNVARFTLAVDRNFKSEQ | 409 |
| | | SSB | | QADFFTVSVWGKQAENTAKYCHKGSLVGVRGHLRSGSYDKN | |
| | | | | GQKVYFVDIEADSVQFLDTRNNSQDAPQSENLGAQSDFGFQSG | |
| | | | | QTYHSGADNSQSTFNSFGGTSQNDYPF | |
| SSB_176 | SSB | Bacterial- | Pedobacter antarcticus, | MSGINKVILVGHLGKDPEVRHLDGGVTVASFPLATSETYNKEG | 410 |
| | | SSB | Pedobacter antarcticus 4BY | KRIEQTEWHNIVLWRGLAEVASKYLQKGKEVYIEGKERTRSFE | |
| | | | | DKERVKKYVTEVVAENFTLLGRKSDFENTPVAAATPKHENDY | |
| | | | | VEPEDNATGDLPF | |
| SSB_177 | SSB | Bacterial- | Salinisphaera hydrothermalis | MASKGVNRAIILGNEGADPEVRHTAGGTAVANISVATSEVWT | 411 |
| | | SSB | C41B8 | DKNSGEKQERTEWHRVVAFARLAEVMGEFLKSGSKVYIEGKI | |
| | | | | QTRKWQNREGQDVYTTEIVANELQMLDSKPQGNAGAQSRAN | |
| | | | | NQAQNYGAASRGGAPARGGQQRQSPPQYQPPAGGDPGFDDDL | |
| | | | | DDDIPL | |
| SSB_178 | SSB | Bacterial- | Endozoicomonas montiporae, | MARGVNKVILIGNLGGDPDVRFTPNGSAVANFNVATSESWTD | 412 |
| | | SSB | Endozoicomonas montiporae CL- | KNTGQRQDRTEWHRVVVFGKLAEICQQYLRKGSKVYLEGKLQ | |
| | | | 33 | TRKWQDQQGQDRYTTEVVVDGFGGQMQMLDSRQDGGMGAV | |
| | | | | PNQMGGGYQQPQAAPQQQAPQQQAPQQRAPQQQYAAPQQQA | |
| | | | | QPQAQQRPAPAQQQAAPQPAAAGFDDFDDDIPF | |
| SSB_179 | SSB | Bacterial- | Nitratireductor basaltis | MAGSVNKVILVGNLGADPEIRRLNSGDPVVNLRIATSETWRDR | 413 |
| | | SSB | | GTGERRERTEWHNVVIFNDNLAKVAEQYLKKGAKVYLEGQL | |
| | | | | QTRKWQDQQGQDRYTTEVVLQKFRGELQMLDTRGQGGDNQI | |
| | | | | GYGGGGGGGQRSDFGQSSPAEPSGGGGGGGYSRDLDDEIPF | |
| SSB_180 | SSB | Bacterial- | Paenibacillus sp FSL R7-0331 | MLNRIILIGRLTRDPELRYTPAGVAVTQFTLAVDRNFTGQNGER | 414 |
| | | SSB | | EADFIPVVTWRQLAETCANYLRKGRLAAVEGRIQVRNYENNE | |
| | | | | GKRVYVTEVIADNVRFLESSQNREGGNAPSGGSMPEEPAFGGG | |
| | | | | NGGNSARGNNNNFSRSNNNQDPFSGDGKPIDISDDDLPF | |
| SSB_181 | SSB | Bacterial- | Burkholderia cenocepacia | MASVNKVILVGNLGADPEVRYLPSGDAVANIRLATTDRYKDK | 415 |
| | | SSB | (strain ATCC BAA-245/DSM | ASGDFKEMTEWHRVAFFGRLAEIVSEYLKKGSSVYIEGRIRTRK | |
| | | | 16553/LMG 16656/NCTC 13227/ | WQGQDGQDRYSTEIVADQMQMLGGRGGSGGGGGGGDEGGY | |
| | | | J2315/CF5610) (Burkholderia | GGGYGGGGGRGGEQMERGGGGGRAGGAARGGAGGGQSRPS | |
| | | | cepacia (strain | APAGGGFDEMDDDIPF | |
| | | | J2315)), Burkholderia | | |
| | | | cenocepacia BC7 | | |
| SSB_182 | SSB | Bacterial- | Bifidobacterium reuteri DSM | MGVNVSFTGNVGRDPETREFDNGRSLTTFSVGVSQGYYDQQN | 416 |
| | | SSB_23975 | | QWHDQGTMWITVECSPTAARQLPYVHKGVKLLVTGRLSQRFY | |
| | | | | QKKDGGQGSELRVYADAIGFLHRKDEQPQTGGFTGAQPQPPAS | |
| | | | | DPWAAPQSDTEPEF | |
| SSB_183 | SSB | Viral- | Lactococcus phage | MAIITVTAQANEKNTRTVNTAKGDKKIISVPLFEKEKGSNVKV | 417 |
| | | SSB | phi311, Lactococcus phage | AYGSAELPDFIQLGDTVTISGRVQAKESGEYVNYNFVFPTIEKV | |
| | | | ul36k1t1, Lactococcus phage | FISNDNGKQAQAKQDLFGGSEPIEVNTEDLPF | |
| | | | ul362, Lactococcus phage | | |
| | | | ul361, Lactococcus lactis, | | |
| | | | Lactococcus phage ul36k1 | | |
| SSB_184 | SSB | Bacterial- | Helicobacter sp MIT 05-5294 | MFNKVILVGNLTRDVELRYLPSGSALAKLGLAVNRRFKKQDG | 418 |
| | | SSB | | SQGDEVCYIDVNLFGRTAEVANQYLKRGSSVLIEGRLVLESWT | |
| | | | | DNNGQKRSKHSITAETMQMLGSRNEGGNYAGNGGGNGNYGN | |
| | | | | SYSNDSYNDMHNGGYNNYGSYQNTQPSAPKPQKPKENDIPEID | |
| | | | | IDDEEIPF | |
| SSB_185 | SSB | Viral- | Prochlorococcus phage P-SSM2 | MNHCLLEVTVKVAPTIRYTQDNQTAIAEMDVEFDGFRADDPP | 419 |
| | | SSB | | GSIKVVGWGNLAQDLQSHVQIGQRLIIEGRLRMNTVPRQDGSK | |
| | | | | EKRAEFTLSKIHSSTPKGTISPNKTSPNQVPSNDSPSLNALTSKEP | |
| | | | | ENPKSDNDSVTWNSSPLIPDTDDIPF | |
| SSB_186 | SSB | Viral- | Flavobacterium phage 1 lb | MNRQEFIGHIGNDAEVKDLGINQVINDSVAVSESYVNKTTNEKI | 420 |
| | | SSB | | TNTTWYECAKWGNNTQIAQYLKKGQQVYIMGKPNNRAWQN | |
| | | | | EQGDIKVVNAVKVTEILLLGGKQSNDNNAQPQQPQQQPQQPQ | |
| | | | | QAPQPQNNEDFNNSEEHDDLPF | |
| SSB_187 | SSB | Bacterial- | Paenibacillus sp P1XP2 | MLNRVILIGRLTKDPELRYTPAGVAVTQFTLAVDRPFTSQGGER | 421 |
| | | SSB | | EADFIPVVTWRQLAETCANYLRKGRLTAVEGRIQVRNYENNE | |
| | | | | GRRVYVTEVIADNVRFLESNREGGSGGGQGPREESSFGGGSRE | |
| | | | | NNNYSRSNSNQDPFFDDGKPIDISDDDLPF | |
| SSB_188 | SSB | Bacterial- | Mameliella alba | MAGSVNKVILVGNEGRDPETRTFQNGGKVCNLRIATSENWKD | 422 |
| | | SSB | | RNTGERRERTEWHSVAIFSEPLARIAEQYLRKGSKVYIEGQLET | |
| | | | | RKWQDQNGQDRYSTEVVLRPYRGELTLLDSRSEGGGGGGFGG | |
| | | | | GSGGGYGGGGGGYDDRGGYDDPGYGGGSGGGSGGGSGPSRA | |
| | | | | PARDIDDEIPF | |
| SSB_189 | SSB | Bacterial- | Sulfurovum sp ES06-10 | MNQFTGLGNLTRDIELRYLQNGSAIATCGLAMNRRFKKQDGT | 423 |
| | | SSB | | QGEEVCFIDITFFGRTAEIANQYLSRGRKVLIIGRLKLDQWTDQ | |
| | | | | QGVKRSKHSIHVESLEMIDSQNSEAQSTHPQPQAHNNQTQRQP | |
| | | | | QNTPRQPQSNPGQYSGHDIPDIDINDDEIPF | |
| SSB_190 | SSB | Bacterial- | Streptomyces cyaneogriseus | MNETMICAVGNVATHPVYRELASGPSARFRLAVTSRYWDREK | 424 |
| | | SSB | | SAWTDGHTNFFTVWAHRQLAANAAASLAVGDPVMVQGRLK | |
| | | | | VRTDVREGQSRTSADIDAVAIGHDETRGTSAFRRTNKPDSASTS | |
| | | | | PRPEPNWETPAAESGDPSEQQPTGEPALM | |
| SSB_191 | SSB | Viral- | Thalassomonas phage BA3 | MAGVNKVIILGNLGKDPEVRFMPNGGGVANLTIATSETWKDK | 425 |
| | | SSB | | QTGEQKEKTEWHRVVMFGKLAEIAGEYLKKGSKVYIEGQLQT | |
| | | | | RKWQNQQGQDQYTTEIVVQGFNGTMQMLDSRGQGGGGQGG | |
| | | | | GFQGQQQGGFSGGQQAPQQQGGFQQQAPKQQGGFSQQAAPQ | |
| | | | | QQGGFAQQAPQQQAPQQGGFSGGQQAPQQGGFQQQQGGFGQ | |
| | | | | QNQQQAAKVNPQEPSIDFDDDIPF | |
| SSB_192 | SSB | Bacterial- | Clostridium sp FS41 | MNSAQLVGRLTRDPEVRYSDGGTTVARFTLAVDRRFKKDGGD | 426 |
| | | SSB | | DADFINCVAFGKTAEFLEKWFRKGQRLGLTGRIQTGSYVNQEG | |
| | | | | TKVYTTEVVVENVEFVESKGASAGDGGPQQRPAPTSAIGDGFM | |
| | | | | NIPDGVEDDGLPFN | |
| SSB_193 | SSB | Bacterial- | Acetobacter orientalis 21F-2 | MAGSVNKVILVGNLGKDPEVRTTQGGQKIVSFSLATSDTWND | 427 |
| | | SSB | | RQSGERRERTEWHRVVVFNEREADVAERFLRKGRKVYLEGAL | |
| | | | | QTRKWTDQSGQERFTTEVIVERFRGELVLLDSRADNEGGQSNQ | |
| | | | | QPQPREQPRQQGGYGQQSNGWGGSSDEDDSIPF | |
| SSB_194 | SSB | Bacterial- | Microbacterium ginsengisoli | MAGETVITVVGNLTADPELRYTQNGLPVANFTIASTPRTFDRQ | 428 |
| | | SSB | | ANEWKDGEALFLRASVWREFAEHVAGSLTKGSRVIATGRLRQ | |
| | | | | RSYQDRDGNNRTAIELEVDEIGPSLRYATAQVTRAAGGSGAGG | |
| | | | | GGSRAQVADEPWSTPGSSNSSADAWSAPGAYGDDTPF | |
| SSB_195 | SSB | Viral- | Staphylococcus phage | MNTVNLIGNLVADPELKGQNNNVVNFVIAVQRQEKNKQTNEY | 429 |
| | | SSB | SA97, Staphylococcus phage | ETDFIRCVAFGKTAEIIANNFNKGNKIGVTGSIQTGSYENNQGQ | |
| | | | phi7247PVL, Staphylococcus | KVFTTDIAVNNITFVERKNTQAKHGLLTRN | |
| | | | phage phiETA3, Staphylococcus | | |
| | | | aureus, Staphylococcus phage | | |
| | | | phi5967PVL | | |
| SSB_196 | SSB | Bacterial- | Salinicoccus halodurans | MLNRVVLVGRLTKDPDLKVSQNNISVATFTLAVNRPFTSSNGE | 430 |
| | | SSB | | RGADFINCVVFRKQAENVNQYLKKGSLAGVDGRLQSRTYDNK | |
| | | | | DGQRVFVTEVVCDSVQFLEPKGQGGNQQQQYNNNSADSYTN | |
| | | | | AYGNQSTGSRPAPQQQSRQDNAEENPFANADGPVDISDDDLPF | |
| SSB_197 | SSB | Viral- | Lactobacillus phage phiadh | MINRVVLVGRLTRDPDELRYTNSGTSVASFTVAVDRNFTNQQG | 431 |
| | | SSB | | NREADFINCVVWGKSAENFANFTHKGSLVGIEGRIQTRSYENQ | |
| | | | | QGNRVYVTEVVTENFSLLESKAESDRYRAQHGGSASSAPRQQS | |
| | | | | QSSFGGNPYGAPANNQGSYQQDNAYGNGNNDAMQDPFAGNG | |
| | | | | SKTDISEDDLPF | |
| SSB_198 | SSB | Bacterial- | Microgenomates group bacterium | MSSRSLNKVQIIGNLTRDPELRYTPQGTAVCQIGVATNRSWTN | 432 |
| | | SSB | GW2011_GWF1_44_10 | DAGEKNEETEFHKVVAWSKLAEICSQLLKKGRKIYLEGRLQTR | |
| | | | | DWTTQDGQKRQTTEIVMDNMILLDSAGRGGDGEGASTSYTND | |
| | | | | DTSSKPVAKKSKKADVADDASSAEEAPVAEDVSDDIPF | |
| SSB_199 | SSB | Bacterial- | Parcubacteria group bacterium | MNLNKAFVLGNLTRDPELRTTTTGQSVAQFGVATNREFTDKS | 433 |
| | | SSB | GW2011_GWA2_42_14 | GQRQKLTEFHNIVAWGKLGELCHQYIKKGQSVFVEGRIQTRSW | |
| | | | | DDKQTGQKKYRTEIVAENVQFGPKPFRNETAGQNQAQPETPKE | |
| | | | | KEEILETVQYPADEEEIKPEEIPF | |
| SSB_200 | SSB | Bacterial- | Labilithrix luteola | MAEGLNKVMLLGNLGADPELKMTAGGQAVLKLRLATTETYL | 434 |
| | | SSB | | DRNNSRQERTEWHSVTLWGKRGEALAKFLSKGERIFVEGSLRT | |
| | | | | SSYEKDGEKRYRTEINATNVILAGRAGRGAGDEMGGGGGGGG | |
| | | | | GFGGGGGGGGGGGGGGFERRAPSRSGGGGGGGFEGGGRGAP | |
| | | | | ASAPPADDFGDYPGGDDEIPF | |
| SSB_201 | SSB | Bacterial- | Sphingopyxis sp (strain 113P3) | MAGSVNKVILIGNLGADPEIKSFQNGGKIANIRIATSESWKDRM | 435 |
| | | SSB | | TGERKERTEWHNVVINGDGLVGVVERYLKKGSKVYIEGSLRT | |
| | | | | RKWQDRDGNDRYTTEVVVAGMGGSLTMLDGAPGGGGSRTSG | |
| | | | | DSWNQGGGSSGGWDQGGSSGGGWNQGGGSSGGGRPPFDDDL | |
| | | | | DDDVPF | |
| SSB_202 | SSB | Bacterial- | Actinobacteria bacterium OK074 | MSNETIITVVGNLVDDPELRWTSSSVAVAKFRIASTPRTFDKQT | 436 |
| | | SSB | | NEWKDGESLFLTCSVWRQAAEHVAESLQRGMRVIVQGRLKQR | |
| | | | | SYEDREGVKRTVYELDVEELGPSLRNATAKVTKAGGSGQARE | |
| | | | | ALQQARTRSSREGREDPWASSGAAAESAQAGAWGDAPPF | |
| SSB_203 | SSB | Viral- | Thermus phage phiYS40 | DFIDPLEGRGRETLEDARGQPRLRHALNQVILMGNLTRDPDLR | 437 |
| | | SSB | | YTPQGTAVARLGLAINERRPGQGPDGERTHFIEVQAWRDLAE | |
| | | | | WAEELKRGEGLLVIGRLVNDSWTSSTGERRFQTRVEALRLERP | |
| | | | | TRGPERTGGSRPQEPERSVQTGGVDIDEGLEDFPPEEDLPF | |
| SSB_204 | SSB | Bacterial- | Lentibacillus amyloliquefaciens | MMNRVVLVGRLTRDPDLRYTPNGVAVANFRIAIDRPDSNQQG | 438 |
| | | SSB | | NRDADFINCVVWRRAAENLATYMKKGSMIGVDGRIQSRSFEG | |
| | | | | RDGNTVFVTEVVADNIQFLESKGTSQSRDQQPSGFQPNQNQNQ | |
| | | | | NQNQTTQTQTNENPFKDNGEPIDISDDDLPF | |
| SSB_205 | SSB | Viral- | Phormidium phage Pf-WMP3 | MAEIVQDPQLRYTSDNQTAITELLVQIDPLRDGDPPETLKVVA | 439 |
| | | SSB | | WRRLAEAVQENFHRGDRVVIEGRLGMVVFDRPEGFREKRAEV | |
| | | | | TAQRIHLLDRAAAGSAPPAAPTAAVPSSAPVTPMNGPANTPAN | |
| | | | | APAPVSSPDEPLSDDIPF | |
| SSB_206 | SSB | Bacterial- | Lactobacillus capillatus DSM | MINRAILVGRLTRDPDLRYTANGVAVATFTVAVNRQFTNQQG | 440 |
| | | SSB | 19910 | EREADFINCVIWRKSAENFANFTHKGSLVGVDGRIQTRSYENQ | |
| | | | | QGNRVYVTEIVVDSFSELESRSQSERYQQQHGADTQGSAPSQN | |
| | | | | SSNPNNDNLFGNSTKNNPPKARENNTDVDPFADSGKQIDISDD | |
| | | | | DLPF | |
| SSB_207 | SSB | Bacterial- | Candidatus Cloacimonas sp SDB | MTSELRLPRVNYVVLSGRLTRDVDLRFIPNGTPVAKLSLAFDR | 441 |
| | | SSB | | NYQKDGEWQQETSVIDVVVWSKRGEQCAEYLQKGSPVLIEGY | |
| | | | | IKTRSYQDKDNNNRKVTEIIASKINFLEKKPYSSEDDSETKNDSS | |
| | | | | DTNKSKADIIDDDVPF | |
| SSB_208 | SSB | Bacterial- | Roseateles depolymerans | MASVNKVIILGNLGRDPELRYTPSGSAVCNVSIATTRNWKSRE | 442 |
| | | SSB | | GGERQEETEWHRVVFYDRLAEIAGEYLKKGRPVYVEGRLKTR | |
| | | | | KWQDKEGKDNYTTEIIAETMQLLGGRDGGDDMGGGGGGGYN | |
| | | | | RERSSGGSRESSGGGSGRDAGDFDSPRAPAPRSAPRPAPAPAAK | |
| | | | | PATGFDDMDDDIPF | |
| SSB_209 | SSB | Bacterial- | Parcubacteria bacterium 32_520 | MNYNRAILCGRVTKAPEILMTPSGHKVAKISLATNEYQGKGKE | 443 |
| | | SSB | | EKTVFHNLIAWDRTADIAQQVIVVGHEIMIEGRIDNRTYKKKD | |
| | | | | GTKGYISEVVIDRLQLGNKPRAVAVPAENTATSNYQEPPADDD | |
| | | | | NVPVIEDMDEIDISSIPF | |
| SSB_210 | SSB | Bacterial- | Streptomyces longwoodensis | MAGETVITVIGNLVDDPELRFTPSGAAVAKFRVASTPRTFDRQT | 444 |
| | | SSB | | NEWKDGESLFLTCSVWRQAAENVAESLQRGMRVLVQGRLKQ | |
| | | | | RSYEDREGVKRTVYELDVEEVGPSLRNATAKVTKTAGRGGQG | |
| | | | | GGGGFGGGGGGQQGGGWGGGPGGGQQGGGAPADDPWATGA | |
| | | | | PAGGAQQGGGGWGGGSGGGGGYSDEPPF | |
| SSB_211 | SSB | Bacterial- | Streptomyces albus | MAGETVITVVGNLVDDPELRFTPSGAAVAKFRVASTPRTFDRQ | 445 |
| | | SSB | | TNEWKDGESEFLTCSVWRQAAENVAESLQRGMRVIVQGRLKQ | |
| | | | | RSYEDREGVKRTVFELDVDEVGASLRNATAKVTKTSGRGGQG | |
| | | | | GYGGGGGQGGGGWGGGSGGGQQGGGAPADDPWATGAPSGG | |
| | | | | QQGGGGGGWGGGSGNSGGYSDEPPF | |
| SSB_212 | SSB | Bacterial- | Pirellula sp SH-Sr6A | MASYNRVILLGNLVRDIELKYTTSRLAVCQNAIAVNERRKNAA | 446 |
| | | SSB | | GEWVDETSFVDVTFFGRTAEVVAEYLGKGSPIFVEGKLKQDT | |
| | | | | WEKDGQKRSKLYVIVDRMQLIGSRNESKGSGAPRPQSNGNRF | |
| | | | | ADQEQHVSPDMHVSEVGDGGFIDEDMPF | |
| SSB_213 | SSB | Bacterial- | Bacillus sporothermodurans | MMNRVVLVGRLTKDPDLRYTPSGVAVATFTLAVNRTFTNQQG | 447 |
| | | SSB | | EREADFINCVVWRKPAENVANFLKKGSLAGVDGRLQTRNYEG | |
| | | | | QDGKRVYVTEVVAESVQFLEPRNANANPNRGGNNDFYGGGQ | |
| | | | | GNQNTPFNQNQNQRNQGYTRVDEDPFSNDGQPIDISDDDLPF | |
| SSB_214 | SSB | Bacterial- | Akkermansia sp KLE1798 | MANLNKVFLMGNLTADPELRYTPKGTAVTDIRLAINRYYAGD | 448 |
| | | SSB | | NSERQEETTFVDVTLWNRQAEVAGNYLSKGRGVFVEGRLQLD | |
| | | | | SWEDKASGQKRTKLRVIGENIQLFPRGGDSSDMGGAPRQQSAP | |
| | | | | RSNNYGQSQAPQNYNPPPMPSNQQQSNDEGDMDDEIPF | |
| SSB_215 | SSB | Bacterial- | Coriobacteriales bacterium | MSINRVILSGNLTRTPELRSTANGSSVLGFGIAVNDRRKNPQTG | 449 |
| | | SSB | DNF00809 | EWEDFPNFIDCTVFGPRAEGLSHCLDKGSKISLEGKERWSQWE | |
| | | | | RDGQRRSKLEVIVDEIELMSTRGGQNFAANDHASADAGSYSQP | |
| | | | | YNAPSTPAAPPAVDPSSYNADLPF | |
| SSB_216 | SSB | Bacterial- | Streptomyces albulus | MAGETVITVVGNLVDDPELRFTPSGAAVAKFRVASTPRTFDRQ | 450 |
| | | SSB | | TNEWKDGESLFLTCSVWRQAAENVAESLQRGMRVVVQGRLK | |
| | | | | QRSYEDREGVKRTVFELDVEEVGPSLKNATAKVTKTTGRGGQ | |
| | | | | GGYGGGQQGGGNWGGAPGGGQQGGGAPADDPWATSAPAGG | |
| | | | | QQQGGGGNWGGSSGGSGGGYSDEPPF | |
| SSB_217 | SSB | Viral- | Salmonella phage SETP3 | MARGVNKVIIVGTLGNDPEVKYSASGSAIVNISVATSEQWKDK | 451 |
| | | SSB | | QTGEKKEQTEWHRIVIFGKLAEVAGEYLRKGSQVYIEGQLRTR | |
| | | | | KWTDSNGIDRYTTEIVIPQMGGVMQMLGGKRDDSGQQPRQQS | |
| | | | | GQQPQGGWVTNQQQQPQKQQSPQGGNEPPMDESDDIPF | |
| SSB_218 | SSB | Bacterial- | Streptomyces noursei | MAGETVITVVGNLVDDPELRFTPSGAAVAKFRVASTPRTFDRQ | 452 |
| | | SSB | | TNEWKDGESLFLTCSVWRQAAENVAESLTRGTRVIVQGRLKQ | |
| | | | | RSYEDREGVKRTVYELDVEEVGASLRNATAKVTKTGGRGGQG | |
| | | | | GGGFGGGQGGGQQGGGWGGGPGGGQQGGGAPADDPWATGG | |
| | | | | PSGGGQGGGGGWGGGSGGSGGGYSDEPPF | |
| SSB_219 | SSB | Viral- | Synechococcus phage Syn5 | MNHCLLEVEVIEAPQLRYTQDNQTPVAEMSVQFEGLRPDDPSG | 453 |
| | | SSB | | QIKVVGWGNLAQDLQNRVQVGQRLMLEGRLRISTITRADGLK | |
| | | | | EKRAEFTLSRLHPLAEGPSPAGDTRPAPLPPAGGQPRSVPRPVP | |
| | | | | ARAAVAGSTTAAAAIPATPAPEVPTWNTSPEVPELSDDDDIPF | |
| SSB_220 | SSB | Viral- | Streptomyces phage VWB | MAGETPITVVGNVVADPELRETPSGAPVANFRVASTPRTFDRV | 454 |
| | | SSB | | TNEWKDGDTLFLSVSVWRQQAENVAESIKRGDRVIVVGRLGQ | |
| | | | | RQYEKDGERKSSYEVQAEDVGPALKNATAQVAKNGQQNGQQ | |
| | | | | RAQGYGQGYGQQAPQQGYGAPQADQWSTQQPRQGYTDEPPF | |
| SSB_221 | SSB | Bacterial- | Lactococcus lactis subsp | MINNVVLVGRITRDPELRYTPQNQAVATFSLAVNRQFKNANGE | 455 |
| | | SSB | cremoris (strain MG1363) | READFINCVIWRQQAENLANWAKKGALIGVTGRIQTRNYENQ | |
| | | | | QGQRVYVTEVVADSFQMLESRSARDGMGGGASAGSYSAPSQS | |
| | | | | TNNTPRPQTNNNSATPNFGRDADPFGSSPMEISDDDLPF | |
| SSB_222 | SSB | Bacterial- | Lactococcus lactis subsp | MNKTMLIGRLTNAPEISKTTNNKSYVRVTLAVNRRFKNEKGER | 456 |
| | | SSB | cremoris (strain MG1363) | EADEISIIIWGKSAETLVSYAKKGSLISVEGEIRTRNYTDKNEQK | |
| | | | | HYITEILGLSYDLLESRATLALRESAVKTEELELEADELPF | |
| SSB_223 | SSB | Bacterial- | Lactococcus lactis subsp | MINNVTLVGRITKKPELRYTPQNKAVATFTLAVNRAFKNANGE | 457 |
| | | SSB | cremoris (strain MG1363) | READFISCVIWGKSAENLANWTHKGQLIGVIGNIQTRNYENQQ | |
| | | | | GQRVYITEVVASNFQVLEKSNQANGERVSNPAAKPQNNDSFGS | |
| | | | | DPMEISDDDLPF | |
| SSB_224 | SSB | Bacterial- | Mycobacterium smegmatis | MNMFETPFTVVGNIITNPVRLRFGDQELYKFRVASNSRRRSPEG | 458 |
| | | SSB | | TWEPGNSLYVTVNCWGNLARGVSASLGKGDSVVVVGHLYTN | |
| | | | | EYEDREGVRRSSVEVRATAVGPDLSRCIARVEKVQPSQGPRAD | |
| | | | | AGGDPERVPDDDVDRRDADDEPDDLADDDVADLAGDGLPLT | |
| | | | | A | |
| SSB_225 | SSB | Bacterial- | Staphylococcus aureus (strain | MLNRTILVGRLTRDPELRTTQSGVNVASFTLAVNRTFTNAQGE | 459 |
| | | SSB | Mu50/ATCC 700699) | READFINIIVFKKQAENVNKYLSKGSLAGVDGRLQTRNYENKE | |
| | | | | GQRVYVTEVIADSIQFLEPKNSNDTQQDLYQQQVQQTRGQSQY | |
| | | | | SNNKPVKDNPFANANGPIELNDDDLPF | |
| SSB_226 | SSB | Bacterial- | Staphylococcus aureus (strain | MLNRVVLVGRLTKDPEYRTTPSGVSVATFTLAVNRTFTNAQGE | 460 |
| | | SSB | Mu50/ATCC 700699) | READFINCVVFRRQADNVNNYLSKGSLAGVDGRLQSRNYENQ | |
| | | | | EGRRVFVTEVVCDSVQFLEPKNAQQNGGQRQQNEFQDYGQGF | |
| | | | | GGQQSGQNNSYNNSSNTKQSDNPFANANGPIDISDDDLPF | |
| SSB_227 | SSB | Bacterial- | Staphylococcus aureus (strain | MLNRTVLVGRLTKDPELRSTPNGVNVGTFTLAVNRTFTNAQG | 461 |
| | | SSB | Mu50/ATCC 700699) | EREADFINVVVFKKQAENVKNYLSKGSLAGVDGRLQTRNYEN | |
| | | | | KDGQRVFVTEVVADSVQFLEPKNNNQQQNNNYQQQRQTQTG | |
| | | | | NNPFDNNADSIEDLPF | |
| SSB_228 | SSB | Bacterial- | Caulobacter vibrioides (strain | MAGSVNKVILVGNEGADPEIRSLGSGDRVANLRIATSETWRDR | 462 |
| | | SSB | ATCC 19089/CB15) | SSGERKEKTEWHRVVIFNDNEVKVAEQYLRKGSTVYIEGALQT | |
| | | | (Caulobacter crescentus) | RKWTDNTGQEKYSTEIVLQKFRGELTMLGGRGGDAGMSSGGG | |
| | | | | DEYGGGYSGGGSSFGGGQRSQPSGPRESFSADLDDEIPF | |
| SSB_229 | SSB | Bacterial- | Vibrio natriegens NBRC 15636 = | MASRGINKVILVGNEGNDPEIRYMPNGGAVANITIATSDSWRD | 463 |
| | | SSB | ATCC 14048 = DSM 759 | KATGEQREKTEWHRVVLFGKLAEVAGEYLRKGSQVYVEGQL | |
| | | | | QTRKWQDQSGQDRYSTEVVVQGFNGVMQMLGGRAQGGAPA | |
| | | | | MGGQAPQQGGWGQPQQPAQPQYNAPQQQAPKQSAPQQPQQQ | |
| | | | | YNEPPMDFDDDIPF | |
| SSB_230 | SSB | Bacterial- | Synechocystis sp PCC 6803 | MSVNSIHLVGRAGRDPEVKYFESGNVVCNFTLAVNRRTSKKD | 464 |
| | | | SSB | EPPDWFDLEIWGKTAEIAGNYVKKGSLIGIQGSLKFDHWEDRN | |
| | | | | SGTPRSKPVIRVNNLDLLGSKRDNAEATMNNYPEEF | |
| SSB_231 | SSB | Bacterial- | Synechocystis sp PCC 6803 | MNSFVLMATVIREPELRFTKENQTPVCEFLVEFPGMRDDSPKES | 465 |
| | | SSB | | LKVVGWGNLANTIKETYHPGDRLIIEGRLGMNMIERQEGFKEK | |
| | | | | RAELTASRISLVDSGNGINPGELSSPPEPEAVDLSNTDDIPF | |
| SSB_232 | SSB | Bacterial- | Streptomyces albus J1074 | MAGETVITVVGNLVDDPELRFTPSGAAVAKFRVASTPRTFDRQ | 466 |
| | | SSB | | TNEWKDGESLFLTCSVWRQAAENVAESLQRGMRVIVQGRLKQ | |
| | | | | RSYEDREGIKRTVYELDVDEVGASLRNATAKVTKTTGRGGQG | |
| | | | | GYSGGGGGGGQQGGWGGGPGGGQQQGGGGAPADDPWATSA | |
| | | | | PSGGGQQQGGGGGWGGSSGGGGGYSDEPPF | |
| SSB_233 | SSB | Bacterial- | Streptomyces albus J1074 | MNETMVTVVGNVATAPVYRESAHGPMARFRMAATPRRWDRE | 467 |
| | | SSB | | RQTWTDGPTSFFTIWTTRQLASNVTASVTVGEPVIVQGRERVR | |
| | | | | ETERGGQQWTTAEIDAASVGHDLTRGTAAFRRVRKPVGTWPG | |
| | | | | GTATEDSRSAAAAQRAAGEPDWSVSGVGAGGDWGVAGGDRS | |
| | | | | GGGGEEPAEAGDRAEASAGTGTGTGTGGGEPEVEAVSPGPGA | |
| | | | | A | |
| SSB_234 | SSB | Bacterial- | Streptomyces coelicolor (strain | MNETMICAVGNVATTPVFRDLANGPSVRFRLAVTARYWDREK | 468 |
| | | SSB | ATCC BAA-471/A3(2)/M145) | NAWTDGHTNFFTVWANRQLATNASGSLAVGDPVVVQGRLKV | |
| | | | | RTDVREGQSRTSADIDAVAIGHDLARGTAAFRRTARTEASTSPP | |
| | | | | RPEPNWEVPAGGTPGEPVPEQRPDPVPVG | |
| SSB_235 | SSB | Bacterial- | Streptomyces coelicolor (strain | MAGETVITVVGNLVDDPELRFTPSGAAVAKFRVASTPRTFDRQ | 469 |
| | | SSB | ATCC BAA-471/A3(2)/M145) | TNEWKDGESLFLTCSVWRQAAENVAESLQRGMRVIVQGRLKQ | |
| | | | | RSYEDREGVKRTVYELDVDEVGASLRSATAKVTKTSGQGRGG | |
| | | | | QGGYGGGGGGQGGGGWGGGPGGGQQGGGAPADDPWATGG | |
| | | | | APAGGQQGGGGQGGGGWGGGSGGGGGYSDEPPF | |
| SSB_236 | SSB | Bacterial- | Synechococcus sp UTEX 2973 | MNSCILQATVVEAPQLRYTQDNQTPVAEMVVQFPGLSSKDAP | 470 |
| | | SSB | | ARLKVVGWGAVAQELQDRCRLNDEVVLEGRLRINSLLKPDGN | |
| | | | | REKQTELTVTRVHHLTLDSATGILAQEESEVSYGRSAAASAPV | |
| | | | | KASPVVTPTAAPDVDYDDIPF | |
| SSB_237 | SSB | Bacterial- | Synechococcus sp UTEX 2973 | MSLNVVNLVGRVGRDPEARYFESGSVVCKFSLAVNRRSRNDE | 471 |
| | | SSB | | PDWFNVEMWGREAQVAIDYVKKGSLIGISGALKIESWTDRNN | |
| | | | | NQRTTPVVRANRLELLGSRRDQEGGMAPRDPDSDLF | |
| PaSSB | SSB | | Pseudomonas aeruginosa | MARGVNKVILVGNVGGDPETRYMPNGNAVTNITLATSESWKD | 472 |
| | | | | KQTGQQQERTEWHRVVFF | |
| | | | | GRLAEIAGEYLRKGSQVYVEGSLRTRKWQGQDGQDRYTTEIV | |
| | | | | VDINGNMQLLGGRPSGDD | |
| | | | | SQRAPREPMQRPQQAPQQQSRPAPQQQPAPQPAQDYDSFDDDI | |
| | | | | PF | |

TABLE 2
Minimum inhibitory concentration (MIC) for various
combinations of ciprofloxacin resistance-conferring alleles.

| | Ciprofloxacin MIC |
|---|---|
| Genotype | (μg/ml) |
| PAO1 wild-type | 0.25 |
| nfxB knockout | 4 |
| GyrA_T83I | 16 |
| GyrA_T83I + | 32 |
| ParC_S87L | |
| GyrA_T83I + | >128 |
| ParC_S87L + NfxB | |
| knockout | |

### Materials and Methods

### Strains and Plasmids

A strain of Escherichia coli, which is derived from MG1655, but which has mutS knocked out, has a mutation in dnaG (Q576A) which decreases its affinity for single-stranded binding protein (SSB) at the replication fork, was used. A plasmid with beta lactamase (carb/amp resistance) on a p15a origin was used. Proteins were cloned by Gibson assembly under the control of the pBAD (arabinose) promoter.

The commonly studied strain of L. lactis, NZ9000, which features a nisin induction system was used. A plasmid with chloramphenicol acetyltransferase (chloramphenicol resistance) was used, built off of pJP005. Proteins were cloned by Gibson assembly under the control of a nisin-inducible promoter.

M. smegmatis strain MC2 155 was studied. A plasmid with a kanamycin resistance gene on a dual origin plasmid (colE1 and oriM) was used. Proteins were cloned by Gibson assembly under the control of a tetracycline-sensitive operator. TetR, the tetracycline operator repressor was also present on the plasmid.

### Culture, Induction and Transformation

E. coli cultures were grown in standard Lysogeny Broth (LB) at 37° C. in a rotating drum. Overnight cultures were diluted 1:100, grown for 90 minutes, and then single-stranded annealing proteins (SSAPs), or pairs of SSAPs and single-stranded binding proteins (SSBs) were induced with arabinose, grown another 30 minutes, and then prepared for transformation. Briefly, cells were put on ice, washed twice with cold water, and resuspended in 1/100^{th} culture volume of water.

L. lactis cultures were grown in M17 media supplemented with 0.5% glucose at 30° C. and not shaken. Overnight cultures were diluted 1:10 into M17 media supplemented with 0.5% w/v glucose, 0.5 M sucrose, and 2.5% w/v glycine. Diluted cultures were grown for three hours and then induced with 5 ng/μl nisin, grown another 30 minutes and then prepared for transformation. Briefly, cells were put on ice, washed twice with a cold buffer containing 0.5 M sucrose and 10% glycerol, resuspended in 1/100^{th} culture volume.

M. smegmatis cultures were grown in 7H9 media supplemented with 0.5% w/v BSA, 0.2% w/v glucose, 0.085% w/v NaCl, 0.05% v/v Tween 80, and 0.2% glycerol. Cultures were grown at 37° C. in a rolling drum for two days until confluent, then diluted 1:100 and grown overnight until OD600 reached 0.4-0.8. Cultures were then induced with 400 μg/ml anhydrotetracycline (ATC), put in the incubator for another hour, and then prepared for transformation. Briefly, cells were put on ice, washed twice with cold water, and resuspended in 1/100^{th} culture volume.

Unless otherwise noted, bacterial cultures were grown in Lysogeny-Broth-Lennox (LB^{L}) (10 g tryptone, 5 g yeast extract, 5 g NaCl in 1 L H₂O). Super optimal broth with catabolite repression (SOC) was used for recovery after electroporation. MacConkey agar (17 g pancreatic digest of gelatin, 3 g peptone, 10 g lactose, 1.5 g bile salt, 5 g NaCl, 13.5 g agar, 0.03 g neutral red, 0.001 g crystal violet in 1 L H₂O) and IPTG-X-gal Mueller-Hinton II agar (3 g beef extract, 17.5 g acid hydrolysate of casein, 1.5 g starch, 13.5 g agar in 1 L H₂O, supplemented with 40 mg/L X-gal and 0.2 μM IPTG) were used to differentiate LacZ(+) and (−) mutants. Cation-adjusted Mueller Hinton II Broth (MHBII) was used for antimicrobial susceptibility tests. Antibiotics were ordered from Sigma-Aldrich. Recombineering oligos were synthesized by Integrated DNA Technologies (IDT) or by the DNA Synthesis Laboratory of the Biological Research Centre (Szeged, Hungary) with standard desalting as purification.

### Oligo-Mediated Recombineering

Bacterial cultures (E. coli, K. pneumoniae, C. freundii, or P. aeruginosa) were grown in LB^{L} at 37° C. in a rotating drum. Overnight cultures were diluted 1:100, grown for 60 minutes or until OD600≈0.3, whereupon expression of SSAPs was induced for 30 minutes with 0.2% arabinose or 1 mM m-toluic acid as appropriate. Cells were then prepared for transformation. Briefly, E. coli, K. pneumoniae, and C. freundii cells were put on ice for approximately ten minutes, washed three times with cold water and resuspended in 1/100^{th} culture volume of cold water. This same procedure was followed for P. aeruginosa with the following differences: (1) Resuspension Buffer (0.5 M sucrose+10% glycerol) was used in place of water and (2) there was no pre-incubation on ice, as competent cell prep was carried out at room temperature, which was found to be much more efficient than preparation at 4° C. After competent cell prep, 9 μl of 100 μM oligo was added to 81 μl of prepared cells for a final oligo concentration of 10 μM in the transformation mixture (2.5 μM final oligo concentration was used for C. freundii and K. pneumoniae). This mixture was transferred to an electroporation cuvette with a 0.1 cm gap and electroporated immediately on a Gene Pulser (BioRad) with the following settings: 1.8 kV (2.2 kV in the case of P. aeruginosa), 200 Ω, 25 Cultures were recovered with SOC media for one hour and then 4 ml of LB with 1.25× selective antibiotic and 1.25× antibiotic for plasmid maintenance were added for outgrowth.

### Engineering of SEER Chassis

The E. coli strain described in this work as the SEER chassis is engineered from EcNR2 (Wang et al., Nature 460, 894-898 (2009)). EcNR2 harbors a small piece of λ-phage integrated at the bioAB locus, which allows expression of λ-Red genes, and a knockout of the methyl-directed mismatch repair (MMR) gene mutS, which improves the efficiency of mismatch inheritance (MG1655 ΔmutS::cat Δ(ybhB-bioAB)::[λc1857 Δ(cro-orf206b)::tetR-bla]). Modifications made to EcNR2 to engineer the SEER chassis include: 1. improvement of MAGE efficiency by mutating DNA primase (dnaG_Q576A) (Lajoie et al., Nucleic Acids Res. 40, e170 (2012)), 2. introduction of a handle for SDS selection (tolC_STOP), 3. introduction of a handle for CHL selection (mutS::cat_STOP), and 4. removal of lambda phage with a zeocin resistance marker Δ[λcI857 Δ(cro-orf206b)::tetR-bla]::zeoR, The final strain which was referred to as the SEER chassis is therefore: MG1.655 Δ(ybhB-bioAB)::zeoR ΔmutS::cat_STOP tolC_STOP dnaG_Q576A.

### Selective Allele Testing in the SEER Chassis

To complement the SEER chassis' two engineered selective handles, following native antibiotic resistance alleles were tested: [TMP: FolA P21→L, A26→G, and L28→R], [KAN/GEN: 16SrRNA U1406→A and A1408→G], [SPT: 16SrRNA A1191→G and C1192→U], [RIF: RpoB S512→P and D516→G], [STR: RpsL K4→R and K88→R], and [CIP: GyrA S83→L] (Novais et al., PLoS Pathog. 6, (2010); Criswell et al., Antimicrob. Agents Chemother. 50, 445-452 (2006); Campbell et al., Cell 104, 901-912 (2001); Okamoto-Hosoya et al., Microbiol. Read. Engl. 149, 3299-3309 (2003); Yoshida et al., Antimicrob. Agents Chemother. 34, 1271-1272 (1990)). 90-bp oligos conferring each mutation, with two PT bonds at their 5′ end and with complementarity to the lagging strand were designed. Two oligos were designed to repair the engineered selective handles: (1) elimination of a stop codon in the chloramphenicol acetyltransferase (cat) to confer CHM resistance and (2) elimination of a stop codon in tolC to confer SDS resistance. Oligo-mediated recombineering was run with Redβ expressed off of the pARC8 plasmid and the cultures were then plated onto a range of concentrations of the antibiotic to which the oligo was expected to confer resistance. Colony counts were made and compared to a water-blank control. Modifications targeted to provide TMP, KAN, and SPT resistance did not work adequately and so were dropped. RpsL_K43R was chosen for STR selection and RpoB_S512P for RIF selection, although in both cases there was not a significant observable difference between the two tested alleles. An antibiotic concentration was chosen that provided the largest selective advantage for those cultures transformed with oligo (Fig S2). The concentrations chosen for the selective antibiotics were: 0.1% v/v SDS, 25 μg/ml STR, 100 μg/ml RIF, 0.1 μg/ml CIP, and 20 μg/ml CHL.

### Identification of SSAP Library Members

To generate Broad SSAP Library a multiple sequence alignment of eight SSAPs was used that had been shown to function in E. coli (Redβ, EF2132 from Enterococcus faecalis, OrfC from Legionella pneumophila, 5065 from Vibrio cholerae, Plu2935 from Photorhabdus luminescens, Orf48 from Listeria monocytogenes, Orf245 from Lactococcus lactis, and Bet from Prochlorococcus siphovirus P-SS2 (Datta et al., Proc. Natl. Acad. Sci. U.S.A. 105, 1626-1631 (2008); Sullivan et al., Environ. Microbiol. 11, 2935-2951 (2009))) to generate a Hidden Markov Model that described the weighted positional variance of these proteins. Then non-redundant nucleotide and environmental metagenomic databases were queried using a web-based search interface (Finn et al., Nucleic Acids Res. 39, W29-W37 (2011)). Candidates were filtered based on gene size and annotation. Those that exhibited intra-sequence similarity of greater than 98% were removed from the group. Three eukaryotic SSAP homologs were added to the library (Eisen et al., Proc. Natl. Acad. Sci. U.S.A. 85, 7481-7485 (1988)). In total, Broad SSAP Library contains 120 members from the homology search, 8 members from the starting sequence alignment, and 3 eukaryotic members, or a total of 131 SSAP homologs.

Broad RecT Library was generated from the full alignment of Pfam family PF03837, containing 576 sequences from Pfam 31.0 (El-Gebali et al., Nucleic Acids Res. 47, D427-D432 (2019)). Using ETE 3, a phylogenetic tree made by FastTree and accessed from the Pfam31.0 database was pruned, and from it a maximum diversity subtree of 100 members was identified (Huerta-Cepas et al., Mol. Biol. Evol. 33, 1635-1638 (2016)). Five members of this group were found in Library 51, and so were excluded, and in their place six RecT variants from Streptomyces phages and eight other RecT variants were added that had previously reported activity or were otherwise of interest (Zhang et al., Nat. Genet. 20, 123-128 (1998); Sun et al., Appl. Microbiol. Biotechnol. 99, 5151-5162 (2015); Datta et al., Proc. Natl. Acad. Sci. U.S.A. 105, 1626-1631 (2008); van Pijkeren et al., Bioengineered 3, 209-217 (2012); van Kessel et al., Nat. Methods 4, 147-152 (2007)), bringing the library size to 109.

### Library Cloning

Diverse collections of SSAPs (PF03837) and SSBs (PF00436) were sourced from the Pfam database. A collection of ˜200 SSAPs was chosen to maximize for diversity of protein sequence. SSBs were then chosen from the organism (or a phylogenetically proximal organism) from which the SSAPs were sourced. Genes were codon-optimized for E. coli and synthesized by Twist Bioscience Corp. Genes were cloned into an entry vector by Gibson Assembly and then moved into vectors compatible with each of the three species by Golden Gate Assembly.

Broad SSAP Library and Broad RecT Library variants with a DNA barcode 22 nucleotides downstream of the stop codon were codon-optimized for E. coli and synthesized by Gen9 (S1) or Twist (S2). Synthesized DNA was amplified by PCR (NEB Q5 polymerase) and cloned into pDONR/Zeo (Thermo) by Gibson Assembly (NEB HiFi DNA Assembly Master Mix) and then moved into pARC8-DEST for arabinose-inducible expression. pARC8-DEST was engineered from a pARC8 plasmid (Choe et al., Biochem. Biophys. Res. Commun. 334, 1233-1240 (2005)) that shows good inducible expression in E. coli by moving Gateway sites (attR1/attR2), a CHL marker, and a ccdB counter-selection marker downstream of the pBAD-araC regulatory region (FIGS. 10A-10B). This enabled easy, one-step cloning of the entire library into pARC8-DEST by Gateway cloning (Thermo). The Gateway reaction was transformed into E. cloni Supreme electrocompetent cells (Lucigen), providing >10,000× coverage of both libraries in total transformants.

### Library Selection

Native resistance alleles were identified in each of the three species for resistance to rifampicin (rif) at the rpoB locus or streptomycin (stm) at the rpsL locus. The concentration of antibiotic necessary to confer a selective benefit to the resistant allele was determined for each strain. Libraries were transformed into the respective strains with at least 10× coverage, and ten successive cycles of MAGE editing followed by antibiotic selection were conducted to select for the SSAPs or SSAP/SSB pairs that most effectively conferred the antibiotic resistant allele via oligonucleotide-mediated recombineering. Rif and stm selections were performed in a non-resistant organism, and following these two rounds of selection, the plasmid library was mini-prepped and transformed back into the naïve parent strain. In this way, ten rounds of selection were performed two at a time. Fresh plasmid preparations and transformations were performed every two selection steps. In E. coli five different selective alleles were used, and so only one mini-prep and retransformation was necessary.

Libraries were mini-prepped (NEB Monarch Kit) and electroporated into the SEER chassis with more than 1,000-fold coverage. Five cycles of oligo-mediated recombineering followed by antibiotic selection were then conducted (FIG. 1B). 5 μl of the 5 ml recovery from the recombineering step was immediately plated onto LBL+selective antibiotic plates to estimate the total throughput of the selective step. This allowed us to ensure that the library was never bottlenecked—the first round of selection was the most stringent, but it was ensured that there was >500× coverage at this stage. Following five rounds of selection, the plasmid library was mini-prepped and transformed back into the naïve parent strain, followed by five further rounds of selection (ten in total). After each selective step a 100 μl aliquot of the antibiotic-selected recovery was frozen down at −80° C. in 25% glycerol for analysis by NGS.

### Efficiency Testing

The efficiency of each SSAP or SSAP/SSB pair was measured by expressing them off of their host-specific plasmid in the naïve parent strain and running a recombineering cycle with an oligo that confers a 4-nucleotide non-coding mismatch in a non-essential gene. The allele was then amplified by PCR and editing efficiency was measured by next-generation sequencing.

### Next Generation Sequencing of Libraries

Primers were designed to amplify a 215 bp product containing the barcode region of the SSAP libraries from the pARC8 plasmid and to add on Illumina adaptors. PCR amplification was done with Q5 polymerase (NEB) performed on a LightCycler 96 System (Roche), with progress tracked by SYBR Green dye and amplification halted during the exponential phase. Barcoding PCR for Illumina library prep was performed as just described, but with NEBNext Multiplex Oligos for Illumina Dual Index Primers Set 1 (NEB). Barcoded amplicons were then purified with AMPure XP magnetic beads (Beckman Coulter), pooled, and the final pooled library was quantified with the NEBNext Library Quant Kit for Illumina (NEB). The pooled library was diluted to 4 nM, denatured, and a paired end read was run with a MiSeq Reagent Kit v3, 150 cycles (Illumina). Sequencing data was downloaded from Illumina, sequences were cleaned with Sickle (Joshi et al., Sickle: A sliding-window, adaptive, quality-based trimming tool for FastQ files. (2019)) and analyzed with custom scripts written in Python.

Measuring recombineering efficiency in E. coli by NGS

To measure single locus editing, a recombineering cycle was run with an oligo that confers a single base pair non-coding mismatch in a non-essential gene. The allele was then amplified by PCR and editing efficiency was measured by NGS as described above. To test multiplex editing, the concentration of oligo was held fixed (10 μM in the final electroporation mixture), but the total number of oligos in the mixture was varied. Pools of oligos to test editing at 5, 10, 15, or 20 alleles simultaneously were designed so as to space the edits relatively evenly around the genome. The 5-oligo pool contained oligo #'s 3,7,11,15,17, the 10-oligo pool added oligo #'s 1,5,9,13,19, the 15-oligo pool added oligo #'s 4,8,12,16,18, and the final 20-oligo pool contained silent mismatch MAGE oligos. One locus (locus 8) showed major irregularities when sequenced, and so it was eliminated from the analysis.

DIvERGE-Based Simultaneous Mutagenesis of gyrA, gyrB, parE, and parC

A single round of DIvERGE mutagenesis was carried out to simultaneously mutagenize gyrA, gyrB, parE, and parC in E. coli MG1655 by the transformation of an equimolar mixture of 130 soft-randomized DIvERGE oligos, tiling the four target genes. The sequences and composition of these oligos were published previously (Nyerges, A., et. al, PNAS, 2018). To perform DIvERGE, 4 μl of this 100 μM oligo mixture was electroporated into E. coli K-12 MG1655 cells expressing Redβ from pORTMAGE311B, PapRecT from pORTMAGE312B, or CspRecT from pORTMAGE-Ec1, in 5 parallel replicates according to a previously described protocol (Szili et al., Antimicrob. Agents Chemother. AAC.00207-19 (2019)). Following electroporation, the replicates were combined into 10 ml fresh TB media. Following recovery for 2 hours, cells were diluted by the addition of 10 ml LB and allowed to reach stationary phase at 37° C., 250 rpm. Library generation experiments were performed in triplicates. Following library generation, 1 mL of outgrowth from each library was subjected to 250, 500, and 1,000 ng/mL ciprofloxacin (CIP) stresses on 145 mm-diameter LB-agar plates. Colony counts were determined after 72 hours of incubation at 37° C., and individual colonies were subjected to further genotypic (i.e., capillary DNA sequencing) analysis and phenotypic (i.e., Minimum Inhibitory Concentration) measurements.

pORTMAGE Plasmid Construction and Optimization

Cloning reactions were performed with Q5 High-Fidelity Master Mix and HiFi DNA Assembly Master Mix (New England Biolabs). pORTMAGE312B (Addgene plasmid) and pORTMAGE-Ec1 (Addgene plasmid) were constructed by replacing the Redβ open reading frame (ORF) of pORTMAGE311B plasmid (Addgene plasmid 120418) (Szili et al., bioRxiv 495630 (2018) doi:10.1101/495630) with PapRecT and CspRecT respectively. pORTMAGE-Pa1 was constructed in many steps: i.) the Kanamycin resistance cassette and the RSF1010 origin-of-replication on pORTMAGE312B with Gentamicin resistance marker and pBBR1 origin-of-replication, amplified from pSEVA631 (Martinez-Garcia et al., Nucleic Acids Res. 43, D1183-D1189 (2015)), ii.) optimization of RBSs in pORTMAGE-Pa1 was done by designing a 30-nt optimal RBS in front of the SSAP ORF and in between the SSAP and MutL ORFs with an automated design program, De Novo DNA (Salis et al., Nat. Biotechnol. 27, 946-950 (2009)), iii.) PaMutL was amplified from Pseudomonas aeruginosa genomic DNA and cloned in place of EcMutL_E32K, and finally iv.) PaMutL was mutated by site-directed mutagenesis to encode E36K. Ssr and Rec2 were ordered as gblocks from IDT and cloned in place of PapRecT into earlier versions of pORTMAGE-Pa1 for the comparisons in FIG. 12.

### Measuring Recombineering Efficiency in Gammaproteobacteria by Selective Plating

Oligos were designed to introduce I) premature STOP codons into lacZ for E. coli, K. pneumoniae, and C. freundii, or II) RpsL K43→R; GyrA T83→I; ParC S83→L; RpoB D521→V, or a premature STOP codon into nfxB for P. aeruginosa. Oligo-mediated recombineering was performed as described above on all bacterial strains. After recovery overnight, cells were plated at empirically-determined dilutions to a density of 200-500 colonies per plate. In the case of LacZ screening, plating was assayed on MacConkey agar plates or on X-Gal/IPTG LB^{L} agar plates in the case of K. pneumoniae. In the case of selective antibiotic screening, cultures were plated onto both selective and non-selective plates. Selective antibiotic concentrations used were the same as those described for the selective testing above, except that in P. aeruginosa 100 μg/ml STR and 1.5 μg/ml CIP were used unless otherwise noted. Variants that were resistant to multiple antibiotics were selected on LB^{L} agar plates that contained the combination of all corresponding antibiotics. Non-selective plates were antibiotic-free LB^{L} agar plates. In all cases, allelic-replacement frequencies were calculated by dividing the number of recombinant CFUs by the number of total CFUs. Plasmid maintenance was ensured by supplementing all media and agar plates with either KAN (50 μg/ml) or GEN (20 μg/ml).
Minimum Inhibitory Concentration (MIC) Measurement in P. aeruginosa

MICs were determined using a standard serial broth microdilution technique according to the CLSI guidelines (ISO 20776-1:2006, Part 1: Reference method for testing the in vitro activity of antimicrobial agents against rapidly growing aerobic bacteria involved in infectious diseases). Briefly, bacterial strains were inoculated from frozen cultures onto MHBII agar plates and were grown overnight at 37° C. Next, independent colonies from each strain were inoculated into 1 ml MHBII medium and were propagated at 37° C., 250 rpm overnight. To perform MIC tests, 12-step serial dilutions using 2-fold dilution-steps of the given antibiotic were generated in 96-well microtiter plates (Sarstedt 96-well microtest plate). Antibiotics were diluted in 100 μl of MHBII medium. Following dilutions, each well was seeded with an inoculum of 5×10⁴ bacterial cells. Each measurement was performed in 3 parallel replicates. Plates were incubated at 37° C. under continuous shaking at 150 rpm for 18 hours in an INFORS HT shaker. After incubation, the OD₆₀₀ of each well was measured using a Biotek Synergy 2 microplate reader. MIC was defined as the antibiotic concentration which inhibited the growth of the bacterial culture, i.e., the drug concentration where the average OD600 increment of the three replicates was below 0.05.

### REFERENCES

1 Yu, D. et al. An efficient recombination system for chromosome engineering in Escherichia coli. Proceedings of the National Academy of Sciences of the United States of America 97, 5978-5983, doi:10.1073/pnas.100127597 (2000).
2 Ellis, H. M., Yu, D., DiTizio, T. & Court, D. L. High efficiency mutagenesis, repair, and engineering of chromosomal DNA using single-stranded oligonucleotides. Proceedings of the National Academy of Sciences of the United States of America 98, 6742-6746, doi:10.1073/pnas.121164898 (2001).
3 Little, J. W. An exonuclease induced by bacteriophage λ. Journal of Biological Chemistry 242, 679-686 (1967).
4 Caldwell, B. J. et al. The Redβ single strand annealing protein of bacteriophage λ carries its own mediator domain to couple DNA end-resection with annealing at the replication fork. In Submission at Nucleic Acids Research (2018).
5 Li, Z., Karakousis, G., Chiu, S. K., Reddy, G. & Radding, C. M. The beta protein of phage lambda promotes strand exchange. Journal of molecular biology 276, 733-744, doi:10.1006/jmbi.1997.1572 (1998).
6 Murphy, K. C. Lambda Gam protein inhibits the helicase and chi-stimulated recombination activities of Escherichia coli RecBCD enzyme. Journal of bacteriology 173, 5808-5821, doi:10.1128/jb.173.18.5808-5821.1991 (1991).
7 Costantino, N. & Court, D. L. Enhanced levels of λ Red-mediated recombinants in mismatch repair mutants. Proceedings of the National Academy of Sciences of the United States of America, doi:10.1073/pnas.2434959100 (2003).
8 Mosberg, J. A., Gregg, C. J., Lajoie, M. J., Wang, H. H. & Church, G. M. Improving lambda red genome engineering in Escherichia coli via rational removal of endogenous nucleases. PloS one 7, doi:10.1371/journal.pone.0044638 (2012).
9 Lajoie, M. J. et al. Genomically recoded organisms expand biological functions. Science (New York, N.Y.) 342, 357-360, doi:10.1126/science.1241459 (2013).
10 Mandell, D. J. et al. Biocontainment of genetically modified organisms by synthetic protein design. Nature 518, 55-60, doi:10.1038/nature14121 (2015).
11 Pirman, N. L. et al. A flexible codon in genomically recoded Escherichia coli permits programmable protein phosphorylation. Nature Communications, doi:10.1038/ncomms9130 (2015).
12 Wannier, T. M. et al. Adaptive evolution of genomically recoded Escherichia coli. Proceedings of the National Academy of Sciences of the United States of America 115, 3090-3095, doi:10.1073/pnas.1715530115 (2018).
13 Wang, H. H. et al. Programming cells by multiplex genome engineering and accelerated evolution. Nature 460, 894 (2009).
14 Ioerger, T. R. et al. Identification of new drug targets and resistance mechanisms in Mycobacterium tuberculosis. PLOS ONE, doi:10.1371/journal.pone.0075245 (2013).
15 Pijkeren, J.-P. & Britton, R. A. High efficiency recombineering in lactic acid bacteria. Nucleic Acids Research 40, doi:10.1093/nar/gks147 (2012).
16 Huttenhower, C. et al. Structure, function and diversity of the healthy human microbiome. Nature 486, 207-214, doi:10.1038/nature11234 (2012).
17 Keseler, I. M. et al. The EcoCyc database: reflecting new knowledge about Escherichia coli K-12. Nucleic Acids Research 45, doi:10.1093/nar/gkw1003 (2016).
18 Yarza, P. et al. Uniting the classification of cultured and uncultured bacteria and archaea using 16S rRNA gene sequences. Nature Reviews Microbiology 12, 635, doi:10.1038/nrmicro3330 (2014).
19 Opijnen, V. T., Bodi, K. L. & Camilli, A. Tn-seq: high-throughput parallel sequencing for fitness and genetic interaction studies in microorganisms. Nature Methods 6 (2009).
20 Gilbert, L. A. et al. Genome-scale CRISPR-mediated control of gene repression and activation. Cell (2014).
21 Baba, T. et al. Construction of Escherichia coli K-12 in-frame, single-gene knockout mutants: the Keio collection. Molecular Systems Biology (2006).
22 Price, M. N. et al. Mutant phenotypes for thousands of bacterial genes of unknown function. Nature 557, 503-509, doi:10.1038/s41586-018-0124-0 (2018).
23 Pawluk, A. et al. Inactivation of CRISPR-Cas systems by anti-CRISPR proteins in diverse bacterial species. Nature Microbiology 1, 16085, doi:10.1038/nmicrobiol.2016.85 (2016).
24 Evers, B. et al. CRISPR knockout screening outperforms shRNA and CRISPRi in identifying essential genes. Nature Biotechnology, doi:10.1038/nbt.3536 (2016).
25 Macosko, E. Z. et al. Highly parallel genome-wide expression profiling of individual cells using nanoliter droplets. Cell 161, 1202-1214 (2015).
26 Kizer, L., Pitera, D. J., Pfleger, B. F. & Keasling, J. D. Application of functional genomics to pathway optimization for increased isoprenoid production. Applied and Environmental Microbiology 74, 3229-3241, doi:10.1128/AEM.02750-07 (2008).
27 Kunjapur, A. M. & Prather, K. L. J. Microbial Engineering for Aldehyde Synthesis. Applied and Environmental Microbiology 81, 1892-1901, doi:10.1128/AEM.03319-14 (2015).
28 Li, Q., Du, W. & Liu, D. Perspectives of microbial oils for biodiesel production. Applied Microbiology and Biotechnology 80, 749-756, doi:10.1007/s00253-008-1625-9 (2008).
29 Kunjapur, A. M., Tarasova, Y. & Prather, K. L. J. Synthesis and Accumulation of Aromatic Aldehydes in an Engineered Strain of Escherichia coli. Journal of the American Chemical Society 136, 11644-11654, doi:10.1021/ja506664a (2014).
30 Pitera, D. J., Paddon, C. J., Newman, J. D. & Keasling, J. D. Balancing a heterologous mevalonate pathway for improved isoprenoid production in Escherichia coli. Metabolic engineering 9 (2007).
31 Temme, K., Zhao, D. & Voigt, C. A. Refactoring the nitrogen fixation gene cluster from Klebsiella oxytoca. Proceedings of the National Academy of Sciences of the United States of America 109, 7085-7090, doi:10.1073/pnas.1120788109 (2012).
32 Wang, L. et al. A minimal nitrogen fixation gene cluster from Paenibacillus sp. WLY78 enables expression of active nitrogenase in Escherichia coli. PLoS Genetics 9, doi:10.1371/journal.pgen.1003865 (2013).
33 Shiba, Y., Paradise, E. M., Kirby, J., Dai-Kyun, R. & Keasling, J. D. Engineering of the pyruvate dehydrogenase bypass in Saccharomyces cerevisiae for high-level production of isoprenoids. Metabolic Engineering 9, 160-168 (2007).
34 Wang, H. H. et al. Genome-scale promoter engineering by coselection MAGE. Nature Methods 9, 591-593, doi:10.1038/nmeth.1971 (2012).
35 Isaacs, F. J. et al. Precise manipulation of chromosomes in vivo enables genome-wide codon replacement. Science (New York, N.Y.) 333, 348-353, doi:10.1126/science.1205822 (2011).
36 Chmielewska-Jeznach, M., Bardowski, J. K. & Szczepankowska, A. K. Molecular, physiological and phylogenetic traits of Lactococcus 936-type phages from distinct dairy environments. Scientific reports 8, 12540, doi:10.1038/s41598-018-30371-3 (2018).

### Example 10

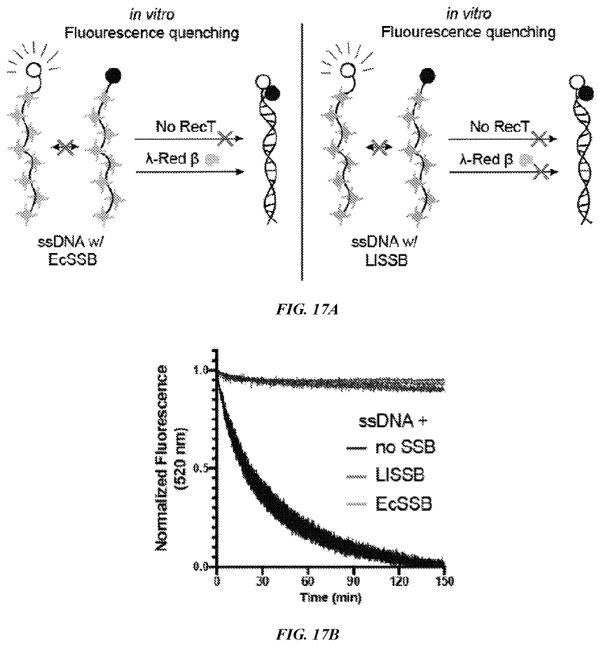

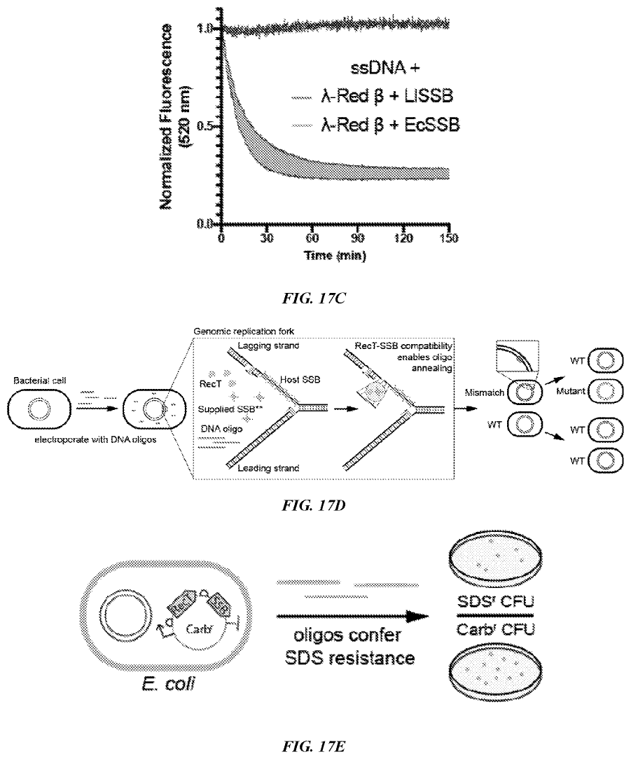

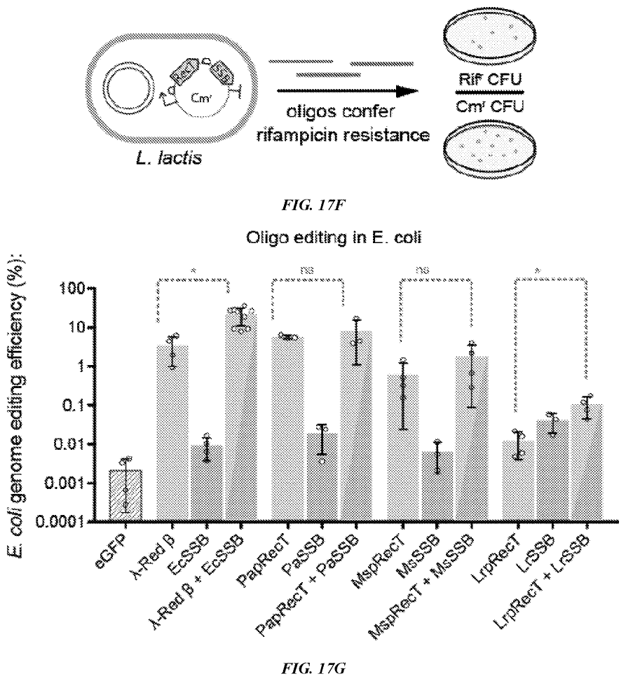

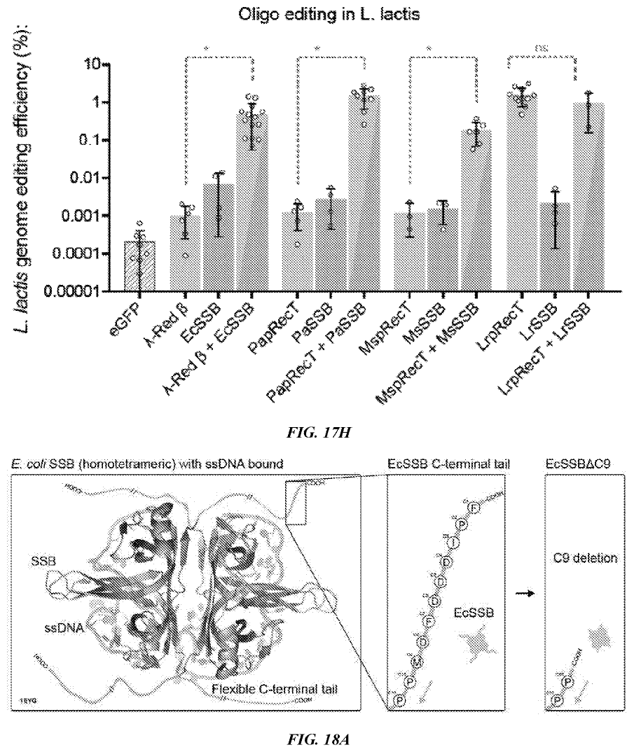

To understand the host-tropism displayed by RecT's, a simplified in-vitro model of oligonucleotide annealing that includes bacterial SSBs, a key host protein that coats ssDNA at the replication fork was developed. It was first whether two 90 bp oligos could anneal if they were pre-coated with SSB. SSBs was purified from E. coli (gram-negative), where most recombineering work has been performed, and L. lactis (gram-positive), a lactic acid bacterium phylogenetically distantly related to E. coli. Using fluorescence quenching to measure annealing, it was found that while the free oligos annealed together slowly (FIGS. 17A-17B), both EcSSB and L1SSB completely inhibited oligonucleotide annealing. It was next tested capacity of a phage RecT protein to overcome this SSB-mediated inhibition of annealing. Thus, Redβ, which is not broadly portable, but mediates efficient oligonucleotide annealing in E. coli, was purified. It was found that adding Redβ overcame the inhibitory effect of EcSSB but not L1SSB, rapidly annealing the EcSSB-coated two oligos together (FIGS. 17A and 17C). These preliminary results gave us an indication that while bacterial SSBs inhibit oligonucleotide annealing in vitro, RecTs overcome the inhibitory effect in an SSB-specific manner.

To validate this result in vivo, an assay was developed to measure the portability of RecT proteins. Four variants known to enable high genome editing efficiency were selected (Redβ and PapRecT in E. coli, LrpRecT in L. lactis, and MspRecT in M. smegmatis), and tested codon optimized versions of all four in both E. coli (gram-negative), and L. lactis (gram-positive). Genome editing efficiency was measured by introducing oligos encoding known antibiotic resistance mutations, and compared the antibiotic resistant cell counts to the total number of viable cells in the population (FIGS. 17E-17F). In E. coli, Redβ and PapRecT functioned well, and improved oligo incorporation 1600-fold and 2700-fold respectively, while MspRecT (290-fold improvement) and LrpRecT (5.6-fold improvement) were less effective (FIG. 17G). In L. lactis, LrpRecT was the only functional homolog, and improved oligo incorporation 7,700-fold, while the three other RecT variants were nearly non-functional, improving oligo incorporation less than 7-fold (FIG. 17H). No RecT protein functioned well both in E. coli and L. lactis. This agrees with previous studies, which have found that RecT proteins are usually not portable between distantly related bacterial species.

If interaction with the bacterial SSB is required for phage RecT functionality, one solution to establishing recombineering in a new species would be to replace the host SSB with one compatible with the chosen RecT. However, SSB proteins are essential, and mutations to SSB can result in severe growth defects. Therefore, it was evaluated if temporary overexpression of an exogenous SSB could supply the necessary requirements for recombineering and improve the activity of non-host compatible RecTs. SSBs corresponding to each RecT protein were synthesized and tested the activity of all four cognate RecT-SSB pairs in L. lactis and E. coli. Co-expression of a cognate bacterial SSB improved the genome editing efficiencies of all RecTs with low host-compatibility (FIGS. 17G-17H). The best performing pairs, Redβ+EcSSB and PapRecT+PaSSB demonstrated 483-fold and 1,168-fold improved editing efficiencies over the RecT proteins alone in L. lactis, and still maintained high activity in E. coli (FIGS. 17G-17H). In E. coli, co-expression with cognate SSBs also significantly reduced the toxicity of these two pairs (data not shown). These results, especially in L. lactis, indicate that the presence of a cognate bacterial SSB can overcome the host incompatibility of RecT proteins if moved to new species.

### Example 11

It was next investigated which domains on SSB were involved in mediating the RecT protein interaction. A SSB domain-specific model for understanding RecT protein portability would be far more informative than previous models, which relied on phylogenetic relationships between the host organisms. RecT proteins have been shown to function in species with SSBs with relatively divergent sequences. Therefore, there was interest in identifying conserved domains responsible for maintaining the RecT protein interaction. For example, while Redβ works well in E. coli, Salmonella enterica, and Citrobacter freundii which have SSBs with 88% identity, PapRecT works in E. coli and Pseudomonas aeruginosa, which have SSBs of only 59% identity. To investigate the specific residues involved, the genome editing assay was used in L. lactis and the effect of co-expressing RecT proteins with non-cognate or mutated SSBs was evaluated.

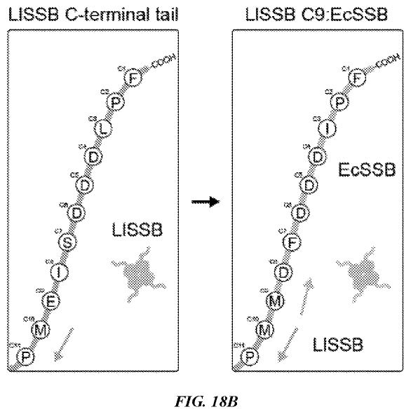

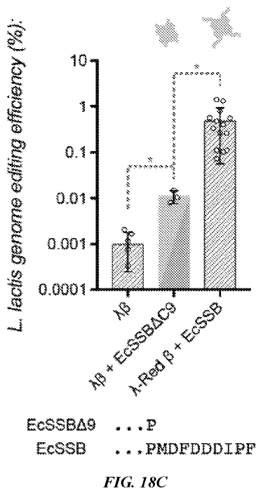

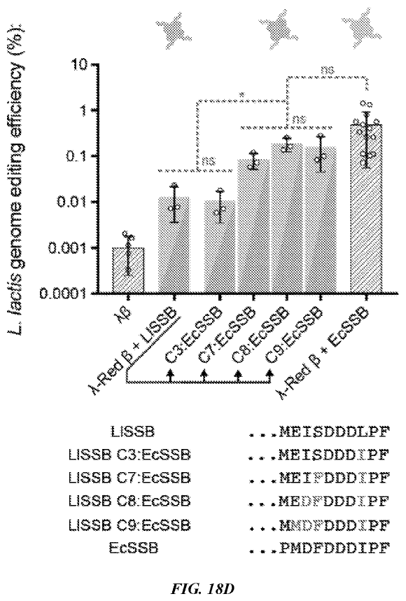

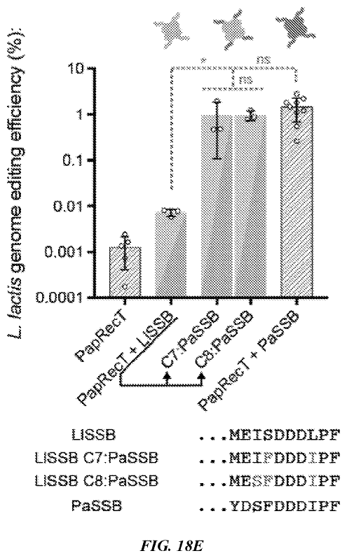

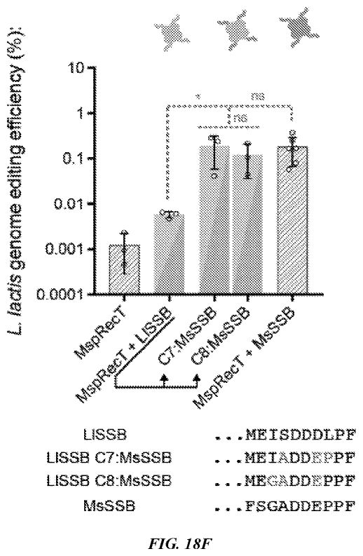

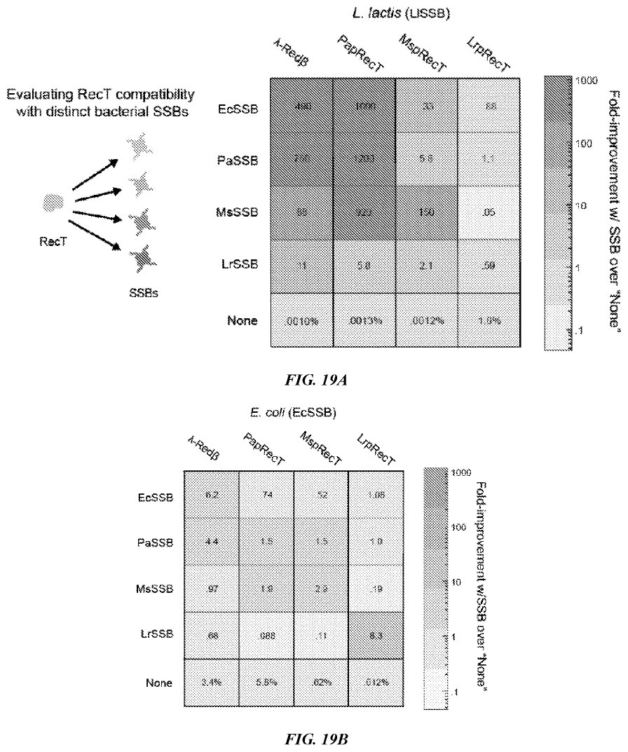

The importance of the SSB C-terminal tail was evaluated by coexpressing Redβ in L. lactis, along with a version of EcSSB that had a 9-amino-acid C-terminal deletion (EcSSBΔ9) (FIG. 18A). In L. lactis, the genome editing efficiency of Redβ with EcSSBΔ9 was 44-fold lower than Redβ with EcSSB, indicating a key role for the C-terminal domain in the SSB-mediated efficiency improvement (FIG. 18c). Next, Redβ was co-expressed with the L. lactis SSB (L1SSB). Co-expression of Redβ with L1SSB performed similarly to Redβ with EcSSBΔ9, and improved genome editing efficiency 38.5-fold less than Redβ with EcSSB. Then, Redβ was co-expressed with chimeric versions of the L1SSB, where up to 9 amino acids of the L1SSB C-terminal tail were replaced with their corresponding residues from EcSSB. Swapping the last 7 C-terminal residues (L1SSB C7:EcSSB) improved editing rates to within 5.9-fold of Redβ with EcSSB, and swapping the last 8 C-terminal residues (L1SSB C8:EcSSB) improved editing rates within 2.6-fold of Redβ with EcSSB. These results support a model where Redβ specifically recognizes at minimum the 7 C-terminal acids of E. coli SSB, but not that of L. lactis SSB.

To evaluate if the SSB C-terminal 7 amino acids also affected the compatibility of the other two non-host compatible RecT proteins, similar SSB-chimera experiments were performed with PapRecT and MspRecT in L. lactis. The genome editing efficiency of PapRecT with the L. lactis SSB was 135-fold less than when using the cognate pair (FIG. 18E). However, this defect was completely recovered when PapRecT was co-expressed with L. lactis SSB chimeras where either the last 7 or 8 C-terminal resides were replaced (L1SSB C7:PaSSB, L1SSB C8:PaSSB) (FIG. 18E). For MspRecT, the genome editing efficiency with L1SSB was 33-fold lower than when using the cognate pair (FIG. 18F). Again, the defect was completely recovered when MspRecT was co-expressed with L. lactis SSB chimeras where either the last 7 or 8 C-terminal resides were replaced (L1SSB C7:MsSSB, L1SSB C8:MsSSB) (FIG. 18F). Since the chimeric L1SSBs greatly improved the functionality of non-host compatible RecT proteins, while the wild-type L1SSB did not, the RecT-SSB interaction seems to be both specific and relatively modular, with the 7 C-terminal amino acids acting as the critical interaction domain.

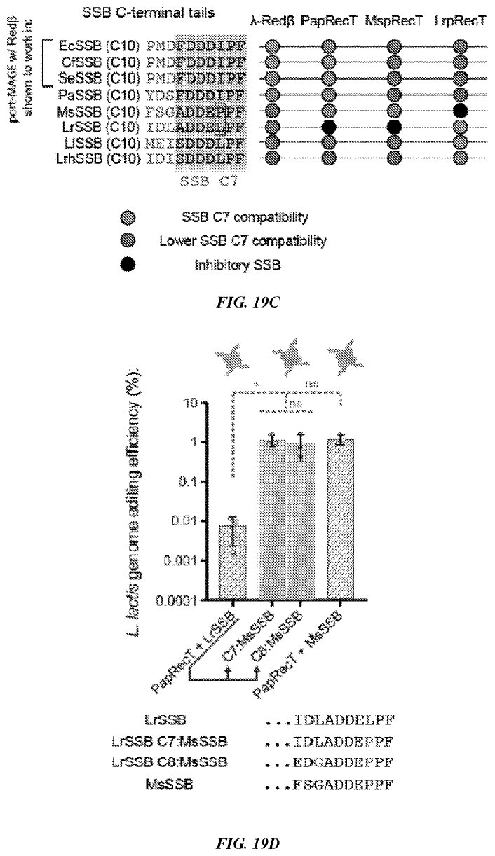

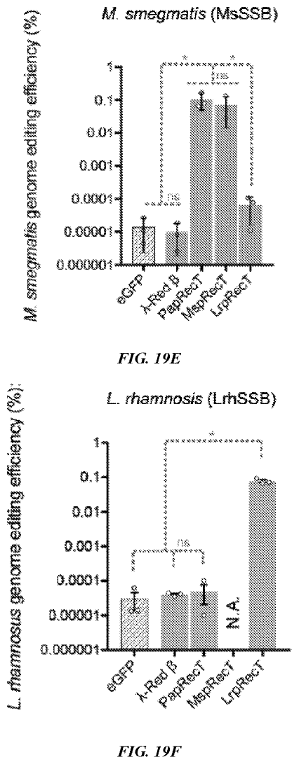

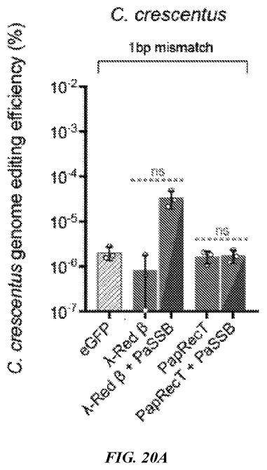

These results provide a molecular basis for the portability of RecT proteins between species which have host SSBs with a conserved C-terminal tail. Specifically, although the SSBs have only 59% identity, the P. aeruginosa and E. Coli SSBs have a perfectly conserved 7 amino acid C-terminal tail domain (FIG. 19C), supporting the functionality of PapRecT in E. coli. Additionally, E. coli, Salmonella enterica and Citrobacter freundii SSBs all have a perfectly conserved 7 amino acid C-terminal tails, supporting the portability of Redβ between these species (FIG. 19C).

### Example 12

Some RecT proteins are known to be portable between species which have distinct SSB C-terminal tails. To better characterize the network of RecT-SSB compatibility among the proteins analyzed here, all four RecTs were co-expressed with all four SSBs in both E. coli and L. lactis (FIGS. 19A-19B). It was found that the effects of PaSSB and EcSSB on RecT-mediated editing efficiency were relatively interchangeable, as might be expected since they share the same 7 amino acid C-terminal tail (FIG. 19C). Interestingly, PapRecT displayed the characteristics of a more portable RecT protein, and showed compatibility with MsSSB and EcSSB/PaSSB, even though their 7AA C-terminal tail sequences are distinct (FIGS. 19A and 19C). Importantly, co-expressing PapRecT with LrSSB did not provide a substantial improvement in genome editing efficiency in L. lactis, even though the 7 C-terminal tail amino acids of LrSSB differ only by a single residue from MsSSB (FIGS. 19A and 19C).

To test if PapRecT specifically interacts with the C-terminal tail of MsSSB, PapRecT was co-expressed with a chimeric version of LrSSB, with either the C7 or C8 amino acids matching that of MsSSB (FIG. 19D). The chimeric constructs demonstrated the same editing efficiency as PapRecT+MsSSB, showing that a single amino acid change was sufficient to enable compatibility between the proteins (FIG. 19D). The compatibility of PapRecT with the distinct EcSSB/PaSSB and MsSSB tails but not the LrSSB tail affirms that while the SSB C-terminal tail has a critical role in the RecT-SSB interaction, there can be flexibility in the specific motif recognized.

We next evaluated if the interaction between PapRecT and MsSSB in L. lactis indicated that PapRecT would function in M. smegmatis, where MsSSB is natively expressed. All four RecT's were tested in this species and indeed found that in M. smegmatis PapRecT enabled high efficiency editing, incorporating oligos at the same rate as MspRecT, while the other two RecT variants had much lower efficiency (FIG. 19E).

Finally, the model for RecT was used to establish recombineering in L. rhamnosus, a well-studied probiotic used to treat a variety of illnesses including diarrhea and bacterial vaginosis. Although the L. rhamnosus SSB and L. lactis SSB only have 47% identity, they share identical SSB C-terminal tail 7 amino acids. It was determined whether LrpRecT (which functions in L. lactis) is portable to L. rhamnosus, while the other RecT proteins would not be functional. The 4 RecT proteins were tested in this species and it was found that LrpRecT incorporated oligonucleotides three orders of magnitude above the background level, while Redβ and PapRecT had negligible activity and MspRecT was toxic.

### Example 13

In L. lactis, the co-expression of PapRecT and Redβ with compatible SSB's improved genome editing efficiency to a level comparable with the host-compatible LrpRecT. It was determined whether for some species, RecT-SSB pairs could provide functional recombineering capacity even if no functional RecT protein had previously been identified. Therefore, the two best-performing RecT-SSB pairs (Redβ+PaSSB, and PapRecT+PaSSB) were tested for activity in Caulobacter crescentus, a model organism for studying cell cycle regulation, replication, and differentiation.

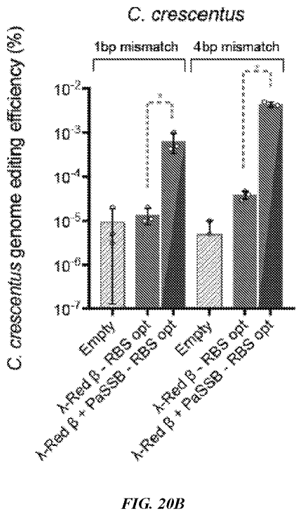

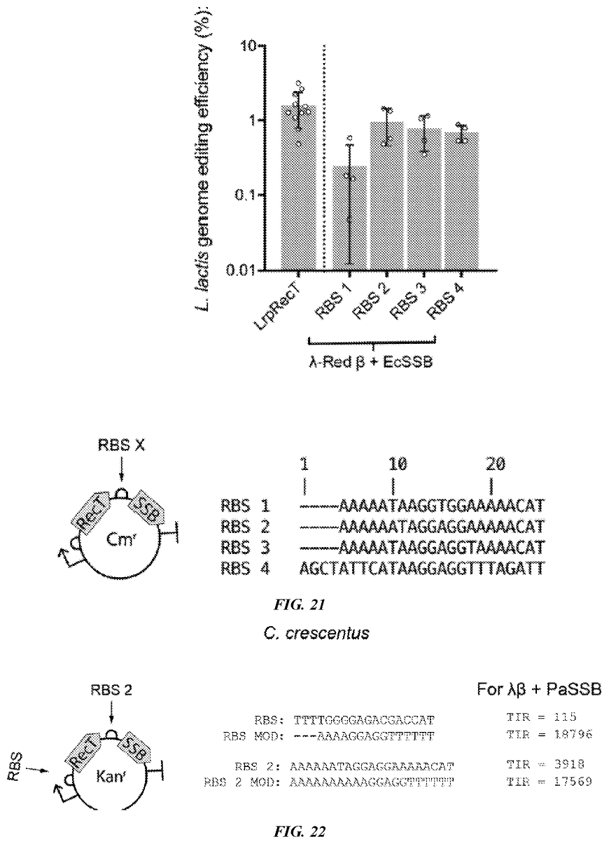

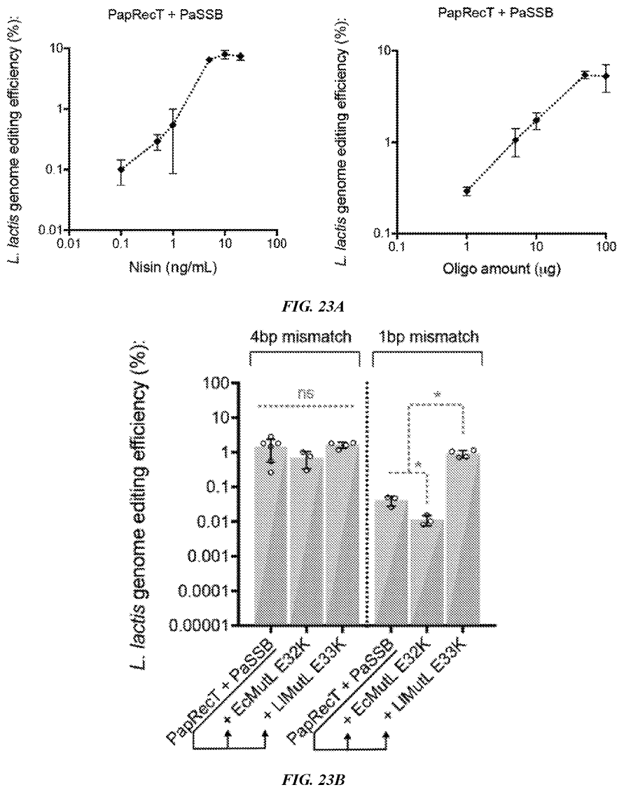

In C. crescentus, no significant editing enhancement was detected over the background with the RecT proteins alone, or PapRecT+PaSSB. As compatibility between PapRecT and PaSSB was previously observed, it seemed likely that additional factors must contribute to the incompatibility of this pair with C. crescentus. However, using Redβ+PaSSB, a 15-fold improvement over Redβ alone was observed (FIG. 20A). After expression optimization (FIG. 22) and evasion of mismatch repair, Redβ+PaSSB demonstrated 873-fold improved editing efficiency over the background level, which was 112-fold higher than Redβ alone (FIG. 20B). These results indicate that while RecT-SSB pairs are not universally portable (data not shown), the co-expression of a RecT protein with a compatible bacterial SSB will equal or surpass the editing efficiencies of RecT proteins alone.

### Example 14

In E. coli, one of the unique capabilities of recombineering is the ability to generate rationally designed or high-coverage genomic libraries. Although this technique (termed MAGE) has been used for a variety of applications including optimizing metabolic pathways, protein evolution, and saturation mutagenesis, it has only been used in a limited capacity in other species. L. lactis, a microbe distantly related to E. coli, was used to demonstrate how mismatch repair evasion and oligonucleotide library design can be used to perform high-coverage genomic mutagenesis after a functional RecT protein has been identified.

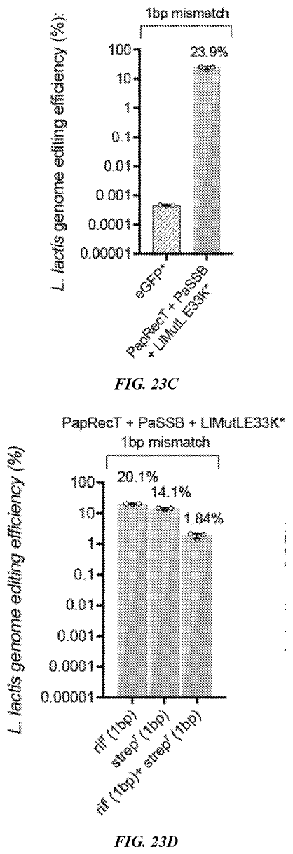

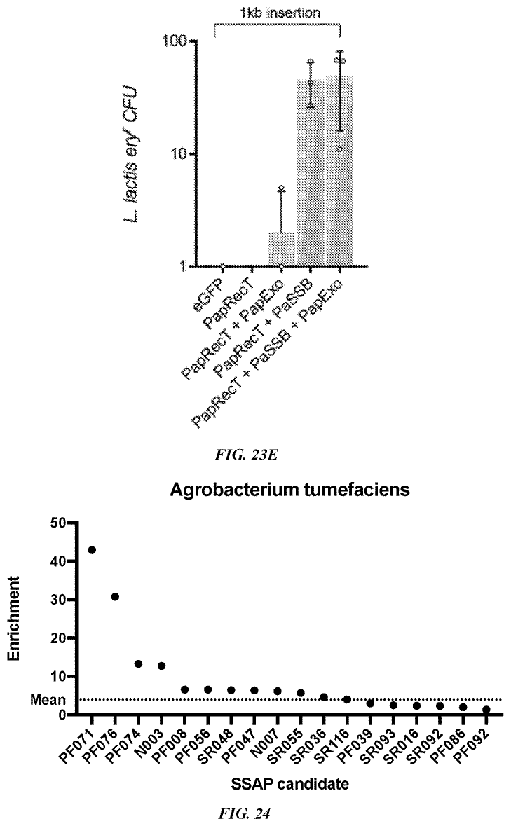

To begin, the assay in L. lactis was adapted to allow the efficient incorporation of single, double, or triple nucleotide mutations, which are normally recognized and corrected by mismatch repair pathways. The cognate pair PapRecT and PaSSB, was used and co-expressed either the dominant negative mismatch repair protein MutL.E32K from E. coli, or the host protein L. lactis MutL carrying the equivalent mutation (L1MutL.E33K, data not shown). While MutL.E32K from E. coli was nonfunctional, co-expression of LlMutL.E33K enabled the efficient introduction of 1 bp pair changes (FIGS. 23A-23E). Optimization of inducer and oligonucleotide concentrations further improved editing efficiency 26-fold (FIGS. 23A-23E).

Table 3 includes sequences that were used in Examples 10-14.

See also, e.g., Filsinger et al., bioRxiv 2020.04.14.041095 (doi.org/10.1101/2020.04.14.041095), which is herein incorporated by reference in its entirety.

TABLE 3
Additional sequences

| Gene or | Codon | | |
|---|---|---|---|
| construct | optimized | | SEQ ID |
| name | for: | Sequence | NO: |
| λBeta | L. lactis; | ATGAGTACTGCACTTGCAACATTAGCTGGCAAGTTAGCAGAG | 473 |
| | L. rhamnosus | CGTGTTGGTATGGATTCAGTCGACCCTCAGGAGCTTATAACT | |
| | | ACCTTACGTCAAACAGCGTTCAAGTGTGACGCCTCTGATGCA | |
| | | CAATTTATCGCTTTGCTTATCGTAGCTAACCAGTATGGGTTGA | |
| | | ATCCTTGGACGAAGGAGATATACGCTTTCCCGGATAAGCAGA | |
| | | ACGGTATTGTTCCTGTAGTAGGTGTCGATGGATGGAGTAGAA | |
| | | TTATCAATGAAAATCAACAGTTCGATGGCATGGACTTCGAGC | |
| | | AGGATAATGAATCATGTACCTGCCGTATATATAGAAAAGACC | |
| | | GAAATCACCCAATTTGTGTGACTGAATGGATGGATGAGTGCA | |
| | | GACGTGAGCCGTTCAAGACCCGAGAAGGCCGTGAAATCACTG | |
| | | GTCCGTGGCAATCACATCCAAAGAGAATGTTGCGTCACAAGG | |
| | | CGATGATTCAGTGCGCCCGTTTAGCTTTTGGGTTTGCTGGCAT | |
| | | TTACGACAAGGACGAAGCTGAAAGAATCGTTGAAAACACTG | |
| | | CATATACCGCTGAACGACAACCGGAGCGTGACATTACGCCAG | |
| | | TGAATGACGAGACAATGCAGGAAATTAACACGTTGTTGATTG | |
| | | CTTTGGACAAAACGTGGGACGACGACTTGTTACCACTTTGTA | |
| | | GCCAAATTTTTCGTCGAGACATTAGAGCTTCATCTGAGCTTAC | |
| | | ACAAGCTGAAGCCGTCAAGGCATTGGGGTTTTTGAAACAAAA | |
| | | AGCTACCGAACAGAAGGTAGCGGCATAA | |
| PapRecT | L. lactis; | ATGGGAACCGCCCTTACACCTCTTTTGACAAAGTTCGCAACC | 476 |
| | L. rhamnosus | AGATATGAGATGGGAACGACCCCTGAAGAGGTTGCTAATACA | |
| | | TTGAAACAAACATGCTTCAAGGGACAGGTCAACGACAGTCAA | |
| | | ATGGTAGCCCTTTTGATAGTCGCTGACCAGTACAAGTTAAAC | |
| | | CCGTTCACCAAGGAGTTGTATGCATTCCCTGACAAGAATAAT | |
| | | GGAATCGTGCCAGTTGTTGGTGTCGATGGATGGGCGAGAATA | |
| | | ATAAACGAGAACCCTCAGTTTGATGGTATGGAATTTTCTATG | |
| | | GACCAGCAGGGCACTGAGTGCACGTGCAAAATCTATCGTAAG | |
| | | GATCGTTCTCACGCAATAAGCGCTACGGAATATATGGCCGAA | |
| | | TGTAAGAGAAATACGCAACCTTGGCAAAGTCACCCACGACGT | |
| | | ATGTTAAGACATAAAGCCATGATCCAGTGTGCGCGATTAGCA | |
| | | TTCGGCTTCGCTGGTATCTACGATCAAGACGAAGCGGAACGA | |
| | | ATAGTCGAAAGAGACGTTACTCCGGCGGAGCAGTACGAAGA | |
| | | TGTCAGCGAAGCTATATGCTTGATTAAGGACAGCCCGACGAT | |
| | | GGAGGATTTACAGGCAGCGTTCAGCAATGCCTGGAAGGCGTA | |
| | | TAAAACCAAAGGTGCAAGAGACCAATTGACAGCCGCCAAGG | |
| | | ATCAGCGTAAAAAGGAATTACTTGATGCCCCAATAGATGTCG | |
| | | AGTTCGAAGAAACTGGCGATGATAGAGCAGCATAA | |
| MspRecT | L. lactis; | ATGGCAGAAAACGCCGTGACGAAACAAGACTCACCTAAAGC | 477 |
| | L. rhamnosus | CCCAGAAACGATATCACAGGTCCTTCAAGTGTTAGTACCTCA | |
| | | ATTAGCTCGAGCCGTACCTAAGGGAATGGATCCTGATAGAAT | |
| | | AGCTCGAATCGTCCAGACCGAGATCAGAAAGAGTAGAAATG | |
| | | CGAAAGCGGCGGGAATCGCCAAACAGTCATTGGATGACTGC | |
| | | ACGCAAGAAAGCTTCGCCGGGGCGTTACTTACAAGTGCGGCA | |
| | | TTGGGCCTTGAGCCAGGCGTTAACGGTGAGTGTTATCTTGTTC | |
| | | CATACAGAGACACAAGAAGAGGTGTCGTCGAGTGCCAGTTA | |
| | | ATTATCGGGTATCAAGGAATCGTTAAATTGTTTTGGCAACATC | |
| | | CGCGAGCCTCTCGAATAGACGCCCAGTGGGTGGGGGCAAAC | |
| | | GATGAATTCCATTATACAATGGGTCTTAACCCAACCTTAAAA | |
| | | CATGTAAAGGCTAAGGGAGACCGAGGAAACCCAGTATATTTT | |
| | | TATGCTATCGTAGAGGTCACGGGTGCCGAGCCTTTGTGGGAT | |
| | | GTCTTCACAGCTGATGAGATTAGAGAGTTGAGAAGAGGTAAA | |
| | | GTCGGTTCAAGCGGGGATATTAAGGATCCGCAGAGATGGATG | |
| | | GAGCGAAAGACAGCGTTAAAACAAGTGCTTAAGCTTGCTCCA | |
| | | AAAACTACTCGTTTGGACGCAGCAATACGAGCGGACGATAGA | |
| | | CCGGGAACAGATTTGTCTCAAAGCCAGGCGTTGGCATTACCT | |
| | | AGTACAGTTAAGCCAACAGCAGACTACATAGACGGTGAGATT | |
| | | GCAGAGCCACACGAGGTTGACACTCCGCCTAAAAGCAGTCGA | |
| | | GCACAACGAGCTCAGAGAGCGACTGCCCCAGCCCCAGACGTT | |
| | | CAGATGGCCAATCCGGATCAATTAAAGCGTTTGGGAGAGATT | |
| | | CAAAAAGCCGAAAAGTACAATGATGCCGACTGGTTTAAGTTC | |
| | | TTAGCTGATAGTGCAGGGGTGAAAGCGACAAGAGCAGCTGA | |
| | | TCTTACATTTGATGAAGCAAAAGCTGTAATAGATATGTTCGA | |
| | | CGGCCCAAATGCTTGA | |
| LrpRecT | L. lactis; | ATGGCTAATCAAGTAGCACAACAGCAGAAACCGACTAAGCT | 478 |
| | L. rhamnosus | AACCGATCTTGTATTAGATCGTGTTAAACAAATGCAAGACAC | |
| | | GCAGGACTTGTCACTACCCAAGAATTACAACGCTTCTAATGC | |
| | | GTTGAATGCAGCCTTTCTCGAATTACAAAAAGTACAAGACCG | |
| | | TAATCATCGGCCAGCCTTAGAAGTATGTTCTCATGACTCGATT | |
| | | GTTAAGTCCTTGTTAGATATGACACTGCAAGGGCTATCCCCA | |
| | | GCAAAAGATCAATGCTACTTCATCGTATACGGCAATGAGCTT | |
| | | CAAATGCAACGGAGCTATTTCGGTACTGTTGCAGCAGTTAAG | |
| | | CGACTGGATGGTGTTAAGAAAGTTAGGGCAGAAGTTGTTCAC | |
| | | GAAAAAGATGACTTTGAAATTGGTGCTAATGAAGACATGGAG | |
| | | CTAGTCGTTAAGAGGTTCGTTCCTAAGTTTGAAAATCAAGAT | |
| | | AATCAAATTATTGGAGCTTTTGCCATGATTAAGACTGATGAA | |
| | | GGTACTGACTTTACTGTTATGACTAAGAAAGAGATTGATCAG | |
| | | TCATGGGCACAAACACGTCAAAAAAATAACAAAGTACAGCA | |
| | | GAATTTTAGCCAAGAAATGGCAAAGCGTACTGTGCTTAATCG | |
| | | TGCCGCTAAGATGTTTATTAACACGTCTGATGATAGTGACCTT | |
| | | TTAACTGGTGCTATCAACGATACAACAAGCAACGAATACGAT | |
| | | GATGAGCGTCGAGATGTAACGCCCGTTGAGGATGAAAAACA | |
| | | AAGTACTGATAAATTGCTAGAAGGATTTCAAAAGTCACAAGA | |
| | | AGCGAAGGCTAAGTGGGTAAGTAATGATGGCAACAGCAACG | |
| | | AAGGCAAAGAAACCAGTGAAGAAGTCGCAGACGGACAAACA | |
| | | GAACTCTTCAGCGAAGGGACAATCAAACCAGCCGATGAAGCT | |
| | | GACAGCTAA | |
| λ Beta | E. coli | ATGAGTACTGCACTCGCAACGCTGGCTGGGAAGCTGGCTGAA | 479 |
| | | CGTGTCGGCATGGATTCTGTCGACCCACAGGAACTGATCACC | |
| | | ACTCTTCGCCAGACGGCATTTAAAGGTGATGCCAGCGATGCG | |
| | | CAGTTCATCGCATTACTGATCGTTGCCAACCAGTACGGCCTTA | |
| | | ATCCGTGGACGAAAGAAATTTACGCCTTTCCTGATAAGCAGA | |
| | | ATGGCATCGTTCCGGTGGTGGGCGTTGATGGCTGGTCCCGCA | |
| | | TCATCAATGAAAACCAGCAGTTTGATGGCATGGACTTTGAGC | |
| | | AGGACAATGAATCCTGTACATGCCGGATTTACCGCAAGGACC | |
| | | GTAATCATCCGATCTGCGTTACCGAATGGATGGATGAATGCC | |
| | | GCCGCGAACCATTCAAAACTCGCGAAGGCAGAGAAATCACG | |
| | | GGGCCGTGGCAGTCGCATCCCAAACGGATGTTACGTCATAAA | |
| | | GCCATGATTCAGTGTGCCCGTCTGGCCTTCGGATTTGCTGGTA | |
| | | TCTATGACAAGGATGAAGCCGAGCGCATTGTCGAAAATACTG | |
| | | CATACACTGCAGAACGTCAGCCGGAACGCGACATCACTCCGG | |
| | | TTAACGATGAAACCATGCAGGAGATTAACACTCTGCTGATCG | |
| | | CCCTGGATAAAACATGGGATGACGACTTATTGCCGCTCTGTT | |
| | | CCCAGATATTTCGCCGCGACATTCGTGCATCGTCAGAACTGA | |
| | | CACAGGCCGAAGCAGTAAAAGCTCTTGGATTCCTGAAACAGA | |
| | | AAGCCGCAGAGCAGAAGGTGGCAGCATGA | |
| PapRecT | E. coli | ATGGGTACTGCTCTAACGCCGTTATTAACCAAGTTTGCCACCC | 480 |
| | | GCTATGAGATGGGAACTACCCCCGAAGAGGTCGCTAATACGC | |
| | | TGAAACAGACTTGTTTCAAGGGCCAAGTGAACGATAGTCAGA | |
| | | TGGTAGCCCTTTTGATCGTTGCGGATCAATATAAGCTCAATCC | |
| | | ATTCACAAAAGAGCTCTACGCGTTCCCTGACAAAAATAATGG | |
| | | TATTGTTCCAGTTGTGGGAGTCGATGGTTGGGCTAGAATTATT | |
| | | AACGAGAATCCCCAGTTTGATGGGATGGAATTCAGTATGGAT | |
| | | CAACAGGGAACTGAATGCACTTGTAAAATTTACCGCAAAGAC | |
| | | CGCTCGCACGCCATCAGCGCCACCGAGTACATGGCTGAGTGC | |
| | | AAAAGGAACACTCAACCTTGGCAGTCTCACCCGCGACGTATG | |
| | | CTGCGTCATAAGGCTATGATTCAATGCGCCAGACTAGCCTTT | |
| | | GGTTTCGCGGGGATCTACGATCAGGATGAGGCCGAACGCATT | |
| | | GTTGAACGAGATGTAACTCCCGCCGAGCAATACGAGGATGTA | |
| | | TCCGAAGCGATTTGTCTGATCAAAGACTCACCAACTATGGAG | |
| | | GACTTGCAGGCCGCGTTCTCAAACGCGTGGAAAGCTTACAAG | |
| | | ACTAAAGGTGCCCGTGATCAACTGACTGCTGCTAAAGACCAG | |
| | | AGAAAAAAGGAGCTGTTGGATGCGCCCATTGATGTCGAATTC | |
| | | GAAGAAACTGGAGATGATCGTGCTGCGTAA | |
| MspRecT | E. coli | ATGGCCGAGAATGCCGTCACGAAACAGGATTCCCCTAAGGCA | 481 |
| | | CCGGAAACCATTAGTCAAGTGCTTCAGGTGCTGGTCCCACAA | |
| | | TTGGCTCGTGCAGTACCTAAAGGCATGGATCCTGATCGTATT | |
| | | GCACGTATCGTACAGACGGAGATTCGCAAATCCCGCAACGCA | |
| | | AAAGCCGCTGGAATCGCAAAGCAAAGTTTAGACGATTGCACA | |
| | | CAGGAGTCCTTTGCGGGAGCCTTACTGACCTCAGCGGCTTTA | |
| | | GGGTTAGAGCCAGGCGTCAATGGGGAGTGTTATCTGGTACCC | |
| | | TATCGTGATACACGCCGTGGTGTGGTCGAGTGCCAACTGATT | |
| | | ATTGGATATCAAGGGATTGTCAAACTTTTTTGGCAACATCCGC | |
| | | GCGCGAGCCGCATCGATGCGCAATGGGTTGGCGCGAACGAC | |
| | | GAGTTCCATTATACGATGGGACTTAATCCTACCTTGAAACAT | |
| | | GTAAAGGCAAAAGGTGATCGTGGAAACCCGGTCTACTTTTAC | |
| | | GCCATCGTCGAGGTGACCGGTGCTGAGCCCTTATGGGATGTT | |
| | | TTTACTGCTGATGAAATTCGTGAACTTCGTCGTGGCAAGGTTG | |
| | | GATCGTCTGGAGATATTAAGGACCCCCAGCGTTGGATGGAAC | |
| | | GCAAGACAGCATTGAAACAGGTACTGAAATTGGCTCCCAAAA | |
| | | CCACACGCCTGGATGCGGCGATCCGCGCTGATGATCGTCCAG | |
| | | GGACTGACCTTTCACAGTCGCAGGCTCTGGCCTTACCGTCTAC | |
| | | CGTTAAGCCTACCGCAGACTATATTGATGGGGAGATCGCCGA | |
| | | ACCGCATGAAGTCGATACACCACCAAAGAGTTCACGTGCTCA | |
| | | ACGCGCACAACGTGCCACGGCACCGGCTCCTGATGTGCAAAT | |
| | | GGCCAACCCCGACCAATTGAAGCGTCTGGGGGAGATCCAAA | |
| | | AGGCGGAGAAGTACAATGATGCCGACTGGTTCAAGTTTTTGG | |
| | | CGGACTCCGCCGGTGTGAAAGCGACGCGTGCTGCTGATCTTA | |
| | | CGTTTGATGAAGCAAAGGCTGTAATCGACATGTTTGATGGGC | |
| | | CAAACGCGTGA | |
| LrpRecT | E. coli | ATGGCGAATCAAGTTGCACAGCAACAAAAACCGACAAAATT | 482 |
| | | AACCGATCTGGTTTTGGATAGAGTCAAGCAGATGCAAGACAC | |
| | | CCAGGACCTTAGCCTTCCGAAAAACTATAACGCATCCAATGC | |
| | | ACTGAATGCCGCGTTTTTAGAATTGCAGAAGGTACAAGACCG | |
| | | GAACCACAGACCAGCACTGGAAGTCTGCTCGCACGATTCTAT | |
| | | TGTAAAATCGCTGTTGGACATGACTTTGCAGGGCTTATCCCCT | |
| | | GCGAAGGATCAGTGTTACTTCATAGTATATGGCAATGAGTTA | |
| | | CAGATGCAGAGATCTTATTTCGGGACTGTCGCGGCAGTTAAA | |
| | | AGATTAGATGGGGTGAAGAAGGTCCGGGCGGAAGTCGTGCA | |
| | | TGAAAAGGACGACTTCGAAATTGGCGCCAATGAAGACATGG | |
| | | AGCTGGTAGTGAAACGGTTTGTACCAAAGTTCGAAAATCAAG | |
| | | ACAACCAAATAATAGGGGCGTTCGCAATGATTAAAACGGATG | |
| | | AAGGTACGGACTTCACAGTTATGACGAAAAAGGAAATAGAT | |
| | | CAAAGTTGGGCGCAAACACGCCAGAAGAACAATAAGGTACA | |
| | | GCAGAACTTTAGTCAAGAAATGGCGAAACGTACAGTCCTTAA | |
| | | TCGTGCCGCTAAGATGTTTATAAACACTTCAGACGATTCGGA | |
| | | CTTATTAACCGGGGCCATAAATGACACGACCTCAAACGAGTA | |
| | | TGACGATGAAAGAAGAGATGTGACACCAGTCGAGGACGAAA | |
| | | AACAGAGCACGGATAAATTACTGGAGGGGTTTCAGAAGTCGC | |
| | | AGGAGGCGAAAGCAAAAGGGGTTAGTAACGACGGAAACAGT | |
| | | AATGAGGGAAAAGAGACAAGCGAGGAGGTGGCCGATGGACA | |
| | | GACGGAACTGTTCTCTGAAGGTACTATTAAACCAGCAGATGA | |
| | | AGCGGATAGCTAA | |
| MspRecT | M. | ATGGCAGAAAACGCTGTAACCAAGCAAGACAGTCCCAAAGC | 483 |
| | smegmatis | GCCCGAGACCATATCGCAGGTATTGCAAGTGTTAGTGCCTCA | |
| | | ATTAGCAAGAGCAGTCCCCAAAGGGATGGATCCTGACAGAAT | |
| | | AGCACGCATAGTGCAGACCGAAATACGTAAGTCCCGTAATGC | |
| | | CAAAGCTGCCGGCATCGCAAAACAATCGTTAGATGATTGTAC | |
| | | CCAGGAGAGTTTTGCCGGGGCGCTGCTTACCTCAGCAGCCTT | |
| | | AGGTCTGGAACCAGGAGTTAACGGAGAGTGTTATTTGGTCCC | |
| | | ATACCGGGATACTCGTCGCGGAGTTGTTGAGTGCCAACTTAT | |
| | | TATCGGTTACCAGGGAATAGTGAAGTTGTTCTGGCAACACCC | |
| | | TCGTGCGTCCCGGATTGACGCGCAGTGGGTAGGTGCAAACGA | |
| | | CGAATTCCACTACACTATGGGCCTTAATCCGACACTTAAACA | |
| | | CGTCAAAGCGAAAGGGGATAGAGGAAACCCGGTGTACTTTTA | |
| | | TGCAATTGTTGAGGTTACTGGAGCAGAGCCGTTATGGGATGT | |
| | | CTTTACTGCCGATGAGATACGCGAGCTGCGTCGTGGCAAGGT | |
| | | CGGGAGTTCAGGGGACATTAAAGATCCCCAACGGTGGATGG | |
| | | AGCGGAAGACTGCGCTGAAACAGGTGTTGAAGTTGGCCCCCA | |
| | | AAACGACCCGCCTTGACGCTGCAATCCGGGCGGATGATCGTC | |
| | | CTGGGACTGACCTGTCCCAAAGCCAAGCCTTAGCCCTTCCAA | |
| | | GTACTGTCAAGCCAACCGCAGATTACATTGACGGGGAAATCG | |
| | | CAGAACCGCACGAAGTTGACACTCCTCCGAAGAGTAGCCGCG | |
| | | CACAACGTGCCCAGCGCGCGACGGCACCAGCGCCGGATGTGC | |
| | | AGATGGCAAATCCTGACCAACTTAAAAGACTGGGAGAGATA | |
| | | CAGAAAGCAGAGAAGTACAACGACGCAGATTGGTTTAAGTTT | |
| | | TTGGCGGACAGCGCTGGCGTCAAAGCAACTCGTGCGGCCGAC | |
| | | TTGACCTTTGACGAAGCGAAGGCGGTCATAGATATGTTTGAT | |
| | | GGTCCAAACGCCTGA | |
| PapRecT | M. smegmatis | ATGGGCACCGCCCTGACCCCACTCTTGACCAAGTTTGCCACG | 484 |
| | | CGGTATGAGATGGGCACCACCCCAGAGGAAGTGGCGAACAC | |
| | | CCTCAAGCAGACCTGCTTTAAGGGTCAGGTCAATGATAGCCA | |
| | | GATGGTGGCCTTGCTGATCGTCGCGGACCAATATAAACTGAA | |
| | | TCCATTTACCAAGGAACTCTATGCGTTTCCGGATAAGAACAA | |
| | | TGGTATTGTCCCCGTCGTCGGCGTGGACGGTTGGGCGCGGAT | |
| | | CATTAACGAGAACCCCCAATTCGATGGCATGGAATTTTCGAT | |
| | | GGACCAGCAAGGGACCGAATGCACCTGCAAAATCTACCGGA | |
| | | AAGACCGTAGCCATGCCATTAGCGCCACGGAGTATATGGCCG | |
| | | AATGTAAACGTAATACGCAGCCATGGCAATCCCATCCACGCC | |
| | | GGATGTTGCGCCACAAGGCGATGATCCAGTGTGCGCGGTTGG | |
| | | CCTTTGGTTTCGCGGGCATCTATGACCAGGACGAAGCGGAAC | |
| | | GCATCGTCGAGCGGGATGTGACCCCGGCCGAACAGTATGAGG | |
| | | ACGTGTCGGAGGCGATTTGTCTCATCAAAGATAGCCCAACGA | |
| | | TGGAGGATTTGCAGGCCGCCTTCAGCAACGCCTGGAAGGCGT | |
| | | ACAAGACCAAAGGTGCCCGTGACCAACTGACGGCCGCGAAG | |
| | | GACCAGCGTAAGAAAGAACTGTTGGATGCCCCAATTGATGTC | |
| | | GAATTTGAGGAAACCGGGGACGATCGGGCGGCGTAA | |
| λ Beta | C. crescentus | ATGAGCACGGCGCTCGCGACGCTCGCGGGGAAGCTGGCCGA | 485 |
| | | GCGTGTGGGCATGGATTCGGTCGATCCGCAGGAGCTCATCAC | |
| | | CACGCTCCGGCAGACGGCCTTTAAGTGTGACGCGAGCGATGC | |
| | | CCAGTTTATCGCCCTCCTGATCGTGGCCAATCAGTACGGCCTG | |
| | | AACCCGTGGACGAAGGAAATCTACGCCTTTCCCGACAAGCAA | |
| | | AACGGGATCGTGCCGGTGGTCGGCGTCGATGGGTGGTCCCGT | |
| | | ATCATCAATGAAAATCAGCAATTTGATGGCATGGATTTCGAG | |
| | | CAAGACAATGAATCCTGCACGTGCCGCATCTATCGGAAGGAC | |
| | | CGCAACCATCCGATCTGCGTGACGGAATGGATGGATGAGTGC | |
| | | CGCCGGGAGCCCTTTAAGACGCGGGAGGGCCGGGAAATCAC | |
| | | CGGGCCCTGGCAGTCGCACCCCAAGCGGATGCTCCGTCATAA | |
| | | GGCGATGATCCAATGTGCCCGCCTCGCCTTCGGGTTCGCGGG | |
| | | CATCTACGACAAGGATGAAGCCGAGCGCATCGTGGAAAATA | |
| | | CGGCCTACACGGCGGAGCGTCAGCCGGAACGGGATATCACG | |
| | | CCGGTCAATGACGAAACGATGCAGGAAATCAATACCCTGCTCA | |
| | | TCGCGCTCGACAAGACCTGGGACGATGATCTGCTGCCCCTGTG | |
| | | TAGCCAAATCTTCCGTCGTGATATCCGCGCCTCGTCCGAACTG | |
| | | ACCCAAGCGGAGGCGGTGAAGGCCCTGGGGTTCCTGAAGCA | |
| | | GAAGGCCACCGAGCAAAAGGTCGCGGCCTAA | |
| PapRecT | C. crescentus | ATGGGCACGGCGCTCACGCCGCTGCTCACCAAGTTTGCCACC | 486 |
| | | CGTTACGAGATGGGGACCACCCCCGAAGAAGTGGCGAACAC | |
| | | CCTGAAGCAAACGTGCTTCAAGGGCCAGGTCAACGACTCGCA | |
| | | GATGGTGGCCCTGCTCATCGTGGCCGATCAGTATAAGCTCAA | |
| | | TCCGTTCACCAAGGAACTCTACGCGTTTCCCGATAAGAACAA | |
| | | TGGGATCGTGCCGGTCGTCGGCGTCGACGGCTGGGCGCGTAT | |
| | | CATCAATGAAAATCCGCAGTTCGACGGCATGGAATTCTCGAT | |
| | | GGACCAACAAGGGACCGAATGTACGTGCAAGATCTATCGTAA | |
| | | GGATCGTTCGCACGCGATCAGCGCCACGGAATACATGGCGGAGT | |
| | | GTAAGCGGAATACGCAGCCGTGGCAATCCCACCCCCGCCGTA | |
| | | TGCTGCGCCATAAGGCGATGATCCAATGTGCCCGCCTGGCGT | |
| | | TTGGGTTCGCCGGCATCTACGATCAAGATGAAGCGGAGCGGA | |
| | | TCGTCGAACGCGATGTGACGCCCGCCGAACAATATGAAGACG | |
| | | TGTCGGAAGCGATCTGCCTGATCAAGGACAGCCCCACGATGG | |
| | | AAGATCTCCAAGCGGCCTTTAGCAATGCCTGGAAGGCCTACA | |
| | | AGACGAAGGGGGCGCGTGACCAACTGACGGCGGCCAAGGAT | |
| | | CAACGGAAGAAGGAGCTGCTGGATGCGCCGATCGATGTCGA | |
| | | ATTCGAGGAAACGGGGGACGATCGTGCCGCGTAA | |
| EcSSB | L. lactis | ATGGCAAGCCGTGGGGTTAACAAAGTTATTCTTGTTGGAAACTTA | 487 |
| | | GGACAAGACCCGGAAGTGCGTTATATGCCTAATGGAGGCGCG | |
| | | GTAGCCAATATCACCTTGGCCACAAGCGAGTCTTGGCGAGAC | |
| | | AAAGCAACAGGTGAAATGAAAGAACAAACTGAATGGCACAG | |
| | | AGTAGTTTTGTTTGGAAAATTGGCAGAGGTAGCCTCAGAATA | |
| | | CTTGCGAAAGGGCAGTCAGGTCTATATAGAGGGCCAATTGCG | |
| | | TACCCGTAAGTGGACAGACCAGAGCGGACAAGATCGTTATAC | |
| | | GACCGAGGTCGTTGTTAATGTAGGAGGCACAATGCAGATGTT | |
| | | GGGGGGGAGACAGGGCGGAGGCGCTCCGGCTGGAGGCAATAT | |
| | | CGGGGGTGGCCAACCTCAAGGTGGGTGGGGGCAGCCACAGCAA | |
| | | CCGCAAGGAGGTAATCAATTTAGTGGAGGAGCCCAATCACGT | |
| | | CCGCAGCAGTCTGCGCCTGCCGCCCCTTCTAATGAACCGCCG | |
| | | ATGGATTTTGACGATGATATACCTTTCTGA | |
| PaSSB | L. lactis | ATGGCCCGTGGAGTGAACAAAGTAATTCTTGTCGGTAATGTG | 488 |
| | | GGTGGGGATCCAGAGACGCGATACATGCCAAACGGGAACGCCG | |
| | | TGACAAATATCACCTTAGCCACGAGCGAATCTTGGAAGGACA | |
| | | AACAAACAGGTCAGCAACAAGAACGAACCGAATGGCATAGA | |
| | | GTTGTATTTTTTGGCCGACTTGCTGAGATCGCGGGTGAGTACC | |
| | | TTAGAAAGGGTTCTCAGGTTTATGTCGAGGGCTCATTAAGAA | |
| | | CACGTAAGTGGCAGGGGCAGGACGGGCAAGACCGATATACA | |
| | | ACTGAAATAGTAGTGGACATAAACGGCAACATGCAACTTCTT | |
| | | GGTGGCAGACCGAGTGGGGACGATTCACAGAGAGCTCCAAG | |
| | | AGAACCTATGCAGCGACCACAGCAGGCTCCTCAACAGCAGTC | |
| | | TCGTCCGGCCCCTCAGCAGCAACCGGCTCCGCAACCTGCACAAG | |
| | | ATTACGATAGTTTTGATGATGATATTCCATTCTAA | |
| MsSSB | L. lactis | ATGGCGGGAGACACAACAATTACGGTTGTGGGAAACTTGACA | 489 |
| | | GCCGATCCTGAATTGCGATTCACCCCATCAGGCGCTGCGGTG | |
| | | GCGAATTTCACAGTCGCGAGCACCCCACGAATGTTTGATAGA | |
| | | CAATCAGGCGAATGGAAGGATGGCGAAGCGTTGTTTTTACGA | |
| | | TGCAACATCTGGAGAGAGGCGGCCGAGAACGTCGCCGAAAG | |
| | | CCTTACCCGTGGCAGTCGAGTGATTGTAACCGGACGATTAAA | |
| | | GCAAAGAAGTTTTGAGACGAGAGAAGGAGAGAAACGAACTGT | |
| | | GGTAGAGGTAGAGGTGGATGAAATAGGTCCTAGTTTGCGTTAT | |
| | | GCCACAGCGAAAGTAAACAAAGCCTCTCGTAGTGGTGGCGG | |
| | | GGGGGGCGGCTTTGGTTCAGGGGGTGGAGGTTCACGACAGA | |
| | | GCGAGCCAAAGGATGATCCTTGGGGCAGTGCCCCTGCATCAG | |
| | | GCAGTTTTAGCGGAGCAGATGACGAGCCGCCTTTTTGA | |
| LrSSB | L. lactis | ATGCTTAATCGTGCAGTCTTAACTGGGCGTTTAACAAGAGAT | 490 |
| | | CCCGAGTTGCGGTACACAACCAGCGGGACAGCAGTTGTTTCA | |
| | | TTTACGTTAGCTGTTGATCGGCAATTCCGAAACCAAAATGGT | |
| | | GATCGTGATGCTGATTTTATCAATTGCGTTATTTGGCGTAAAT | |
| | | CCGCTGAAAACTTTAGTAACTTTACGCATAAGGGTTCACTTGT | |
| | | TGGAATTGAAGGGCGTATTCAAACCCGGAATTATGAAAACCA | |
| | | ACAGGGTAACCGTGTGTATGTTACCGAAGTTGTTGTAGATAA | |
| | | TTTTGCATTGTTAGAACCTCGTCAAAATGGTGGCATGAACCA | |
| | | ATCAGGGGTTCAACAACCATTTAACAGCAACCAACAATCATT | |
| | | TGGTGCTCAGGCTCCACAATATGGTAGTCAACCACAACCTGG | |
| | | AAATAATGCTCCTCAAAGTAATCCGTCACCAAGTATGGATAATG | |
| | | GTTTCGATCCCAATCAAAATGCTGGCAACCAGTTCCCTGGAAGC | |
| | | AGTGATGATGGTGGTCAATCCATTGATTTAGCTGATGACGAATTA | |
| | | CCATTCTAA | |
| LlSSB | L. lactis | ATGATTAACAATGTTGTATTAGTGGGACGCATTACTCGCGAT | 491 |
| | | CCTGAACTTCGTTACACCCCTCAAAATCAAGCTGTTGCAACTTTT | |
| | | TCATTGGCTGTAAATCGTCAATTTAAAAATGCTAACGGTGAAC | |
| | | GTGAGGCTGATTTCATTAACTGCGTTATTTGGCGCCAACAAG | |
| | | CTGAAAATTTGGCAAATTGGGCTAAAAAAGGAGCTTTGATCG | |
| | | GTGTAACTGGTCGAATTCAAACACGTAATTATGAAAATCAAC | |
| | | AAGGTCAACGCGTTTATGTGACTGAGGTTGTGGCTGATAGTT | |
| | | TCCAAATGTTGGAAAGTAGATCTGCTCGCGATGGTATGGGAG | |
| | | GCGGAGCTTCTGCCGGTTCATATTCTGCACCAAGCCAATCTAC | |
| | | AAATAATACTCCACGTCCACAAACGAATAATAATAGTGCAAC | |
| | | ACCGAATTTCGGTCGTGATGCTGACCCATTTGGTAGCTCACCT | |
| | | ATGGAAATCTCGGATGATGATCTTCCATTCTAA | |
| PapSSB | L. lactis | ATGCGTGGGGTTAATAAGGTAATCTTAGTTGGTAACGTGGGT | 492 |
| | | GGGGACCCGGAGACCCGATATATGCCAAATGGAAACGCGGT | |
| | | AACAAACATCACCCTTGCAACTAGTGAGAGTTGGAAAGATAA | |
| | | ACAAACTGGCCAACAGCAAGAACGTACTGAATGGCACAGAGTG | |
| | | GTGTTTTTTGGCAAATTAGCCGAAATTGTCGGCCAACACGTTAA | |
| | | GAAAGGCCAGCAGCTTTACGTCGAAGGGTCATTGCGAACCCG | |
| | | TAAGTGGCAAGCGCAGGATGGTCAGGACAGATATACGACAG | |
| | | AAATCATAGTAGATATGCACGGACAAATGCAAATGTTCGGGG | |
| | | GAAAACCTGGGAATGAGCAGGCCGCACAGTCAAGATCATCT | |
| | | ACCCAACAACAAAGCGCCCCGCAACAACGATCAGCACAGGA | |
| | | TGAATTTGATGATGATATACCTTTATAA | |
| EcSSB | E. coli | ATGGCCAGCAGAGGCGTAAACAAGGTTATTCTCGTTGGTAAT | 493 |
| | | CTGGGTCAGGACCCGGAAGTACGCTACATGCCAAATGGTGGC | |
| | | GCAGTTGCCAACATTACGCTGGCTACTTCCGAATCCTGGCGTGA | |
| | | TAAAGCGACCGGCGAGATGAAAGAACAGACTGAATGGCACCG | |
| | | CGTTGTGCTGTTCGGCAAACTGGCAGAAGTGGCGAGCGAATA | |
| | | TCTGCGTAAAGGTTCTCAGGTTTATATCGAAGGTCAGCTGCGT | |
| | | ACCCGTAAATGGACCGATCAATCCGGTCAGGATCGCTACACC | |
| | | ACAGAAGTCGTGGTGAACGTTGGCGGCACCATGCAGATGCTG | |
| | | GGTGGTCGTCAGGGTGGTGGCGCTCCGGCAGGTGGCAATATC | |
| | | GGTGGTGGTCAGCCGCAGGGCGGTTGGGGTCAGCCTCAGCAG | |
| | | CCGCAGGGTGGCAATCAGTTCAGCGGCGGCGCGCAGTCTCGC | |
| | | CCGCAGCAGTCCGCTCCGGCAGCGCCGTCTAACGAGCCGCCG | |
| | | ATGGACTTTGATGATGACATTCCGTTCTGA | |
| PaSSB | E. coli | ATGGCTCGCGGGGTAAATAAGGTCATTTTGGTTGGCAATGTT | 494 |
| | | GGTGGTGATCCCGAGACACGCTATATGCCTAACGGGAACGCC | |
| | | GTCACTAATATCACACTGGCAACGTCCGAGTCATGGAAGGAT | |
| | | AAACAGACAGGTCAACAGCAAGAACGCACGGAGTGGCACCG | |
| | | CGTGGTATTTTTCGGGCGTCTTGCTGAGATCGCCGGAGAGTAT | |
| | | TTACGCAAAGGATCGCAGGTATACGTTGAGGGTTCTTTACGC | |
| | | ACGCGCAAGTGGCAGGGTCAGGATGGTCAGGACCGTTATACT | |
| | | ACCGAAATTGTAGTCGACATTAACGGGAACATGCAATTATTA | |
| | | GGTGGTCGTCCGAGCGGAGATGACTCCCAGCGCGCCCCCCGC | |
| | | GAGCCCATGCAGCGTCCGCAACAGGCTCCACAGCAGCAGAG | |
| | | CCGCCCTGCCCCTCAACAACAACCCGCTCCTCAACCCGCGCA | |
| | | AGATTACGATTCGTTTGACGACGATATTCCTTTTTAA | |
| MsSSB | E. coli | ATGGCAGGGGATACCACGATAACCGTTGTCGGTAACTTAACC | 495 |
| | | GCGGACCCTGAACTTCGTTTCACACCATCCGGTGCAGCGGTT | |
| | | GCAAACTTCACGGTCGCTTCTACGCCTCGTATGTTCGACAGAC | |
| | | AGTCTGGTGAGTGGAAAGATGGGGAAGCACTGTTTTTAAGAT | |
| | | GCAATATATGGCGCGAAGCAGCAGAGAATGTAGCCGAGAGTT | |
| | | TAACCAGAGGTTCACGTGTGATCGTAACTGGCCGTTTGAAACA | |
| | | ACGCTCCTTTGAAACACGCGAAGGCGAGAAACGCACGGTAGTT | |
| | | GAGGTCGAAGTCGACGAGATAGGCCCGTCCTTACGCTATGCC | |
| | | ACAGCGAAAGTCAACAAAGCGTCTCGCAGCGGAGGCGGTGG | |
| | | GGGCGGGTTTGGTAGTGGTGGGGGGGGTAGTCGTCAATCGGA | |
| | | ACCCAAGGATGACCCGTGGGGGTCGGCACCAGCTTCAGGAA | |
| | | GTTTTTCTGGGGCCGATGACGAGCCGCCATTTTGA | |
| LrSSB | E. coli | ATGCTGAACCGTGCCGTGCTTACTGGTCGCCTTACTCGTGACC | 496 |
| | | CTGAATTGCGCTATACGACATCAGGGACTGCAGTAGTGTCCT | |
| | | TTACATTGGCGGTCGATCGTCAATTTCGTAACCAAAACGGCG | |
| | | ACCGCGACGCCGATTTTATCAACTGTGTGATTTGGAGAAAGA | |
| | | GCGCCGAGAACTTTAGCAATTTCACTCATAAAGGGAGTTTAG | |
| | | TTGGAATCGAGGGGCGTATCCAAACGAGAAACTACGAAAACC | |
| | | AGCAAGGCAATCGCGTCTACGTAACCGAAGTCGTAGTAGATA | |
| | | ACTTCGCCCTGTTGGAACCACGGCAAAACGGTGGGATGAACC | |
| | | AATCTGGAGTTCAACAACCCTTCAACAGTAACCAGCAGTCTT | |
| | | TCGGGGCTCAGGCACCTCAATATGGCAGTCAGCCACAACCTG | |
| | | GAAACAATGCCCCACAGTCTAACCCAAGTCCCTCTATGGACAAT | |
| | | GGGTTTGACCCCAACCAGAATGCGGGGAACCAATTCCCTGGG | |
| | | AGCTCGGATGACGGCGGCCAATCAATTGATCTGGCTGACGATGA | |
| | | ATTACCCTTTTAA | |
| PaSSB | M. | ATGGCGCGTGGGGTGAACAAAGTCATCCTCGTGGGGAATGTC | 497 |
| | smegmatis | GGTGGCGATCCCGAAACGCGTTATATGCCGAATGGGAATGCG | |
| | | GTCACCAATATCACGCTCGCCACCAGCGAGTCCTGGAAAGAT | |
| | | AAACAAACGGGTCAACAGCAGGAGCGTACGGAGTGGCATCG | |
| | | GGTGGTCTTCTTCGGGCGCCTCGCCGAGATCGCCGGGGAATA | |
| | | CCTCCGTAAAGGTTCGCAGGTCTATGTGGAGGGCTCGCTGCG | |
| | | GACCCGTAAATGGCAAGGTCAGGATGGCCAGGATCGGTACA | |
| | | CGACGGAAATCGTCGTGGACATTAACGGTAATATGCAATTGC | |
| | | TCGGTGGCCGCCCCTCCGGCGATGATAGCCAGCGTGCCCCGC | |
| | | GTGAACCGATGCAACGCCCGCAACAAGCGCCCCAACAGCAA | |
| | | TCGCGGCCCGCGCCGCAGCAGCAGCCGGCCCCGCAACCAGCC | |
| | | CAGGACTACGATTCGTTTGATGATGACATTCCATTTTAA | |
| PaSSB | C. | ATGGCGCGTGGGGTCAATAAGGTGATCCTGGTCGGCAACGTG | 498 |
| | crescentus | GGGGGCGATCCCGAAACCCGGTACATGCCGAACGGCAACGC | |
| | | GGTCACCAACATCACCCTGGCGACCAGCGAGAGCTGGAAGG | |
| | | ATAAGCAAACGGGCCAGCAGCAAGAACGTACGGAATGGCAT | |
| | | CGTGTGGTCTTTTTCGGCCGGCTGGCGGAGATCGCGGGGGAA | |
| | | TACCTCCGTAAGGGGTCCCAAGTCTACGTGGAGGGCTCGCTG | |
| | | CGGACCCGGAAGTGGCAAGGGCAAGATGGGCAAGATCGCTA | |
| | | CACCACGGAGATCGTCGTCGACATCAACGGGAACATGCAGCT | |
| | | CCTCGGGGGGCGTCCCTCGGGGGACGATTCCCAACGCGCCCC | |
| | | CCGTGAGCCCATGCAACGCCCGCAGCAAGCGCCCCAGCAACA | |
| | | ATCGCGTCCCGCCCCCCAGCAACAGCCGGCGCCCCAACCGGC | |
| | | GCAGGACTACGACTCGTTCGACGATGATATCCCCTTTTAA | |
| PapExo | L. lactis | ATGATAGAACAGCGTAGTGATGAATGGTTCGCGCAGCGACTT | 499 |
| | | GGCCGAGTCACCGCGAGTAAAGTAAAGGATGTCATGGCGAA | |
| | | GGGGCGATCAGGTGCGCCATCAGCCACCAGACAGAATTACATG | |
| | | ATGCAATTGTTATGTGAGAGACTTACCGGGAAACGAGAAGAGGG | |
| | | GTTCACGAGTGCGGCGATGCAGCGTGGGACGGACCTTGAACC | |
| | | AATAGCGCGATCAGCTTATGAGTTTAACGCAGGAGTAATGAC | |
| | | TATAGAAACAGGCCTTATTATCCATCCACGTATCGACGGTTTCG | |
| | | GAGCTAGTCCGGATGGGCTTGCGGGAGAGCATGGATTAGTGGA | |
| | | AATTAAGTGCCCGTCAACAGCAACGCACATTTATACCATGCA | |
| | | AAGTGGTAAGCACGACCCTCAGTACGAATGGCAAATGCTTGC | |
| | | TCAAATGAGTTGCTCAGGCAGAGAGTGGGTGGATTTCGTGTCAT | |
| | | TCGACGATAGATTGCCAGACGAATTGCAATATGTTTGTTTCCG | |
| | | TTATCACCGTGATGAAGAGAGAATAAGAGAAATGGAAAGCG | |
| | | AAGTTAAGGCATTCTTGGAGGAATTAGCTGAATTGGAACACC | |
| | | AAATGCGTGAACGTATGAGAAAGGCGGCCTAA | |
| LrpExo | L. lactis | ATGAAACTTACGGCCAACAATTACTATAGCCATGAGACTGAC | 500 |
| | | TGGCAATATATGTCAGTTTCATTGTTCAAAGACTTCGAAAAGT | |
| | | GCGAAGCGCGTGCATTAGCAAAGTTGAAGGAAGATTGGCAA | |
| | | CCTGTTTCTAGTCCAGTTCCGCTTTTGGTTGGGAACTATGTAC | |
| | | ACAGTTATTTCGAAAGTGCTAAGAGCCACCAAGATTTTATAG | |
| | | AGGCGAATAAGAAAGAGCTTATGACCAGACCTACTAAGACA | |
| | | AACCCGAACGGCCATCTTAGAGCGGAATTTAAGGGGGCAAAC | |
| | | TCAATGATTCAGACCTTGCAAGCCGACGATATGTTTAACTACT | |
| | | TTTATGCACCAGGGGACAAAGAAGTTATCGTTACCGGAGAGA | |
| | | TAGACGGCTATTTGTGGAAGGGAAAAATAGACTCTTTAGTTC | |
| | | TTGACAAAGGCTATTTTTGCGATCTTAAGACGGTAGACGACA | |
| | | TTCATAAGGGACATTGGAATACGTATGAACACAGATACGTCC | |
| | | CGTTCATTCAAGACCGAGAATATGATTTACAAATGGCTGTTT | |
| | | ATAGAGAGTTAATCAAGCAGACGTTCGGGAAAAAGTGCCAA | |
| | | CCTTTAATTTTTGCCATCTCTAAGCAAACTCCGCCTGACAAGAT | |
| | | GGCCATCGACTTTAATGGCGTTGATGACGACTATCAGATGCAG | |
| | | GCCGATCTTGATAAGGTCAAAGAGCTTCAACCACACTTTTGG | |
| | | AAAGTAATGACGGGAGAGGAAGAGCCTGTCCACTGTGGTAA | |
| | | GTGCGACTATTGTAGAGAAACGAAAATGTTGAGCGGCTTCAT | |
| | | CCACGCATCAGAAATAGAGGTTTAA | |
| mCardinal | | ATGCACCATCATCACCACCACGGTTCCGGCATGGTTTCTAAA | 501 |
| RBS eGFP | | GGTGAAGAACTGATCAAGGAAAACATGCACATGAAGCTGTA | |
| | | TATGGAAGGTACCGTTAACAACCACCATTTCAAATGCACCAC | |
| | | TGAAGGTGAAGGTAAACCGTACGAGGGTACGCAGACCCAAC | |
| | | GTATTAAAGTTGTTGAGGGTGGTCCGCTGCCGTTCGCGTTCGA | |
| | | CATCCTGGCGACCTGTTTCATGTACGGCTCTAAAACCTTCATC | |
| | | AACCACACCCAGGGTATCCCTGACTTCTTCAAACAGTCTTTCC | |
| | | CGGAGGGTTTCACCTGGGAACGTGTTACCACCTACGAAGACG | |
| | | GTGGTGTACTGACCGTTACCCAGGACACTTCTCTGCAGGACG | |
| | | GTTGCCTGATCTACAACGTTAAACTCCGCGGTGTTAATTTCCC | |
| | | GTCTAACGGTCCGGTTATGCAAAAAAAGACGCTGGGTTGGGA | |
| | | AGCGACTACGGAAACTCTCTACCCTGCCGATGGCGGCCTCGA | |
| | | AGGTCGTTGTGATATGGCGCTGAAACTGGTTGGTGGCGGTCA | |
| | | CCTGCACTGCAATCTGAAAACTACCTACCGTTCTAAAAAACC | |
| | | AGCTAAAAACCTCAAAATGCCGGGTGTTTACTTTGTTGATCGT | |
| | | CGTCTGGAACGTATCAAAGAAGCAGACAACGAAACTTACGTT | |
| | | GAACAGCACGAAGTTGCGGTGGCGCGTTACTGCGACCTGCCA | |
| | | TCTAAACTGGGTCACAAAGGTATGGACGAACTGTACAAATAA | |
| | | AAAAAATAGGAGGAAAAACATATGGGTTCTCACCACCATCAC | |
| | | CACCACAGCGGCTCTAAAGGTGAAGAATTATTCACTGGTGTT | |
| | | GTCCCAATTTTGGTTGAATTAGATGGTGATGTTAATGGTCACA | |
| | | AATTTTCTGTCTCCGGTGAAGGTGAAGGTGATGCTACGTACG | |
| | | GTAAATTGACCTTAAAATTTATTTGTACTACTGGTAAATTGCC | |
| | | AGTTCCATGGCCAACCTTAGTCACTACTTTCACTTATGGTGTT | |
| | | CAATGTTTTTCTAGATACCCAGATCATATGAAACAACATGAC | |
| | | TTTTTCAAGTCTGCCATGCCAGAAGGTTATGTTCAAGAAAGA | |
| | | ACTATTTTTTTCAAAGATGACGGTAACTACAAGACCAGAGCT | |
| | | GAAGTCAAGTTTGAAGGTGATACCTTAGTTAATAGAATCGAA | |
| | | TTAAAAGGTATTGATTTTAAAGAAGATGGTAACATTTTAGGT | |
| | | CACAAATTGGAATACAACTATAACTCTCACAATGTTTACATC | |
| | | ATGGCTGACAAACAAAAGAATGGTATCAAAGTTAACTTCAAA | |
| | | ATTAGACACAACATTGAAGATGGTTCTGTTCAATTAGCTGAC | |
| | | CATTATCAACAAAATACTCCAATTGGTGATGGTCCAGTCTTGT | |
| | | TACCAGACAACCATTACTTATCCACTCAATCTGCCTTATCCAA | |
| | | AGATCCAAACGAAAAGAGAGACCACATGGTCTTGTTAGAATT | |
| | | TGTTACTGCTGCTGGTATTACCCATGGTATGGATGAATTGTAC | |
| | | AAATAA | |
| E. coli MutL | L. lactis | ATGCCTATACAAGTGTTGCCTCCACAGTTGGCCAACCAAATC | 502 |
| E32K | | GCGGCAGGCGAGGTGGTCGAACGTCCGGCTTCAGTCGTTAAG | |
| | | GAATTGGTAAAAAATTCTTTGGATGCAGGGGCAACGAGAATT | |
| | | GATATTGACATCGAACGAGGCGGGGCCAAGTTAATCAGAATC | |
| | | CGAGACAATGGGTGTGGGATTAAAAAGGATGAACTTGCTTTG | |
| | | GCGTTGGCACGTCACGCGACCAGCAAAATAGCGTCTCTTGAC | |
| | | GACTTGGAAGCTATTATCAGTCTTGGTTTCCGTGGGGAAGCCT | |
| | | TAGCATCTATTAGCTCTGTGTCACGTTTGACTTTGACTAGCAG | |
| | | AACGGCGGAACAGCAGGAAGCATGGCAAGCGTATGCGGAAG | |
| | | GACGAGACATGAACGTCACGGTTAAGCCGGCAGCCCACCCG | |
| | | GTCGGCACGACCTTGGAGGTCTTGGACTTGTTCTATAATACCC | |
| | | CTGCACGTCGTAAATTCTTACGAACCGAAAAGACCGAATTTA | |
| | | ACCATATAGATGAGATAATAAGAAGAATTGCGTTAGCACGTT | |
| | | TCGATGTTACTATAAATTTGAGTCATAACGGAAAAATCGTTA | |
| | | GACAGTATCGAGCCGTGCCTGAGGGCGGGCAGAAGGAAAGA | |
| | | AGATTAGGGGCTATTTGTGGCACTGCTTTTCTTGAACAAGCAC | |
| | | TTGCGATCGAATGGCAACATGGGGACCTTACCTTGCGAGGTT | |
| | | GGGTAGCGGACCCGAATCATACAACACCAGCGTTGGCAGAG | |
| | | ATACAATATTGCTATGTAAACGGACGAATGATGAGAGATCGT | |
| | | TTGATCAACCACGCAATACGACAGGCTTGCGAAGATAAGTTG | |
| | | GGGGCGGATCAACAGCCAGCTTTCGTCCTTTATCTTGAAATTG | |
| | | ACCCTCATCAGGTAGATGTGAATGTACATCCGGCCAAACACG | |
| | | AGGTTCGTTTTCATCAAAGTCGACTTGTGCATGATTTTATATA | |
| | | CCAGGGTGTCTTAAGTGTCTTGCAGCAGCAGCTTGAGACACC | |
| | | TTTACCTTTAGATGATGAGCCGCAGCCAGCTCCGCGTAGTATC | |
| | | CCTGAGAATCGAGTTGCCGCCGGCAGAAATCATTTCGCAGAA | |
| | | CCGGCAGCCCGTGAACCTGTAGCACCGAGATACACCCCGGCT | |
| | | CCTGCCTCTGGATCACGTCCTGCTGCCCCGTGGCCTAACGCAC | |
| | | AACCGGGCTATCAGAAGCAGCAGGGTGAAGTTTATCGTCAAT | |
| | | TGTTACAAACTCCGGCACCAATGCAAAAACTTAAGGCCCCGG | |
| | | AGCCGCAGGAACCGGCGCTTGCTGCAAATTCACAATCTTTCG | |
| | | GACGAGTTTTAACAATAGTGCATAGTGACTGCGCATTACTTG | |
| | | AGCGTGACGGCAACATTAGTTTGCTTTCATTGCCTGTTGCCGA | |
| | | GCGTTGGTTGAGACAAGCACAATTAACCCCTGGTGAAGCACC | |
| | | AGTCTGTGCACAGCCATTATTGATCCCATTGCGTTTAAAGGTC | |
| | | TCAGCCGAGGAAAAGAGTGCTTTGGAAAAAGCCCAAAGTGC | |
| | | CCTTGCAGAGCTTGGAATTGATTTCCAAAGCGACGCACAACA | |
| | | CGTTACGATAAGAGCGGTTCCATTACCGTTAAGACAGCAAAA | |
| | | CTTACAAATTCTTATACCAGAGCTTATCGGGTATTTGGCGAAA | |
| | | CAGAGCGTATTCGAACCAGGTAATATCGCCCAGTGGATAGCG | |
| | | CGTAACCTTATGTCAGAACACGCGCAGTGGAGTATGGCGCAA | |
| | | GCTATCACATTGTTAGCCGACGTTGAGCGTTTGTGCCCACAGT | |
| | | TGGTGAAAACGCCTCCGGGTGGACTTCTTCAAAGTGTGGACT | |
| | | TACATCCAGCAATTAAGGCTCTTAAAGATGAATAA | |
| L. lactis MutL | L. lactis | GTGGGAAAAATTATTGAACTAAATGAAGCGCTCGCCAATCAA | 503 |
| E33K | | ATTGCTGCTGGAGAGGTGGTTGAGCGGCCTGCTAGTGTTGTC | |
| | | AAAGAATTAGTCAAAAACTCAATTGATGCTGGAAGCAGTAAA | |
| | | ATTATTATCAATGTTGAAGAAGCAGGTTTGCGATTAATTGAA | |
| | | GTCATTGATAATGGTTTGGGCTTAGAAAAAGAAGATGTGGCT | |
| | | TTGGCTTTGCGTCGTCATGCGACAAGTAAAATCAAAGATTCA | |
| | | GCTGATTTATTTCGAATTAGAACGCTCGGTTTTCGGGGTGAGG | |
| | | CTCTGCCGTCAATCGCTTCTGTCAGTCAGATGACGATTGAAAC | |
| | | AAGTAATGCTCAGGAAGAAGCTGGGACAAAACTGATTGCTA | |
| | | AAGGTGGGACGATTGAAACTTTAGAACCTCTTGCAAAGCGGT | |
| | | TAGGGACAAAAATTTCTGTTGCGAATCTTTTTTATAATACACC | |
| | | AGCAAGGCTCAAGTATATCAAGTCTTTACAGGCTGAACTTTC | |
| | | TCATATTACAGATATTATCAATCGTTTGAGCCTCGCTCATCCA | |
| | | GAGATTTCTTTTACTTTAGTTAATGAGGGTAAAGAATTTTTGA | |
| | | AAACGGCGGGAAATGGAGACTTGCGCCAAGTGATTGCTGCA | |
| | | ATTTATGGCATTGGAACGGCGAAAAAAATGCGTGAGATTAAT | |
| | | GGCTCGGACTTAGATTTTGAACTGACAGGTTATGTCAGTTTAC | |
| | | CCGAGCTGACAAGAGCGAATCGCAACTATATCACGATTTTGA | |
| | | TTAATGGTCGATTTATCAAGAATTTTTTGTTGAATCGAGCAAT | |
| | | TTTAGAAGGTTACGGGAACCGATTGATGGTTGGACGTTTTCCT | |
| | | TTTGCTGTTTTATCAATTAAAATTGACCCTAAATTAGCAGATG | |
| | | TCAATGTCCATCCGACAAAACAAGAAGTACGTTTGTCTAAGG | |
| | | AACGTGAATTGATGACTTTAATTTCTAAAGCGATTGATGAGA | |
| | | CCTTATCAGAAGGGGTTTTGATTCCAGAAGCTTTGGAAAATTT | |
| | | GCAAGGTAGAGCCAAGGAAAAGGGGACTGTTTCTGTTCAAAC | |
| | | GGAACTTCCTTTACAGAATAATCCTTTATACTATGACAATGTT | |
| | | CGTCAAGATTTTTTTGTCAGAGAAGAAGCGATTTTTGAAATC | |
| | | AATAAAAACGATAATTCAGATTCTCTGACTGAACAAAATTCT | |
| | | ACTGATTATACAGTTAATCAGCCAGAAACTGGTTCTGTCAGT | |
| | | GAAAAAATTACGGACAGAACTGTCGAAAGTTCAAATGAATTT | |
| | | ACTGACAGAACCCCAAAAAATTCTGTCAGTAACTTTGGAGTT | |
| | | GATTTTGATAATATTGAGAAGCTGAGTCAGCAATCAACTTTTC | |
| | | CCCAACTAGAATACTTGGCACAATTGCATGCGACTTATTTACT | |
| | | TTGTCAGTCAAAAGAGGGTCTTTATTTGGTTGACCAACATGC | |
| | | GGCTCAGGAGCGAATCAAGTATGAATATTGGAAAGATAAAA | |
| | | TCGGCGAAGTGAGCATGGAGCAACAAATTTTACTTGCGCCAT | |
| | | ATTTATTTACTTTACCCAAAAATGATTTTATTGTTTTAGCTGA | |
| | | GAAAAAGGATTTATTACATGAAGCAGGGGTTTTCTTGGAAGA | |
| | | ATACGGAGAAAATCAATTCATATTAAGAGAGCATCCGATTTG | |
| | | GTTAAAAGAAACTGAGATAGAGAAATCAATTAATGAAATGA | |
| | | TTGATATTATTCTCTCATCAAAAGAATTTTCACTCAAAAAATA | |
| | | TCGGCATGATTTAGCCGCAATGGTTGCTTGTAAAAGCTCAAT | |
| | | CAAAGCCAACCATCCCCTTGATGCCGAGTCTGCTAGAGCTTT | |
| | | GCTTAGAGAATTATCAACTTGTAAAAATCCTTATAGTTGTGCG | |
| | | CATGGACGGCCAACGATTGTCCATTTTTCAGGAGATGACATT | |
| | | CAAAAAATGTTCCGCAGAATTCAAGAAACGCATCGTTCAAAA | |
| | | GCGGCCTCTTGGAAAGATTTTGAGTAA | |
| L. lactis | | ATGATTGAACTTAGTGGCAAAGATAGAAAGTATTTGTATAAA | 504 |
| dsDNA | | CTAGTAAAATCCAAAAAACTAAATTATGAACAAGGTAATTTA | |
| template | | TCGCATCAAGTTTTAATTGAAAACAAGTTAGCAAAAGTTTAC | |
| (Erythromycin | | TTTACAAGCGATAAATATGATCCTGACTTAGGGGAACACATA | |
| resistance | | AATCCACAAAATATTATTGCTCCAACTAGTACAGGTTTAAGA | |
| gene) | | TATAAAAATATTTATCGTGAACAATTATGGGAAAAATATTTT | |
| Homology | | ACTCCTATTTGGGTATCTACGGCAACAACGACTCTAATATGGT | |
| arm, promoter, | | TAGCAAAATATTTACTAGAGAACTTGCTGTAACGCTAAGTAA | |
| gene, | | GATTACTATCCATAGCTCTTTTTTATCTTTTCTCATCTTTCCAC | |
| terminator, | | CTCCTAGCCCACTCGGGCTTTTTAATTTAAAAATTGTTTAATC | |
| homology arm | | TCATGAAACGCCATGCCTATTTCTAACAGTAAGATAATGCTG | |
| | | TCAGTATAGCGCCTAAGCGTTTCTTTTTGTTCTGATTTTTTAAT | |
| | | GTGGTCTTTATTCTTCAACTAAAGCACCCATTAGTTCAACAAA | |
| | | CGAAAATTGGATAAAGTGGGATATTTTTAAAATATATATTTA | |
| | | TGTTACAGTAATATTGACTTTTAAAAAAGGATTGATTCTAATG | |
| | | AAGAAAGCAGACAAGTAAGCCTCCTAAATTCACTTTAGATAA | |
| | | AAATTTAGGAGGCATATCAAATGAACAAAAATATAAAATATT | |
| | | CTCAAAACTTTTTAACGAGTGAAAAAGTACTCAACCAAATAA | |
| | | TAAAACAATTGAATTTAAAAGAAACCGATACCGTTTACGAAA | |
| | | TTGGAACAGGTAAAGGGCATTTAACGACGAAACTGGCTAAA | |
| | | ATAAGTAAACAGGTAACGTCTATTGAATTAGACAGTCATCTA | |
| | | TTCAACTTATCGTCAGAAAAATTAAAACTGAATACTCGTGTC | |
| | | ACTTTAATTCACCAAGATATTCTACAGTTTCAATTCCCTAACA | |
| | | AACAGAGGTATAAAATTGTTGGGAGTATTCCTTACCATTTAA | |
| | | GCACACAAATTATTAAAAAAGTGGTTTTTGAAAGCCATGCGT | |
| | | CTGACATCTATCTGATTGTTGAAGAAGGATTCTACAAGCGTA | |
| | | CCTTGGATATTCACCGAACACTAGGGTTGCTCTTGCACACTCA | |
| | | AGTCTCGATTCAGCAATTGCTTAAGCTGCCAGCGGAATGCTTT | |
| | | CATCCTAAACCAAAAGTAAACAGTGTCTTAATAAAACTTACC | |
| | | CGCCATACCACAGATGTTCCAGATAAATATTGGAAGCTATAT | |
| | | ACGTACTTTGTTTCAAAATGGGTCAATCGAGAATATCGTCAA | |
| | | CTGTTTACTAAAAATCAGTTTCATCAAGCAATGAAACACGCC | |
| | | AAAGTAAACAATTTAAGTACCGTTACTTATGAGCAAGTATTG | |
| | | TCTATTTTTAATAGTTATCTATTATTTAACGGGAGGAAATAAT | |
| | | AATATGAGATAATGCCGACTGTACTTTTTACAGTCGGTTTTCT | |
| | | AATGTCACTAACCTGCCCCGTTAGTTGAAGAAGGTTTTTATAT | |
| | | TACAGCTCCACGGTTAAATTTGTCGCCTGACTGTTTAAAGCTC | |
| | | GTTAGACTACGATATTTTCCGCTTGTCGTAAGTTGTACAAGTA | |
| | | AATCAAGAATGATTTTGTGATAGTACGGTTTAGACTGCCTGCT | |
| | | TTGCATGATTGCGGTGTCTAGTTTGTTCATGGTTAGTTATCCT | |
| | | TAACTTGCAAAAAAATCAAGTTAATAGTTAAAATTTTTCATC | |
| | | AAGTCATAAATAGAATTTTCTTCTAAATTTGCTGCTCTTTCTA | |
| | | ATTCTTTAACCTTATCAAGTGTTAATTTATTCGGAGCTAATCT | |
| | | AATGCGATATAGAGCATTATATGTGATTCCCATATTCTTCGCT | |
| | | ATCGCCTCATATCTTACCCCTGATTGTTTTAAAATCTCATCAA | |
| | | GTGGTTTATAAGTTTTACTCATTTTATCTCCTTTCTGATTTTTA | |
| | | TGTTTTTCATTCTAACATTAACTTGATTTTTATGCAAGTAATA | |
| | | ACTTTACTTTTTTGCAAGTTTTCTCTTGAAAGTAGTT | |
| RBS | | TTTTGGGGAGACGACCAT | 505 |
| RBS Mod | | AAAAGGAGGTTTTTT | 506 |
| RBS 2 | | AAAAAATAGGAGGAAAAACAT | 507 |
| RBS 2 Mod | | AAAAAAAAAAGGAGGTTTTTT | 508 |
| RBS 1 | | AAAAATAAGGTGGAAAAACAT | 509 |
| RBS 3 | | AAAAATAAGGAGGTAAAACAT | 510 |
| RBS 4 | | AGCTATTCATAAGGAGGTTTAGATT | 511 |
| L. Lactis MutL | | MGKIIELNEALANQIAAGEVVERPASVVKELVENSIDAGSSKIIIN | 512 |
| | | VEEAGLRLIEVIDNGLGLEKEDVALALRRHATSKIKDSADLFRIR | |
| | | TLGFRGEALPSIASVSQMTIETSNAQEEAGTKLIAKGGTIETLEPL | |
| | | AKRLGTKISVANLFYNTPARLKYIKSLQAELSHITDIINRLSLAHP | |
| | | EISFTLVNEGKEFLKTAGNGDLRQVIAAIYGIGTAKKMREINGSD | |
| | | LDFELTGYVSLPELTRANRNYITILINGRFIKNFLLNRAILEGYGN | |
| | | RLMVGRFPFAVLSIKIDPKLADVNVHPTKQEVRLSKERELMTLIS | |
| | | KAIDETLSEGVLIPEALENLQGRAKEKGTVSVQTELPLQNNPLYY | |
| | | DNVRQDFFVREEAIFEINKNDNSDSLTEQNSTDYTVNQPETGSVS | |
| | | EKITDRTVESSNEFTDRTPKNSVSNFGVDFDNIEKLSQQSTFPQLE | |
| | | YLAQLHATYLLCQSKEGLYLVDQHAAQERIKYEYWKDKIGEVS | |
| | | MEQQILLAPYLFTLPKNDFIVLAEKKDLLHEAGVFLEEYGENQFI | |
| | | LREHPIWLKETEIEKSINEMIDIILSSKEFSLKKYRHDLAAMVACK | |
| | | SSIKANHPLDAESARALLRELSTCKNPYSCAHGRPTIVHFSGDDI | |
| | | QKMFRRIQETHRSKAASWKDFE | |
| L. Lactis MutL | | MGKIIELNEALANQIAAGEVVERPASVVKELVKNSIDAGSSKIIIN | 513 |
| E33 | | KVEEAGLRLIEVIDNGLGLEKEDVALALRRHATSKIKDSADLFRIR | |
| | | TLGFRGEALPSIASVSQMTIETSNAQEEAGTKLIAKGGTIETLEPL | |
| | | AKRLGTKISVANLFYNTPARLKYIKSLQAELSHITDIINRLSLAHP | |
| | | EISFTLVNEGKEFLKTAGNGDLRQVIAAIYGIGTAKKMREINGSD | |
| | | LDFELTGYVSLPELTRANRNYITILINGRFIKNFLLNRAILEGYGN | |
| | | RLMVGRFPFAVLSIKIDPKLADVNVHPTKQEVRLSKERELMTLIS | |
| | | KAIDETLSEGVLIPEALENLQGRAKEKGTVSVQTELPLQNNPLYY | |
| | | DNVRQDFFVREEAIFEINKNDNSDSLTEQNSTDYTVNQPETGSVS | |
| | | EKITDRTVESSNEFTDRTPKNSVSNFGVDFDNIEKLSQQSTFPQLE | |
| | | YLAQLHATYLLCQSKEGLYLVDQHAAQERIKYEYWKDKIGEVS | |
| | | MEQQILLAPYLFTLPKNDFIVLAEKKDLLHEAGVFLEEYGENQFI | |
| | | LREHPIWLKETEIEKSINEMIDIILSSKEFSLKKYRHDLAAMVACK | |
| | | SSIKANHPLDAESARALLRELSTCKNPYSCAHGRPTIVHFSGDDI | |
| | | QKMFRRIQETHRSKAASWKDFE | |
| E. Coli MutL | | MPIQVLPPQLANQIAAGEVVERPASVVKELVENSLDAGATRIDID | 514 |
| | | IERGGAKLIRIRDNGCGIKKDELALALARHATSKIASLDDLEAIIS | |
| | | LGFRGEALASISSVSRLTLTSRTAEQQEAWQAYAEGRDMNVTV | |
| | | KPAAHPVGTTLEVLDLFYNTPARRKFLRTEKTEFNHIDEIIRRIAL | |
| | | ARFDVTINLSHNGKIVRQYRAVPEGGQKERRLGAICGTAFLEQA | |
| | | LAIEWQHGDLTLRGWVADPNHTTPALAEIQYCYVNGRMMRDR | |
| | | LINHAIRQACEDKLGADQQPAFVLYLEIDPHQVDVNVHPAKHEV | |
| | | RFHQSRLVHDFIYQGVLSVLQQQLETPLPLDDEPQPAPRSIPENR | |
| | | VAAGRNHFAEPAAREPVAPRYTPAPASGSRPAAPWPNAQPGYQ | |
| | | KQQGEVYRQLLQTPAPMQKLKAPEPQEPALAANSQSFGRVLTIV | |
| | | HSDCALLERDGNISLLSLPVAERWLRQAQLTPGEAPVCAQPLLIP | |
| | | LRLKVSAEEKSALEKAQSALAELGIDFQSDAQHVTIRAVPLPLRQ | |
| | | QNLQILIPELIGYLAKQSVFEPGNIAQWIARNLMSEHAQWSMAQ | |
| | | AITLLADVERLCPQLVKTPPGGLLQSVDLHPAIKALKDE | |
| E. coli MutL | | MPIQVLPPQLANQIAAGEVVERPASVVKELVKNSLDAGATRIDI | 515 |
| E32K | | DIERGGAKLIRIRDNGCGIKKDELALALARHATSKIASLDDLEAII | |
| | | SLGFRGEALASISSVSRLTLTSRTAEQQEAWQAYAEGRDMNVTV | |
| | | KPAAHPVGTTLEVLDLFYNTPARRKFLRTEKTEFNHIDEIIRRIAL | |
| | | ARFDVTINLSHNGKIVRQYRAVPEGGQKERRLGAICGTAFLEQA | |
| | | LAIEWQHGDLTLRGWVADPNHTTPALAEIQYCYVNGRMMRDR | |
| | | LINHAIRQACEDKLGADQQPAFVLYLEIDPHQVDVNVHPAKHEV | |
| | | RFHQSRLVHDFIYQGVLSVLQQQLETPLPLDDEPQPAPRSIPENR | |
| | | VAAGRNHFAEPAAREPVAPRYTPAPASGSRPAAPWPNAQPGYQ | |
| | | KQQGEVYRQLLQTPAPMQKLKAPEPQEPALAANSQSFGRVLTIV | |
| | | HSDCALLERDGNISLLSLPVAERWLRQAQLTPGEAPVCAQPLLIP | |
| | | LRLKVSAEEKSALEKAQSALAELGIDFQSDAQHVTIRAVPLPLRQ | |
| | | QNLQILIPELIGYLAKQSVFEPGNIAQWIARNLMSEHAQWSMAQ | |
| | | AITLLADVERLCPQLVKTPPGGLLQSVDLHPAIKALKDE | |
| Pa MutL | | MSEAPRIQLLSPRLANQIAAGEVVERPASVAKELLENSLDAGSRR | 548 |
| (wild-type) | | IDVEVEQGGIKLLRVRDDGRGIPADDLPLALARHATSKIRELEDL | |
| | | ERVMSLGFRGEALASISSVARLTMTSRTADAGEAWQVETEGRD | |
| | | MQPRVQPAAHPVGTSVEVRDLFFNTPARRKFLRAEKTEFDHLQE | |
| | | VIKRLALARFDVAFHLRHNGKTIFALHEARDELARARRVGAVC | |
| | | GQAFLEQALPIEVERNGLHLWGWVGLPTFSRSQPDLQYFYVNG | |
| | | RMVRDKLVAHAVRQAYRDVLYNGRHPTFVLFFEVDPAVVDVN | |
| | | VHPTKHEVRFRDSRMVHDFLYGTLHRALGEVRPDDQLAPPGAT | |
| | | SLTEPRPTGAAAGEFGPQGEMRLAESVLESPAARVGWSGGSSAS | |
| | | GGSSGYSAYTRPEAPPSLAEAGGAYKAYFAPLPAGEAPAALPES | |
| | | AQDIPPLGYALAQLKGIYILAENAHGLVLVDMHAAHERITYERL | |
| | | KVAMASEGLRGQPLLVPESIAVSEREADCAEEHSSWFQRLGFEL | |
| | | QRLGPESLAIRQIPALLKQAEATQLVRDVIADLLEYGTSDRIQAH | |
| | | LNELLGTMACHGAVRANRRLTLPEMNALLRDMEITERSGQCNH | |
| | | GRPTWTQLGLDELDKLFLRGR | |
| Pa MutL | | MSEAPRIQLLSPRLANQIAAGEVVERPASVAKELLKNSLDAGSR | 549 |
| (E36K) | | RIDVEVEQGGIKLLRVRDDGRGIPADDLPLALARHATSKIRELED | |
| | | LERVMSLGFRGEALASISSVARLTMTSRTADAGEAWQVETEGR | |
| | | DMQPRVQPAAHPVGTSVEVRDLFFNTPARRKFLRAEKTEFDHL | |
| | | QEVIKRLALARFDVAFHLRHNGKTIFALHEARDELARARRVGA | |
| | | VCGQAFLEQALPIEVERNGLHLWGWVGLPTFSRSQPDLQYFYV | |
| | | NGRMVRDKLVAHAVRQAYRDVLYNGRHPTFVLFFEVDPAVVD | |
| | | VNVHPTKHEVRFRDSRMVHDFLYGTLHRALGEVRPDDQLAPPG | |
| | | ATSLTEPRPTGAAAGEFGPQGEMRLAESVLESPAARVGWSGGSS | |
| | | ASGGSSGYSAYTRPEAPPSLAEAGGAYKAYFAPLPAGEAPAALP | |
| | | ESAQDIPPLGYALAQLKGIYILAENAHGLVLVDMHAAHERITYE | |
| | | RLKVAMASEGLRGQPLLVPESIAVSEREADCAEEHSSWFQRLGF | |
| | | ELQRLGPESLAIRQIPALLKQAEATQLVRDVIADLLEYGTSDRIQ | |
| | | AHLNELLGTMACHGAVRANRRLTLPEMNALLRDMEITERSGQC | |
| | | NHGRPTWTQLGLDELDKLFLRGR | |
| EcSSB (C10) | | PMDFDDDIPF | 516 |
| CFSSB (C10) | | | |
| SeSSB (C10) | | | |
| EcSSB (C9) | | MDFDDDIPF | 517 |
| CFSSB (C9) | | | |
| SeSSB (C9) | | | |
| EcSSB (C8) | | DFDDDIPF | 518 |
| CFSSB (C8) | | | |
| SeSSB (C8) | | | |
| EcSSB (C7) | | FDDDIPF | 519 |
| CFSSB (C7) | | | |
| SeSSB (C7) | | | |
| PaSSB (C7) | | | |
| PaSSB (C10) | | YDSFDDDIPF | 520 |
| PaSSB (C9) | | DSFDDDIPF | 521 |
| PaSSB (C8) | | SFDDDIPF | 522 |
| MsSSB (C10) | | FSGADDEPPF | 524 |
| MsSSB (C9) | | SGADDEPPF | 525 |
| MsSSB (C8) | | GADDEPPF | 526 |
| MsSSB (C7) | | ADDEPPF | 527 |
| LrSSB (C10) | | IDLADDELPF | 528 |
| LrSSB (C9) | | DLADDELPF | 529 |
| LrSSB (C8) | | LADDELPF | 530 |
| LrSSB (C7) | | ADDELPF | 531 |
| LlSSB (C10) | | MEISDDDLPF | 532 |
| LlSSB (C9) | | EISDDDLPF | 533 |
| LlSSB (C8) | | ISDDDLPF | 534 |
| LrhSSB (C8) | | | |
| LrhSSB (C10) | | IDISDDDLPF | 535 |
| LrhSSB (C9) | | DISDDDLPF | 536 |
| LlSSB (C7) | | SDDDLPF | 537 |
| LrhSSB (C7) | | | |
| LlSSB | | MEISDDDIPF | 538 |
| C3:EcSSB | | | |
| LlSSB | | MEIFDDDIPF | 539 |
| C7:EcSSB | | | |
| LlSSB | | MEDFDDDIPF | 540 |
| C8:EcSSB | | | |
| LlSSB | | MMDFDDDIPF | 541 |
| C9:EcSSB | | | |
| LlSSB | | MEIFDDDIPF | 542 |
| C7:PaSSB | | | |
| LlSSB | | MESFDDDIPF | 543 |
| C8:PaSSB | | | |
| LlSSB | | MEIADDEPPF | 544 |
| C7:MsSSB | | | |
| LlSSB | | MEGADDEPPF | 545 |
| C8:MsSSB | | | |
| LrSSB | | IDLADDEPPF | 546 |
| C7:MsSSB | | | |
| LrSSB | | EDGADDEPPF | 547 |
| C8:MsSSB | | | |

### Materials and Methods

### Bacterial Strains and Culturing Conditions

The E. coli strain used was derived from EcNR2 with some modifications (EcNR2.dnaG_Q576A.tolC_mut.mutS::cat_mut.dlambda::zeoR)6. L. lactis strain NZ9000 was provided as a kind gift from Jan Peter Van Pijkeren. M. smegmatis strain mc(2)155 was purchased from ATCC. The C. crescentus strain used was NA1000.

All chemicals were purchased from Sigma Aldrich, unless stated otherwise. E. coli and its derivatives were cultured in Lysogeny broth—Low sodium (Lb-L) (10 g/L tryptone, 5 g/L yeast extract (Difco), PH 7.5 with NaOH), in a roller drum at 34° C. L. lactis was cultured in M17 broth (Difco, BD BioSciences) supplemented with 0.5% (w/v) D-glucose, static at 30° C. M. smegmatis was cultured in Middlebrook 7H9 Broth (Difco, BD BioSciences) with AD Enrichment (10× stock: 50 g/L BSA, 20 g/L D-glucose, 8.5 g/L NaCl), supplemented with glycerol and Tween 80 to a final concentration of 0.2% (v/v) and 0.05% (v/v), respectively, in a roller drum at 37° C. C. crescentus was cultured in peptone-yeast extract (PYE) broth (2 g/L peptone, 1 g/L yeast extract (Difco), 0.3 g/L MgSO4, 0.5 mM 0.5M CaCl2), shaking at 30° C.

Plating was done on petri dishes of LB agar for E. coli, M17 Agar (Difco, BD BioSciences) supplemented with 0.5% (w/v) D-glucose for L. lactis, 7H10 (Difco, BD BioSciences) supplemented with AD Enrichment and 0.2% (v/v) glycerol for M. smegmatis, and PYE agar for C. crescentus. Antibiotics were added to the media when appropriate, at the following concentrations: 50 μg/mL carbenicillin for E. coli, 10 μg/mL chloramphenicol for L. lactis, and 100m/mL hygromycin B for M. smegmatis, 5 μg/ml kanamycin for C. crescentus. For the selective plates used to determine allelic recombination frequency, antibiotics were added as follows: 0.005% SDS for E. coli, 50 μg/mL rifampicin for L. lactis, 20 μg/mL streptomycin for M. smegmatis, and 5 μg/m1rifampicin for C. crescentus.

### Construction and Transformation of Plasmids

Plasmids were constructed using PCR fragments and Gibson Assembly. All primers and genes were obtained from Integrated DNA Technologies (IDT). Plasmids were derived from pARC8 for use in E. coli, pjp005 for use in L. lactis—a gift from Jan Peter Van Pijkeren, pKM444 for use in M. smegmatis—a gift from Kenan Murphy (Addgene plasmid #108319), and pBXMCS-2 for use in C. crescentus. Genes were codon optimized for each of the host organisms using IDT's online Codon Optimization Tool. E. coli and L. lactis plasmid constructs were Gibson assembled, then directly transformed into electrocompetent E. coli and L. lactis strains. M. smegmatis plasmids were first cloned in NEB 5-alpha Competent E. coli (New England Biolabs) for plasmid verification before transformation into electrocompetent M. smegmatis. All cloning was verified by Sanger sequencing (Genewiz). Plasmids will be deposited in Addgene. All data is available from the authors upon reasonable request.

### Protein Purification

To prepare Redβ for in vitro analysis, it was first cloned by Gibson cloning into pET-53-DEST, with a 6× poly-histidine tag followed by a glycine-serine linker and a TEV protease site (MHHHHHHGSGENLYFQG) appended to its N-terminus. After purification and treatment with TEV protease, this leaves only an N-terminal glycine before the start codon. Overnight cultures of E. coli BL21 (DE3) (NEB) with the expression construct were diluted 1:100 into Fernbach flasks, grown to an OD of −0.5, and induced with 1 mM IPTG at 37° C. for 4 h. Cultures were pelleted at 10,000×g in a fixed angle rotor for 10 min and the supernatant decanted. Bacterial pellets were resuspended in 30 mL of lysis buffer (150 mM NaCl, 0.1% v/v Triton-X, 50 mM TRIS-HCl pH 8.0) and sonicated at 80% power, 50% duty cycle for 5 minutes on ice. The lysed cultures were again centrifuged for 10 min at 15,000×g in a fixed angle rotor. The supernatant was then incubated for 30 minutes at room temperature with HisPur cobalt resin (Thermo) and column purified on disposable 25 ml polypropylene columns (Thermo). The protein-bound resin was washed with four column volumes of wash buffer (150 mM NaCl, 10 mM imidazole, 50 mM TRIS-HCl pH 8.0) and bound protein was eluted with two column volumes of elution buffer (150 mM NaCl, 250 mM imidazole, 50 mM TRIS-HCl pH 8.0). Protein eluates were dialyzed overnight against 25 mM TRIS-HCl pH 7.4 with 10,000 MWCO dialysis cassettes (Thermo), concentration was measured by Qubit (Thermo) and 1.5 mg of protein was cleaved in a 2 ml reaction with 240 Units of TEV protease (NEB) for two hours at 30° C. The TEV cleavage reaction was re-purified with cobalt resin, except that in this case the flow-through was collected, as the His tag and the TEV protease were bound to the resin. Expression and successful TEV cleavage were confirmed by SDS-PAGE. Protein was concentrated in 10,000 MWCO Amicon protein concentrators (Sigma), protein concentration was assayed by Qubit, and an equal volume of glycerol was added to allow storage at −20° C. E. coli and L. lactis SSBs were prepared according to previously published protocol (Lohman, Green, and Beyer, 1986) without the use of an affinity tag.

### Oligonucleotide Annealing and Quenching Experiments

Fluorescent (tolC-r.null.mut-3′FAM) and quenching (tolC-f.null.mut-5′IBFQ) oligos were ordered from Integrated DNA Technologies. Unless otherwise indicated, 50 nM of each oligo was incubated in 25 mM TRIS-HCl pH 7.4 with 1.0 μM Ec_SSB or Ll_SSB at 30° C. for 30 minutes. 100 μl of each oligo mixture were then combined into a 96-well clear-bottom black assay plate (Costar), incubated a further 60 minutes at 30° C., and annealing was tracked on a Synergy H4 microplate reader (BioTek) with fluorescence excitation set to 495 nm and emission set to 520 nm. After 60 minutes, 20 μl of a solution with or without 25 μM Redβ and containing 100 mM MgCl₂ was added to achieve a final reaction concentration of 2.5 μM Redβ and 10 mM MgCl₂. The annealing was then tracked over 10 hours in a the Synergy H4 microplate reader with the setting indicated above.

Preparation of Electrocompetent E. coli

A single colony of E. coli was grown overnight to saturation. In the morning 30 μL of dense culture was inoculated into 3 mL of fresh media and grown for 1 hour. To induce gene expression of the pARC8 vector for recombineering experiments, L-arabinose was added to a final concentration of 0.2% (w/v) and the cells were grown an additional hour. 1 mL of cells were pelleted at 4° C. by centrifugation at 12,000×g for 2.5 minutes and washed twice with 1 mL of ice-cold dH₂O. Cells were resuspended in 50 μL ice-cold dH₂O containing DNA and transferred to a pre-chilled 0.1 cm electroporation cuvette.

Preparation of Electrocompetent L. lactis

A single colony of L. lactis was grown overnight to saturation. 500 μL of dense culture was inoculated into 5 mL of fresh media, supplemented with 500 mM sucrose and 2.5% (w/v) glycine, and grown for 3 hours. To induce gene expression of the pJP005 vector for recombineering experiments, the cells were grown for an additional 30 min after adding 1 ng/mL freshly diluted nisin, unless stated otherwise. For the optimized condition (FIG. 20B), 10 ng/mL nisin was used. Cells were pelleted at 4° C. by centrifugation at 5,000×g for 5 minutes and washed twice with 2 mL of ice-cold electroporation buffer (500 mM sucrose containing 10% (w/v) glycerol) by centrifugation at 13,200×g for 2.5 minutes. Cells were resuspended in 80 μL ice-cold electroporation buffer containing DNA and transferred to a pre-chilled 0.1 cm electroporation cuvette.

Preparation of Electrocompetent M. smegmatis

A single colony of M. smegmatis was grown overnight to saturation. The next day 25 μL of dense culture was inoculated into 5 mL of fresh media in the evening and grown overnight to an OD600 of 0.9. Cells were pelleted at 4° C. by centrifugation at 3,500×g for 10 minutes and washed twice with 10 mL ice-cold 10% glycerol. Cells were resuspended in 360 μL ice-cold 10% glycerol and transferred along with 10 μL of DNA to a pre-chilled 0.2 cm electroporation cuvette.

Preparation of Electrocompetent C. crescentus

A single colony of C. crescentus was grown overnight. The next day cells were diluted back to OD ˜0.001 in 25 mL PYE, and grown overnight. The next day, 250 μL of 30% xylose was added to cells at OD ˜0.2. Cells were harvested at between OD=0.5 and OD=0.7, spun at 10,000 rpm for 10 min, and then washed twice in 12.5 ml of ice-cold dH₂O, washed once in 12.5 ml of ice-cold 10% glycerol, then washed and resuspended in 2.5 ml of ice-cold 10% glycerol. 90 μL of cells were added along with DNA to 0.1 cm cuvettes and incubated on ice for 10 min.

### Recombineering Experiments

Electrocompetent cells were electroporated with 90-mer oligos at: 1 uM for E. coli, 50 μg for L. lactis, and 10 uM for C. crescentus. 70-mer oligos were used at 1 μg for M. smegmatis. All oligos were obtained from IDT and can be found under “Oligonucleotides for genome editing” in materials and methods. For dsDNA experiments L. lactis was electroporated with 1.5 μg purified linear dsDNA. Cells were electroporated using a Bio-Rad gene pulser set to 25 μF, 200 S2, and 1.8 kV for E. coli, 2.0 kV for L. lactis, and 1.5 kV for C. crescentus and to 1000Ω and 2.5 kV for M. smegmatis. Immediately after electroporation, cells were recovered in fresh media for 3 hours for E. coli, 1 hour for L. lactis, overnight for M. smegmatis and overnight for C. crescentus. L. lactis recovery media was supplemented with MgCl₂ and CaCl₂) at a concentration of 20 mM and 2 mM, respectively. E. coli recovery media was supplemented with carbenicillin. M. smegmatis recovery media was supplemented with hygromycin. C. crescentus recovery media was supplemented with 0.3% xylose and kanamycin. After recovery, the cells were serial diluted and plated on non-selective vs. selective agar plates to obtain approximately 50-500 CFU/plate. Colonies were counted using a custom script in Fiji, and allelic recombination frequency was calculated by dividing the number of colonies on selective plates, with the number of colonies on non-selective plates.

### Protein Structures

Protein structure images (FIG. 18A) were downloaded from PyMOL: Schrodinger LLC, The PyMOL Molecular Graphics System, Version 1.8 (2015).

### Example 15

The editing efficiency of SSAP candidates was also tested in Agrobacterium tumefaciens and in Staphylococcus aureus using the methods described above.

As shown in FIG. 24, PF071 (SEQ ID NO: 205), PF076 (SEQ ID NO: 210), PF074 (SEQ ID NO: 208), and N003 (SEQ ID NO: 3) showed an increase in editing efficiency (as indicated by enrichment on the Y-axis) relative to other SSAP candidates in Agrobacterium tumefaciens.

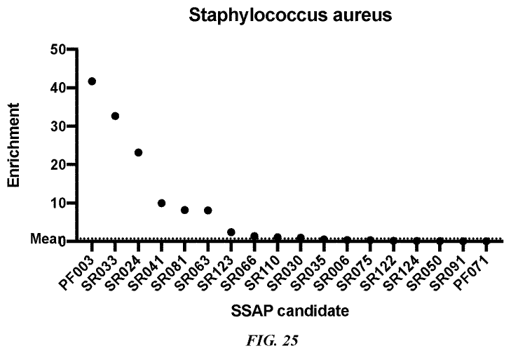

As shown in FIG. 25, PF003 (SEQ ID NO: 143), SR033 (SEQ ID NO: 41), SR024 (SEQ ID NO: 32), SR041 (SEQ ID NO: 49), SR081 (SEQ ID NO: 89), and SR063 (SEQ ID NO: 71) showed an increase in editing efficiency (as indicated by enrichment on the Y-axis) relative to other SSAP candidates in Staphylococcus aureus.

What is claimed is:

1. A recombinant bacterial cell of a first genus comprising a single-stranded annealing protein (SSAP) from a bacteriophage that can infect, or from a prophage that is stably integrated into the genome of, a bacterial cell of a second genus different from the first genus, optionally wherein the SSAP is expressed from a non-native promoter.

2. The recombinant bacterial cell of claim 1, wherein the recombinant bacterial cell of a first genus is gram negative, and the bacterial cell of a second genus is gram positive, or wherein the recombinant bacterial cell of a first genus is gram positive, and the bacterial cell of a second genus is gram negative.

3. The recombinant bacterial cell of claim 1, wherein the recombinant bacterial cell of a first genus is gram positive, and the bacterial cell of a second genus is gram positive, or wherein the recombinant bacterial cell of a first genus is gram negative, and the bacterial cell of a second genus is gram negative.

4. The recombinant bacterial cell of claim 2 or 3, wherein the gram-negative bacterial cell is an Escherichia coli (E. coli) cell, a Klebsiella pneumoniae (K. pneumoniae) cell, a Salmonella enterica (S. enterica) cell, a Pseudomonas aeruginosa (P. aeruginosa), a Citrobacter freundii (C. freundii), and a Agrobacterium tumefaciens (A. tumefaciens) cell.

5. The recombinant bacterial cell of claim 4, wherein:
the recombinant bacterial cell is a gram-negative E. coli cell, optionally wherein the SSAP comprises the amino acid sequence of SEQ ID NO: 19, 63, 128, 157, 201, or 210; or
the recombinant bacterial cell is a gram-negative A. tumefaciens cell, optionally wherein the SSAP comprises the amino acid sequence of SEQ ID NO: 3, 205, 208, or 210.

6. The recombinant bacterial cell of any one of claims 1-5, wherein the gram-positive bacterial cell is selected from the group consisting of a Lactococcus lactis (L. lactis) cell, a Lactobacillus rhamnosus (L. rhamnosus) cell, a Mycobacterium smegmatis (M. smegmatis) cell, a Collinsella stercoris (C. stercoris) cell, and a Staphylococcus aureus (S aureus) cell.

7. The recombinant bacterial cell of claim 6, wherein
the recombinant bacterial cell is a gram-positive L. lactis cell, optionally wherein the SSAP comprises the amino acid sequence of SEQ ID NO: 5 or 143;
the recombinant bacterial cell is a gram-positive M. smegmatis cell, optionally wherein the SSAP comprises the amino acid sequence of SEQ ID NO: 44; or
the recombinant bacterial cell is a gram-positive S. aureus cell, optionally wherein the SSAP comprises the amino acid sequence of SEQ ID NO: 32, 41, 49, 71, 89, or 143.

8. The recombinant bacterial cell of any one of claims 1-7 further comprising a single-stranded binding protein (SSB).

9. The recombinant bacterial cell of claim 8, wherein the SSB is from a bacteriophage that can infect or from a prophage that is stably integrated into the genome of Clostridium botulinum, Gordonia soli, Paeniclostridium sordellii, or Enterococcus faecalis.

10. The recombinant bacterial cell of claim 8, wherein:
the recombinant bacterial cell is a gram-negative E. coli cell;
the SSAP comprises the amino acid sequence of SEQ ID NO: 157; and
the SSB comprises the amino acid sequence of SEQ ID NO: 300, 382, 384, or 389.

11. The recombinant bacterial cell of claim 6, wherein:
the recombinant bacterial cell is a gram-positive L. lactis cell;
the SSAP comprises the amino acid sequence of SEQ ID NO: 5; and
the SSB comprises the amino acid sequence of SEQ ID NO: 366, 381, or 395.

12. The recombinant bacterial cell of claim 6, wherein:
the recombinant bacterial cell is a gram-positive L. lactis cell;
the SSAP comprises the amino acid sequence of SEQ ID NO: 143; and
the SSB comprises the amino acid sequence of SEQ ID NO: 262, 325, 366, or 381.

13. A recombinant bacterial cell comprising a single-stranded annealing protein (SSAP) from a bacteriophage that can infect or from a prophage that is stably integrated into the genome of Pseudomonas aeruginosa, wherein the SSAP is expressed from a non-native promoter.

14. The recombinant bacterial cell of claim 13, wherein the SSAP comprises the amino acid sequence of SEQ ID NO: 24.

15. The recombinant bacterial cell of claim 13 or 14 wherein the recombinant bacterial cell is selected from the group consisting of a recombinant Klebsiella pneumoniae cell, a recombinant Salmonella enterica cell, and a recombinant Citrobacter freundii cell.

16. The recombinant bacterial cell of any one of claims 13-15, wherein the cell further comprises a single-stranded binding protein (SSB).

17. The recombinant bacterial cell of any one of claims 13-16, wherein the cell further comprises an exogenous nucleic acid comprising a sequence of interest that binds to a target locus of the cell, wherein the sequence of interest comprises a nucleotide modification relative to the target locus.

18. A recombinant bacterial cell comprising a single-stranded annealing protein (SSAP) and/or a single-stranded binding protein (SSB) of Table 1 expressed from a non-native promoter.

19. A recombinant bacterial cell comprising:
(a) a single-stranded annealing protein (SSAP) from a bacteriophage that can infect or from a prophage that is stably integrated into the genome of a first type of bacterial cell; and
(b) a chimeric single-stranded binding protein (SSB), wherein the chimeric SSB comprises a sequence encoding a first SSB from a second type of bacterial cell, wherein the C-terminus of the first SSB is substituted with at least 7 amino acids from the C-terminus of a second SSB from the first type of bacterial cell.

20. The recombinant bacterial cell of claim 19, wherein the C-terminus of the chimeric SSB comprises a sequence selected from SEQ ID NOs: 516-537 and 539-547.

21. The recombinant bacterial cell of any one of claims 1-20 further comprising an exogenous nucleic acid that comprises a sequence of interest that binds to a target locus of the cell, wherein the sequence of interest comprises a nucleotide modification relative to the target locus.

22. The recombinant bacterial cell of claim 21, wherein the nucleic acid is a single-stranded DNA or a double-stranded DNA.

23. The recombinant bacterial cell of claim 21 or 22, wherein the exogenous nucleic acid is integrated in the genome of the cell.

24. The recombinant bacterial cell of any one of claims 1-23, wherein the SSAP is encoded by a nucleic acid that is codon-optimized for expression in the recombinant bacterial cell.

25. The recombinant bacterial cell of any one of claims 8-24, wherein the SSB is encoded by a nucleic acid that is codon-optimized for expression in the recombinant bacterial cell.

26. The recombinant bacterial cell of any one of claims 1-25 further comprising a dominant negative MutL protein, optionally wherein the dominant negative MutL protein comprises an amino acid substitution corresponding to E32K in E. coli wild-type MutL (SEQ ID NO: 514), E33K in L. lactis wild-type MutL (SEQ ID NO: 512), or E36K in P. aeruginosa wild-type MutL (SEQ ID NO: 548).

27. The recombinant bacterial cell of any one of claims 1-26, wherein the SSAP is expressed from a vector comprising a ribosome binding site (RBS).

28. The recombinant bacterial cell of any one of claims 8-27, wherein the SSB is expressed from a vector comprising a ribosome binding site (RBS).

29. The recombinant bacterial cell of claim 27 or 28, wherein the RBS comprises a sequence selected from SEQ ID NOs: 505-511.

30. A method, comprising
culturing the recombinant bacterial cell of any one of claims 1-29 and producing a modified recombinant bacterial cell comprising the sequence of interest at the target locus.

31. A method, comprising:
culturing the recombinant bacterial cell of any one of claims 1-20, wherein the recombinant bacterial cell further comprises a nucleic acid comprising a sequence of interest that binds to a target locus of the recombinant bacterial cell, and wherein the sequence of interest comprises a nucleotide modification relative to the target locus; and
producing a modified recombinant bacterial cell comprising the sequence of interest at the target locus.

32. The method of claim 31, wherein the modification is a mutation (substitution), insertion, and/or deletion.

33. A method of editing the genome of bacterial cells, comprising
performing multiplexed automatable genome engineering (MAGE) in recombinant bacterial cells of any one of claims 1-20, wherein the recombinant bacterial cells further comprise at least two exogenous nucleic acids, each comprising a sequence of interest that binds to at least one target locus of the recombinant bacterial cells, wherein the sequence of interest comprises a nucleotide modification relative to the target locus, and
producing modified recombinant bacterial cells comprising the sequence of interest at the target locus.

34. The method of claim 33, wherein the recombinant bacterial cells comprise an SSB from a bacteriophage that can infect or from a prophage that is stably integrated into the genome of Paeniclostridium sordellii, optionally wherein the SSB comprises the amino acid sequence of SEQ ID NO: 384.

35. The method of claim 33 or 34, wherein at least 50% or at least 75% of the cells comprise the sequence of interest, optionally following 5-10 cycles of MAGE.

36. The method of claim 35, wherein at least 95% of the cells comprise the sequence of interest following 15 cycles of MAGE.

37. The method of claim 36, wherein following 15 cycles of MAGE, the percentage of cells comprising the sequence of interest is at least four-fold greater as compared to control E. coli cells that comprise (a) a Redβ SSAP from Enterobacteria phage X, (SEQ ID NO: 474) and (b) the at least two exogenous nucleic acids, each comprising the sequence of interest that binds to a different target locus of the control E. coli cell genome, wherein the sequence of interest comprises the nucleotide modification relative to the target locus.

38. A method, comprising
(i) introducing into a recombinant cell: (a) a single-stranded annealing protein (SSAP), (b) a single-stranded binding protein (SSB), and (c) a double-stranded nucleic acid comprising a sequence of interest that binds to a genomic target locus of the recombinant cell, wherein the sequence of interest comprises a nucleotide modification relative to the target locus, and
(ii) producing a modified recombinant cell comprising the sequence of interest at the target locus, wherein the modified recombinant cell does not express an exogenous exonuclease.

39. The method of claim 38, wherein (a) and (b) are from the same species of bacteria or from different species of bacteria.

40. The method of claim 38 or 39, wherein the SSAP comprises SEQ ID NO: 24 and/or the SSB comprises SEQ ID NO: 472.
